(19) United States
(12) Patent Application Publication
(10) Pub. No.: US 2024/0076293 A1
(43) Pub. Date: Mar. 7, 2024

(54) Compositions and Methods for Inhibiting Fibrosis, Inflammation and Cancer

(71) Applicant: Georgia Tech Research Corporation, Atlanta, GA (US)

(72) Inventors: Adegboyega Oyelere, Atlanta, GA (US); Bocheng Wu, Atlanta, GA (US)

(21) Appl. No.: 18/266,508

(22) PCT Filed: Dec. 28, 2021
(86) PCT No.: PCT/US2021/065304

§ 371 Date: Jun. 9, 2023

Related U.S. Application Data
(60) Provisional application No. 63/131,133, filed on Dec. 28, 2020.

(51) Int. Cl. C07D 413/14; C07D 405/14; C07D 407/14; C07D 409/14

(52) U.S. Cl. CPC C07D 413/14; C07D 405/14; C07D 407/14; C07D 409/14

(57) ABSTRACT

Provided herein are macrocycle-based compounds or diastereomers, solvate, or a pharmaceutically acceptable salt thereof. Also provided are pharmaceutical compositions and medicaments that include the compounds described herein as well as methods of treating fibrosis, inflammatory disease, and cancer.

### FIELD OF THE INVENTION

Aspects of the invention are generally directed to macrocycle-based compounds for inhibiting fibrosis, inflammation and cancer and methods of use thereof.

### BACKGROUND OF THE INVENTION

Idiopathic Pulmonary Fibrosis (IPF) is the most common interstitial lung disease (ILD). IPF is a chronic and fatal disease which progressively declines the lung function. The cause of IPF is ambiguous, and the disease affects 50 per 100,000 people worldwide. In the US, about 100,000 people are affected by IPF, and approximately 30,000 to 40,000 new cases are diagnosed annually. The prognosis of IPF is the worst among ILD, and its median survival range from 2-5 years. A study has shown the IPF patients have only 28% 5-year survival rate which is much lower than the survival rate for many types of cancers. Therefore, there is an unmet medical need for potent drugs to treat IPF.

Therefore, it is an object of the invention to provide macrocycle-based compounds for inhibiting fibrosis, inflammation and cancer.

It is still another object of the invention to provide pharmaceutical compositions containing macrocycle-based compounds that specifically treat fibrosis, inflammation and cancer.

### SUMMARY OF THE INVENTION

Macrocycle-based compounds, pharmaceutical compositions and methods of their use are provided. The compounds can inhibit TGF-beta and perturb the phosphorylation of pyruvate dehydrogenase E1-alpha among its targets.

In one aspect, the invention provides a compound of Formula I:

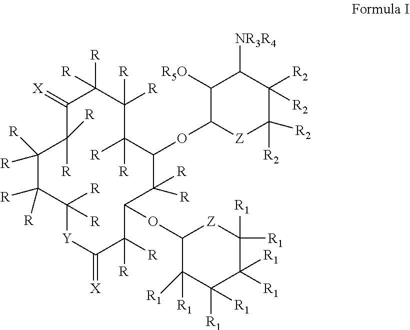

wherein
R is each independently H, OH, OCOCH₃, —CH₂OH, C₁₋₁₀ alkyl, C₁₋₁₀ alkoxy;
X is each independently O;
R₁ and R₂ are each independently H, OH, OCOCH₃, CH₃, and OCH₃;
Y is each independently O;
R₃ and R₄ are each independently H, C₁₋₁₀ alkyl, C₁₋₁₀ alkoxy, aryl, heteroaryl,

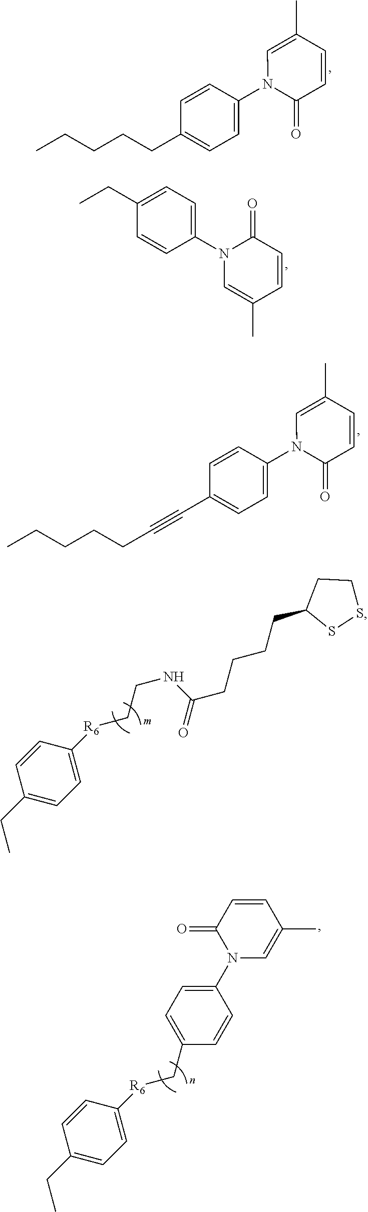

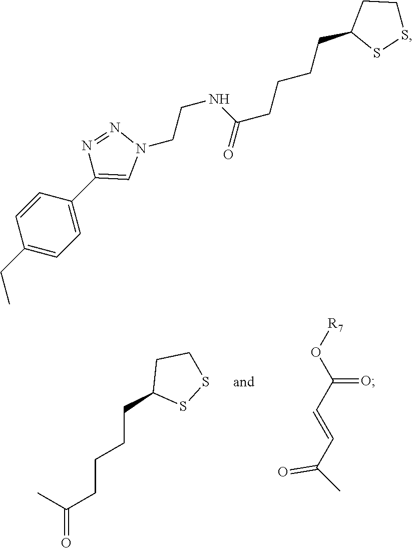

R₅ is each independently H, C₁₋₁₀ alkanoates, C₁₋₁₀ arylalkanoates, C₁₋₁₀ heteroarylalkanoates,

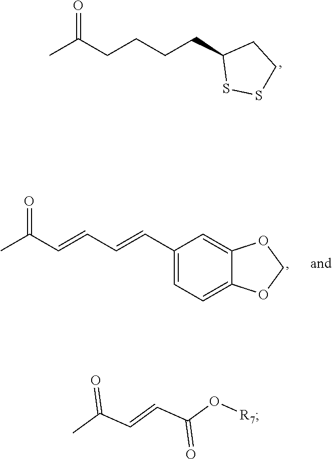

R₆ is each independently amide, 5 or 6-membered heteroaryl ring;
R₇ is each independently C₁₋₁₀ alkyl, C₁₋₁₀ arylalkyl, and C₁₋₁₀ heteroarylalkyl;
m is 1, 2, 3, 4, 5, or 6;
n is C₁₋₆ alkyl group, optionally substituted with double or triple bond; and
Z is each independently 0;
or diastereomers, solvate, or a pharmaceutically acceptable salt thereof.

In another aspect, the invention provides a compound of Formula II:

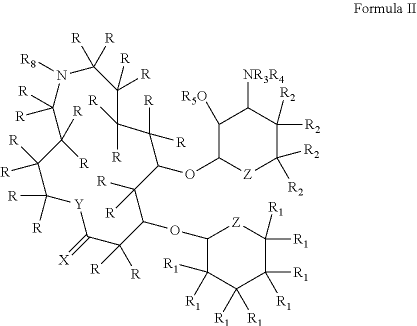

wherein
R is each independently H, OH, OCOCH₃, —CH₂OH, C₁₋₁₀ alkyl, C₁₋₁₀ alkoxy;
X is each independently O;
R₁ and R₂ are each independently H, OH, OCOCH₃, CH₃, and OCH₃;
Y is each independently O;
R₃ and R₄ are each independently H, C₁₋₁₀ alkyl, C₁₋₁₀ alkoxy, aryl, heteroaryl,

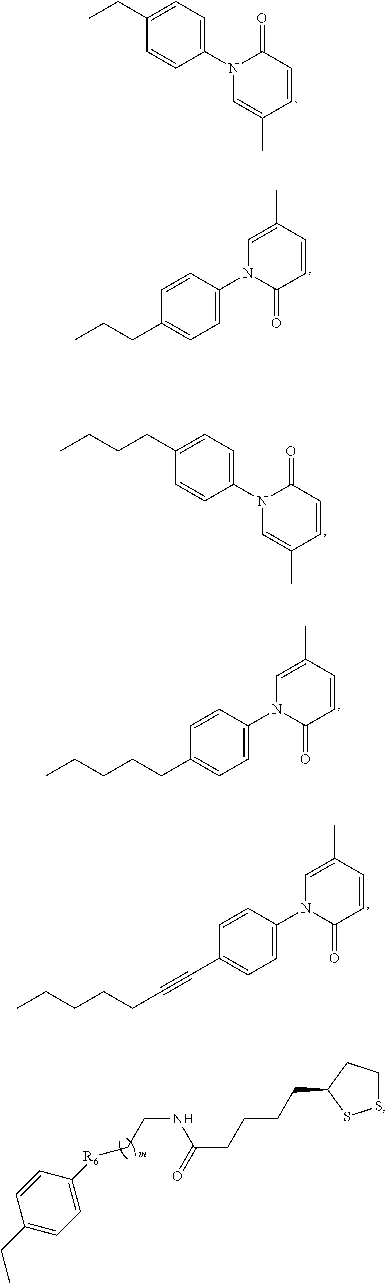

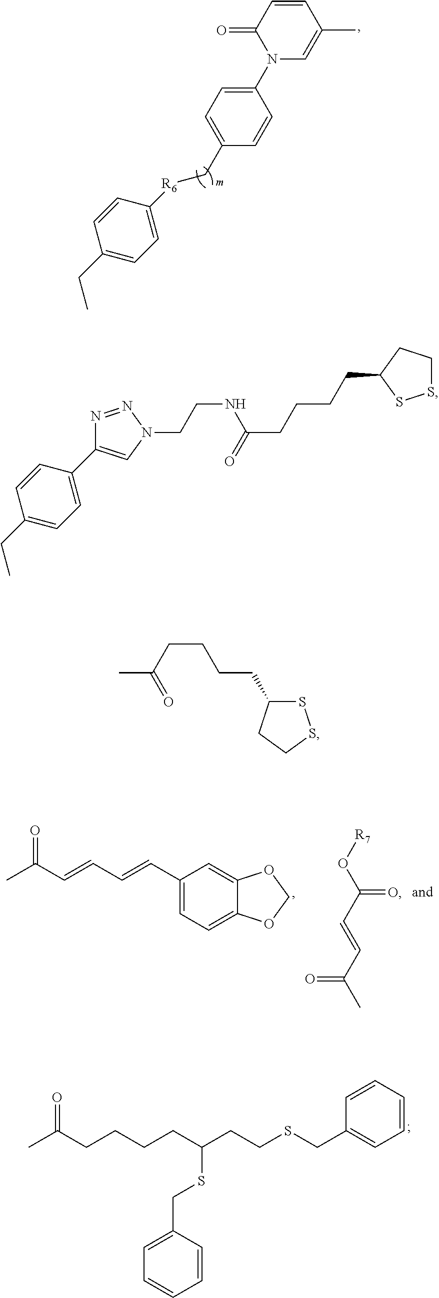

R₅ is each independently H, C₁₋₁₀ alkanoates, C₁₋₁₀ arylalkanoates, C₁₋₁₀ heteroarylalkanoates,

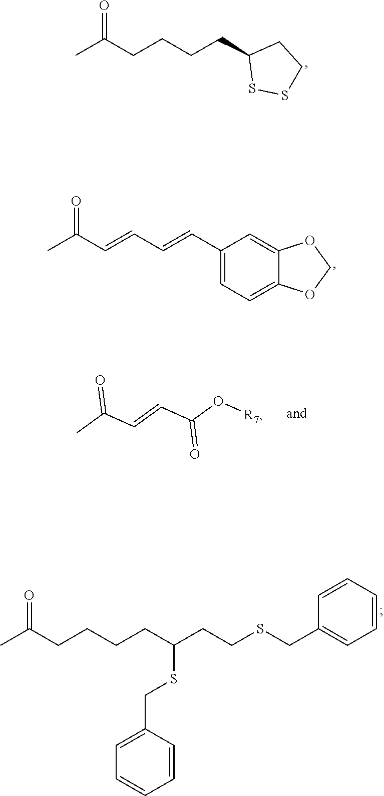

R₆ is each independently amide, 5 or 6-membered heteroaryl ring;
R₇ is each independently C₁₋₁₀ alkyl, C₁₋₁₀ arylalkyl, and C₁₋₁₀ heteroarylalkyl;
m is 1, 2, 3, 4, 5, or 6;
n is C₁₋₆ alkyl group, optionally substituted with double or triple bond;
Z is each independently O; and
R₈ is each independently H, and C₁₋₁₀ alkyl;
   or diastereomers, solvate, or a pharmaceutically acceptable salt thereof.

In other aspects, the invention provides a process for preparing a compound of formula I and formula II.

In another aspect, the invention provides a pharmaceutical composition comprising a compound or a pharmaceutically acceptable salt thereof, and a pharmaceutically acceptable carrier, adjuvant or vehicle.

In one aspect, the invention provides a method for treating a fibrotic disease, disorder or condition in a subject in need thereof comprising administering to the subject a therapeutically effective amount of the compound or a pharmaceutical composition thereof.

In other aspects, the invention provides a method for treating an inflammatory disease, disorder or condition in a subject in need thereof, comprising administering to the subject a therapeutically effective amount of the compound or pharmaceutical composition thereof.

In a further aspect, the invention provides a method for treating a cancer in a subject in need thereof comprising administering to the subject a therapeutically effective amount of the compound or pharmaceutical composition thereof.

### BRIEF DESCRIPTION OF THE DRAWINGS

FIG. 1 shows chemical structures of target lipoyl and 6,8-bis(benzylthio)octanoyl compounds.

FIG. 2 shows chemical structures of target piperine compounds.

FIG. 3 shows chemical structures of target fumaryl compounds.

### DETAILED DESCRIPTION OF THE INVENTION

### I. Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term ‘substituted’ is to be defined as set out below. It should be further understood that the terms “groups” and “radicals” can be considered interchangeable when used herein.

The articles “a” and “an” may be used herein to refer to one or to more than one (i.e., at least one) of the grammatical objects of the article. By way of example “an analogue” means one analogue or more than one analogue.

As used herein, the term “pharmaceutical composition” means a mixture comprising a pharmaceutically acceptable active ingredient, in combination with suitable pharmaceutically acceptable excipients.

Pharmaceutical excipients are substances other than the pharmaceutically acceptable active ingredient which have been appropriately evaluated for safety and which are intentionally included in an oral solid dosage form. For example, excipients can aid in the processing of the drug delivery system during its manufacture, protect, support or enhance stability, bioavailability or patient acceptability, assist in product identification, or enhance any other attribute of the overall safety, effectiveness or delivery of the drug during storage or use. Examples of excipients include, for example but without limitation inert solid diluents (bulking agent e.g., lactose), binders (e.g., starch), glidants (e.g., colloidal silica), lubricants (e.g., non-ionic lubricants such as vegetable oils), disintegrants (e.g., starch, polivinylpyrrolidone), coating better polymers (e.g., hydroxypropyl methylcellulose), colorants (e.g., iron oxide), and/or surfactants (e.g., non-ionic surfactants).

As used herein, the term “pharmaceutical formulation” means a composition in which different chemical substances, including the active drug, are combined to produce a final medicinal product. Examples of formulation include enteral formulations (tablets, capsules), parenteral formulations (liquids, lyophilized powders), or topical formulations (cutaneous, inhalable).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt or derivatives thereof that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. More particularly, such salts are formed with hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid.

The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Solvate” refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

The terms “inert solid diluent” or “solid diluent” or “diluents” refer to materials used to produce appropriate dosage form size, performance and processing properties for tablets and/or capsules. An inert solid diluent can be also referred to as filler or filler material. Particular examples of diluents include cellulose powdered, silicified microcrystalline cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose, anhydrous, monohydrate and corn starch, spray dried monohydrate and microcrystalline cellulose, maltodextrin, maltose, mannitol, medium-chain triglycerides, microcrystalline cellulose, polydextrose, polymethacrylates, simethicone, sorbitol, pregelatinized starch, sterilizable maize, sucrose, sugar spheres, sulfobutylether β-cyclodextrin, talc, tragacanth, trehalose, or xylitol. More particular examples of diluents include cellulose powdered, silicified microcrystalline cellulose acetate, compressible sugar, corn starch and pregelatinized starch, dextrose, fructose, glyceryl palmitostearate, anhydrous, monohydrate and corn starch, spray dried monohydrate and microcrystalline cellulose, maltodextrin, maltose, mannitol, medium-chain triglycerides, microcrystalline cellulose, polydextrose, sorbitol, starch, pregelatinized, sucrose, sugar spheres, trehalose, or xylitol.

“Lubricant” refers to materials that prevent or reduce ingredients from clumping together and from sticking to the tablet punches or capsule filling machine. Lubricants also ensure that tablet formation and ejection can occur with low friction between the solid and die wall. Particular examples of lubricants include canola oil, hydrogenated castor oil, cottonseed oil, glyceryl behenate, glyceryl monostearate, glyceryl palmitostearate, medium-chain triglycerides, mineral oil, light mineral oil, octyldodecanol, poloxamer, polyethylene glycol, polyoxyethylene stearates, polyvinyl alcohol, starch, or hydrogenated vegetable oil. More particular examples of diluents include glyceryl behenate, glyceryl monostearate, or hydrogenated vegetable oil.

“Disintegrant” refers to material that dissolve when wet causing the tablet to break apart in the digestive tract, releasing the active ingredients for absorption. They ensure that when the tablet is in contact with water, it rapidly breaks down into smaller fragments, facilitating dissolution. Particular examples of disintegrants include alginic acid, powdered cellulose, chitosan, colloidal silicon dioxide, corn starch and pregelatinized starch, crospovidone, glycine, guar gum, low-substituted hydroxypropyl cellulose, methylcellulose, microcrystalline cellulose, or povidone.

The term “colorant” describes an agent that imparts color to a formulation. Particular examples of colorants include iron oxide, or synthetic organic dyes (US Food and Drug administration, Code of Federal Regulations, Title 21 CFR Part73, Subpart B).

The term “plasticizing agent” or “plasticizer” refers to an agent that is added to promote flexibility of films or coatings. Particular examples of plasticizing agent include polyethylene glycols or propylene glycol.

The term “pigment” in the context of the present invention refers to an insoluble coloring agent.

The term “film-coating agent” or ‘coating agent’ or ‘coating material’ refers to an agent that is used to produce a cosmetic or functional layer on the outer surface of a dosage form. Particular examples of film-coating agent include glucose syrup, maltodextrin, alginates, or carrageenan.

“Glidant” refers to materials that are used to promote powder flow by reducing interparticle friction and cohesion. These are used in combination with lubricants as they have no ability to reduce die wall friction. Particular examples of glidants include powdered cellulose, colloidal silicon dioxide, hydrophobic colloidal silica, silicon dioxide, or talc. More particular examples of glidants include colloidal silicon dioxide, hydrophobic colloidal silica, silicon dioxide, or talc.

“Flavoring agents” refers to material that can be used to mask unpleasant tasting active ingredients and improve the acceptance that the patient will complete a course of medication. Flavorings may be natural (e.g., fruit extract) or artificial. Non limiting examples of flavoring agents include mint, cherry, anise, peach, apricot, licorice, raspberry, or vanilla.

The term “Subject” includes mammals such as humans. The terms “human”, “patient” and “subject” are used interchangeably herein.

“Effective amount” means the amount of a compound of the invention that, when administered to a subject for treating a disease, is sufficient to effect such treatment for the disease. The ‘effective amount’ can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron, and 13 Emission Topography (PET) studies for examining substrate receptor occupancy.

All isotopic variants of the compounds provided herein, radioactive or not, are intended to be encompassed within the scope of the invention.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The term “alkyl” as used herein, whether used alone or as part of another group, refers to a substituted or unsubstituted aliphatic hydrocarbon chain and includes, but is not limited to, straight and branched chains containing from 1 to 20 carbon atoms, preferably from 2 to 20, from 1 to 10, from 2 to 10, from 1 to 8, from 2 to 8, from 1 to 6, from 2 to 6, from 1 to 4, from 2 to 4, from 1 to 3 carbon atoms, unless explicitly specified otherwise. Illustrative alkyl groups can include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

The term “alkenyl” as used herein, whether used alone or as part of another group, refers to a substituted or unsubstituted aliphatic hydrocarbon chain and includes, but is not limited to, straight and branched chains having 2 to 8 carbon atoms and containing at least one carbon-carbon double bond.

The term “alkynyl” as used herein, whether used alone or as part of another group, refers to a substituted or unsubstituted aliphatic hydrocarbon chain and includes, but is not limited to, straight and branched chains having 1 to 6 carbon atoms and containing at least one carbon-carbon triple bond.

The term “alkoxy” as used herein, whether used alone or as part of another group, refers to alkyl-O— wherein alkyl is hereinbefore defined.

The term “cycloalkyl” as used herein, whether used alone or as part of another group, refers to a monocyclic, bicyclic, tricyclic, fused, bridged or spiro monovalent saturated hydrocarbon moiety, wherein the carbon atoms are located inside or outside of the ring system. Any suitable ring position of the cycloalkyl moiety may be covalently linked to the defined chemical structures. Illustrative cycloalkyl groups can include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantly, spiro[4,5]decanyl, and homologs, isomers and the alike.

The term “aryl” as used herein, whether used alone or as part of another group, refers to an aromatic carbocyclic ring system having 6 to 30 carbon atoms, preferably 6 to 10 carbon atoms, optionally substituted with 1 to 3 substituents independently selected from halogen, nitro cyano, hydroxy, alkyl, alkenyl, alkoxy, cycloalkyl, amino, alkylamino, dialkylamino, carboxy, alkoxycarbonyl, haloalkyl, and phenyl.

The term “phenyl” as used herein, whether used alone or as part of another group, refers to a substituted or unsubstituted phenyl group.

The term “heteroaryl” as used herein, whether used alone or as part of another group, refers to a 3 to 30 membered aryl heterocyclic ring, which contains from 1 to 4 heteroatoms selected from the group consisting of O, N, Si, P and S atoms in the ring and may be fused with a carbocyclic or heterocyclic ring at any possible position.

The term “heterocycloalkyl” as used herein, whether used alone or as part of another group, refers to a 5 to 7 membered saturated ring containing carbon atoms and from 1 to 2 heteroatoms selected from the group consisting of O, N and S atoms.

The term “halogen or halo” as used herein, refers to fluoro, chloro, bromo or iodo.

The term “haloalkyl” as used herein, whether used alone or as part of another group, refers to an alkyl as hereinbefore defined, independently substituted with 1 to 3, F, Cl, Br or I.

The term “about” as used herein, refers that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, in phrase “about X to Y,” is the same as “about X to about Y,” that is the term “about” modifies both “X” and “Y.”

The term “compound” as used herein, refers to salts, solvates, complexes, isomers, stereoisomers, diastereoisomers, tautomers, and isotopes of the compound or any combination thereof.

The term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

The terms “substantially optically pure,” “optically pure,” and “optically pure enantiomers,” as used herein, mean that the composition contains greater than about 90% of a single stereoisomer by weight, preferably greater than about 95% of the desired enantiomer by weight, and more preferably greater than about 99% of the desired enantiomer by weight, based upon the total weight.

The term “diastereomers” refers to a stereoisomer that is a non-superimposable mirror image of each other. A diastereomer is a stereoisomer with two or more stereocenters, and the isomers are not mirror images of each other.

Macrocycle-based compounds represent an attractive target for treating chronic inflammatory disorders. Indeed, a role for macrocycle-based compounds has been demonstrated in a variety of inflammatory disorders, including, but not limited to, acute and chronic inflammatory disorders, sepsis, acute endotoxemia, encephalitis, Chronic Obstructive Pulmonary Disease (COPD), asthma, liver fibrosis, pulmonary fibrosis, pulmonary hypertension, ulcerative colitis, Crohn's disease, and in particular Inflammatory Bowel Disease (IBD).

Inflammatory bowel disease (IBD) covers a group of disorders in which the intestines become inflamed (red and swollen), probably as a result of an immune reaction of the body against its own intestinal tissue. Two major types of IBD are described: ulcerative colitis (UC) and Crohn's disease (CD). Ulcerative colitis is limited to the colon (large intestine). Crohn disease can involve any part of the gastrointestinal tract from the mouth to the anus, but it most commonly affects the small intestine and/or the colon. Both ulcerative colitis and Crohn disease vary in the intensity and severity during the course of the disease. When there is severe inflammation, the disease is considered to be in an active stage, and the person experiences a flare-up of the condition. When the degree of inflammation is less (or absent), the person usually is without symptoms, and the disease is considered to be in remission. In IBD factor or factors trigger the body's immune system to produce an inflammatory reaction in the intestinal tract that continues without control. As a result of the inflammatory reaction, the intestinal wall is damaged leading to bloody diarrhea and abdominal pain.

It will be appreciated that compounds of the invention may be metabolized to yield biologically active metabolites.

### II. Macrocycle-Based Compounds

A. Genus Structure Based on Formula I

One embodiment provides a compound of Formula I:

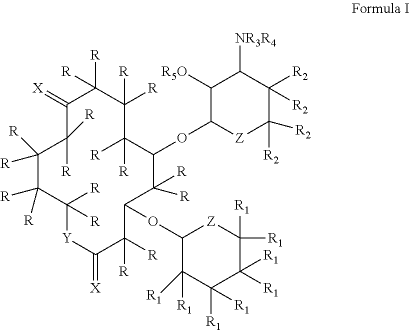

wherein
R is each independently H, OH, OCOCH₃, —CH₂OH, C₁₋₁₀ alkyl, C₁₋₁₀ alkoxy;
X is each independently O;
R₁ and R₂ are each independently H, OH, OCOCH₃, CH₃, and OCH₃;
Y is each independently O;
R₃ and R₄ are each independently H, C₁₋₁₀ alkyl, C₁₋₁₀ alkoxy, aryl, heteroaryl,

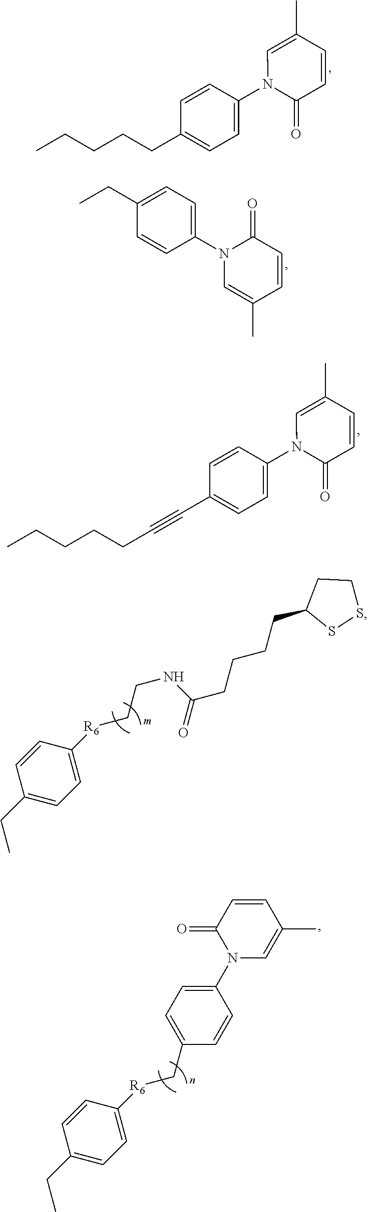

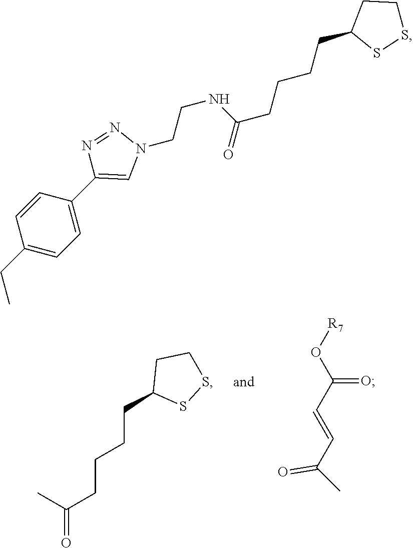

R₅ is each independently H, C₁₋₁₀ alkanoates, C₁₋₁₀ arylalkanoates, C₁₋₁₀ heteroarylalkanoates,

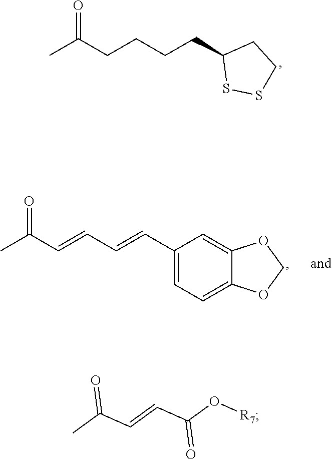

R₆ is each independently amide, 5 or 6-membered heteroaryl ring;
R₇ is each independently C₁₋₁₀ alkyl, C₁₋₁₀ arylalkyl, and C₁₋₁₀ heteroarylalkyl;
m is 1, 2, 3, 4, 5, or 6;
n is C₁₋₆ alkyl group, optionally substituted with double or triple bond; and
Z is each independently O;
or diastereomers, solvate, or a pharmaceutically acceptable salt thereof.

In some embodiments, R is H, OH, CH₃, OCH₃, or C₂H₅.

In another embodiment, X is O.

In some embodiments, R₁ is H, OH, CH₃, or OCH₃.

In other embodiment, Y is O.

In some embodiments, R₂ is H, OH, CH₃, or OCH₃.

In one embodiment, Z is O.

In another embodiment, R₃ is CH₃.

In other embodiment, R₄ is CH₃.

In one embodiment, R₅ is H, C₁₋₁₀ alkanoates, C₁₋₁₀ arylalkanoates, or C₁₋₁₀ heteroarylalkanoates.

In further embodiment, R₆ is amide.

In one embodiment, R₆ is 5 or 6-membered heteroaryl ring.

In another embodiment, R₇ is C₁₋₁₀ alkyl.

B. Genus Structure Based on Formula II

Another embodiment provides a compound of Formula II:

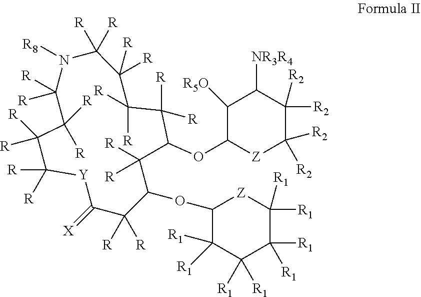

wherein
R is each independently H, OH, OCOCH₃, —CH₂OH, C₁₋₁₀ alkyl, C₁₋₁₀ alkoxy;
X is each independently O;
R₁ and R₂ are each independently H, OH, OCOCH₃, CH₃, and OCH₃;
Y is each independently O;
R₃ and R₄ are each independently H, C₁₋₁₀ alkyl, C₁₋₁₀ alkoxy, aryl, heteroaryl,

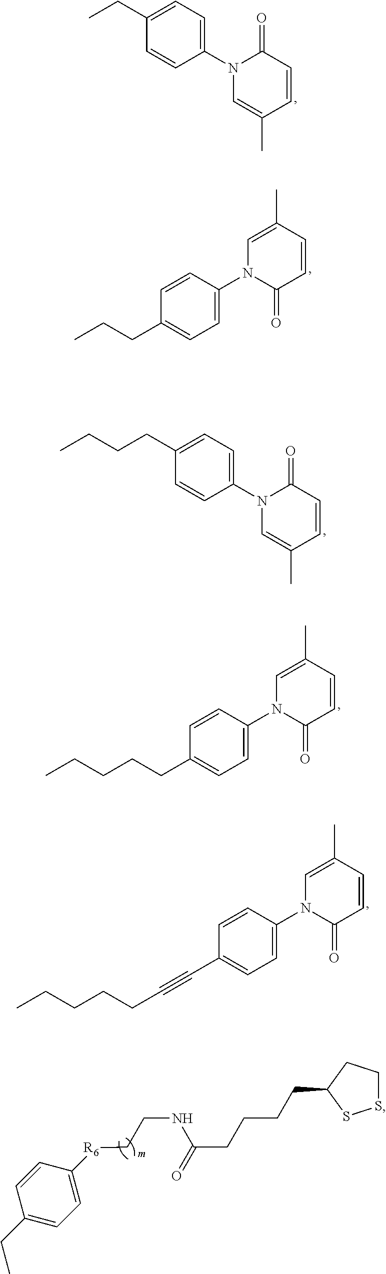

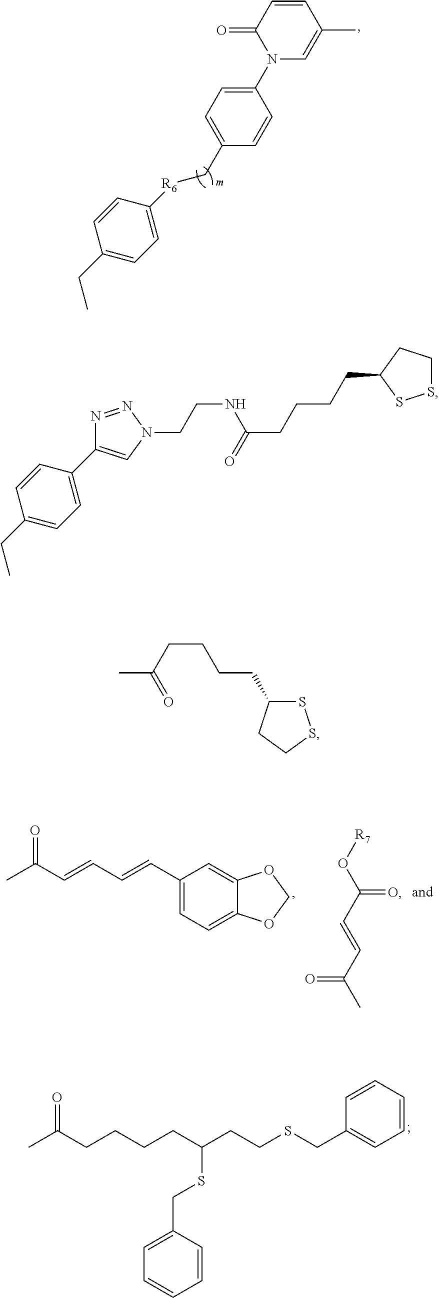

R₅ is each independently H, C₁₋₁₀ alkanoates, arylalkanoates, heteroarylalkanoates,

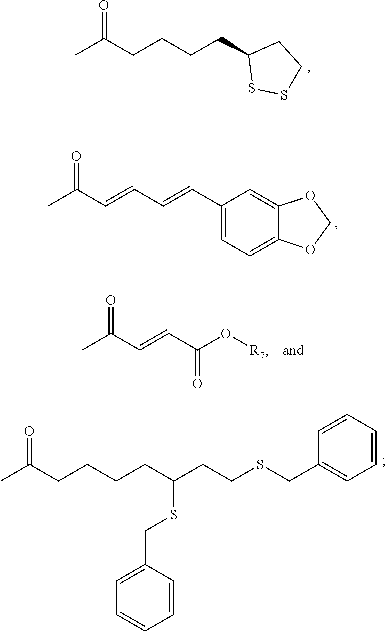

R₆ is each independently amide, 5 or 6-membered heteroaryl ring;
R₇ is each independently C₁₋₁₀ alkyl, C₁₋₁₀ arylalkyl, and C₁₋₁₀ heteroarylalkyl;
m is 1, 2, 3, 4, 5, or 6;
n is C₁₋₆ alkyl group, optionally substituted with double or triple bond;
Z is each independently O; and
R₈ is each independently H, and C₁₋₁₀ alkyl;
or diastereomers, solvate, or a pharmaceutically acceptable salt thereof.

In some embodiments, R is H, OH, CH₃, OCH₃, or C₂H₅.

In one embodiment, X is O.

In some embodiments, R₁ is H, OH, CH₃, or OCH₃.

In one embodiment, Y is O.

In some embodiments, R₂ is H, OH, CH₃, or OCH₃.

In a further embodiment, Z is O.

In another embodiment, R₃ is CH₃.

In one embodiment, R₄ is CH₃.

In another embodiment, R₅ is H, C₁₋₁₀ alkanoates, C₁₋₁₀ arylalkanoates, or C₁₋₁₀ heteroarylalkanoates.

In one embodiment, R₆ is amide.

In other embodiments, R₆ is 5 or 6-membered heteroaryl ring.

In another embodiment, R₇ is C₁₋₁₀ alkyl.

In yet another embodiment, R₈ is CH₃.

In some embodiments, the compound is selected from the group consisting of

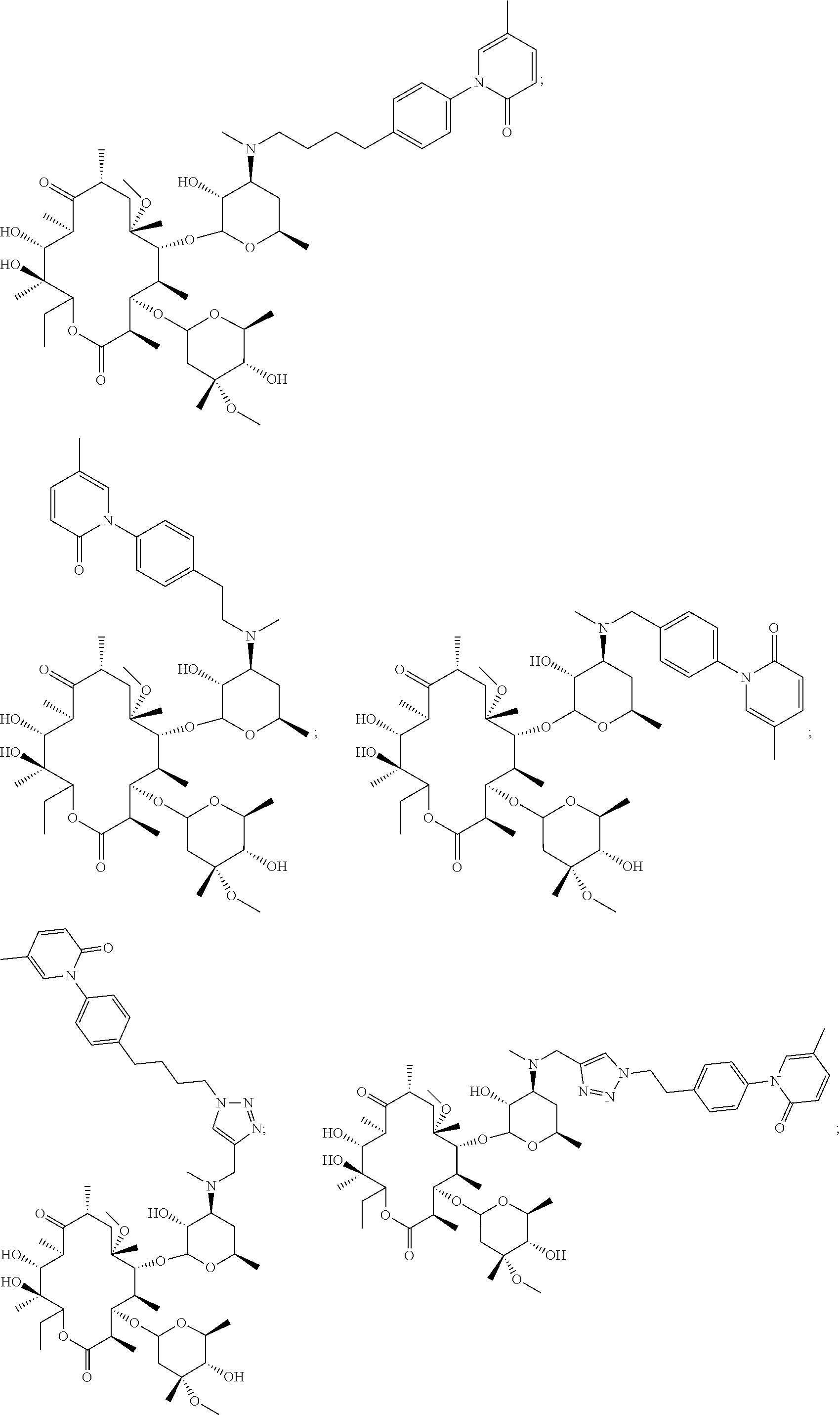

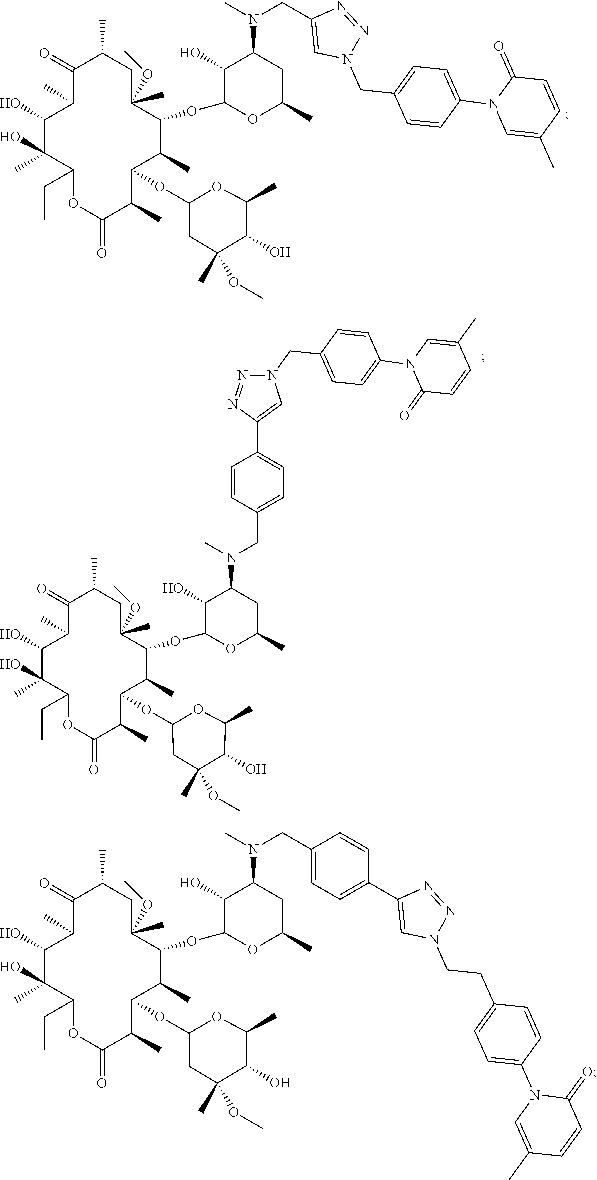

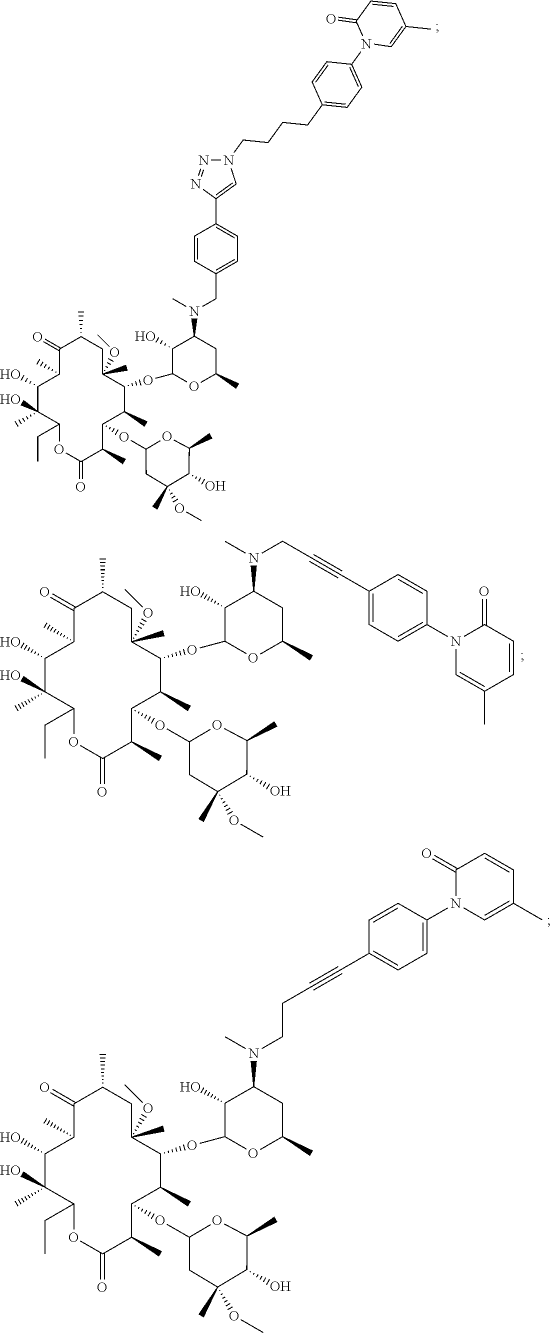

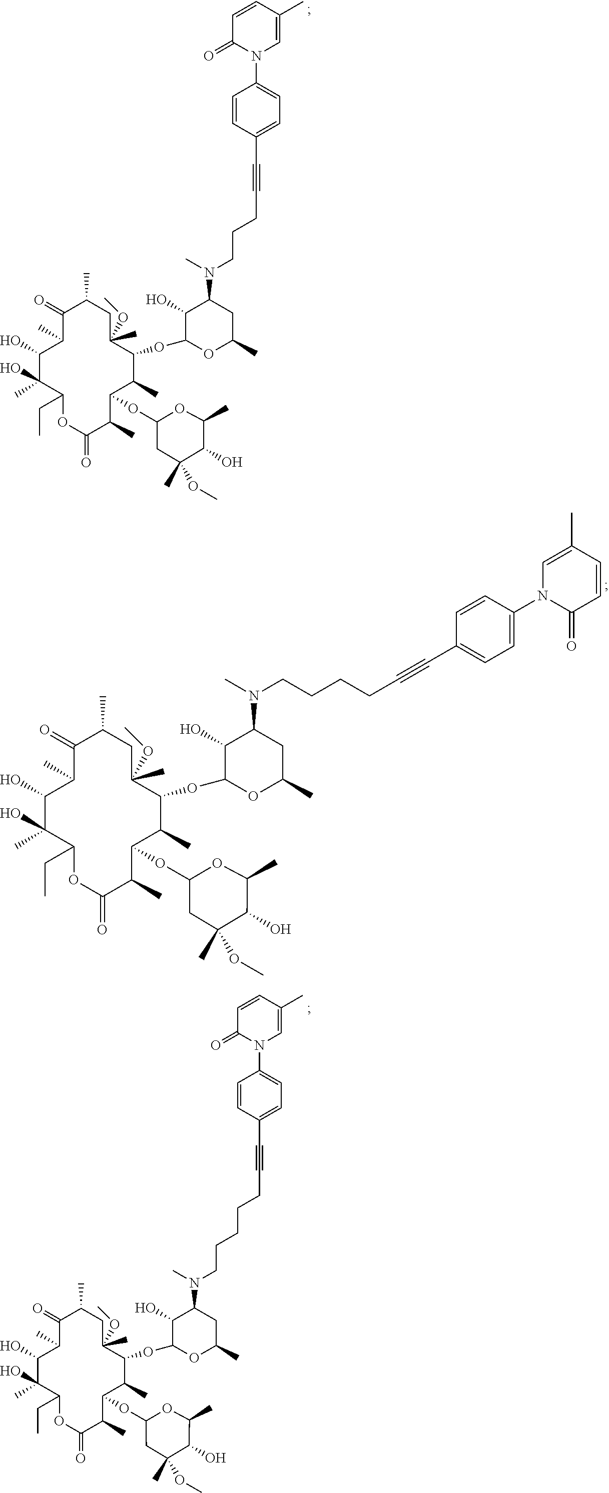

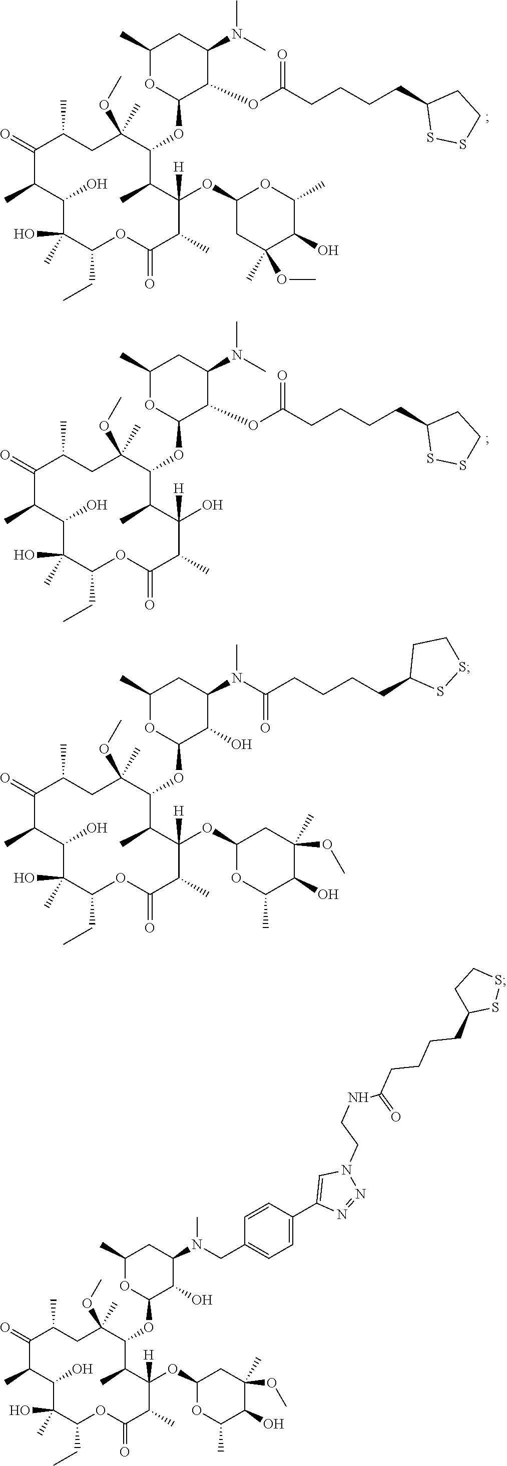

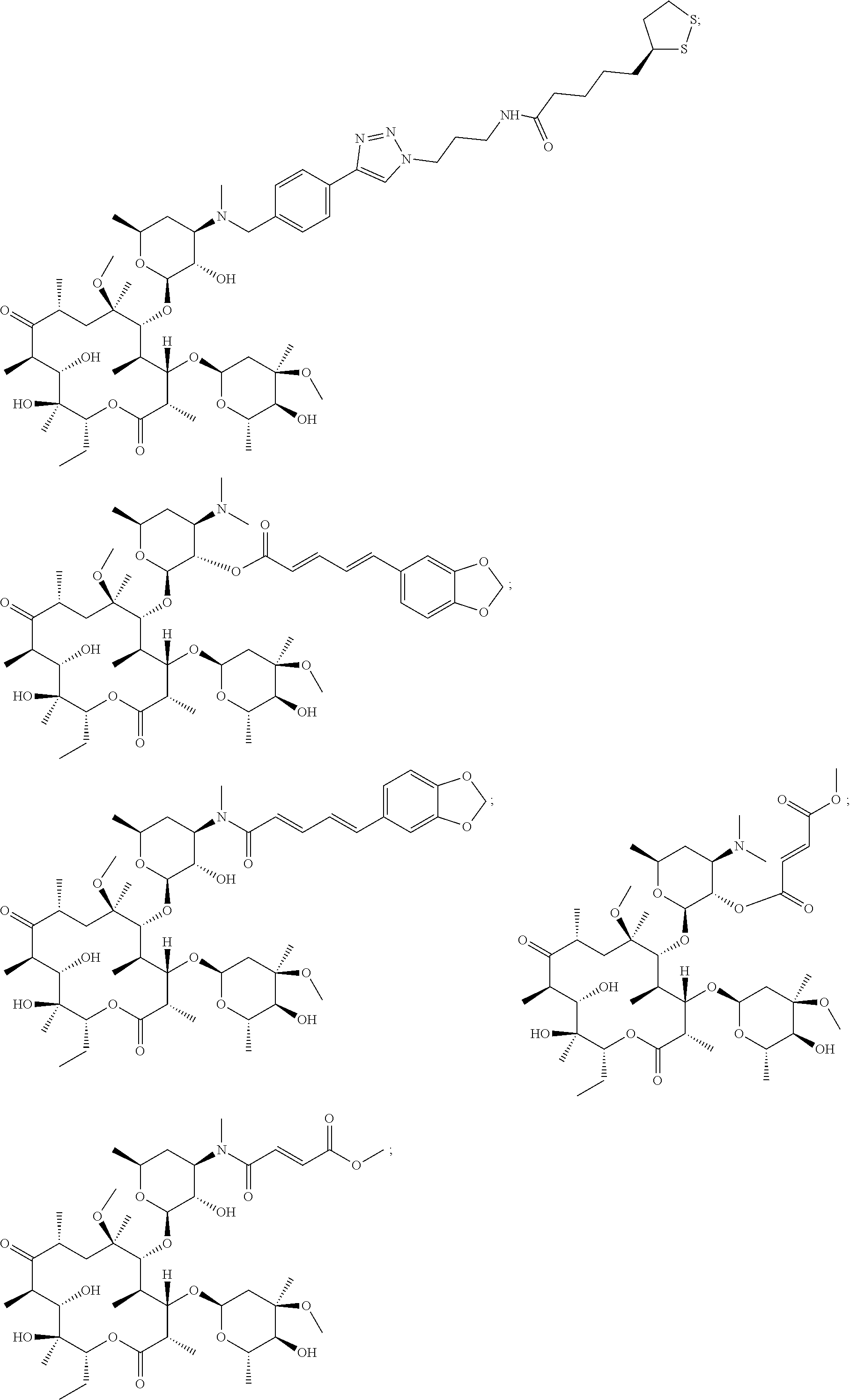

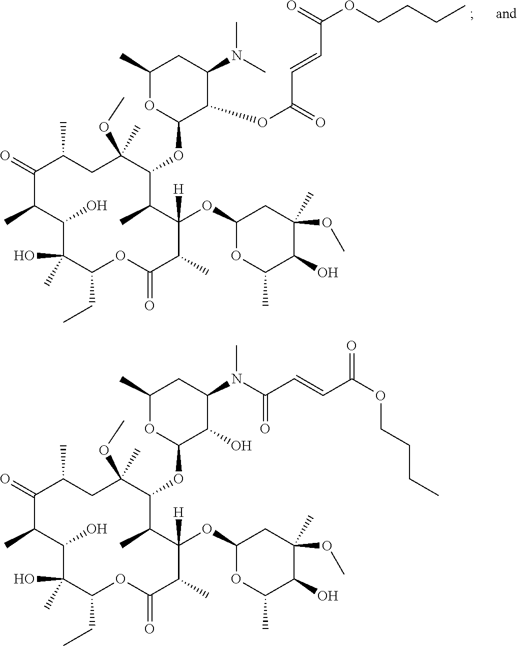

or diastereomers, solvates, or a pharmaceutically acceptable salt thereof

In some embodiments, the compound is selected from the group consisting of

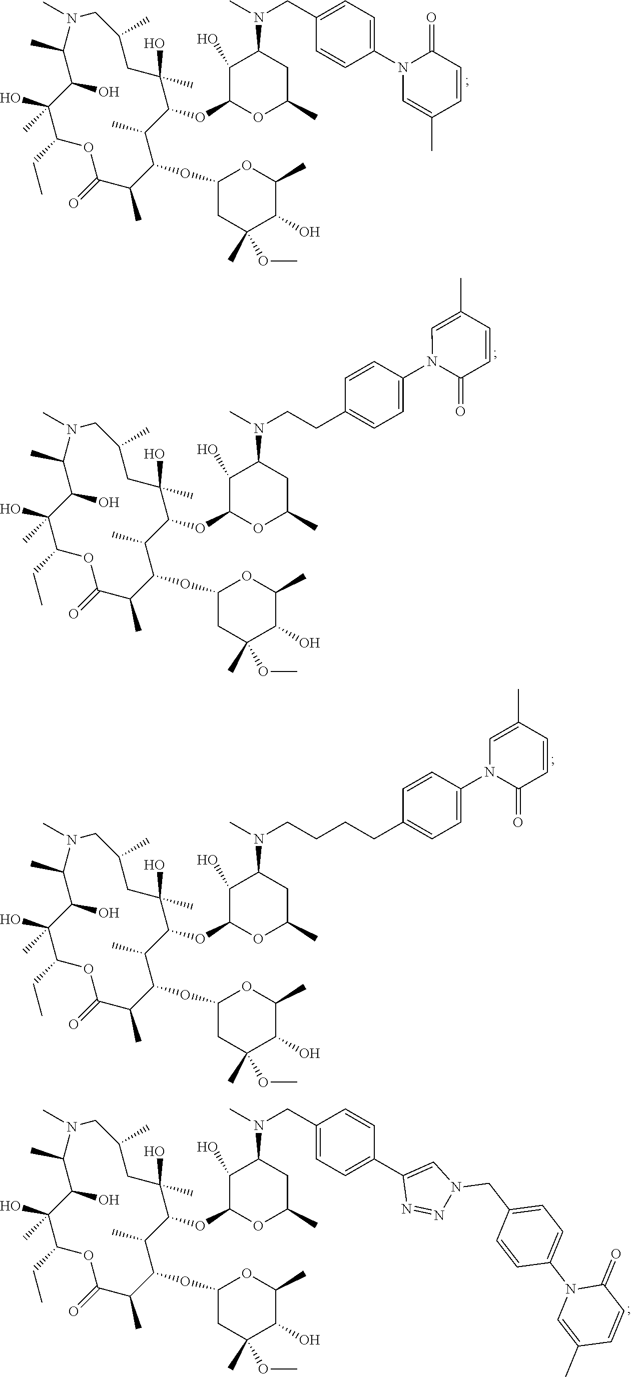

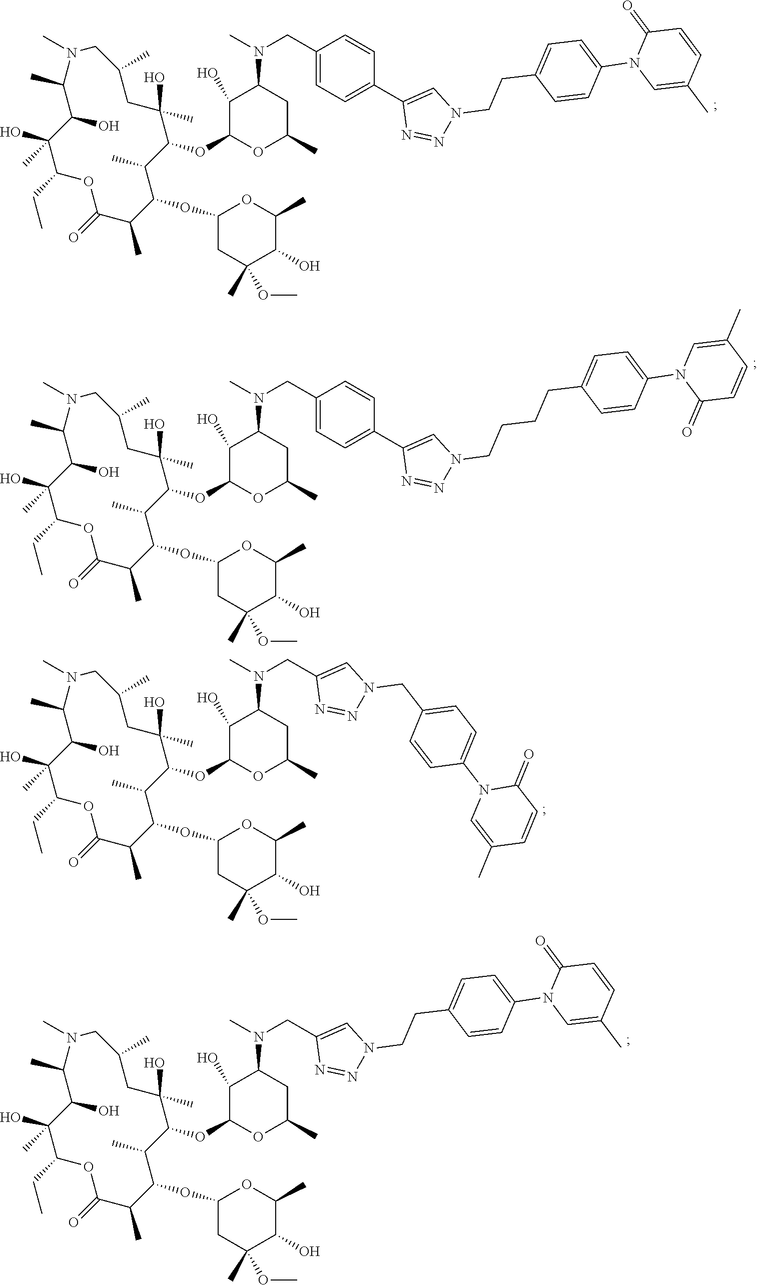

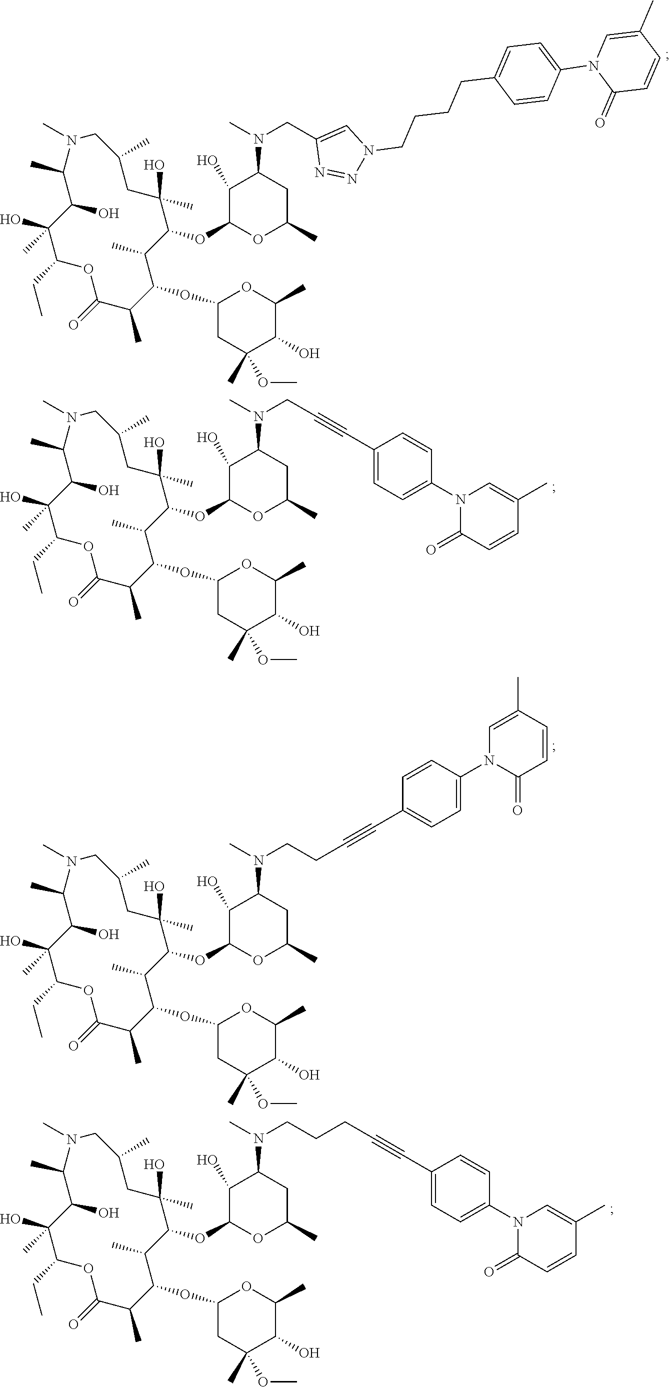

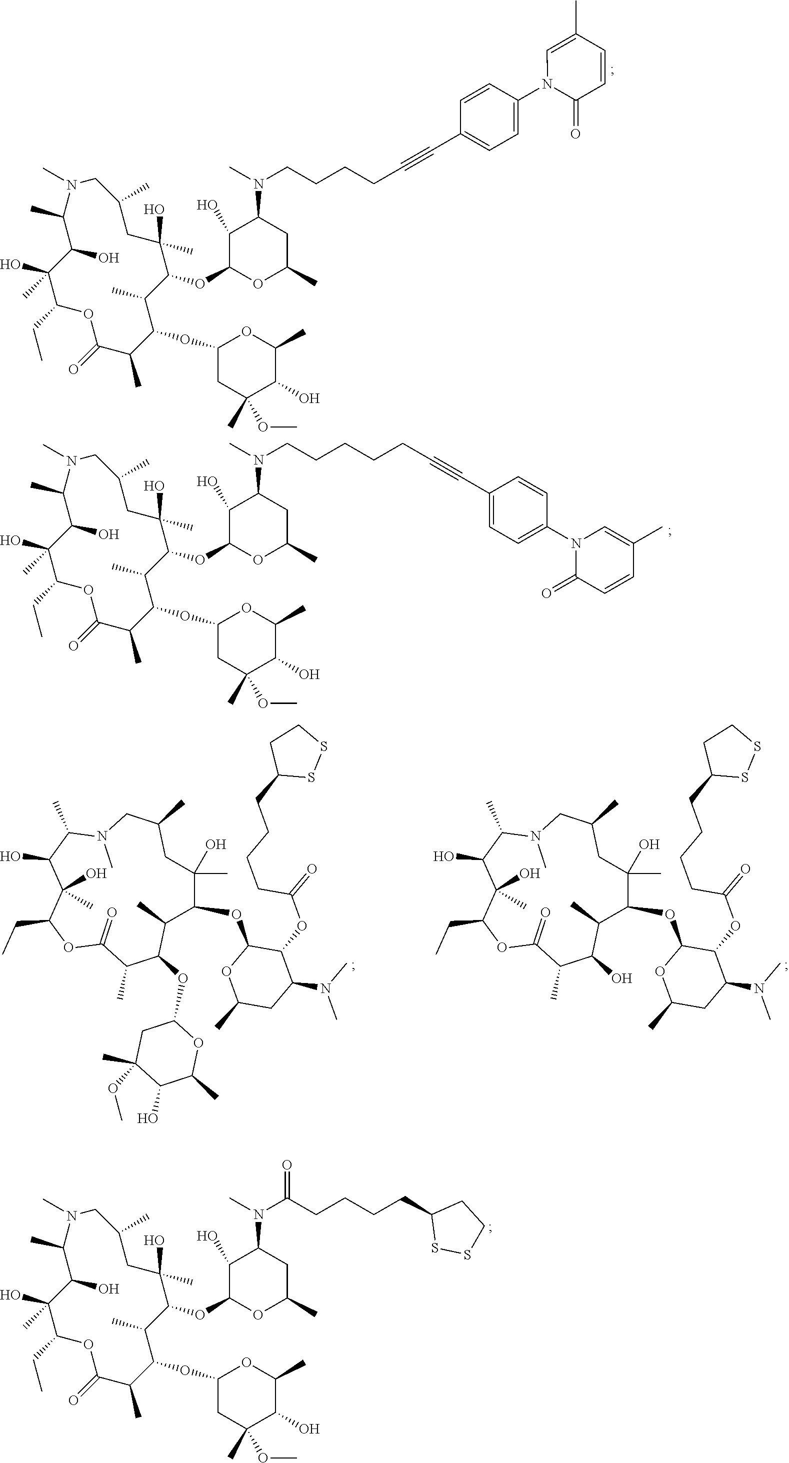

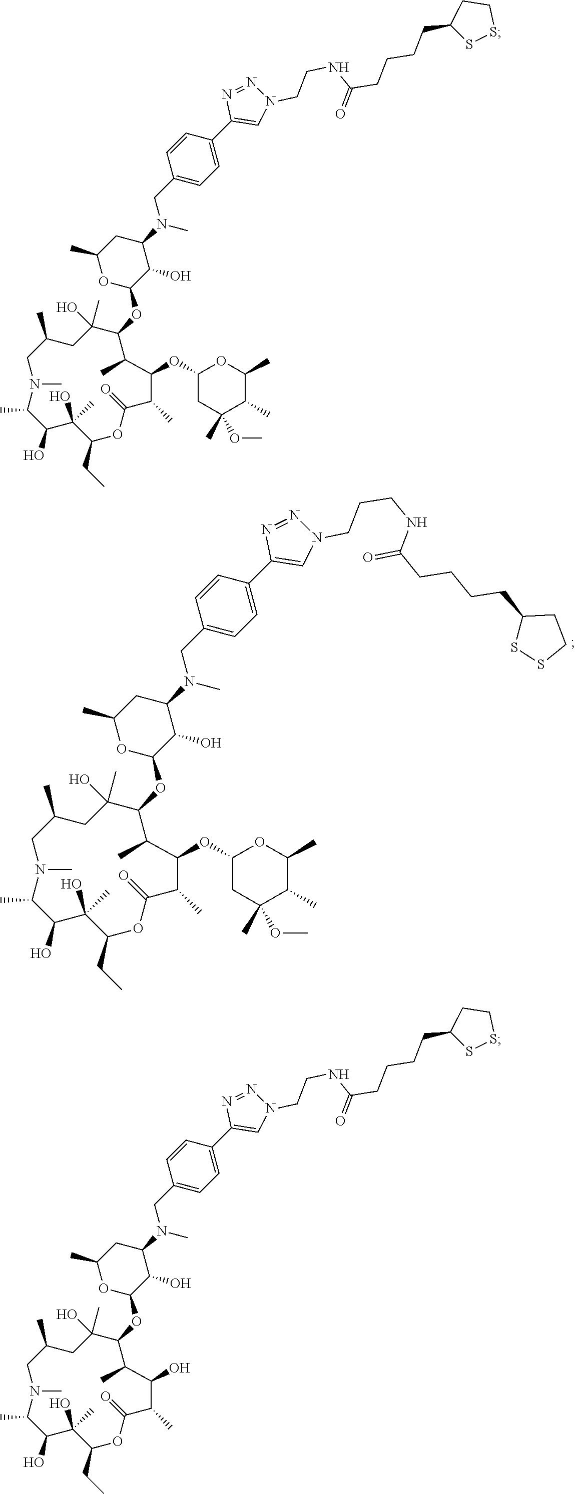

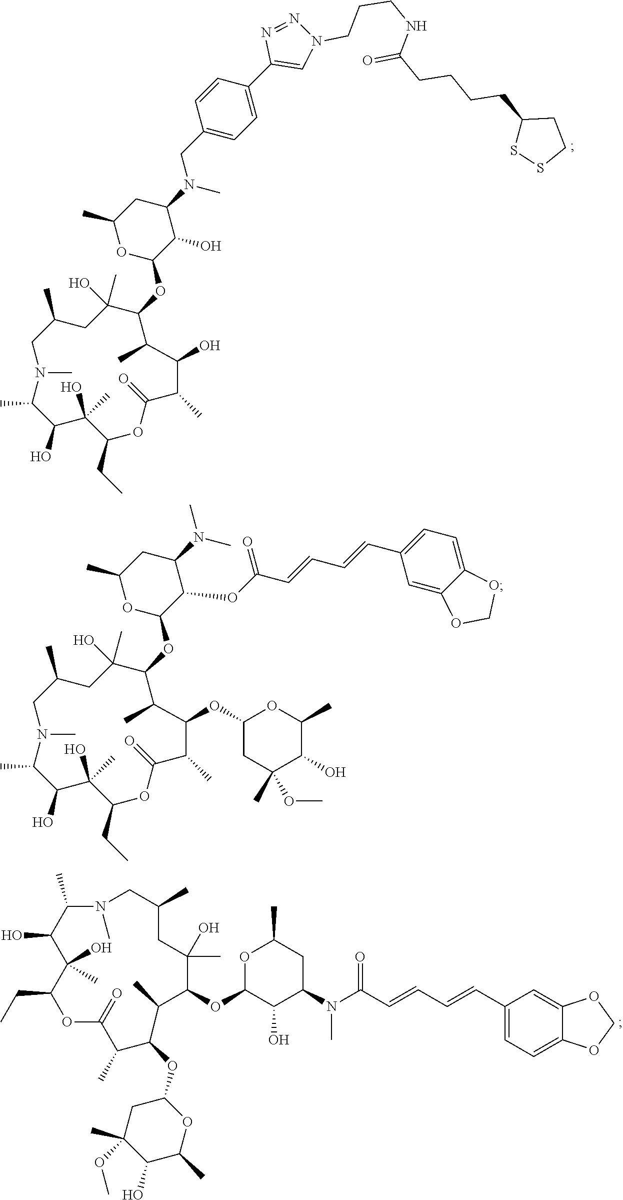

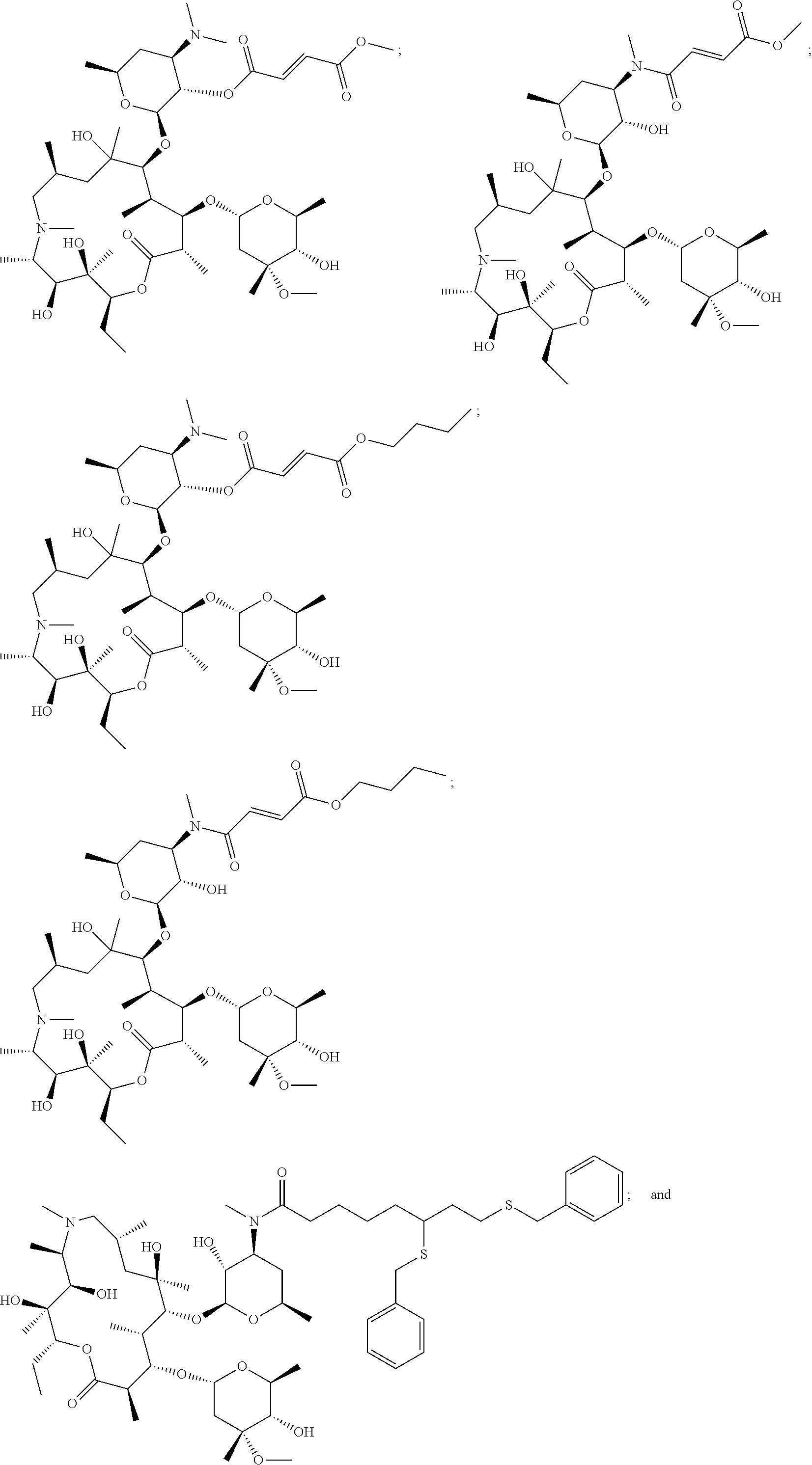

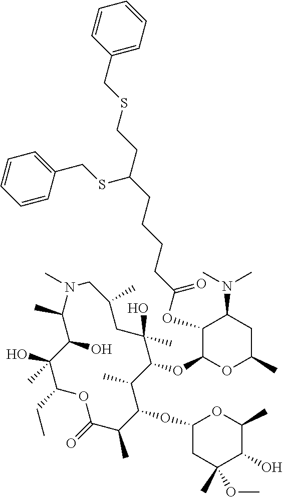 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=O)CCCCC(CCSCc2ccccc2)SCc2ccccc2)[C@](C)(O)C[C@@H](C)CN(C)[C@H](C)[C@@H](O)[C@]1(C)O]

or diastereomers, solvates, or a pharmaceutically acceptable salt thereof.

### III. Pharmaceutical Formulations

The compounds of invention and mixtures thereof can be formulated into a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. The compositions can be administered systemically.

The compounds can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6^{th} Edition, Ansel et. al., (Media, P A: Williams and Wilkins, 1995) which provides information on carriers, materials, equipment and process for preparing tablets and capsules and delayed release dosage forms of tablets, capsules, and granules.

The compounds can be administered to a subject with or without the aid of a delivery vehicle. Appropriate delivery vehicles for the compounds are known in the art and can be selected to suit the particular active agent. For example, in some embodiments, the active agent(s) is/are incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

A. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

B. Oral Immediate Release Formulations

Suitable oral dosage forms of the compounds include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

C. Extended Release Dosage Forms

The extended release formulations of compounds are generally prepared as diffusion or osmotic systems, for example, as described in “Remington—The science and practice of pharmacy” (20th ed., Lippincott Williams & Wilkins, Baltimore, M D, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations of the compounds can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

D. Delayed release dosage forms

In some embodiments delayed release formulations of compounds are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUIDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

E. Formulations for Mucosal and Pulmonary Administration

The compounds and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

One embodiment provides for nasal delivery for administration of the compounds of invention.

The compounds can be formulated as an aerosol. The term aerosol refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+nebulizer (PART Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

F. Topical and Transdermal Formulations

Transdermal formulations containing the compounds may also be prepared. These will typically be gels, ointments, lotions, sprays, or patches, all of which can be prepared using standard technology. Transdermal formulations can include penetration enhancers.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ ° 76 (stearyl poly(10 oxyethylene ether), BRIJ ° 78 (stearyl poly(20)oxyethylene ether), BRIJ ° 96 (oleyl poly(10)oxyethylene ether), and BRIJ ° 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

In some embodiments, the composition is formulated for transdermal delivery and administered using a transdermal patch. In some embodiments, the formulation, the patch, or both are designed for extended release of the curcumin conjugate.

Exemplary symptoms, pharmacologic, and physiologic effects are discussed in more detail below.

G. Methods of Manufacture

As will be appreciated by those skilled in the art and as described in the pertinent texts and literature, a number of methods are available for preparing formulations containing the compounds including but not limited to tablets, beads, granules, microparticle, or nanoparticles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

An exemplary method for preparing extended release tablets includes compressing a drug-containing blend, e.g., blend of drug-containing granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### IV. Methods of Use

The compounds and pharmaceutical compositions thereof are useful for the treatment of fibrosis, inflammatory disease, and cancer. In other embodiments, the present invention provides methods of inhibiting fibrosis, inflammatory disease, and cancer comprising contacting the cells with a compound and pharmaceutical composition thereof.

A. Fibrotic Diseases

In one embodiment, the present invention provides methods of treating a fibrotic disease, disorder or condition in a subject in need thereof comprising administering to the subject a therapeutically effective amount of the compound or a pharmaceutical composition thereof.

Representative fibrotic diseases, disorders or conditions that can be inhibited or treated by the disclosed compounds or pharmaceutical composition thereof include, but are not limited to, pulmonary fibrosis, liver fibrosis, skin fibrosis, renal fibrosis, pancreas fibrosis, systemic sclerosis, cardiac fibrosis and macular degeneration.

In some subjects, chronic pulmonary inflammation and fibrosis develop without an identifiable cause. Many of these subjects have a condition called idiopathic pulmonary fibrosis (IPF), a chronic progressive pulmonary fibrosis of unknown etiology. Prednisone is the usual treatment for IPF but other immunosuppressive therapies can be usede with the objective of reducing the inflammation that is the prelude to lung fibrosis. Although prednisone has a modest measurable effect on improving lung function, the scarce evidence for its long-term efficacy, as well as concerns regarding its safety, limits its use. Indeed, most immunosuppressive drugs have little therapeutic effects on IPF and lung transplantation may be necessary.

This invention describes compounds that are anticipated to have tissue-targeting property for sustained levels at disease sites, resulting in the inhibition of fibrosis and inflammation and growth of cancer cells while avoiding or minimizing the off target toxicities of systemic exposure. Representative compounds of invention are about 1000-fold more potent than Prednisone, the standard of care for IPF.

B. Inflammatory Diseases

In one embodiment, the present invention provides methods of treating an inflammatory disease, disorder or condition in a subject in need thereof, comprising administering to the subject a therapeutically effective amount of the compound or a pharmaceutical composition thereof.

Representative inflammatory diseases, disorders or conditions that can be inhibited or treated by the compound or pharmaceutical composition thereof include, but are not limited to, acute and chronic inflammatory disorders, sepsis, acute endotoxemia, encephalitis, Chronic Obstructive Pulmonary Disease (COPD), allergic inflammatory disorders, asthma, pulmonary fibrosis, pneumonia, Community acquired pneumonia (CAP), Ventilator associated pneumonia (VAP), Acute respiratory infection, Acute respiratory distress syndrome (ARDS), Infectious lung diseases, Pleural effusion, Peptic ulcer, Helicobacter pylori infection, hepatic granulomatosis, arthritis, rheumatoid arthritis, osteoarthritis, inflammatory osteolysis, ulcerative colitis, psoriasis, vasculitis, autoimmune disorders, thyroiditis, Meliodosis, (mesenteric) Ischemia reperfusion, Filovirus infection, Infection of the urinary tract, Bacterial meningitis, Salmonella enterica infection, Marburg and Ebola viruses infections and, in particular, Inflammatory Bowel Disease (IBD).

C. Cancers

In some embodiments, the present invention provides methods of treating a cancer in a subject in need thereof, comprising administering to the subject a therapeutically effective amount of the compound or a pharmaceutical composition thereof.

Representative cancers that can be inhibited or treated by the disclosed compounds or pharmaceutical composition thereof include, but are not limited to, squamous cell carcinoma, small-cell lung cancer, non-small cell lung cancer (NSCLC), lung adenocarcinoma, squamous cell lung cancer, peritoneum cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

### EXAMPLES

To provide a better understanding of the foregoing discussion, the following non-limiting examples are provided. Although the examples may be directed to specific embodiments, they are not to be viewed as limiting the invention in any specific respect.

### Chemistry:

Synthesis of Target Compounds 10a-c, 11a-c, 12a-c, 13a-c, 14a-c, 15a-c, 19a-e and 20a-e.

Key intermediate compounds 1a-c, were synthesized through Cu(I)-mediated coupling of commercially available 5-methyl-2-pyridine and bromophenyl alcohols. Treatment of 1a-c with methanesulfonyl chloride afforded mesylated compounds 2a-c. The reaction of 2a-c with sodium azide in DMF at elevated temperatures, furnished azide 3a-c (Scheme 1).

Desmethylclarithromycin 4 and desmethylazithromycin 7 were obtained azithromycin (AZM) and clarithromycin (CLM) using published protocol. The reactions of 4 and 7 with propargyl bromide or 4-ethynylbenzyl methanesulfonate furnished alkynyl-macrolides 5, 6, 8 and 9 (Scheme 2a-b).

The copper(I)-catalyzed cycloaddition reaction of azide 3a-c with alkynyl-macrolides 5, 6, 8 and 9 facilely furnished target compounds 10a-c, 11a-c, 12a-c and 13a-c (Scheme 3a-d). The reactions of 4 and 7 with mesylated compounds 2a-c in a CH₃CN/DMSO co-solvent in the presence of Hunig's base at elevated temperatures furnished target compounds 14a-c and 15a-c respectively (Scheme 3e-g).

To synthesize compounds 19a-e and 20-a-e, 5-methyl-2-pyridone was coupled with 1-iodo-4-methoxybenzene using the same condition as in step a of Scheme 1 to afford compound 16 which was subsequently demethylated with boron tribromide at −30 to 0° C. to give compound 17. Compound 17 was first triflated and then coupled with alkynyl alcohols (n=1-5) via Sonogashira coupling to furnish alkynyl-alcohols 18a-e. Mesylation of 18a-e was achieved by reacting with methanesulfonyl chloride and the reaction of resulting mesylates with compound 4 and 7 in a CH₃CN/DMSO in the presence of Hunig's base at elevated temperatures furnished the target compounds 19a-e and 20a-e respectively (Scheme 4). All compounds were characterized using ¹H NMR, ¹³C NMR, and mass spectroscopy prior to biological testing.

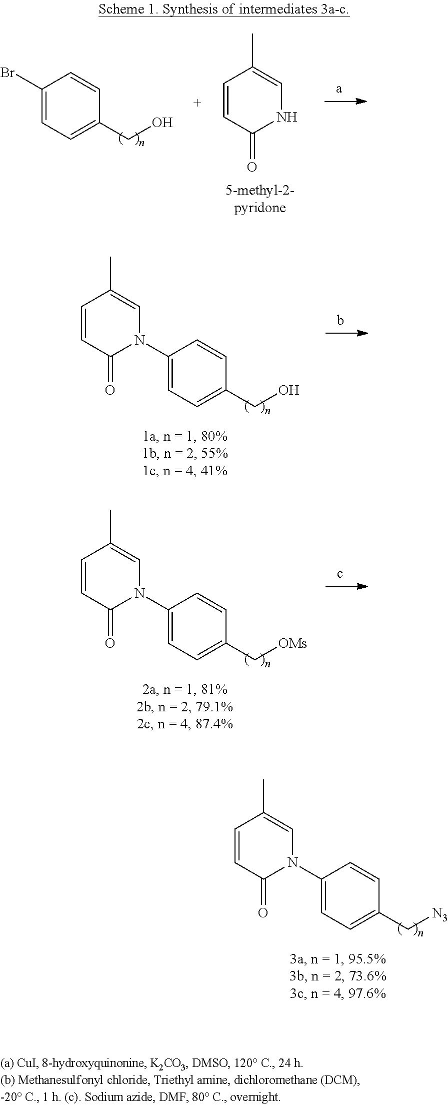

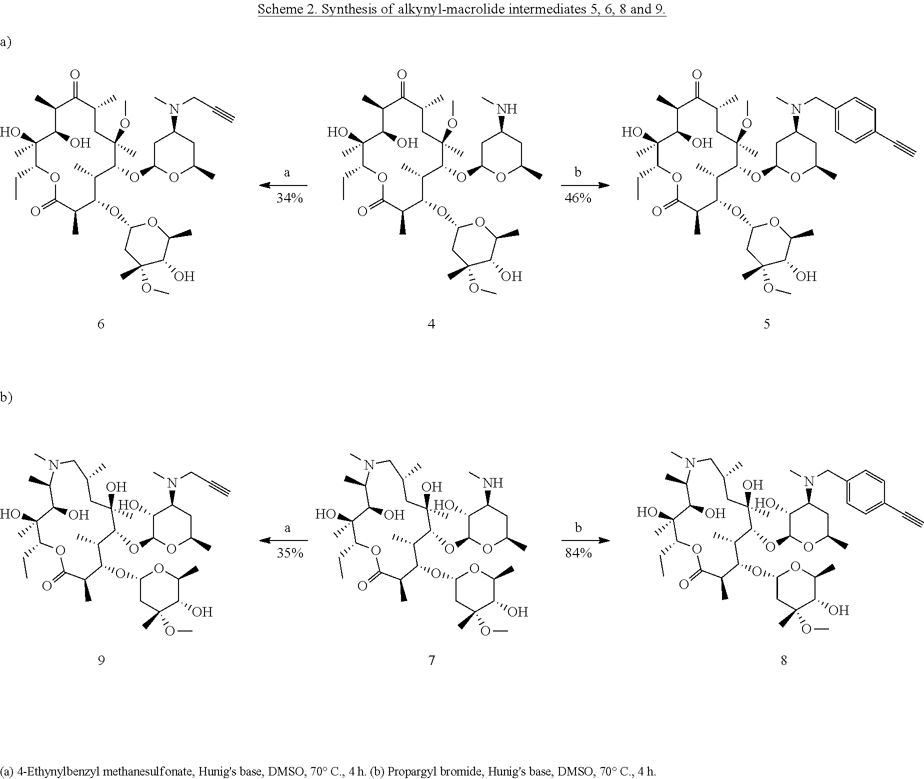

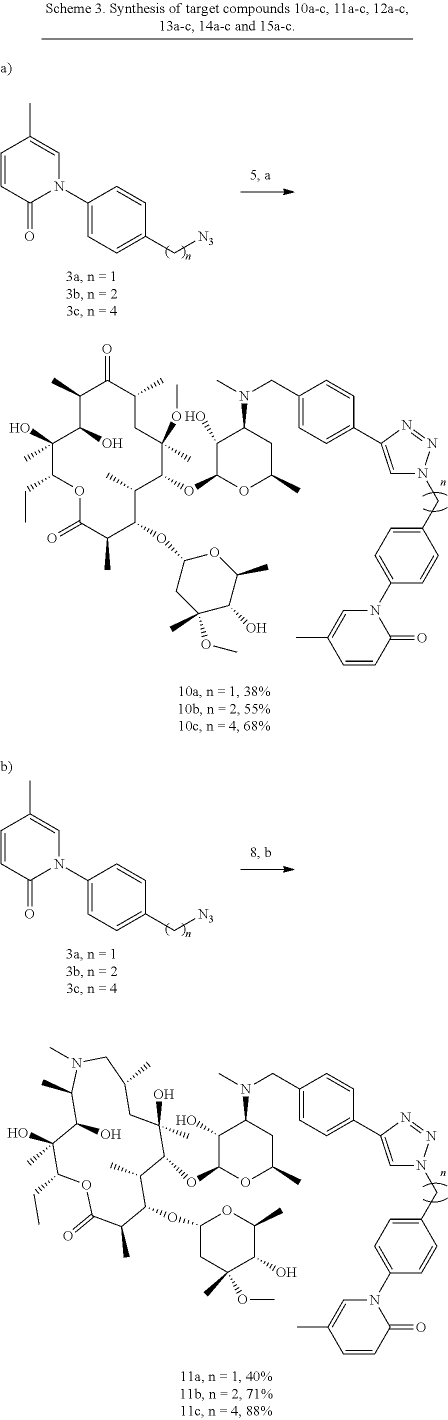

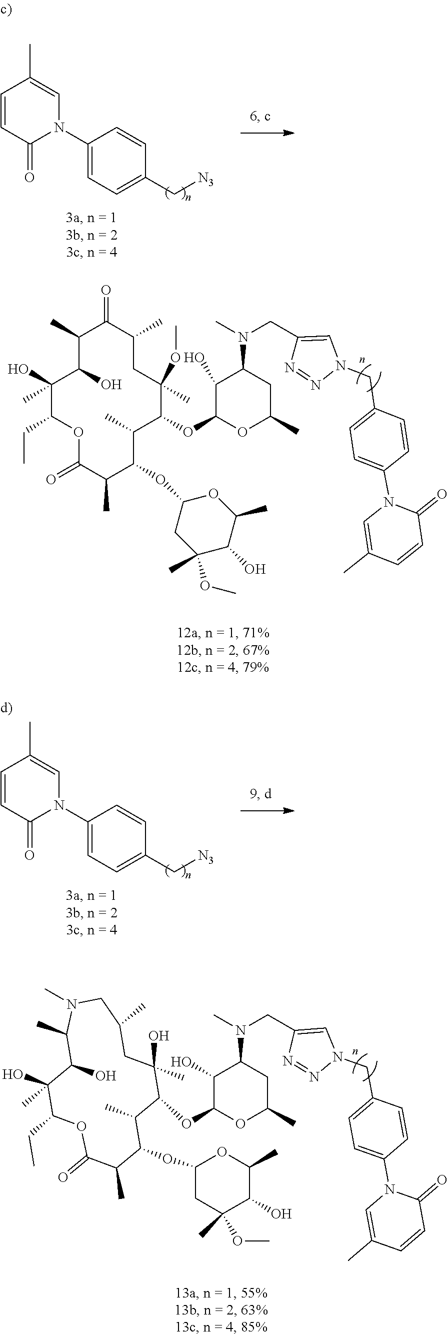

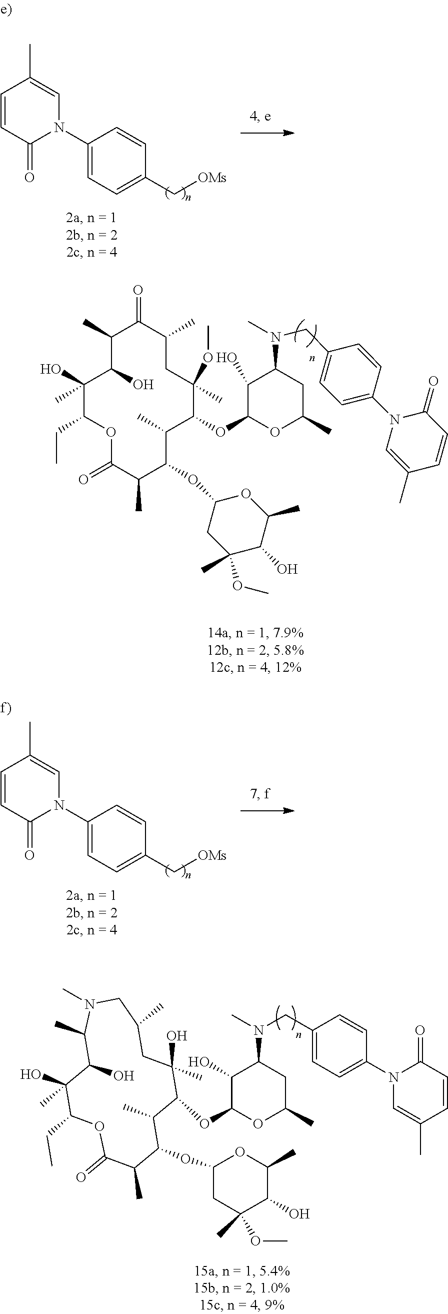

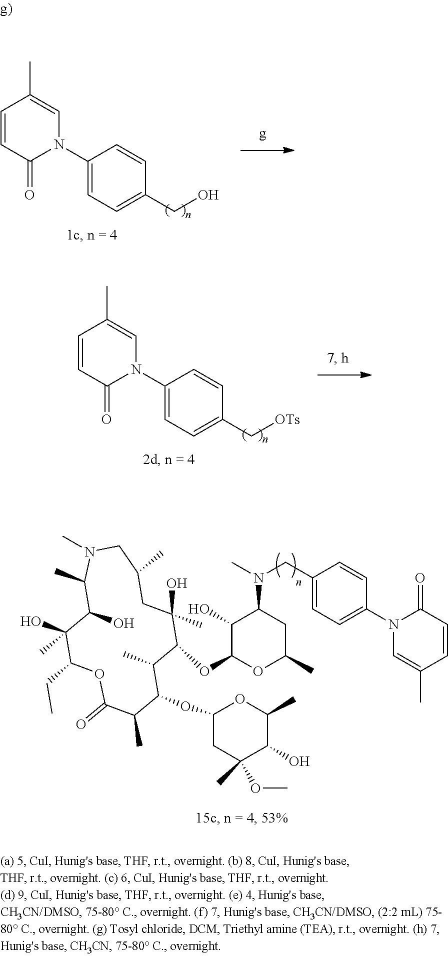

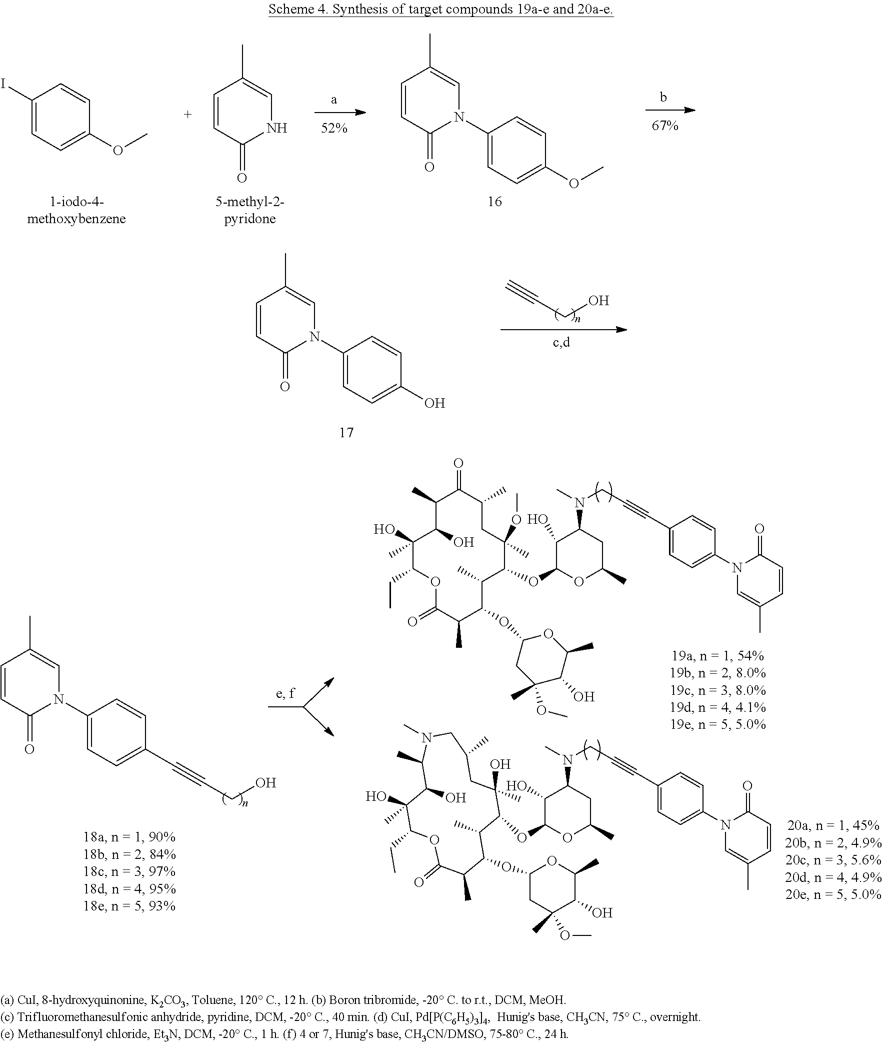

Synthesis Procedures for Target Compounds 10a-c, 11a-c, 12a-c, 13a-c, 14a-c, 15a-c, 19a-e and 20a-e.

Synthesis of Compound 1a (Ma, Z.; Pan, Y.; Huang, W.; Yang, Y.; Wang, Z.; Li, Q.; Zhao, Y.; Zhang, X.; Shen, Z. Synthesis and biological evaluation of the pirfenidone derivatives as antifibrotic agents. Bioorg. Med. Chem. Lett. 2014, 24, 220-223). The procedure described in this reference was adapted.

2-Hydroxy-5-methyl pyridine (1.32 g, 12.1 mmol) was added to potassium carbonate (1.67 g, 12.1 mmol), 8-hydroxyquinoline (348 mg, 2.4 mmol) and (4-bromophenyl) methanol (4 mL, 4.17 g, 22.02 mmol) in a 100 mL pressure flask. Dimethyl sulfoxide (DMSO) (25 mL) was added into the mixture and purged with argon for 30 min. Then, copper (I) iodide (696 mg, 3.66 mmol) was added into the solution. The pressure flask was capped after another 15 min of argon purge. The reaction was covered with aluminum foil and heated to 120° C. for 24 h. The reaction was cooled to room temperature and the resulting green mixture was worked up with DCM (100 mL×3) with water (150 mL). The combined DCM layer was washed with HCl solution (1M, 100 mL) and the green color turned back to pale yellow. The aqueous layer also turned yellow. So, more DCM (50 mL×5) was used to extract product in the aqueous layer. The combined DCM layer was dried over Na₂SO₄, evaporated in vacuo to give the crude product as yellow power. The crude was purified using silica gel chromatography (EtOAc:methanol=10:0.7) to furnish 1a as pale yellow solid (2.08 g, 80%). ¹H NMR (400 MHz, CDCl₃) δ 7.52-7.41 (d, 2H), 7.34 (d, J=8.4 Hz, 2H), 7.27 (d, J=9.7 Hz, 1H), 7.15-7.05 (m, 1H), 6.61 (d, J=9.3 Hz, 1H), 4.71 (d, J=5.8 Hz, 2H), 2.20 (t, J=6.0 Hz, 1H), 2.10 (d, J=1.1 Hz, 3H).

Synthesis of Compound 1b (Ma, Z.; Pan, Y.; Huang, W.; Yang, Y.; Wang, Z.; Li, Q.; Zhao, Y.; Zhang, X.; Shen, Z. Synthesis and Biological Evaluation of the Pirfenidone Derivatives as Antifibrotic Agents. Bioorg. Med. Chem. Lett. 2014, 24, 220-223).

The reaction of 2-hydroxy-5-methyl pyridine (0.2 g, 1.83 mmol), potassium carbonate (0.5 g, 3.6 mmol), 8-hydroxyquinoline (60 mg, 0.423 mmol) and 2-(4-bromophenyl) ethanol (0.36 mL, 0.37 g, 1.84 mmol) in DMSO, as described for the synthesis of 1a, furnished 1b as pale yellow solid (125 mg, 55%). ¹H NMR (400 MHz, CDCl₃) δ 7.33 (d, J=4.5 Hz, 3H), 7.28-7.27 (d, J=2.5 Hz, 1H), 7.25 (d, J=2.5 Hz, 1H), 7.11 (d, J=2.5 Hz, 1H), 6.60 (d, J=9.3 Hz, 1H), 3.88 (q, J=6.4 Hz, 2H), 2.91 (t, J=6.5 Hz, 2H), 2.10 (d, J=1.1 Hz, 3H).

Synthesis of Compound 1c (Ma, Z.; Pan, Y.; Huang, W.; Yang, Y.; Wang, Z.; Li, Q.; Zhao, Y.; Zhang, X.; Shen, Z. Synthesis and Biological Evaluation of the Pirfenidone Derivatives as Antifibrotic Agents. Bioorg. Med. Chem. Lett. 2014, 24, 220-223).

The reaction of 2-hydroxy-5-methyl pyridine (0.35 mg, 3.21 mmol), potassium carbonate (0.500 g, 3.6 mmol), 8-hydroxyquinoline (38 mg, 0.26 mmol) and 4-(4-bromophenyl) butanol (0.3 mL, 1.37 mmol) in DMSO (8 mL), as described for the synthesis of 1a, furnished 1c as yellow oil (145 mg, 41%). ¹H NMR (400 MHz, CDCl₃) δ 7.45 (d, J=7.2 Hz, 5H), 7.28 (dt, J=2.7, 1.0 Hz, 1H), 6.78 (d, J=9.3 Hz, 1H), 3.85 (t, J=6.6 Hz, 2H), 2.86 (t, J=7.5 Hz, 2H), 2.27 (d, J=1.1 Hz, 3H), 1.94-1.86 (m, 2H), 1.79 (t, J=3.1 Hz, 2H).

### Synthesis of Compound 2a.

Compound 1a (2.7 g, 12.6 mmol) was dissolved in anhydrous DCM (50 mL). Triethylamine (4 mL, 30.88 mmol) was added and the mixture was purged with argon at −15° C. 15 min later, methanelsulfonyl chloride (2 g, 20.4 mmol) was added into the solution. The reaction was running for 1-2 h until all starting materials have reacted. The solution was worked up by adding saturated NaHCO₃ solution (30 mL) and the two layers separated. The aqueous layer was extracted with (30 mL×3). The combined DCM layer was washed with brine (30 mL), and dried over Na₂SO₄. The mixture was evaporated off resulting in compound 2a (2.7 g, 81%) as yellow liquid. The crude product was used for the next step reaction without purification.

### Synthesis of Compound 2b.

The reaction of 1b (0.154 g, 0.67 mmol) with methanelsulfonyl chloride (0.147 g, 1.2 mmol) in anhydrous DCM (20 mL) and triethylamine (0.17 mL, 1.2 mmol) mixture, as described for 2a, furnished 2b (150 mg, 79%) as yellow liquid. The crude product was used for the next step reaction without purification.

### Synthesis of Compound 2c.

The reaction of 1c (2 g, 7.8 mmol) with methanelsulfonyl chloride (1.75 g, 16.8 mmol) in anhydrous DCM solution (30 mL) and triethylamine (2 mL, 15.44 mmol) mixture, as described for 2a, furnished 2c (2.1 g, 87%) as yellow liquid. The crude product was used for the next step reaction without purification.

### Synthesis of Compound 3a.

Compound 2a (600 mg, 2.23 mmol) was dissolved in to DMF (10 mL), NaN₃ (200 mg, 3.08 mmol) was added and the mixture was heated to 80° C. for 24 h. The reaction was allowed to cool down to room temperature, partitioned between DCM (50 mL) and water (100 mL) and the two layers separated. The aqueous layer was extracted with DCM (50 mL×3). The combined DCM layer was washed again with water (100 mL×2), dried over Na₂SO₄ and evaporated off to give 3a (503 mg, 95% as yellow liquid. ¹H NMR (400 MHz, CDCl₃) δ 7.49-7.38 (m, 4H), 7.29 (d, J=2.6 Hz, 1H), 7.16-7.08 (m, 1H), 6.62 (d, J=9.3 Hz, 1H), 4.40 (s, 2H), 2.11 (d, J=1.1 Hz, 3H).

### Synthesis of Compound 3b.

The reaction of 2b (680 mg, 2.2 mmol) with NaN₃ (175 mg, 2.69 mmol) in DMF (10 mL), as described for the synthesis of 3a, furnished 3b (405 mg, 74%) as yellow liquid. ¹H NMR (400 MHz, CDCl₃) δ 7.24-7.10 (m, 5H), 7.04-6.94 (m, 1H), 6.45 (d, J=9.3 Hz, 1H), 3.42 (t, J=7.2 Hz, 2H), 2.80 (d, J=7.2 Hz, 2H), 1.97 (d, J=1.3 Hz, 3H).

### Synthesis of Compound 3c.

The reaction of 2c (2.5 g, 6.08 mmol) with NaN₃ (700 mg, 10.8 mmol)) in DMF (10 mL), as described for the synthesis of 3a, furnished 3c (1.65 g, 98%) as yellow liquid. ¹H NMR (400 MHz, CDCl₃) δ 7.27 (m, 5H), 7.10 (m, 1H), 6.60 (d, J=9.3 Hz, 1H), 3.56 (t, J=6.2 Hz, 2H), 2.73-2.64 (m, 2H), 2.09 (d, J=1.1 Hz, 3H), 1.89-1.74 (m, 4H).

Synthesis of Compound 4 (Oyelere, A. K.; Chen, P. C.; Guerrant, W.; Mwakwari, S. C.; Hood, R.; Zhang, Y.; Fan, Y. Non-peptide macrocyclic histone deacetylase inhibitors. J. Med. Chem. 2009, 52, 456-468).

Clarithromycin (CLM) (15.00 g, 20.04 mmol) was added to 500 mL round bottom flask (RBF). Sodium acetate (14.04 g, 171 mmol) was added. Then 250 mL methanol with 10 mL chloroform solvent was added to completely dissolve CLM. The mixture was heated to 80° C. with addition of water just to completely dissolve NaOAc. The solution was cooled to 60° C. and iodine (5.24 g, 20.60 mmol) was added to the solution in three aliquots at about 3 min interval. The solution turned from cloudy white to dark yellow in 10 min. Sodium hydroxide (1 M, 10 mL) was added into the solution in the first 10 min. Then the solution was stirred for 30 min. Another 2 aliquots of sodium hydroxide (1 M, 10 mL) were added into the solution. The solution turned clear and stirred for another 2 h at 60° C. The reaction was allowed to cool to room temperature, and water (500 mL) and NH₄OH (10 M, 15 mL) were added. Extraction was done using DCM (150 mL×4). The combined DCM solution was washed with ammonium hydroxide (1 M, 200 mL). The aqueous layer is extracted with DCM (100 mL×2). The combined DCM solution was evaporated and the product was recrystallized using acetone:NH₄OH (15:1, 20 mL) to give 4 (12.5 g, 85%) as white powder.

Synthesis of Compound 5 (Oyelere, A. K.; Chen, P. C.; Guerrant, W.; Mwakwari, S. C.; Hood, R.; Zhang, Y.; Fan, Y. Non-peptide macrocyclic histone deacetylase inhibitors. J. Med. Chem. 2009, 52, 456-468).

Compound 4 (1 g, 1.3 mmol) and 4-ethynylbenzyl methanesulfonate (280 mg, 1.33 mmol) were dissolved in DMSO (10 mL). Hunig's base (0.6 mL, 3.25 mmol) was added and the mixture was heated to 75° C. for 4 h. The reaction was partitioned between DCM (50 mL) and water (100 mL) and the two layers were separated. The aqueous layer was extracted with DCM (50 mL×3). The combined DCM layer was dried over Na₂SO₄ and evaporated off. The crude was purified using column chromatography eluting with ethyl acetate:hexanes (4:6) to give compound 5 (512 mg, 46%) as pale-yellow solid.

### Synthesis of Compound 6.

The reaction of compound 4 (2 g, 2.6 mmol) with propargyl bromide (376.8 mg, 2.6 mmol) in DMSO (10 mL) and Hunig's base (1.2 mL, 6.5 mmol) at 50° C. (note: over 50° C. will generate multiple byproducts) for 4 h, followed by work up as described for the synthesis of 5, furnished a crude product. The crude was purified using column chromatography eluting with CHCl₃:MeOH:NH₄OH=20:1:0.1 to furnish 6 (650 mg, 34%) as pale yellow solid. ¹H NMR (400 MHz, CDCl₃) δ 7.26 (s, 3H), 5.05 (dd, J=11.0, 2.5 Hz, 1H), 4.93 (d, J=4.8 Hz, 1H), 4.46 (d, J=7.1 Hz, 1H), 3.98 (s, 2H), 3.80-3.73 (m, 2H), 3.66 (d, J=7.5 Hz, 1H), 3.53-3.45 (m, 1H), 3.40 (dd, J=3.9, 2.5 Hz, 2H), 3.33 (d, J=1.1 Hz, 4H), 3.24 (d, J=7.4 Hz, 1H), 3.21 (d, J=2.9 Hz, 1H), 3.18 (s, 1H), 3.07-2.94 (m, 6H), 2.94-2.82 (m, 1H), 2.74-2.54 (m, 2H), 2.40-2.31 (m, 5H), 2.29-2.21 (m, 1H), 2.19 (d, J=10.3 Hz, 1H), 1.95-1.85 (m, 2H), 1.85-1.77 (m, 2H), 1.74-1.65 (m, 1H), 1.62-1.53 (m, 4H), 1.53-1.40 (m, 3H), 1.37-1.17 (m, 19H), 1.15-1.05 (m, 16H), 0.84 (t, J=7.5 Hz, 4H).

Synthesis of Compound 7 (Oyelere, A. K.; Chen, P. C.; Guerrant, W.; Mwakwari, S. C.; Hood, R.; Zhang, Y.; Fan, Y. Non-peptide macrocyclic histone deacetylase inhibitors. J. Med. Chem. 2009, 52, 456-468).

Azithromycin (AZM) (15.00 g, 20.05 mmol) was added to 500 mL round bottom flask. Sodium acetate (14.04 g, 171 mmol) and 120 mL 80%:20% V/V % methanol:H₂O were added. The mixture was heated to 50° C. Iodine (5.24 g, 20.60 mmol) was added to the solution in three aliquots within 3 min. The solution turned from cloudy white to clear yellow in 10 min. Sodium hydroxide (1 M, 10 mL) was added into the solution in the first 10 min. Then the solution was stirred for 30 min and another 2 aliquots of sodium hydroxide solution (1 M, 10 mL) were added into the reaction. The solution turned clear and was stirred for another 2 h at 50° C. The reaction was allowed to cool to room temperature, poured into water (500 mL) and NH₄OH (10 M, 15 mL) and extracted with DCM (150 mL×4). The combined DCM layer was washed with ammonium hydroxide (1 M, 200 mL). The combined DCM layer was dried over Na₂SO₄ and evaporated. The product was recrystallized using acetone:NH₄OH (15:1, 20 mL) to yield 7 as white powder (10.8 g, 73%).

### Synthesis of Compound 8.

Compound 7 (1 g, 1.3 mmol) and 4-ethynylbenzyl methanesulfonate (280 mg, 1.33 mmol) were dissolved in DMSO (10 mL) and Hunig's base (0.6 mL, 3.25 mmol). The reaction mixture was heated to 75° C. for 4 h, cooled to room temperature, partitioned between DCM (50 mL) and water (100 mL) and the two layers were separated. The aqueous layer was extracted by DCM (500 mL×3), the combined DCM layer was dried over Na₂SO₄ and evaporated off. The crude was purified using column chromatography eluting with DCM:MeOH:NH₄OH=15:1:0.1. to give 8 (512 mg, 46.3%) as whitish-yellow solid.

### Synthesis of Compound 9.

The reaction of compound 7 (2 g, 2.6 mmol) and propargyl bromide (376.8 mg, 2.6 mmol) in DMSO (10 mL) and Hunig's base (1.2 mL, 6.5 mmol) at 50° C. (note: over 50° C. will generate multiple byproducts) for 2 h, followed by work up as described for the synthesis of 8, furnished a crude product. The crude was purified using column chromatography eluting with CHCl₃:MeOH:NH₄OH=10:1:0.1 to furnish 9 (710 mg, 34.6%) as whitish-yellow solid. ¹H NMR (400 MHz, CDCl₃) δ 7.30-7.23 (m, 2H), 5.12 (d, J=7.6 Hz, 1H), 4.75 (s, 1H), 4.46 (d, J=7.1 Hz, 1H), 4.26 (s, 1H), 4.05 (d, J=6.8 Hz, 1H), 3.77-3.69 (m, OH), 3.63 (d, J=6.8 Hz, 1H), 3.54 (s, 2H), 3.40 (s, 2H), 3.34 (d, J=2.3 Hz, 3H), 3.25 (t, J=8.6 Hz, 1H), 3.04 (t, J=9.7 Hz, 1H), 2.73 (s, 1H), 2.24 (s, 1H), 2.15 (d, J=10.5 Hz, 1H), 1.95-1.87 (m, 3H), 1.79 (t, J=14.2 Hz, 2H), 1.59 (dd, J=15.0, 4.6 Hz, 1H), 1.46 (dd, J=15.8, 8.1 Hz, 1H), 1.39-1.30 (m, 4H), 1.25 (s, 8H), 1.33-1.18 (m, 5H), 1.18 (s, 1H), 1.13-1.08 (m, 2H), 1.04 (d, J=7.8 Hz, 3H), 0.95 (s, 1H), 0.91 (d, J=7.0 Hz, 2H), 0.87 (d, J=8.2 Hz, 1H).

### Synthesis of Compound 10a.

Compounds 3a (60 mg, 0.25 mmol) and 5 (150 mg, 0.178 mmol) were dissolved in THF (2 mL) and DMSO (1 mL). Hunig's base (0.3 mL, 1.77 mmol) was added and the mixture was purged with argon for 20 min. CuI (5 mg, 0.03 mmol) was added and the reaction mixture was at room temperature overnight. The reaction was partitioned between DCM (30 mL) and water (50 mL) and the two layers separated. The aqueous layer was extracted with DCM (50 mL); the combined DCM was dried by Na₂SO₄ and evaporated off. The crude was purified in preparative TLC, eluting with DCM:methanol=15:1 to furnish 10a (74 mg, 38%) as white powder. ¹H NMR (400 MHz, CDCl₃) δ 7.79-7.74 (m, 2H), 7.73 (s, 1H), 7.33 (d, J=8.0 Hz, 2H), 7.30-7.23 (m, 4H), 7.11-7.04 (m, 2H), 6.59 (dd, J=9.3, 1.5 Hz, 2H), 5.03 (dt, J=11.1, 2.3 Hz, 2H), 4.89 (t, J=6.2 Hz, 2H), 4.41 (d, J=7.2 Hz, 2H), 4.34 (t, J=7.1 Hz, 1H), 4.00-3.93 (m, 2H), 3.81-3.68 (m, 4H), 3.63 (dd, J=11.6, 7.0 Hz, 2H), 3.45 (dd, J=18.1, 10.4 Hz, 3H), 3.36-3.25 (m, 1H), 3.23 (s, 2H), 3.14 (d, J=27.8 Hz, 5H), 3.02 (d, J=3.8 Hz, 5H), 2.88-2.79 (m, 2H), 2.68 (t, J=7.5 Hz, 1H), 2.55 (d, J=10.3 Hz, 3H), 2.29 (dd, J=13.6, 5.1 Hz, 1H), 2.09 (d, J=1.3 Hz, 6H), 1.96-1.78 (m, 5H), 1.74-1.59 (m, 2H), 1.56-1.44 (m, 2H), 1.39 (d, J=2.5 Hz, 5H), 1.31-1.13 (m, 8H), 1.13-1.03 (m, 17H), 0.82 (td, J=7.5, 1.6 Hz, 6H). ¹³C NMR (101 MHz, CDCl₃) δ 220.1, 174.8, 160.8, 160.6, 147.1, 141.8, 140.4, 133.9, 128.2, 127.8, 126.4, 125.5, 124.8, 120.5, 118.6, 114.2, 113.9, 101.7, 94.9, 79.9, 77.3, 73.2, 71.5, 68.0, 67.6, 64.6, 62.9, 56.5, 52.6, 49.6, 48.4, 44.2, 44.0, 38.1, 36.2, 35.9, 33.7, 28.7, 20.5, 20.3, 18.8, 17.6, 17.0, 16.0, 15.0, 14.9, 11.3, 9.6, 8.1. HRMS (ESI) m/z Calcd. for C₅₄H₈₆O₁₆N₇ [M+H⁺]: 1088.6126, found 1088.6149.

### Synthesis of Compound 10b.

The reaction of 3b (200 mg, 0.75 mmol), 5 (500 mg, 0.59 mmol), CuI (20 mg, 0.11 mmol) in THF (2 mL), DMSO (1 mL) and Hunig's base (0.3 mL, 1.77 mmol), as described for the synthesis of 10a, after purification by column chromatography eluting with DCM:MeOH=15:1, furnished 10b (359 mg, 55%) as white powder. ¹H NMR (400 MHz, CDCl₃) δ 7.73 (d, J=7.8 Hz, 2H), 7.64 (s, 1H), 7.36-7.18 (m, 8H), 7.05 (d, J=2.5 Hz, 1H), 6.56 (d, J=9.3 Hz, 1H), 5.28 (s, 2H), 5.01 (dd, J=11.0, 2.3 Hz, 1H), 4.86 (d, J=4.9 Hz, 1H), 4.61 (t, J=7.3 Hz, 2H), 4.39 (d, J=7.1 Hz, 1H), 3.97-3.88 (m, 2H), 3.79-3.67 (m, 3H), 3.60 (d, J=7.1 Hz, 1H), 3.50-3.37 (m, 2H), 3.28 (q, J=7.1, 5.8 Hz, 3H), 3.17 (s, 1H), 3.09 (s, 3H), 3.01-2.89 (m, 5H), 2.84 (t, J=8.2 Hz, 1H), 2.55 (d, J=21.1 Hz, 3H), 2.27 (d, J=15.2 Hz, 1H), 2.21 (s, 3H), 2.13 (d, J=9.9 Hz, 1H), 2.06 (s, 3H), 1.93-1.79 (m, 2H), 1.71 (dt, J=21.9, 13.4 Hz, 2H), 1.53-1.42 (m, 1H), 1.37 (s, 3H), 1.32-1.18 (m, 8H), 1.18-1.01 (m, 17H), 0.81 (q, J=8.4, 7.4 Hz, 6H). ¹³C NMR (101 MHz, CDCl₃) δ 221.1, 175.8, 161.8, 147.4, 142.8, 140.0, 137.3, 135.1, 129.6, 127.0, 125.8, 121.4, 119.9, 115.2, 102.7, 95.9, 80.9, 78.3, 77.9, 74.3, 72.5, 70.7, 69.1, 68.6, 65.6, 63.9, 57.6, 51.4, 50.6, 49.4, 45.2, 45.0, 39.2, 39.1, 37.2, 36.9, 36.3, 34.8, 29.7, 21.5, 21.3, 21.0, 19.8, 18.6, 18.0, 17.0, 15.9, 12.3, 10.6, 9.1. HRMS (ESI) m/z Calcd. for C₆₀H₈₈O₁₄N₅ [M+H⁺]: 1102.6322, found 1102.6310.

### Synthesis of Compound 10c.

The reaction of 3c (200 mg, 0.79 mmol), 5 (500 mg, 0.59 mmol), CuI (20 mg, 0.11 mmol) in THF (2 mL), DMSO (1 mL) and Hunig's base (0.3 mL, 1.77 mmol), as described for the synthesis of 10a, after purification by column chromatography eluting with DCM:MeOH=15:1, furnished 10c (453 mg, 68%) as pale-yellow powder. ¹H NMR (400 MHz, CDCl₃) δ 7.82-7.75 (m, 2H), 7.73 (s, 1H), 7.33 (d, J=7.9 Hz, 2H), 7.30-7.20 (m, 5H), 7.11-7.05 (m, 1H), 6.58 (d, J=9.3 Hz, 1H), 5.28 (s, 1H), 5.03 (dd, J=11.0, 2.2 Hz, 1H), 4.88 (d, J=4.8 Hz, 1H), 3.94 (d, J=20.0 Hz, 1H), 3.81-3.69 (m, 3H), 3.61 (d, J=7.1 Hz, 1H), 3.50-3.39 (m, 2H), 3.30 (dd, J=10.2, 7.1 Hz, 1H), 3.17 (s, 1H), 3.11 (s, 3H), 3.03-2.91 (m, 5H), 2.86 (dd, J=9.2, 7.0 Hz, 1H), 2.69 (t, J=7.5 Hz, 2H), 2.62-2.52 (m, 2H), 2.32-2.24 (m, 4H), 2.08 (d, J=1.1 Hz, 3H), 1.98 (q, J=7.5 Hz, 1H), 1.92-1.84 (m, 1H), 1.84-1.74 (m, 1H), 1.74-1.63 (m, 3H), 1.55-1.41 (m, 1H), 1.39 (s, 3H), 1.24 (dd, J=13.2, 6.2 Hz, 6H), 1.16 (d, J=7.2 Hz, 2H), 1.11 (dd, J=6.1, 2.0 Hz, 15H), 1.06 (d, J=7.5 Hz, 2H), 0.82 (t, J=7.4 Hz, 3H). ¹³C NMR (101 MHz, CDCl₃) δ 175.8, 161.8, 147.5, 142.6, 141.6, 139.1, 135.4, 129.2, 126.5, 125.7, 121.3, 119.4, 114.9, 102.7, 95.9, 80.9, 78.3, 75.4, 72.5, 70.7, 69.0, 68.9, 65.6, 50.6, 50.2, 49.4, 45.6, 45.3, 45.0, 40.4, 39.3, 36.8, 34.8, 31.5, 29.8, 28.0, 21.5, 21.3, 21.0, 19.8, 18.6, 18.3, 18.0, 16.8, 16.0, 15.9, 12.3, 10.6. HRMS (ESI) m/z Calcd. for C₆₂H₉₂O₁₄N₅ [M+H⁺]: 1130.6635, found 1130.6625.

### Synthesis of Compound 11a.

Compounds 3a (60 mg, 0.25 mmol) and 8 (150 mg, 0.18 mmol) were dissolved in THF (2 mL) and DMSO (1 mL). Hunig's base (0.3 mL, 1.77 mmol) was added and the mixture was purged with argon for 20 min. CuI (5 mg, 0.03 mmol) was added and the reaction was stirred at room temperature overnight. The reaction was partitioned between DCM (30 mL) and water (50 mL) and the two layers separated. The aqueous layer was extracted with DCM (50 mL); the combined DCM was dried by Na₂SO₄ and evaporated off. The crude was purified using preparative TLC (prep TLC) eluting with DCM:methanol=15:1 to furnish 11a (80 mg, 40%) as white powder. ¹H NMR (400 MHz, CDCl₃) δ 7.71 (d, J=7.8 Hz, 2H), 7.64 (s, 1H), 7.34-7.23 (m, 4H), 7.21 (d, J=8.2 Hz, 2H), 7.04 (s, 1H), 6.55 (d, J=9.3 Hz, 1H), 5.27 (s, 2H), 5.12 (d, J=4.8 Hz, 1H), 4.71-4.55 (m, 3H), 4.38 (d, J=7.2 Hz, 1H), 4.19 (t, J=2.6 Hz, 1H), 4.02 (dq, J=12.6, 6.4 Hz, 1H), 3.75 (d, J=13.1 Hz, 1H), 3.62 (s, 1H), 3.55 (d, J=7.3 Hz, 1H), 3.44 (q, J=13.3, 12.0 Hz, 3H), 3.36-3.22 (m, 3H), 3.13 (d, J=24.8 Hz, 3H), 2.97 (t, J=9.7 Hz, 1H), 2.72-2.60 (m, 2H), 2.53 (dd, J=20.6, 8.7 Hz, 2H), 2.26 (d, J=6.9 Hz, 4H), 2.21 (s, 3H), 2.12 (d, J=10.3 Hz, 1H), 2.05 (s, 2H), 1.99 (q, J=6.9 Hz, 1H), 1.73 (dd, J=14.6, 4.9 Hz, 2H), 1.28 (t, J=3.2 Hz, 6H), 1.20 (d, J=6.2 Hz, 3H), 1.12 (d, J=7.7 Hz, 5H), 1.09-1.01 (m, 5H), 0.99 (d, J=7.5 Hz, 2H), 0.85 (q, J=7.4, 6.8 Hz, 6H). ¹³C NMR (176 MHz, CDCl₃) δ 179.1, 161.7, 148.2, 142.9, 141.4, 139.1, 134.9, 129.3, 128.9, 127.4, 125.8, 121.5, 119.6, 115.4, 102.9, 94.4, 83.5, 78.1, 77.7, 74.1, 73.7, 73.3, 72.8, 70.6, 70.1, 68.7, 65.6, 64.3, 62.6, 57.7, 53.6, 49.4, 45.5, 42.7, 42.1, 36.9, 36.1, 34.6, 29.6, 27.7, 26.8, 18.1, 17.0, 16.3, 14.5, 11.2, 8.9, 7.1. HRMS (ESI) m/z Calcd. for C₅₉H₈₉O₁₃N₆ [M+H⁺]: 1089.6482, found 1089.6474.

### Synthesis of Compound 11b.

The reaction of compounds 3b (65 mg, 0.28 mmol), 8 (150 mg, 0.19 mmol), CuI (20 mg, 0.11 mmol) in THF (2 mL), DMSO (1 mL) and Hunig's base (0.2 mL, 0.49 mmol), as described for the synthesis of 11a, followed by prep TLC eluting with DCM:methanol:NH₄OH=10:1:0.2, furnished 11b (150 mg, 71%) as white powder. ¹H NMR (400 MHz, CDCl₃) δ 7.77-7.70 (m, 2H), 7.64 (s, 1H), 7.36-7.19 (m, 7H), 7.05 (dd, J=2.5, 1.3 Hz, 1H), 6.56 (d, J=9.3 Hz, 1H), 5.27 (s, 2H), 5.11 (d, J=4.8 Hz, 1H), 4.71-4.58 (m, 3H), 4.40 (d, J=7.3 Hz, 1H), 4.21 (dd, J=3.9, 2.0 Hz, 1H), 4.14-3.96 (m, 1H), 3.77 (d, J=13.1 Hz, 1H), 3.66 (s, 1H), 3.57 (d, J=7.2 Hz, 1H), 3.48-3.43 (m, 2H), 3.40-3.17 (m, 3H), 3.09 (d, J=18.3 Hz, 4H), 2.97 (t, J=9.8 Hz, 1H), 2.70 (dd, J=7.5, 3.6 Hz, 2H), 2.62-2.50 (m, 2H), 2.34-2.20 (m, 7H), 2.13-2.05 (m, 4H), 2.01 (s, 1H), 1.97-1.81 (m, 1H), 1.75 (d, J=14.8 Hz, 2H), 1.55-1.39 (m, 2H), 1.35 (t, J=7.3 Hz, 1H), 1.28 (d, J=6.2 Hz, 2H), 1.19-1.05 (m, 11H), 1.00 (d, J=7.5 Hz, 3H), 0.92-0.81 (m, 6H). ¹³C NMR (101 MHz, CDCl₃) δ 161.7, 147.4, 142.8, 140.1, 137.3, 135.1, 129.6, 129.3, 127.0, 125.8, 121.4, 119.8, 115.1, 102.9, 94.4, 83.5, 78.1, 74.2, 73.7, 72.8, 70.6, 69.9, 68.7, 65.6, 64.3, 57.7, 51.4, 49.3, 45.4, 42.5, 36.9, 36.3, 34.6, 31.6, 29.7, 27.5, 26.7, 22.6, 22.0, 21.5, 21.4, 18.1, 17.0, 16.3, 14.6, 14.1, 11.2, 9.0, 7.3. HRMS (ESI) m/z Calcd. for C₆₀H₉₁O₁₃N₆ [M+H⁺]: 1103.6639, found 1103.6634.

### Synthesis of Compound 11c.

The reaction of compounds 3c (200 mg, 0.70 mmol), 8 (150 mg, 0.18 mmol), CuI (32 mg, 0.17 mmol) in THF (2 mL) and DMSO (2 mL) and Hunig's base (0.5 mL, 2.86 mmol), as described for the synthesis of 11a, furnished 11c (170 mg, 88%) as white powder. ¹H NMR (400 MHz, CDCl₃) δ 7.76 (d, J=7.8 Hz, 2H), 7.72 (s, 1H), 7.32 (d, J=7.8 Hz, 2H), 7.24 (p, J=4.5, 3.9 Hz, 5H), 7.07 (s, 1H), 6.57 (d, J=9.3 Hz, 1H), 5.27 (s, 2H), 5.06 (d, J=4.8 Hz, 1H), 4.66 (d, J=9.5 Hz, 2H), 4.44-4.35 (m, 3H), 4.22 (d, J=4.4 Hz, 1H), 4.00 (dq, J=12.4, 6.4 Hz, 1H), 3.77 (d, J=13.0 Hz, 1H), 3.68-3.56 (m, 2H), 3.47 (q, J=15.1, 11.4 Hz, 3H), 3.38-3.29 (m, 1H), 3.11 (s, 3H), 3.01-2.87 (m, 2H), 2.77-2.64 (m, 4H), 2.61-2.47 (m, 2H), 2.33-2.20 (m, 8H), 2.12-2.02 (m, 4H), 1.96 (q, J=7.6 Hz, 3H), 1.81-1.62 (m, 3H), 1.54-1.40 (m, 2H), 1.36-1.25 (m, 8H), 1.22 (d, J=6.1 Hz, 4H), 1.08 (d, J=18.8 Hz, 7H), 1.01 (d, J=7.4 Hz, 2H), 0.87 (q, J=7.5, 6.5 Hz, 7H). ¹³C NMR (101 MHz, CDCl₃) δ 179.1, 161.8, 147.5, 142.7, 141.7, 139.1, 135.4, 129.7, 129.3, 126.5, 125.7, 121.3, 119.5, 115.0, 102.9, 94.4, 83.4, 78.0, 77.7, 74.1, 73.7, 73.2, 72.8, 70.6, 70.0, 68.7, 65.6, 64.2, 62.7, 57.7, 50.2, 49.3, 45.5, 42.6, 29.7, 28.0, 27.6, 26.7, 22.0, 21.4, 21.4, 18.1, 17.0, 16.3, 14.5, 11.2, 8.9, 7.1. HRMS (ESI) m/z Calcd. for C₆₂H₉₅O₁₃N₆ [M+H⁺]: 1131.6952, found 1131.6942.

### Synthesis of Compound 12a.

Compound 3a (105 mg, 0.20 mmol) and 6 (150 mg, 0.2 mmol) were dissolved in THF (1 mL). Hunig's base (0.1 mL, 0.25 mmol) was added and the mixture was purged with argon for 20 min. CuI (10 mg, 0.53 mmol) was added and the reaction was stirred at room temperature overnight. The reaction was partitioned between DCM (30 mL) and water (50 mL) and the two layers separated. The aqueous layer was extracted with DCM (50 mL); the combined DCM was dried by Na₂SO₄ and evaporated off. The crude was purified in prep TLC, eluting with ethyl acetate:hexanes:MeOH=16:1:1 to furnish 12a (145 mg, 71%) as pale-yellow powder. ¹H NMR (400 MHz, CDCl₃) δ 7.45 (s, 1H), 7.39-7.29 (m, 4H), 7.26 (s, 2H), 7.23 (s, OH), 7.05 (s, 1H), 6.56 (d, J=9.3 Hz, 1H), 5.52 (d, J=1.6 Hz, 2H), 5.01 (dd, J=11.1, 2.3 Hz, 1H), 4.87 (d, J=4.8 Hz, 1H), 4.38 (d, J=7.2 Hz, 1H), 3.96 (d, J=8.2 Hz, 2H), 3.80 (d, J=14.0 Hz, 1H), 3.74-3.59 (m, 4H), 3.52-3.43 (m, 1H), 3.28-3.15 (m, 5H), 2.99 (s, 4H), 2.87-2.77 (m, 1H), 2.56 (dtd, J=22.5, 10.4, 7.7, 4.7 Hz, 2H), 2.36-2.19 (m, 5H), 2.07 (s, 3H), 1.94-1.61 (m, 5H), 1.53 (dd, J=15.2, 5.0 Hz, 1H), 1.36 (s, 3H), 1.31-1.12 (m, 12H), 1.11-1.00 (m, 11H), 0.80 (t, J=7.3 Hz, 3H). ¹³C NMR (101 MHz, CDCl₃) δ 220.9, 175.8, 161.6, 146.5, 142.9, 141.3, 134.8, 128.9, 127.3, 122.4, 121.4, 115.3, 102.8, 96.1, 81.0, 78.3, 77.9, 77.3, 76.6, 74.2, 72.7, 71.0, 69.0, 68.6, 65.7, 64.1, 53.5, 50.6, 49.3, 48.9, 45.2, 45.0, 39.3, 38.2, 37.2, 36.8, 34.9, 30.0, 21.4, 21.4, 21.0, 19.7, 18.7, 18.0, 17.0, 15.9, 12.3, 10.6, 9.1. HRMS (ESI) m/z Calcd. for C₅₃ H₈₂ O₁₄ N₅ [M+H⁺]: 1012.5853, found 1012.5822.

### Synthesis of Compound 12b.

The reaction of compounds 3b (65 mg, 0.26 mmol), 6 (150 mg, 0.2 mmol) and CuI (10 mg, 0.53 mmol) in THF (1 mL) and Hunig's base (0.1 mL, 0.25 mmol), as described for the synthesis of 12a, followed by prep TLC eluting with DCM:MeOH:NH₄OH=10:1:0.2 furnished 12b (135 mg, 67%) as white powder. ¹H NMR (400 MHz, CDCl₃) δ 7.32-7.21 (m, 3H), 7.19 (d, J=8.1 Hz, 2H), 7.06 (s, 1H), 6.56 (d, J=9.3 Hz, 1H), 5.06-4.99 (m, 1H), 4.88 (d, J=4.8 Hz, 1H), 4.56 (t, J=7.3 Hz, 2H), 4.39 (d, J=7.2 Hz, 1H), 3.96 (d, J=9.7 Hz, 2H), 3.72 (d, J=8.9 Hz, 2H), 3.63 (d, J=7.2 Hz, 1H), 3.48 (s, 1H), 3.22 (d, J=4.6 Hz, 5H), 2.99 (d, J=11.3 Hz, 6H), 2.59 (d, J=0.8 Hz, 30H), 2.36-2.27 (m, 1H), 2.25 (s, 4H), 2.14 (d, J=0.8 Hz, 1H), 2.08 (s, 3H), 1.92-1.75 (m, 2H), 1.67 (d, J=14.5 Hz, 1H), 1.54 (dd, J=15.1, 5.1 Hz, 1H), 1.37 (s, 3H), 1.22 (td, J=15.2, 13.5, 6.9 Hz, 13H), 1.12-1.01 (m, 13H), 0.81 (t, J=7.3 Hz, 3H). ¹³C NMR (101 MHz, CDCl₃) δ 221.1, 175.8, 161.8, 142.8, 140.0, 137.3, 135.2, 129.6, 127.0, 121.4, 115.2, 102.83, 96.1, 81.1, 78.4, 78.0, 77.2, 76.6, 74.3, 72.7, 70.9, 69.0, 68.6, 65.7, 63.7, 51.4, 50.7, 49.4, 49.0, 45.2, 45.0, 40.9, 39.3, 39.1, 37.2, 36.6, 36.4, 34.9, 29.9, 21.5, 21.4, 21.0, 19.7, 18.7, 18.0, 17.0, 16.0, 12.3, 10.6, 9.1. HRMS (ESI) m/z Calcd. for C₅₄ H₈₄ O₁₄ N₅ [M+H⁺]: 1026.6009, found 1026.5978.

### Synthesis of Compound 12c.

The reaction of compounds 3c (150 mg, 0.53 mmol), 6 (80 mg, 0.10 mmol) and CuI (9 mg, 0.05 mmol) in THF (2 mL) DMSO (2 mL) and Hunig's base (0.4 mL, 2.2 mmol), as described for the synthesis of 12a, followed by prep TLC eluting with DCM:MeOH:NH₄OH=10:1:0.2 furnished 12c (85 mg, 79%) as white powder. ¹H NMR (400 MHz, CDCl₃) δ 7.43 (s, 1H), 7.32-7.15 (m, 5H), 7.08 (td, J=1.8, 1.1 Hz, 1H), 6.57 (d, J=9.3 Hz, 1H), 5.02 (dd, J=11.1, 2.2 Hz, 1H), 4.89 (d, J=4.8 Hz, 1H), 4.40 (d, J=7.1 Hz, 1H), 4.32 (td, J=7.0, 1.7 Hz, 2H), 3.96 (s, 1H), 3.83 (d, J=14.0 Hz, 1H), 3.76-3.59 (m, 4H), 3.48 (dd, J=10.6, 5.7 Hz, 1H), 3.30-3.12 (m, 5H), 3.00 (s, 4H), 2.88-2.82 (m, 1H), 2.66 (t, J=7.5 Hz, 2H), 2.62-2.51 (m, 1H), 2.35-2.29 (m, 1H), 2.26 (s, 3H), 2.14 (d, J=0.8 Hz, 7H), 2.12-1.99 (m, 3H), 1.90 (tt, J=14.7, 7.0 Hz, 4H), 1.72-1.59 (m, 3H), 1.54 (dd, J=15.1, 5.0 Hz, 1H), 1.38 (s, 3H), 1.26 (d, J=6.2 Hz, 3H), 1.23-1.14 (m, 8H), 1.11-1.02 (m, 12H), 0.81 (t, J=7.3 Hz, 3H). ¹³C NMR (101 MHz, CDCl₃) δ 221.0, 175.8, 161.8, 142.6, 141.6, 139.1, 135.4, 129.2, 126.5, 121.3, 114.8, 102.7, 96.0, 80.9, 78.3, 78.3, 77.9, 76.5, 74.2, 72.7, 70.9, 69.0, 68.6, 65.7, 64.1, 50.6, 50.1, 49.3, 45.2, 45.0, 39.3, 39.1, 34.9, 34.7, 30.9, 29.7, 28.0, 21.4, 21.0, 19.7, 18.7, 18.0, 17.0, 15.9, 12.3, 10.6, 9.1.

### Synthesis of Compound 13a.

The reaction of compounds 3a (65 mg, 0.27 mmol), 9 (150 mg, 0.2 mmol) and CuI (10 mg, 0.53 mmol) in THF (1 mL) and Hunig's base (0.1 mL, 0.25 mmol), as described for the synthesis of 12a, followed by prep TLC eluting with ethyl acetate:hexane:MeOH=16:1:1 furnished 13a (110 mg, 55%) as pale-yellow powder. ¹H NMR (400 MHz, CDCl₃) δ 7.47 (s, 1H), 7.41-7.31 (m, 4H), 7.30-7.23 (m, 1H), 7.07 (dt, J=2.1, 1.0 Hz, 1H), 6.59 (d, J=9.3 Hz, 1H), 5.28 (s, 1H), 5.15 (d, J=4.7 Hz, 1H), 4.68 (dd, J=9.8, 2.6 Hz, 1H), 4.39 (d, J=7.3 Hz, 2H), 4.22 (dd, J=3.7, 2.0 Hz, 1H), 4.12-4.00 (m, 1H), 3.85 (d, J=13.9 Hz, 1H), 3.72-3.62 (m, 2H), 3.59 (d, J=7.2 Hz, 1H), 3.55-3.46 (m, 1H), 3.35-3.26 (m, 1H), 3.24 (s, 3H), 3.02 (d, J=9.4 Hz, 1H), 2.73-2.65 (m, 2H), 2.61 (d, J=5.8 Hz, 1H), 2.55 (d, J=10.4 Hz, 1H), 2.30 (d, J=15.8 Hz, 7H), 2.16 (d, J=2.6 Hz, 1H), 2.11-2.01 (m, 5H), 1.99-1.83 (m, 1H), 1.79-1.71 (m, 2H), 1.56 (dd, J=15.2, 5.0 Hz, 1H), 1.50-1.39 (m, 1H), 1.31 (d, J=5.5 Hz, 6H), 1.25-1.19 (m, 10H), 1.15 (d, J=7.5 Hz, 3H), 1.08 (t, J=3.4 Hz, 6H), 0.99 (d, J=7.5 Hz, 3H), 0.87 (q, J=7.4, 6.8 Hz, 6H). ¹³C NMR (176 MHz, CDCl₃) δ 178.7, 161.6, 142.9, 141.4, 134.9, 128.9, 127.5, 127.4, 121.5, 115.3, 102.9, 94.6, 83.6, 78.1, 77.8, 74.2, 73.7, 73.5, 73.0, 70.8, 67.0, 69.5, 68.7, 65.6, 64.5, 53.8, 53.5, 49.4, 49.1, 45.3, 42.2, 36.9, 36.3, 34.7, 31.8, 29.7, 29.3, 27.5, 26.7, 22.0, 21.6, 21.3, 18.2, 17.1, 16.3, 14.7, 14.1, 11.3, 9.1, 7.4. HRMS (ESI) m/z Calcd. for C₅₃ H₈₅ O₁₃ N₆ [M+H⁺]: 1013.6169, found 1013.6157.

### Synthesis of Compound 13b.

The reaction of compounds 3b (65 mg, 0.24 mmol), 9 (150 mg, 0.2 mmol) and CuI (10 mg, 0.53 mmol) in THF (1 mL) and Hunig's base (0.1 mL, 0.25 mmol), as described for the synthesis of 12a, followed by prep TLC eluting with DCM:MeOH:NH₄OH=10:1:0.2 furnished 13b (127 mg, 63%) as white powder. ¹H NMR (400 MHz, CDCl₃) δ 7.33 (s, 1H), 7.31-7.24 (m, 3H), 7.19 (d, J=8.1 Hz, 2H), 7.06 (d, J=2.5 Hz, 1H), 6.56 (d, J=9.3 Hz, 1H), 5.12 (d, J=4.8 Hz, 1H), 5.02 (s, 1H), 4.66 (dd, J=9.8, 2.6 Hz, 1H), 4.56 (t, J=7.3 Hz, 2H), 4.38 (d, J=7.2 Hz, 1H), 4.27-4.21 (m, 1H), 4.04 (dt, J=12.4, 6.2 Hz, 1H), 3.81 (d, J=14.0 Hz, 1H), 2.76-2.62 (m, 1H), 2.59 (d, J=1.1 Hz, 2H), 2.51 (d, J=10.3 Hz, 1H), 2.31 (d, J=15.6 Hz, 4H), 2.24 (s, 3H), 2.08 (s, 3H), 1.99 (dd, J=25.6, 8.9 Hz, 2H), 1.80-1.69 (m, 2H), 1.54 (dd, J=15.2, 5.0 Hz, 1H), 1.43 (ddt, J=16.8, 14.3, 7.3 Hz, 1H), 1.28 (d, J=5.7 Hz, 8H), 1.26-1.18 (m, 7H), 1.15 (d, J=7.4 Hz, 3H), 1.12-0.96 (m, 10H), 0.94-0.82 (m, 6H). ¹³C NMR (101 MHz, cdcl3) δ 161.7, 142.7, 140.0, 137.2, 135.1, 129.5, 126.9, 121.4, 115.0, 102.9, 78.1, 77.7, 77.4, 77.1, 76.7, 74.2, 73.6, 73.0, 70.8, 68.6, 65.5, 64.0, 51.3, 49.3, 49.1, 45.2, 42.2, 41.0, 36.7, 36.3, 36.2, 34.7, 27.5, 26.7, 22.0, 21.6, 21.3, 18.2, 17.0, 16.2, 14.6, 11.2, 9.0. HRMS (ESI) m/z Calcd. for C₅₄ H₈₇ O₁₃ N₆ [M+H⁺]: 1027.6326, found 1027.6318.

### Synthesis of Compound 13c.

The reaction of compounds 3c (100 mg, 0.35 mmol), 9 (50 mg, 0.07 mmol) and CuI (10 mg, 0.53 mmol) in THF (1 mL), DMSO (1 mL) and Hunig's base (0.1 mL, 0.57 mmol), as described for the synthesis of 12a, followed by prep TLC eluting with DCM:MeOH: NH₄OH=10:1:0.2 furnished 13c (58 mg, 85%) as white powder. ¹H NMR (400 MHz, CDCl₃) δ 7.45 (s, 1H), 7.32-7.20 (m, 7H), 7.11-7.07 (m, 1H), 6.59 (d, J=9.3 Hz, 1H), 5.15 (d, J=4.8 Hz, 1H), 4.69 (dd, J=9.8, 2.7 Hz, 1H), 4.40 (d, J=7.3 Hz, 1H), 4.34 (td, J=7.0, 1.2 Hz, 2H), 4.26 (dd, J=4.0, 2.0 Hz, 1H), 4.14-4.02 (m, 1H), 3.86 (d, J=13.9 Hz, 1H), 3.75-3.58 (m, 3H), 3.51 (dt, J=13.8, 6.9 Hz, 1H), 3.33 (dd, J=10.2, 7.2 Hz, 2H), 3.25 (s, 3H), 3.01 (d, J=8.8 Hz, 2H), 2.70 (ddd, J=19.3, 8.5, 5.7 Hz, 4H), 2.53 (d, J=9.8 Hz, 1H), 2.30 (d, J=8.8 Hz, 7H), 2.18 (d, J=5.8 Hz, OH), 2.09 (d, J=1.1 Hz, 3H), 2.04-1.84 (m, 3H), 1.77 (t, J=11.3 Hz, 2H), 1.71-1.53 (m, 3H), 1.45 (ddd, J=14.1, 9.7, 7.1 Hz, 1H), 1.31 (t, J=3.2 Hz, 6H), 1.27-1.20 (m, 8H), 1.17 (d, J=7.4 Hz, 3H), 1.08 (t, J=3.4 Hz, 6H), 1.02 (d, J=7.5 Hz, 3H), 0.89 (td, J=7.4, 6.8, 2.6 Hz, 7H). ¹³C NMR (176 MHz, CDCl₃) δ 161.9, 161.8, 149.7, 142.6, 141.7, 139.2, 135.4, 129.2, 126.6, 121.4, 114.9, 102.9, 94.5, 83.5, 78.2, 77.6, 77.5, 74.2, 73.7, 73.0, 70.9, 68.7, 65.6, 64.3, 50.1, 49.3, 45.3, 42.4, 36.9, 36.2, 34.8, 30.1, 29.7, 28.0, 27.6, 26.8, 22.0, 21.6, 21.3, 18.2, 17.0, 16.2, 14.6, 11.3, 9.0, 7.3. HRMS (ESI) m/z Calcd. for C₅₆ H₉₁ O₁₃ N₆ [M+H⁺]: 1055.6639, found 1055.6629.

### Synthesis of Compound 14a.

Compounds 2a (60 mg, 0.20 mmol) and 4 (150 mg, 0.20 mmol) were dissolved in CH₃CN (0.5 mL), DMSO (0.5 mL) and Hunig's base (0.07 mL, 0.39 mmol). The mixture was purged with argon for 20 min, KI (5 mg, 0.03 mmol) was added and the reaction was heat to 75° C. overnight covered with aluminum foil. The reaction was quenched with Na₂S₂O₃ (10 mL) and DCM (30 mL). The two layers were separated and the DCM layer was washed with water (50 mL). The aqueous layer was back extracted with DCM (50 mL) and the combined DCM layer was dried with Na₂SO₄ and evaporated off. The crude was purified using preparative TLC eluting with DCM:MeOH:NH₄OH=10:1:0.2 to furnish 14a (15 mg, 8%) as white powder. ¹H NMR (400 MHz, CDCl₃) δ 7.47-7.38 (m, 1H), 7.36-7.31 (m, 1H), 7.30-7.24 (m, 1H), 7.11 (d, J=1.9 Hz, 1H), 6.61 (d, J=9.3 Hz, 1H), 5.06 (dd, J=11.1, 2.3 Hz, 1H), 4.93 (d, J=4.8 Hz, 1H), 4.48 (d, J=7.1 Hz, 1H), 4.06-3.91 (m, 2H), 3.79-3.74 (m, 2H), 3.68 (d, J=7.4 Hz, 1H), 3.57-3.43 (m, 1H), 3.40-3.30 (m, 1H), 3.27 (s, 2H), 3.20 (s, 1H), 3.07-2.97 (m, 5H), 2.94-2.80 (m, 1H), 2.67-2.55 (m, 2H), 2.36 (d, J=15.2 Hz, 1H), 2.25 (s, 2H), 2.18 (s, 1H), 2.16-2.04 (m, 3H), 1.92 (ddd, J=14.5, 7.4, 2.1 Hz, 1H), 1.87-1.68 (m, 1H), 1.66-1.46 (m, 1H), 1.42 (s, 3H), 1.31 (d, J=6.2 Hz, 2H), 1.28-1.17 (m, 10H), 1.18-1.08 (m, 10H), 0.85 (dd, J=9.6, 5.0 Hz, 3H). ¹³C NMR (101 MHz, CDCl₃) δ 175.9, 161.8, 142.7, 135.3, 130.0, 129.5, 126.6, 121.4, 115.0, 102.7, 96.1, 80.9, 78.4, 77.9, 74.3, 72.7, 71.0, 69.1, 68.7, 65.7, 57.2, 50.6, 49.5, 45.2, 45.1, 39.2, 37.2, 37.0, 34.9, 30.1, 29.7, 21.5, 21.0, 19.8, 18.7, 18.0, 17.0, 16.0, 12.3, 10.6, 9.1. HRMS (ESI) m/z Calcd. for C₅₀ H₇₉ O₁₄ N₂ [M+H⁺]: 931.5526, found 931.5506.

### Synthesis of Compound 14b.

The reaction of compounds 2b (60 mg, 0.20 mmol), 4 (150 mg, 0.20 mmol) and KI (5 mg, 0.03 mmol) dissolved in CH₃CN (0.5 mL), DMSO (0.5 mL) and Hunig's base (0.07 mL, 0.39 mmol), as described for the synthesis of 14a, followed by prep TLC eluting with DCM:MeOH:NH₄OH=10:1:0.2 furnished 14b (11 mg, 6%) as white powder. ¹H NMR (400 MHz, CDCl₃) δ 7.27 (d, J=10.8 Hz, 6H), 7.07 (s, 1H), 6.59 (d, J=9.3 Hz, 1H), 5.08-5.00 (m, 1H), 4.91 (d, J=4.8 Hz, 1H), 4.43 (d, J=7.2 Hz, 1H), 3.97 (d, J=6.5 Hz, 2H), 3.75 (t, J=4.9 Hz, 2H), 3.65 (d, J=7.2 Hz, 1H), 3.48 (s, 1H), 3.31 (s, 3H), 3.18 (s, 1H), 3.02 (s, 5H), 2.87 (t, J=8.1 Hz, 2H), 2.58 (dt, J=12.1, 6.4 Hz, 1H), 2.35 (d, J=15.1 Hz, 3H), 2.19 (d, J=10.5 Hz, 1H), 2.09 (s, 3H), 1.93-1.78 (m, 3H), 1.68 (d, J=14.7 Hz, 1H), 1.58 (dd, J=15.1, 4.8 Hz, 1H), 1.39 (s, 3H), 1.34-1.16 (m, 14H), 1.15-1.04 (m, 12H), 0.83 (t, J=7.3 Hz, 4H). ¹³C NMR (176 MHz, CDCl₃) δ 174.8, 160.8, 141.6, 138.3, 134.2, 128.5, 125.5, 120.4, 113.8, 101.7, 95.1, 79.7, 77.4, 77.0, 73.3, 71.7, 69.8, 68.0, 67.7, 64.9, 54.3, 49.6, 48.5, 44.3, 44.0, 38.3, 38.2, 36.2, 35.7, 33.9, 28.7, 20.5, 20.0, 18.7, 17.7, 17.0, 16.0, 14.9, 11.3, 9.6, 8.1. HRMS (ESI) m/z Calcd. for C₄₆ H₈₁ O₁₆ N₄ [M+H⁺]: 945.5642, found 945.5667.

### Synthesis of Compound 14c.

The reaction of compounds 2c (60 mg, 0.16 mmol), 4 (120 mg, 0.17 mmol) and KI (5 mg, 0.03 mmol) dissolved in CH₃CN (0.5 mL), DMSO (0.5 mL) and Hunig's base (0.1 mL, 0.5.8 mmol), as described for the synthesis of 14a, followed by prep TLC eluting with DCM:MeOH:NH₄OH=10:1:0.2 furnished 14c (19 mg, 12%) as white powder. ¹H NMR (400 MHz, CDCl₃) δ 7.27-7.23 (m, 6H), 7.09 (d, J=2.6 Hz, 1H), 6.58 (d, J=9.3 Hz, 1H), 5.02 (d, J=16.4, 5.2 Hz, 1H), 4.90 (d, J=4.9 Hz, 1H), 4.42 (d, J=7.2 Hz, 1H), 4.04-3.93 (m, 4H), 3.77-3.70 (m, 3H), 3.64 (d, J=7.4 Hz, 1H), 3.49-3.44 (m, 1H), 3.30 (s, 4H), 3.18 (q, J=6.3, 5.6 Hz, 2H), 3.05-2.91 (m, 8H), 2.86 (dd, J=9.2, 7.1 Hz, 1H), 2.72-2.48 (m, 6H), 2.35 (d, J=15.2 Hz, 1H), 2.21 (d, J=4.7 Hz, 5H), 2.08 (d, J=1.1 Hz, 3H), 1.94-1.78 (m, 3H), 1.72-1.52 (m, 4H), 1.38 (s, 2H), 1.28 (d, J=6.2 Hz, 2H), 1.24-1.13 (m, 8H), 1.13-1.00 (m, 12H), 0.82 (t, J=7.4 Hz, 2H). ¹³C NMR (101 MHz, CDCl₃) δ 175.9, 161.9, 142.6, 138.9, 135.5, 129.2, 126.4, 121.3, 114.9, 102.8, 96.1, 80.7, 78.4, 74.3, 72.7, 70.7, 69.0, 68.8, 65.7, 52.9, 50.6, 49.5, 45.3, 45.0, 39.2, 37.0, 35.4, 34.9, 29.5, 28.8, 27.9, 21.5, 21.0, 19.8, 18.7, 18.0, 17.0, 15.9, 12.3, 10.6, 9.0. HRMS (ESI) m/z Calcd. for C₅₃ H₈₅ O₁₄ N₂ [M+H⁺]: 973.5995, found 973.5978.

### Synthesis of Compound 15a.

The reaction of compounds 2a (60 mg, 0.20 mmol), 7 (150 mg, 0.20 mmol) and KI (5 mg, 0.03 mmol) dissolved in CH₃CN (2 mL), DMSO (2 mL) and Hunig's base (0.07 mL, 0.42 mmol), as described for the synthesis of 14a, followed by prep TLC eluting with DCM:MeOH:NH₄OH=10:1:0.2 furnished 15a (10.5 mg, 5%) as pale-yellow powder. ¹H NMR (400 MHz, CDCl₃) δ 7.40-7.13 (m, 5H), 7.03 (s, 1H), 6.53 (d, J=9.3 Hz, 1H), 5.12 (d, J=4.8 Hz, 1H), 4.63 (d, J=9.7 Hz, 1H), 4.40 (d, J=7.4 Hz, 1H), 4.20 (s, 1H), 4.06-3.97 (m, 1H), 3.74 (d, J=13.4 Hz, 1H), 3.61 (s, 1H), 3.56 (d, J=7.4 Hz, 1H), 3.44 (t, J=13.8 Hz, 1H), 3.31 (dd, J=17.2, 8.7 Hz, 1H), 3.21 (s, 2H), 3.08-3.03 (m, 1H), 2.97 (s, 1H), 2.70-2.56 (m, 1H), 2.55 (s, 3H), 2.48 (d, J=9.7 Hz, 1H), 2.33-2.20 (m, 5H), 2.18 (s, 2H), 2.04 (d, J=10.4 Hz, 4H), 1.72 (d, J=13.9 Hz, 1H), 1.44 (dd, J=55.6, 15.0, 6.3 Hz, 1H), 1.27 (d, J=6.4 Hz, 5H), 1.17 (d, J=4.4 Hz, 5H), 1.12 (d, J=7.4 Hz, 2H), 1.06-0.91 (m, 7H), 0.82 (q, J=7.5, 6.4 Hz, 6H). ¹³C NMR (176 MHz, CDCl₃) δ 161.8, 142.6, 140.1, 139.4, 135.3, 129.4, 126.5, 121.5, 114.9, 102.9, 94.5, 83.5, 78.2, 74.2, 73.7, 73.00 71.0, 70.0, 68.7, 65.6, 62.5, 57.43, 49.5, 45.3, 42.3, 41.0, 37.1, 36.2, 34.7, 30.3, 29.7, 27.6, 26.8, 22.0, 21.7, 21.4, 18.2, 17.0, 16.2, 14.6, 11.3, 9.0, 7.3. HRMS (ESI) m/z Calcd. for C₅₀ H₈₂ O₁₃ N₃ [M+H⁺]: 932.5842, found 932.5824.

### Synthesis of Compound 15b.

The reaction of compounds 2b (500 mg, 1.4 mmol), 7 (200.5 mg, 0.28 mmol) and KI (40 mg, 0.24 mmol), dissolved in CH₃CN (2 mL), DMSO (2 mL) and Hunig's base (0.5 mL, 2.92 mmol), as described for the synthesis of 14a, followed by prep TLC eluting with DCM:MeOH:NH₄OH=10:1:0.2 furnished 15b (12 mg, 1%) as pale-yellow powder. ¹H NMR (400 MHz, CDCl₃) δ 7.37-7.13 (m, 5H), 7.07 (d, J=6.4 Hz, 1H), 6.57 (d, J=9.3 Hz, 1H), 5.27 (s, 1H), 5.02 (d, J=4.8 Hz, 1H), 4.65 (d, J=9.9 Hz, 1H), 4.42 (d, J=7.2 Hz, 1H), 4.25 (d, J=5.2 Hz, 1H), 4.04 (dq, J=12.6, 6.3 Hz, 1H), 3.84 (t, J=6.9 Hz, OH), 3.68-3.59 (m, 2H), 3.47 (dq, J=16.5, 9.1, 7.5 Hz, 1H), 3.30 (s, 2H), 3.21 (dd, J=10.3, 7.4 Hz, OH), 3.00 (t, J=9.8 Hz, 1H), 2.81 (tq, J=20.7, 7.0 Hz, 3H), 2.75-2.56 (m, 1H), 2.47 (d, J=11.2 Hz, 1H), 2.39-2.15 (m, 6H), 2.07 (s, 5H), 1.89-1.72 (m, 1H), 1.72-1.40 (m, 2H), 1.28 (d, J=6.9 Hz, 5H), 1.25-1.12 (m, 9H), 1.12-0.95 (m, 7H), 0.95-0.71 (m, 5H). ¹³C NMR (101 MHz, CDCl₃) δ 161.8, 142.5, 139.2, 135.4, 129.9, 129.5, 126.4, 121.4, 114.8, 102.8, 95.0, 83.7, 78.4, 78.1, 74.3, 73.6, 72.9, 70.7, 68.7, 65.8, 65.5, 55.3, 53.5, 49.5, 45.1, 42.4, 41.7, 38.6, 36.8, 36.5, 34.9, 29.7, 26.7, 22.0, 21.6, 21.4, 18.3, 17.0, 16.3, 15.2, 11.3, 9.2. HRMS (ESI) m/z Calcd. for C₅₁ H₈₄ O₁₃ N₃ [M+H⁺]: 946.5999, found 946.5982.

### Synthesis of Compound 15c.

### Route 1

The reaction of compounds 2c (300 mg, 0.95 mmol), 7 (700 mg, 0.95 mmol) and KI (40 mg, 0.24 mmol) dissolved in CH₃CN (2 mL), DMSO (2 mL) and Hunig's base (0.4 mL, 1.4 mmol), as described for the synthesis of 14a, followed by prep TLC eluting with DCM:MeOH:NH₄OH=10:1:0.2 furnished 15c (75.8 mg, 9%) as pale-yellow powder.

### Synthesis of Compound 15c.

### Route 2 (Scheme 3 g)

A mixture of compound 1c (400 mg, 1.55 mmol), tosyl chloride (441 mg, 2.33 mmol), TEA (0.31 mL, 3.1 mmol) DCM (10 mL) was stirred at rt overnight. The reaction was partitioned between sat. NaHCO₃ (50 mL) and DCM (30 mL) and the two layers separated. The aqueous layer was extracted with DCM (30 mL×2), the combined organic layer was washed with water (100 mL), brine (50 mL) and dried over Na₂SO₄. Solvent was evaporated and the crude was purified using column chromatography eluting with EtOAc:MeOH=15:1 to 10:1 to furnish 2d as a clear yellow oil (560 mg, 88%).

A mixture of 2d (500 mg, 1.58 mmol), 7 (1500 mg, 2.03 mmol) in Hunig's Base (7 mL) and CH₃CN (3 mL) was stirred with heating at 75° C.-80° C. overnight. The reaction was partitioned between water (100 mL) and DCM (50 mL) and the two layers separated. The aqueous layer was extracted with DCM (30 mL×2), the combined organic layer was washed with brine (30 mL) and dried over Na₂SO₄. Solvent was evaporated off and the crude was purified using column chromatography eluting with a gradient of DCM:MeOH:NH₄OH=10:1:0.2 to DCM:MeOH:NH₄OH=8:1.5:0.1 to furnish 15c (820 mg, 53.3%) as pale-yellow powder. ¹H NMR (400 MHz, CDCl₃) δ 7.31-7.23 (m, 6H), 7.12 (s, 1H), 6.60 (d, J=9.3 Hz, 1H), 5.33-5.28 (m, 1H), 5.05 (d, J=4.7 Hz, 1H), 4.71-4.64 (m, 1H), 4.47 (d, J=7.2 Hz, 1H), 4.32-4.26 (m, 1H), 4.07 (dt, J=12.6, 6.3 Hz, 1H), 3.71-3.63 (m, 2H), 3.52 (s, 1H), 3.34 (s, 3H), 3.26 (t, J=8.9 Hz, 1H), 3.04 (t, J=9.7 Hz, 1H), 2.85-2.77 (m, 2H), 2.62 (s, 7H), 2.50 (d, J=11.2 Hz, 1H), 2.34 (s, 3H), 2.23 (d, J=15.2 Hz, 3H), 2.10 (s, 3H), 2.00 (t, J=7.2 Hz, 1H), 1.93-1.77 (m, 1H), 1.61 (ddd, J=30.4, 15.1, 6.6 Hz, 3H), 1.32 (d, J=6.4 Hz, 6H), 1.26 (s, 4H), 1.12-1.03 (m, 8H), 1.00-0.81 (m, 7H). ¹³C NMR (101 MHz, CDCl₃) δ 161.3, 142.0, 138.3, 128.7, 125.8, 120.8, 114.2, 102.4, 94.5, 83.3, 77.9, 77.6, 73.8, 73.0, 72.4, 70.1, 68.2, 65.0, 53.0, 48.9, 44.5, 40.4, 36.0, 34.9, 34.4, 31.1, 29.2, 28.4, 26.2, 22.1, 21.5, 21.1, 20.9, 20.6, 17.8, 16.5, 15.7, 14.7, 13.6, 10.8, 8.7, 7.2. HRMS (ESI) m/z Calcd. for C₅₃ H₈₈ O₁₃ N₃ [M+H⁺]: 974.6312, found 974.6288.

Synthesis of Compound 16 (Ma, Z.; Pan, Y.; Huang, W.; Yang, Y.; Wang, Z.; Li, Q.; Zhao, Y.; Zhang, X.; Shen, Z. Synthesis and biological evaluation of the pirfenidone derivatives as antifibrotic agents. Bioorg. Med. Chem. Lett. 2014, 24, 220-223).

2-Hydroxy-5-methyl pyridine (500 mg, 4.58 mmol) was added to potassium carbonate (900 mg, 6.42 mmol), 8-hydroxyquinoline (220 mg, 1.49 mmol) and 4-iodoanisole (700 mg, 2.97 mmol) in a round bottom flask. Dimethyl sulfoxide (DMSO) (30 mL) was added to the mixture and the mixture was purged with argon for 15 min. Then, copper (I) iodide (CuI) (250 mg, 1.49 mmol) was added to the mixture. The pressure tube was capped after another 15 min of argon purge. The reaction was covered with aluminum foil and heated to 120° C. for 12 h. The reaction was cooled to room temperature and the resulting green mixture was worked up as described for the synthesis of 1a. The crude was purified using silica gel chromatography eluting with EtOAc:methanol=10:0.7 to furnish 16 (510 mg, 52%) as pale-yellow solid. ¹H NMR (400 MHz, CDCl₃) δ 7.30-7.22 (m, 3H), 7.09 (ddd, J=2.6, 1.2, 0.7 Hz, 1H), 7.01-6.95 (m, 2H), 6.60 (d, J=9.6 Hz, 1H), 3.84 (s, 3H), 2.10 (s, 3H).

### Synthesis of Compound 17.

Compound 16 (500 mg, 2.37 mmol) was dissolved in DCM (10 mL) and the solution was purged with argon gas for 15 min and cooled to −30° C. BBr₃ (2 mL) was added to the solution dropwisely and stirring continued at −30° C. for 1 h. The reaction temperature was raised to 0° C. (ice bath) and stirring continued overnight. MeOH (20 mL) was added dropwisely, followed by NaHCO₃ to adjust pH to 8-9 and precipitate resulted. The suspension was filtered through vacuum and the filtrate was extracted by DCM:MeOH=10:1 (100 mL×5). The combined DCM layer was dried over Na₂SO₄ and evaporated off to furnish 17 (320 mg, 67%). ¹H NMR (400 MHz, CDCl₃) δ 7.35 (ddd, J=9.2, 2.6, 0.4 Hz, 1H), 7.16-7.11 (m, 1H), 7.04-6.95 (m, 2H), 6.65 (t, J=9.0 Hz, 3H), 2.12 (d, J=1.0 Hz, 3H).

### Synthesis of Compound 18a.

Compound 17 (300 mg, 1.49 mmol) was dissolved in DCM (5 mL) and cooled to −10 to −20° C. Pyridine (0.7 mL, 8.6 mmol) was added to the solution and triflic anhydride (0.4 mL, 2.38 mmol) was added dropwisely. The reaction was complete within 30 min and 1M HCl solution (5 mL) was added to the solution to quench the reaction. Then DCM (30 mL) and water (50 mL) were added and the two layers separated. The DCM layer was washed water (30 mL), dried over Na₂SO₄ and evaporated off. The crude product (dark yellow solid) was purified using column chromatography eluting with neat EtOAc to furnish the triflate intermediate product as pale-yellow solid (320 mg, 51%). The intermediate triflate compound (300 mg, 0.9 mmol) was treated with prop-2-yn-1-ol (100.1 mg, 1.8 mmol), Tetrakis(triphenylphosphine)palladium (520 mg, 0.45 mmol) and CuI (85.5 mg, 0.45 mmol) in CH₃CN (7 mL). The mixture was purged with argon for 5 min and after the addition of Hunig's base (0.7 ml, 4 mmol), heated to 75° C. and kept stirring overnight. The mixture was filtered through celite bed and the filtrate was evaporated off. The crude compound was purified using column chromatography, eluting with EtOAc:MeOH=13:1 to furnish 18a (323 mg, 90%) as yellow oil. ¹H NMR (400 MHz, CDCl₃) δ 7.57-7.51 (m, 2H), 7.41-7.30 (m, 2H), 7.30-7.23 (m, 1H), 7.09 (ddd, J=2.6, 1.2, 0.8 Hz, 1H), 6.64-6.57 (m, 1H), 4.51 (d, J=6.2 Hz, 2H), 2.11 (d, J=1.1 Hz, 4H), 1.88 (t, J=6.2 Hz, 1H).

### Synthesis of Compound 18b.

The triflate intermediate (400 mg, 1.2 mmol) was prepared as described for the synthesis of 18a. The triflate intermediate was treated with 3-butyn-1-ol (500 mg, 6.84 mmol), Tetrakis(triphenylphosphine)palladium (200 mg, 0.17 mmol) and CuI (33 mg, 0.17 mmol) in CH₃CN (10 mL). The mixture was purged with argon for 5 min and after the addition of Hunig's base (1 mL, 10 v/v %), heated to 65° C. and kept stirring overnight. The mixture was filtered through celite bed and the filtrate was evaporated off. The crude compound was purified using column chromatography, eluting with neat EtOAc to furnish 18b (370 mg, 84%) as yellow oil. ¹H NMR (400 MHz, CDCl₃) δ 7.57-7.43 (d, J=8.3 Hz, 2H), 7.40-7.29 (d, J=8.3 Hz, 2H), 7.26 (d, J=0.6 Hz, 1H), 7.09 (d, J=2.3 Hz, 1H), 6.60 (d, J=9.4 Hz, 1H), 3.83 (q, J=6.3 Hz, 2H), 2.71 (t, J=6.3 Hz, 2H), 2.11 (d, J=1.1 Hz, 3H), 1.83 (t, J=6.3 Hz, 1H).

### Synthesis of Compound 18c.

The triflate intermediate (200 mg, 0.58 mmol) was prepared as described for the synthesis of 18a. The triflate intermediate was treated with pent-4-yn-1-ol (0.135 g, 1.6 mmol), Tetrakis(triphenylphosphine)palladium (320 mg, 0.25 mmol) and CuI (68 mg, 0.25 mmol) in CH₃CN (10 mL). The mixture was purged with argon for 5 min and after the addition of Hunig's base (0.8 mL, 8 v/v %), heated to 65° C. and kept stirring overnight. The mixture was filtered through celite bed and the filtrate was evaporated off. The crude compound was purified using column chromatography, eluting with neat EtOAc to furnish 18c (150 mg, 97%) as yellow oil. ¹H NMR (400 MHz, CDCl₃) δ 7.52-7.44 (d, J=8.3 Hz, 2H), 7.34-7.28 (d, J=8.3 Hz, 2H), 7.26 (m, 1H), 7.08 (dt, J=2.7, 1.0 Hz, 1H), 6.60 (d, J=9.3 Hz, 1H), 3.83 (q, J=6.0 Hz, 2H), 2.56 (t, J=7.0 Hz, 2H), 2.10 (s, J=1.1 Hz, 3H), 1.87 (tt, J=7.0, 6.2 Hz, 2H), 1.51 (t, J=5.4 Hz, 1H).

### Synthesis of Compound 18d.

The triflate intermediate (500 mg, 1.5 mmol) was prepared as described for the synthesis of 18a. The triflate intermediate was treated with hex-5-yn-1-ol (294 mg, 3 mmol), Tetrakis(triphenylphosphine)palladium (433 mg, 0.375 mmol) and CuI (71 mg, 0.375 mmol) in CH₃CN (8 mL). The mixture was purged with argon for 5 min and after the addition of Hunig's base (0.8 mL, 8 v/v %), heated to 65° C. and kept stirring overnight. The mixture was filtered through celite bed and the filtrate was evaporated off. The crude compound was purified using column chromatography, eluting with neat EtOAc to furnish 18d (400 mg, 95%) as yellow oil. ¹H NMR (400 MHz, CDCl₃) δ 7.48 (d, J=8.6 Hz, 2H), 7.32-7.28 (d, J=8.6 Hz, 2H), 7.29-7.24 (m, 1H), 7.09-7.05 (m, 1H), 6.60 (d, J=9.3 Hz, 1H), 3.73 (s, 2H), 2.48 (t, J=6.6 Hz, 2H), 2.14-2.03 (m, 3H), 1.80-1.65 (m, 2H), 1.28 (d, J=18.0 Hz, 3H).

### Synthesis of Compound 18e.

The triflate intermediate (200 mg, 0.58 mmol) was prepared as described for the synthesis of 18a. The triflate intermediate was treated with hept-6-yn-1-ol (180 mg, 1.6 mmol), Tetrakis(triphenylphosphine)palladium (320 mg, 0.25 mmol) and CuI (68 mg, 0.25 mmol) in CH₃CN (10 mL). The mixture was purged with argon for 5 min and after the addition of Hunig's base (0.8 mL, 8 v/v %), heated to 65° C. and kept stirring overnight. The mixture was filtered through celite bed and the filtrate was evaporated off. The crude compound was purified using column chromatography, eluting with neat EtOAc to furnish 18e (160 mg, 0.54 mmol, 93.2%) as yellow oil. ¹H NMR (400 MHz, CDCl₃) δ 7.48 (d, J=8.6 Hz, 2H), 7.33-7.28 (d, J=8.6 Hz, 1H), 7.27-7.24 (m, 1H), 7.10-7.05 (m, 1H), 6.60 (d, J=9.4 Hz, 1H), 3.69 (q, J=6.0 Hz, 2H), 2.44 (t, J=6.9 Hz, 2H), 2.10 (d, J=1.1 Hz, 3H), 1.65 (h, J=6.9 Hz, 4H), 1.69-1.51 (m, 4H).

### Synthesis of Compound 19a.

Compound 18a (170 mg, 0.7 mmol) was dissolved in DCM (15 mL) and triethylamine (360 mg, 3.5 mmol) and stirred at −20° C. Methanesulfonyl chloride (0.20 g, 3.0 mmol) was added slowly to the solution under argon; the reaction was kept stirring for 45-60 min and quenched with NaHCO₃ (1 mL). The reaction was partitioned between DCM (50 mL) and water (50 mL) and the two layers separated. The organic layer was washed with water (30 mL), dried over Na₂SO₄ and evaporated off to give a crude mesylated intermediate that was used without purification. The mesylated intermediate (85 mg, 0.27 mmol) was dissolved in THF/DMSO solution (1:1 mL); and compound 4 (170 mg, 0.26 mmol) and Hunig's base (0.68 mL, 4 mmol) were added to the solution. The mixture was heated to 50° C. for 2 h during which the starting materials were consumed. The solution was worked up with water (50 mL) and chloroform (30 mL×3). The organic layer was dried over Na₂SO₄ and the solvent was evaporated off. The crude product was purified using prep TLC plate eluting with EtOAc:MeOH=15:1 to furnish 19a as pale-yellow solid (135 mg, 54%). ¹H NMR (400 MHz, CDCl₃) δ 7.52-7.44 (m, 2H), 7.35-7.28 (m, 2H), 7.27-7.22 (m, 1H), 7.07 (d, J=7.1 Hz, 1H), 6.58 (d, J=9.4 Hz, 1H), 5.04 (dd, J=11.2, 2.3 Hz, 1H), 4.91 (d, J=4.9 Hz, 1H), 4.47 (d, J=7.2 Hz, 1H), 3.98 (d, J=11.4 Hz, 2H), 3.74 (q, J=6.4, 5.3 Hz, 2H), 3.57-3.45 (m, 1H), 3.26 (s, 3H), 3.17 (s, 1H), 3.01 (d, J=10.6 Hz, 4H), 2.93-2.81 (m, 1H), 2.81-2.67 (m, 1H), 2.57 (dt, J=7.7, 4.1 Hz, 1H), 2.40 (s, 2H), 2.34 (d, J=15.2 Hz, 1H), 2.19 (d, J=10.0 Hz, 1H), 2.10 (d, J=6.0 Hz, 3H), 1.97-1.78 (m, 3H), 1.74-1.62 (m, 1H), 1.60-1.42 (m, 1H), 1.42 (s, OH), 1.38-1.32 (m, 1H), 1.29 (d, J=6.1 Hz, 3H), 1.25-1.16 (m, 8H), 1.16-1.00 (m, 10H), 0.83 (t, J=7.3 Hz, 3H). ¹³C NMR (101 MHz, CDCl₃) δ 175.8, 161.5, 142.7, 140.7, 134.7, 132.4, 126.8, 126.6, 123.2, 121.5, 115.1, 102.7, 96.0, 87.2, 83.9, 80.8, 78.4, 77.9, 74.2, 72.6, 71.1, 69.0, 68.6, 65.7, 64.3, 50.6, 49.5, 45.2, 45.0, 44.5, 39.3, 37.2, 35.9, 21.5, 21.0, 19.8, 18.7, 18.0, 17.0, 16.0, 12.3, 10.6, 9.1. HRMS (ESI) m/z Calcd. for C₅₂ H₇₉ O₁₄ N₂ [M+H⁺]: 955.5526, found 955.5505.

### Synthesis of Compound 19b.

The preparation of mesylated intermediate was as described for synthesis of mesylated intermediate to 19a. The prepared intermediate (100 mg, 0.32 mmol) was reacted with compound 4 (200 mg, 0.28 mmol) in THF/DMSO (1:1 mL) and Hunig's base (0.7 mL, 7 V/V %) at 75-80° C. overnight. The work-up procedure was described in synthesis procedure of 19a. The crude product was dried and purified through prep TLC eluting with EtOAc:MeOH=20:1 to furnish 19b as yellow solid (22 mg, 8%). ¹H NMR (400 MHz, CDCl₃) δ 7.54-7.47 (d, J=6.8, 2H), 7.35-7.22 (m, 2H), 7.26 (m, 1H), 7.07 (s, 1H), 6.60 (d, J=9.4 Hz, 1H), 5.04 (d, J=9.3 Hz, 1H), 4.92 (d, J=4.8 Hz, 1H), 4.48 (d, J=7.1 Hz, 1H), 3.98 (d, J=7.4 Hz, 2H), 3.87-3.74 (m, 2H), 3.75 (d, J=4.7 Hz, 1H), 3.66 (d, J=7.1 Hz, 1H), 3.31 (s, 3H), 3.25 (s, 1H), 3.19 (d, J=2.4 Hz, 1H), 3.03 (s, 4H), 2.90-2.80 (m, 2H), 2.71 (t, J=6.3 Hz, 1H), 2.60 (s, 6H), 2.36 (d, J=15.3 Hz, 1H), 2.33 (s, 4H), 2.10 (d, J=1.2 Hz, 4H), 1.86 (dd, J=26.5, 11.6 Hz, 4H), 1.58 (dd, J=15.0, 4.9 Hz, 1H), 1.40 (s, 3H), 1.30 (d, J=6.2 Hz, 3H), 1.25 (s, 8H), 1.26-1.16 (m, 8H), 1.15-1.08 (m, 12H), 0.84 (q, J=7.6 Hz, 3H). ¹³C NMR (176 MHz, CDCl₃) δ 220.7, 188.0, 175.4, 164.3, 161.6, 142.7, 140.0, 135.1, 128.7, 127.3, 121.4, 115.1, 102.5, 96.1, 81.9, 78.3, 78.1, 77.6, 76.7, 74.2, 73.1, 71.8, 69.1, 67.9, 66.2, 58.8, 50.6, 49.6, 45.1, 45.0, 39.1, 38.6, 37.3, 35.0, 21.5, 20.9, 19.7, 18.7, 18.0, 17.0, 16.1, 12.3, 10.6, 9.9. HRMS (ESI) m/z Calcd. for C₅₃ H₈₁ O₁₄ N₂ [M+H⁺]: 969.5682, found 969.5662.

### Synthesis of Compound 19c.

The preparation of mesylated intermediate was as described for synthesis of mesylated intermediate to 19a. The prepared intermediate (75 mg, 0.23 mmol) was reacted with compound 4 (200 mg, 0.27 mmol) in THF/DMSO (5:2 mL) and Hunig's base (0.8 mL, 8 V/V %) at 75-80° C. overnight. The work-up procedure was described in synthesis procedure of 19a. The crude product was dried and purified through prep TLC eluting with EtOAc:MeOH=20:1 to furnish 19c as yellow solid (19 mg, 8%). ¹H NMR (400 MHz, CDCl₃) δ 7.48 (d, J=8.7, 2.6 Hz, 2H), 7.32-7.21 (m, 3H), 7.06 (s, 1H), 6.59 (d, J=9.4 Hz, 1H), 5.04 (d, J=11.1, 2.3 Hz, 1H), 4.91 (d, J=4.8 Hz, 1H), 4.46 (d, J=7.2 Hz, 1H), 4.11 (q, J=7.1 Hz, 1H), 3.99 (s, 1H), 3.97 (s, 1H), 3.79-3.72 (m, 2H), 3.66 (d, J=7.2 Hz, 1H), 3.49 (s, 1H), 3.28 (s, 2H), 3.17 (s, 1H), 3.01 (d, J=14.2 Hz, 5H), 2.93-2.83 (m, 1H), 2.55 (d, J=9.1 Hz, 2H), 2.48 (d, J=7.5 Hz, 2H), 2.35 (d, J=15.2 Hz, 1H), 2.29 (s, 2H), 2.15 (s, 1H), 2.06 (d, J=22.8 Hz, 4H), 1.95-1.82 (m, 2H), 1.81 (s, 1H), 1.69 (d, J=15.7 Hz, 2H), 1.57 (dd, J=15.2, 4.9 Hz, 1H), 1.39 (s, 3H), 1.26 (s, 3H), 1.32-1.15 (m, 13H), 1.14-1.07 (m, 10H), 0.84 (dd, J=14.8, 7.3 Hz, 6H), ¹³C NMR (101 MHz, CDCl₃) δ 221.0, 175.8, 161.7, 142.8, 134.0, 137.3, 135.2, 129.6, 127.0, 121.3, 115.2, 102.8, 96.1, 81.0, 78.3, 78.0, 76.6, 74.2, 72.7, 70.9, 69.0, 68.5, 65.7, 63.7, 51.4, 50.6, 49.3, 49.0, 45.2, 45.0, 39.3, 39.1, 37.2, 36.5, 36.3, 34.9, 29.7, 21.4, 21.0, 19.7, 18.7, 18.0, 17.0, 16.0, 12.3, 10.6, 9.1. HRMS (ESI) m/z Calcd. for C₅₄ H₈₃ O₁₄ N₂ [M+H⁺]: 983.5839, found 983.5817.

### Synthesis of Compound 19d.

The preparation of mesylated intermediate was as described for synthesis of mesylated intermediate to 19a. The prepared intermediate (170 mg, 0.54 mmol) was reacted with compound 4 (370 mg, 0.52 mmol) in THF/DMSO (5:1 mL) and Hunig's base (0.6 mL, 8 V/V %) at 75-80° C. overnight. The work-up procedure was described in synthesis procedure of 19a. The crude product was dried and purified through prep TLC eluting with EtOAc:MeOH=20:1 to furnish 19d as yellow solid (21 mg, 4%). ¹H NMR (400 MHz, CDCl₃) δ 7.47 (d, J=8.5 Hz, 2H), 7.30 (d, J=8.5 Hz, 2H), 7.25 (d, J=5.0 Hz, 1H), 7.09-7.05 (m, 1H), 6.60 (d, J=9.3 Hz, 1H), 5.04 (dd, J=11.1, 2.2 Hz, 1H), 4.92 (d, J=5.0 Hz, 2H), 4.47 (d, J=7.2 Hz, 2H), 3.99 (q, J=5.5, 5.1 Hz, 3H), 3.79-3.70 (m, 4H), 3.66 (d, J=7.3 Hz, 1H), 3.50 (s, 2H), 3.31 (d, J=4.3 Hz, 6H), 3.02 (d, J=5.4 Hz, 9H), 2.88 (dd, J=9.3, 7.1 Hz, 2H), 2.64-2.53 (m, 2H), 2.48-2.25 (m, 9H), 2.11-2.05 (m, 5H), 1.94-1.79 (m, 4H), 1.74-1.48 (m, 2H), 1.39 (s, 3H), 1.33-1.16 (m, 13H), 1.15-1.05 (m, 10H), 0.93-0.75 (m, 6H). ¹³C NMR (101 MHz, CDCl₃) δ 221.04, 175.8, 161.6, 142.7, 140.1, 134.9, 132.4, 126.4, 124.1, 121.5, 115.1, 96.1, 78.3, 77.9, 77.3, 77.0, 76.7, 74.3, 72.7, 70.7, 69.1, 50.6, 49.5, 45.3, 45.0, 39.3, 37.2, 34.9, 31.9, 29.7, 29.4, 26.1, 22.7, 21.5, 21.4, 21.0, 19.8, 19.3, 18.7, 18.0, 17.0, 16.0, 14.1, 12.3, 10.6, 9.1. HRMS (ESI) m/z Calcd. for C₅₅ H₈₅ O₁₄ N₂ [M+H⁺]: 997.5995, found 997.5977.

### Synthesis of Compound 19e.

The preparation of mesylated intermediate was as described for synthesis of mesylated intermediate to 19a. The prepared intermediate (85 mg, 0.236 mmol) was reacted with compound 4 (230 mg, 0.31 mmol) in THF/DMSO (5:5 mL) and Hunig's base (0.8 mL, 8 V/V %) at 75-80° C. overnight. The work-up procedure was described in synthesis procedure of 19a. The crude product was dried and purified through prep TLC eluting with EtOAc:MeOH=20:1 to furnish 19e as yellow solid (11 mg, 5%). ¹H NMR (400 MHz, CDCl₃) δ 7.46 (d, J=8.5 Hz, 2H), 7.28 (d, J=8.5 Hz, 2H), 7.26-7.22 (m, 1H), 7.06 (dt, J=2.3, 1.1 Hz, 1H), 6.57 (d, J=9.3 Hz, 1H), 5.03 (dd, J=11.1, 2.3 Hz, 1H), 4.90 (d, J=4.8 Hz, 1H), 4.43 (d, J=7.2 Hz, 1H), 4.04-3.93 (m, 3H), 3.78-3.71 (m, 2H), 3.64 (d, J=7.3 Hz, 1H), 3.52-3.42 (m, 1H), 3.31 (s, 4H), 3.24-3.16 (m, 2H), 3.02-2.93 (m, 6H), 2.62-2.51 (m, 2H), 2.40 (t, J=7.0 Hz, 2H), 2.35 (d, J=15.2 Hz, 1H), 2.25 (s, 3H), 2.08 (d, J=1.1 Hz, 3H), 1.94-1.78 (m, 4H), 1.73-1.43 (m, 5H), 1.38 (s, 3H), 1.28 (d, J=6.1 Hz, 3H), 1.26-1.15 (m, 12H), 1.09 (dt, J=7.6, 4.0 Hz, 13H), 0.88-0.78 (m, 6H). ¹³C NMR (101 MHz, CDCl₃) δ 175.8, 161.6, 142.7, 140.0, 134.9, 132.3, 126.4, 124.2, 121.5, 115.0, 102.7, 96.0, 91.4, 80.7, 80.1, 78.3, 77.9, 74.2, 72.7, 70.7, 69.0, 65.7, 50.6, 49.5, 45.3, 45.0, 39.2, 36.9, 34.9, 31.6, 29.7, 28.5, 26.4, 22.6, 21.5, 21.0, 19.8, 19.4, 18.7, 18.0, 17.0, 15.9, 14.1, 12.3, 10.6, 9.0. HRMS (ESI) m/z Calcd. for C₅₆ H₈₇ O₁₄N₂ [M+H⁺]: 1011.6152, found 1011.6123.

### Synthesis of Compound 20a.

The preparation of mesylated intermediate was as described for synthesis of mesylated intermediate to 19a. Subsequently, the mesylated intermediate (85 mg, 0.27 mmol) was reacted with compound 7 (170 mg, 0.27 mmol) in THF/DMSO (5:5 mL) and Hunig's base (0.68 mL, 4 mmol) at 50° C. for 2 h. The reaction was worked up with water (50 mL) and chloroform (30 mL×3). The crude product was purified through prep TLC eluting with EtOAc:MeOH=15:1 to furnish 20a as pale-yellow solid (115 mg, 45%). ¹H NMR (400 MHz, CDCl₃) δ 7.52-7.46 (m, 2H), 7.36-7.29 (m, 2H), 7.25 (d, J=9.8 Hz, 1H), 7.06 (d, J=2.6 Hz, 1H), 6.59 (d, J=9.3 Hz, 1H), 5.12 (d, J=4.8 Hz, 1H), 4.82 (s, 1H), 4.68 (dd, J=9.9, 2.6 Hz, 1H), 4.47 (d, J=7.2 Hz, 1H), 4.27 (dd, J=4.4, 2.0 Hz, 1H), 4.06 (dt, J=12.5, 6.4 Hz, 1H), 3.68 (s, 1H), 3.55 (dt, J=11.0, 7.5 Hz, 1H), 3.28 (s, 4H), 3.21 (s, 1H), 3.02 (t, J=9.9 Hz, 1H), 2.92 (s, 1H), 2.81-2.63 (m, 3H), 2.53 (d, J=10.5 Hz, 1H), 2.41 (s, 3H), 2.31 (s, 4H), 2.18-2.14 (m, 3H), 2.09 (s, 3H), 2.08-1.95 (m, 2H), 1.87 (ddd, J=15.6, 9.2, 4.1 Hz, 2H), 1.79 (d, J=14.6 Hz, 1H), 1.56 (dd, J=15.2, 5.0 Hz, 1H), 1.50-1.35 (m, 1H), 1.32 (t, J=3.1 Hz, 6H), 1.26-1.14 (m, 8H), 1.13-1.00 (m, 8H), 0.89 (q, J=7.4, 6.6 Hz, 6H). ¹³C NMR (101 MHz, CDCl₃) δ 178.9, 161.7, 142.8, 140.8, 134.9, 132.5, 126.7, 123.4, 121.7, 115.2, 103.0, 94.7, 87.7, 83.7, 78.3, 77.9, 77.4, 74.4, 73.8, 73.1, 71.1, 70.2, 68.8, 65.7, 64.6, 62.5, 49.6, 45.4, 44.7, 42.5, 42.2, 36.4, 36.3, 34.9, 31.2, 31.1, 27.7, 26.9, 22.1, 21.7, 21.5, 18.4, 17.2, 16.3, 14.9, 11.4, 9.3, 7.6. HRMS (ESI) m/z Calcd. for C₅₂ H₈₂ O₁₃ N₃ [M+H⁺]: 956.5842, found 956.5822.

### Synthesis of Compound 20b.

The preparation of mesylated intermediate was as described for synthesis of mesylated intermediate to 19a. Subsequently, the mesylated intermediate (100 mg, 0.32 mmol) was reacted with compound 7 (200 mg, 0.27 mmol) in THF/DMSO (5:5 mL) and Hunig's base (0.7 mL, 4 mmol) at 50° C. overnight. The reaction was worked up as described for 19a. The crude product was purified through prep TLC eluting with EtOAc:MeOH=15:1 to furnish 20b as pale-yellow solid (13 mg, 5%). ¹H NMR (400 MHz, CDCl₃) δ 7.52 (d, J=8.4 Hz, 2H), 7.30 (dd, J=8.6, 2.0 Hz, 2H), 7.26 (d, J=2.6 Hz, 1H), 7.08 (d, J=2.5 Hz, 1H), 6.61 (d, J=9.4 Hz, 1H), 5.12 (d, J=4.9 Hz 1H), 5.04 (s, 1H), 4.70 (d, J=9.8 Hz, 1H), 4.52 (dd, J=15.7, 7.3 Hz, 2H), 4.30 (d, J=4.7 Hz, 1H), 4.23 (d, J=6.6 Hz, 1H), 4.08 (d, J=6.6 Hz, 2H), 3.83 (s, 1H), 3.74-3.58 (m, 4H), 3.55 (s, 2H), 3.44-3.22 (m, 8H), 3.17-2.98 (m, 2H), 2.96 (s, 2H), 2.86 (d, J=6.5 Hz, 4H), 2.80 (s, 3H), 2.72 (d, J=7.5 Hz, 2H), 2.65-2.46 (m, 6H), 2.35 (d, J=3.8 Hz, 13H), 2.23-1.96 (m, 2H), 1.94-1.83 (m, 4H), 1.82-1.43 (m, 10H), 1.41-1.03 (m, 10H), 1.02-0.69 (m, 6H). ¹³C NMR (176 MHz, CDCl₃) δ 188.1, 164.3, 161.6, 142.7, 140.2, 135.1, 132.5, 128.8, 127.2, 126.4, 123.9, 121.5, 113.8, 103.2, 95.1, 80.8, 78.2, 77.8, 74.3, 73.3, 73.0, 68.2, 65.9, 65.6, 52.6, 49.5, 45.1, 41.0, 36.7, 35.9, 34.9, 31.9, 29.7, 27.1, 26.7, 21.6, 18.3, 17.0, 11.3, 7.7. HRMS (ESI) m/z Calcd. for C₅₃ H₈₄ O₁₃ N₃ [M+H⁺]: 970.4432, found 970.4513.

### Synthesis of Compound 20c.

The preparation of mesylated intermediate was as described for synthesis of mesylated intermediate to 19a. Subsequently, the mesylated intermediate (100 mg, 0.30 mmol) was reacted with compound 7 (170 mg, 0.23 mmol) in THF/DMSO (5:5 mL) and Hunig's base (0.7 mL, 4.1 mmol) at 70° C. overnight. The reaction was worked up as described for 19a. The crude product was purified through prep TLC eluting with EtOAc:MeOH=15:1 to furnish 20c as pale-yellow solid (15 mg, 6%). ¹H NMR (400 MHz, CDCl₃) δ 7.48 (d, J=8.2 Hz, 2H), 7.32-7.22 (m, 3H), 7.07 (s, 1H), 6.59 (d, J=9.4 Hz, 1H), 5.10 (s, 2H), 4.68 (s, 1H), 4.50-4.39 (m, 1H), 4.25 (s, 1H), 4.07 (s, 2H), 3.64 (d, J=7.3 Hz, 3H), 3.53 (s, 1H), 3.36-3.28 (m, 4H), 2.74 (s, 2H), 2.54 (s, 2H), 2.47 (s, 1H), 2.34 (d, J=13.6 Hz, 6H), 2.29 (s, 2H), 2.14-2.07 (m, 3H), 2.00 (s, 3H), 1.77-1.65 (m, 3H), 1.59 (s, 2H), 1.33 (s, 10H), 1.31-1.10 (m, 13H), 1.08 (d, J=1.8 Hz, 3H), 1.04 (dd, J=17.7, 8.5 Hz, 2H), 0.98-0.80 (m, 10H). ¹³C NMR (176 MHz, CDCl₃) δ 161.6, 142.7, 140.2, 134.9, 132.4, 126.4, 121.5, 115.1, 102.9, 94.6, 78.2, 74.3, 73.0, 70.6, 65.6, 49.5, 45.3, 42.2, 36.3, 34.7, 31.9, 29.4, 26.7, 22.7, 22.0, 21.6, 21.4, 18.2, 17.1, 16.3, 14.6, 14.1, 11.3, 9.1, 7.4. HRMS (ESI) m/z Calcd. for C₅₄ H₈₆ O₁₃ N₃ [M+H⁺]: 984.5712, found 984.5863.

### Synthesis of Compound 20d.

The preparation of mesylated intermediate was as described for synthesis of mesylated intermediate to 19a. Subsequently, the mesylated intermediate (100 mg, 0.32 mmol) was reacted with compound 7 (170 mg, 0.23 mmol) in THF/DMSO (5:5 mL) and Hunig's base (0.7 mL, 4.1 mmol) at 50° C. overnight. The reaction was worked up as described for 19a. The crude product was purified through prep TLC eluting with EtOAc:MeOH=15:1 to furnish 20d as pale-yellow solid (13 mg, 5%). ¹H NMR (400 MHz, CDCl₃) δ 7.47 (d, J=8.5 Hz, 1H), 7.29 (d, J=8.5 Hz, 1H), 7.26 (s, 4H), 7.07 (q, J=1.5, 1.0 Hz, 1H), 6.59 (d, J=9.3 Hz, 1H), 5.10 (d, J=4.8 Hz, 2H), 4.68 (dd, J=9.9, 2.6 Hz, 2H), 4.45 (d, J=7.2 Hz, 2H), 4.29-4.26 (m, 1H), 4.12-4.01 (m, 2H), 3.69 (d, J=1.6 Hz, 2H), 3.67-3.62 (m, 3H), 3.47 (q, J=7.0 Hz, 2H), 3.32 (d, J=8.1 Hz, 6H), 3.29-3.22 (m, 1H), 3.04 (d, J=8.4 Hz, 2H), 2.77 (dd, J=7.5, 4.8 Hz, 1H), 2.70 (d, J=6.9 Hz, 1H), 2.51 (d, J=10.9 Hz, 2H), 2.43 (s, 1H), 2.37 (s, 1H), 2.32 (s, 8H), 2.25 (d, J=6.1 Hz, 4H), 2.17 (s, OH), 2.09 (d, J=1.1 Hz, 3H), 2.07-1.93 (m, 4H), 1.93-1.84 (m, 1H), 1.80 (d, J=14.6 Hz, 1H), 1.70-1.53 (m, 6H), 1.46 (ddd, J=14.4, 10.0, 7.3 Hz, 1H), 1.32 (t, J=3.1 Hz, 8H), 1.28-1.15 (m, 13H), 1.13-1.01 (m, 10H), 0.89 (td, J=7.4, 7.0, 2.0 Hz, 10H). ¹³C NMR (176 MHz, CDCl₃) δ 179.1, 161.8, 142.8, 140.2, 135.1, 132.5, 126.6, 124.4, 121.6, 115.2, 103.1, 94.6, 91.3, 83.5, 80.2, 78.3, 77.8, 77.6, 74.4, 73.8, 73.6, 73.2, 70.7, 70.2, 69.0, 66.2, 65.7, 62.7, 49.6, 45.5, 42.6, 42.4, 36.4, 34.8, 32.1, 29.8, 27.7, 26.9, 26.4, 22.1, 21.8, 21.5, 19.5, 18.3, 17.2, 16.4, 14.7, 11.4, 9.1, 7.4. HRMS (ESI) m/z Calcd. for C₅₅ H₈₈ O₁₃ N₃ [M+H⁺]: 998.6312, found 998.6296.

### Synthesis of Compound 20e.

The preparation of mesylated intermediate was as described for synthesis of mesylated intermediate to 19a. Subsequently, the mesylated intermediate (85 mg, 0.24 mmol) was reacted with compound 7 (170 mg, 0.23 mmol) in THF/DMSO (5:5 mL) and Hunig's base (0.8 mL, 4.65 mmol) at 70° C. overnight. The reaction was worked up as described for 19a. The crude product was purified through prep TLC eluting with EtOAc:MeOH=15:1 to furnish 20e as pale-yellow solid (13 mg, 5%). ¹H NMR (400 MHz, CDCl₃) δ 7.48 (d, J=8.6 Hz, 2H), 7.30 (d, J=8.6 Hz, 2H), 7.26 (m, 1H), 7.07 (d, J=2.4 Hz, 1H), 6.60 (d, J=9.5 Hz, 1H), 5.13 (s, 1H), 4.68 (d, J=10.5 Hz, 2H), 4.45 (d, J=7.0 Hz, 1H), 4.29 (s, 1H), 4.07 (s, 1H), 3.70-3.61 (m, 2H), 3.52 (s, 2H), 3.34 (s, 3H), 3.25 (s, 1H), 3.04 (t, J=9.9 Hz, 1H), 2.86 (s, 1H), 2.81-2.64 (m, 1H), 2.52 (d, J=10.6 Hz, 1H), 2.41 (t, J=7.0 Hz, 2H), 2.32 (s, 4H), 2.25 (s, 3H), 2.17 (d, J=0.6 Hz, 4H), 2.10 (d, J=1.1 Hz, 3H), 1.83 (s, 12H), 1.69-1.42 (m, 8H), 1.32 (d, J=5.4 Hz, 6H), 1.28-1.15 (m, 7H), 1.07 (q, J=7.7, 7.2 Hz, 9H), 0.93-0.83 (m, 8H). ¹³C NMR (176 MHz, CDCl₃) δ 161.7, 142.8, 140.2, 135.1, 132.5, 126.6, 124.4, 121.7, 115.2, 103.1, 94.6, 91.5, 83.5, 80.1, 78.3, 77.8, 74.4, 73.8, 73.2, 70.7, 65.7, 62.6, 53.4, 49.6, 45.4, 42.4, 36.4, 34.8, 29.8, 28.7, 27.7, 26.9, 22.2, 21.8, 21.5, 19.5, 18.4, 17.2, 16.4, 14.8, 11.4, 9.1, 7.5. HRMS (ESI) m/z Calcd. for C₅₆ H₉₀ O₁₃ N₃ [M+H⁺]: 1012.6468, found 1012.6451.

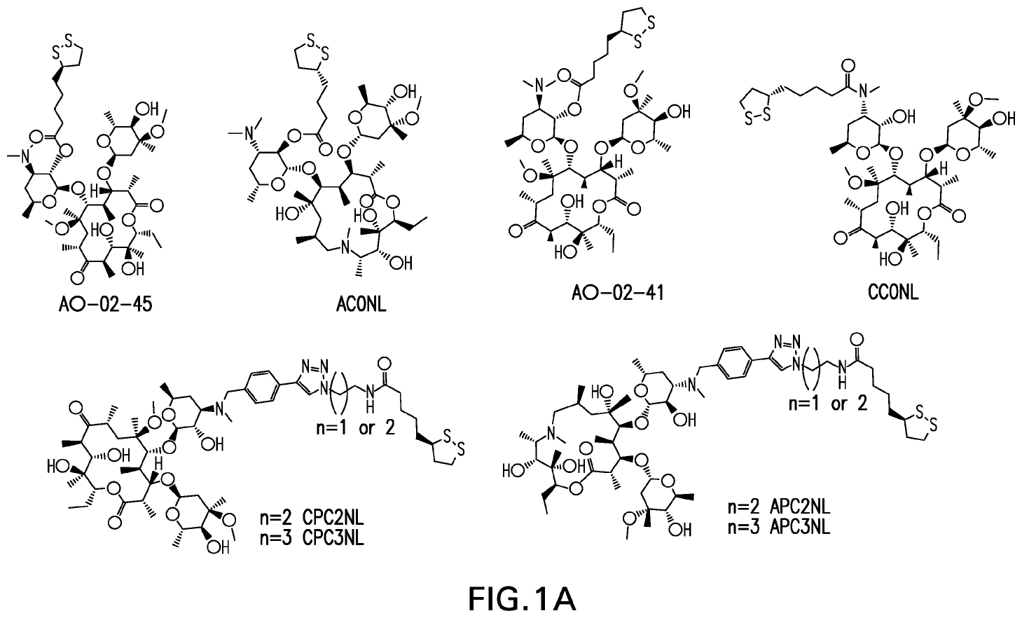

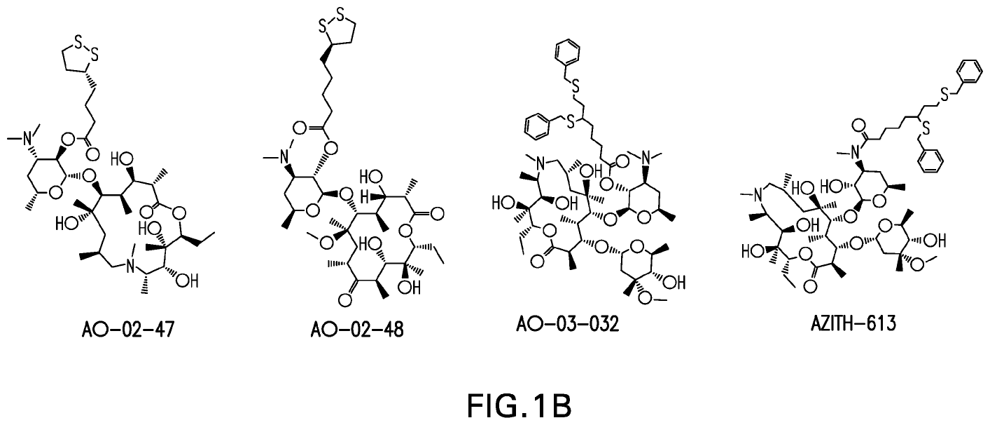

The target lipoyl- and 6,8-bis(benzylthio)octanoyl-compounds (FIG. 1) were synthesized as shown in Schemes 5-10. Lipoic acid anhydride was synthesized by reacting alpha-lipoic acid (ALA) with EDCI in DCM overnight. The anhydride was then reacted with AZM or CLM in DCM, overnight at room temperature to furnish AO-02-41 or AO-02-45 (Scheme 5).

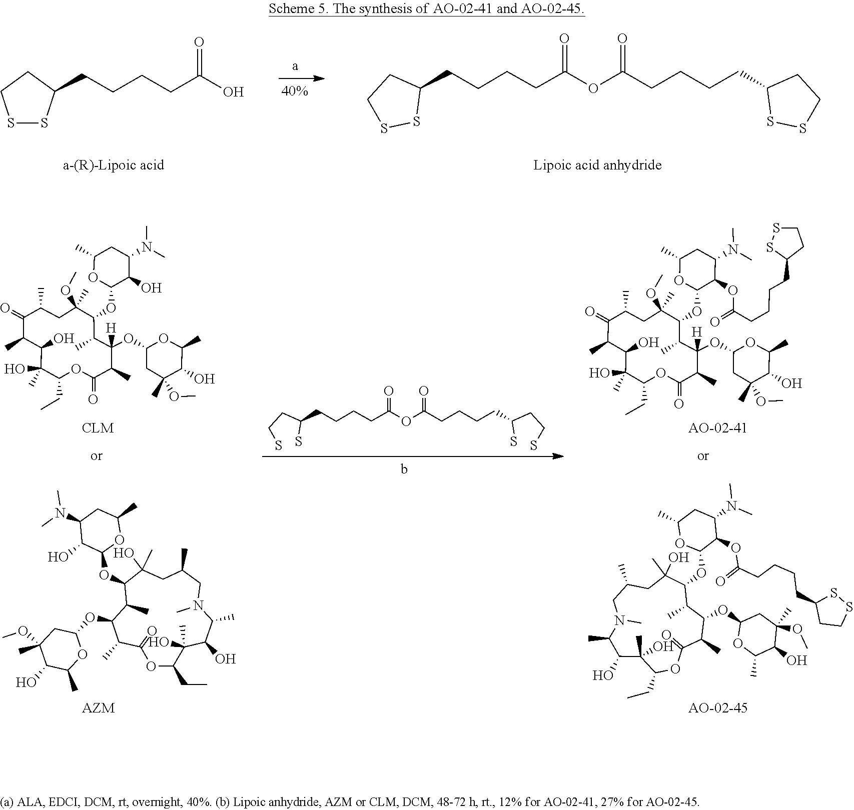

Mild acid treatment of AO-02-41 and AO-02-45 afforded AO-02-47 and AO-02-48, analogs lacking the cladinose sugar, respectively (Scheme 6).

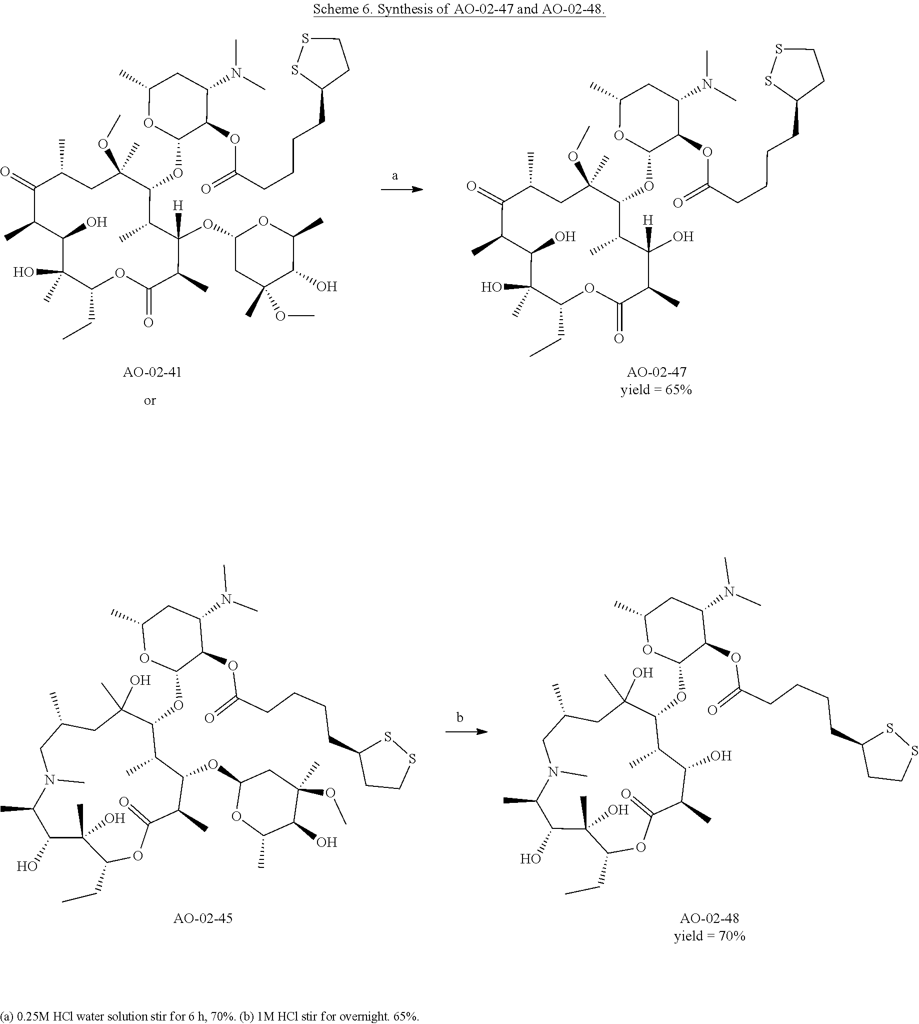

The synthesis of CC0NL and AC0NL was accomplished by reacting compound 4 or 7 with ALA and EDCI in Hunig's base (10 v/v %)/DCM mixture in the presence of 0.1 eq DMAP (Scheme 7).

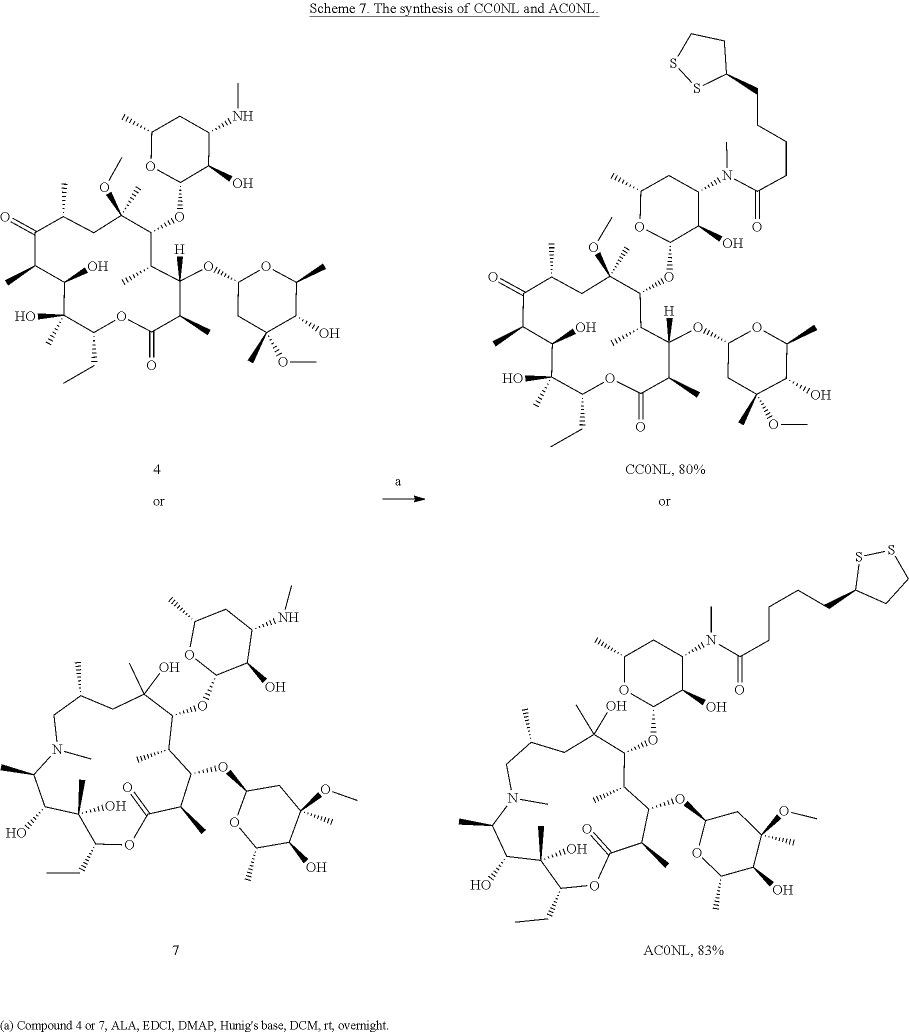

To synthesize CPC2NL, CPC3NL, APC2NL and APC3NL, 2-bromoethylamine hydrobromide or 3-bromopropylamine hydrobromide were converted to their azido derivatives which were subsequently reacted with ALA via EDCI coupling to furnish the azido-ALA compounds C2NL and C3NL. Cu (I) catalyzed Huisgen azide-alkyne cycloaddition reaction between C2NL or C3NL and compound 5 or 8, following literature protocol (Rostovtsev, V. V.; Green, L. G.; Fokin, V. V.; Sharpless, K. B.; A stepwise Huigsen cycloaddition process: Copper(I)-catalyzed regioselective “Ligation” of azides and terminal alkynes. Angew. Chem. Int. Ed. 2002, 41, 2596-2599; Oyelere, A. K.; Chen, P. C.; Guerrant, W.; Mwakwari, S. C.; Hood, R.; Zhang, Y.; Fan, Y. Non-peptide macrocyclic histone deacetylase inhibitors. J. Med Chem. 2009, 52, 456-468), furnished the target compounds CPC2NL, CPC3NL, APC2NL and APC3NL (Scheme 8).

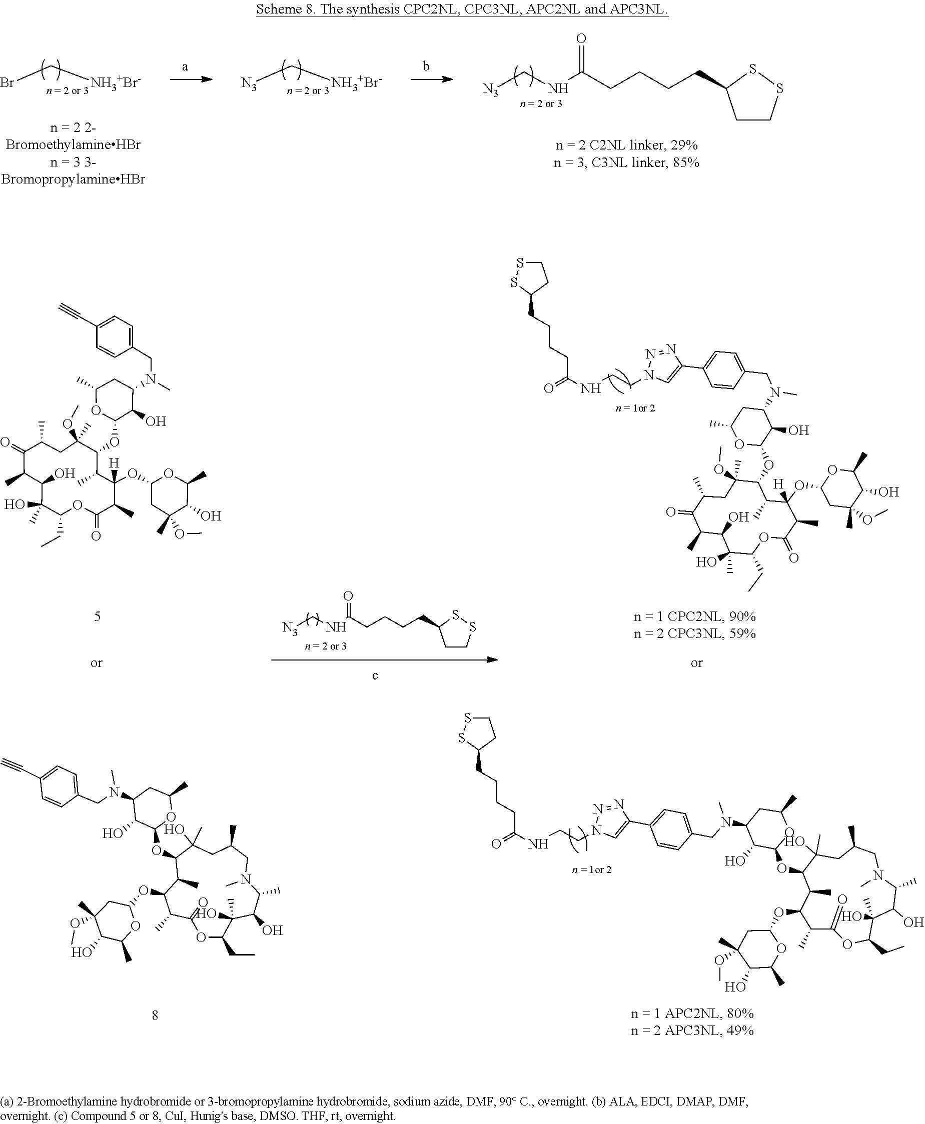

The synthesis of AZM-613 was accomplished by reacting compound 7 with CPI-613 (CAS 95809-78-2), EDCI, and DMAP in DCM at rt for 24 h (Scheme 9)

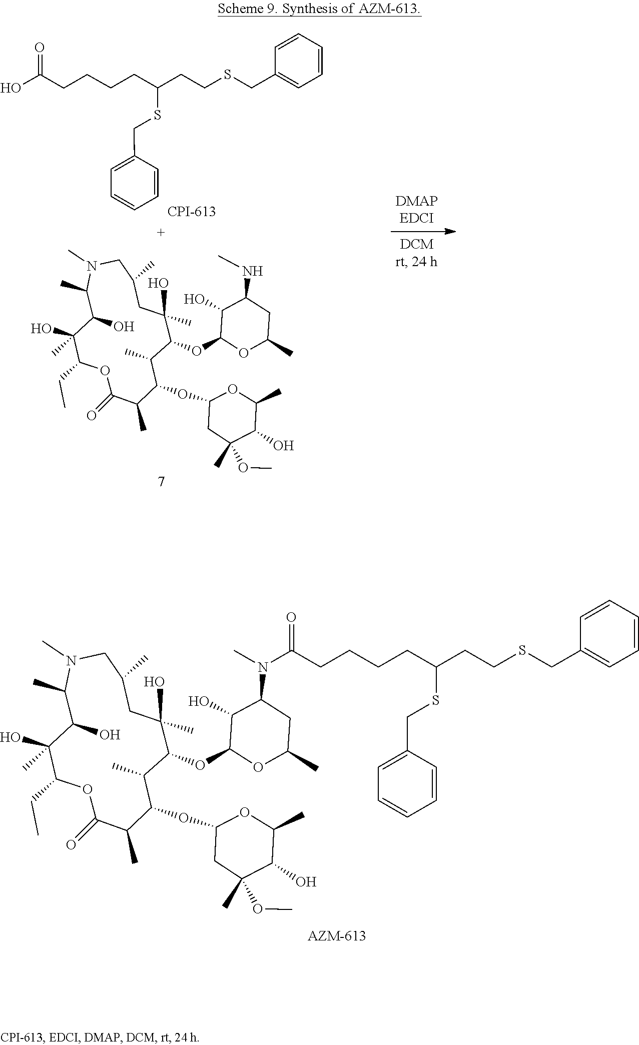 [SMILES: O=C(O)CCCCC(CCSCc1ccccc1)SCc1ccccc1 ; CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](NC)[C@H]2O)[C@](C)(O)C[C@@H](C)CN(C)[C@H](C)[C@@H](O)[C@]1(C)O ; CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C(=O)CCCCC(CCSCc3ccccc3)SCc3ccccc3)[C@H]2O)[C@](C)(O)C[C@@H](C)CN(C)[C@H](C)[C@@H](O)[C@]1(C)O]

To synthesize AO-03-032, CPI-613 was reacted with EDCI in DCM for 2 h; AZM was added and the reaction was stirred at rt for 24 h to furnish AO-03-032 (Scheme 10).

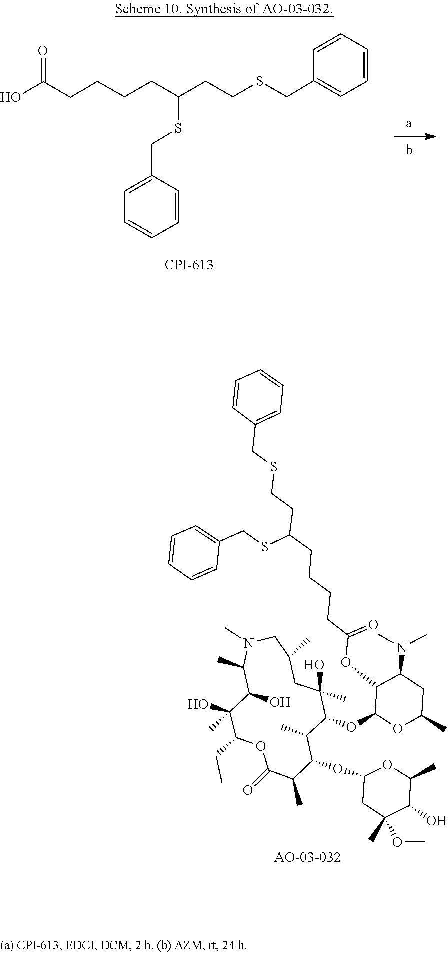

### Synthesis Procedures for Target Compounds AO-02-41, AO-02-45, AO-02-47, AO-02-48, CC0NL, AC0NL, CPC2NL, CPC3NL, APC2NL, APC3NL, AZM-613 and AO-03-032.

### Synthesis of Lipoic Acid Anhydride:

Lipoic acid (400 mg, 1.94 mmol) was dissolved in DCM (10 mL) and EDCI (181 mg, 1.16 mmol) was added. The mixture was stirred at rt overnight under argon, water (30 mL) and DCM (20 mL) were added and the two layers separated. The DCM layer was extracted with water (20 mL×3), dried over Na₂SO₄ and evaporated in vacuo to yield crude lipoic acid anhydride as yellow oil (160 mg, 40%) which was used for the next reaction without purification.

### Synthesis of AO-02-41:

CLM (500 mg, 0.67 mmol) was mixed with the lipoic acid anhydride (100 mg, 0.25 mmol) in DCM (15 mL). The mixture was stirred at rt overnight under argon. The reaction was partitioned between water (50 mL) and (30 mL), the two layers were separated and the DCM layer extracted with water (30 mL×2). The organic layer was dried over Na₂SO₄ and evaporated in vacuo. The crude was purified using column chromatography eluting with CHCl₃:MeOH ratio 30:1 to 24:1 to 20:1 to 18:1 to 14:1 to furnish AO-02-41 as white solid (200 mg, 84%). ¹H NMR (400 MHz, CDCl₃) δ 4.99 (d, J=10.9 Hz, 1H), 4.87 (d, J=4.9 Hz, 1H), 4.67 (dd, J=10.4, 7.5 Hz, 1H), 4.52 (d, J=7.4 Hz, 1H), 3.98-3.83 (m, 2H), 3.69 (d, J=8.1 Hz, 2H), 3.51 (dt, J=20.9, 7.1 Hz, 2H), 3.42 (dd, J=11.1, 6.0 Hz, 1H), 3.31 (s, 4H), 3.23-3.02 (m, 3H), 3.02-2.85 (m, 6H), 2.79 (p, J=7.8 Hz, 1H), 2.50 (t, J=9.8 Hz, 2H), 2.45-2.36 (m, 1H), 2.36-2.07 (m, 12H), 1.84 (ddt, J=21.2, 14.3, 6.9 Hz, 4H), 1.72-1.49 (m, 10H), 1.43 (h, J=8.3, 7.6 Hz, 3H), 1.32 (s, 4H), 1.28-0.95 (m, 27H), 0.94-0.68 (m, 7H). ¹³C NMR (101 MHz, CDCl₃) δ 175.6, 172.2, 100.4, 95.8, 80.4, 78.3, 78.0, 76.5, 74.1, 72.8, 71.5, 69.0, 68.2, 65.8, 63.7, 56.5, 50.4, 49.4, 45.2, 40.6, 40.3, 38.9, 38.4, 37.2, 34.7, 34.4, 28.6, 24.7, 21.5, 19.8, 18.6, 17.9, 16.1, 12.3, 10.6, 9.0. HRMS (ESI) m/z Calcd. C₄₆ H₈₂ O₁₄ N S₂ [M+H⁺]: 936.5171, found 936.5149.

### Synthesis of AO-02-45:

AZM (500 mg, 0.67 mmol) was mixed with the Lipoic acid anhydride (100 mg, 0.26 mmol) in DCM (15 mL). The mixture was stirred at rt overnight under argon. The reaction was partitioned between water (50 mL) and (30 mL), the two layers were separated and the DCM layer extracted with water (30 mL×2). The organic layer was dried over Na₂SO₄ and evaporated in vacuo. The crude was purified using column chromatography eluting with CHCl₃:MeOH ratio 30:1 to 24:1 to 20:1 to 18:1 to 14:1 to 12:1 to furnish AO-02-045 as white solid (206 mg, 87%). ¹H NMR (400 MHz, CDCl₃) δ 5.10 (d, J=4.8 Hz, 1H), 4.72 (dd, J=10.6, 7.5 Hz, 1H), 4.64 (dd, J=9.9, 2.7 Hz, 1H), 4.49 (d, J=7.6 Hz, 1H), 4.19 (dd, J=4.2, 2.0 Hz, 1H), 4.12-3.89 (m, 1H), 3.66-3.57 (m, 1H), 3.57-3.40 (m, 3H), 3.33 (s, 2H), 3.23-3.03 (m, 2H), 2.99 (d, J=8.0 Hz, 1H), 2.74-2.60 (m, 2H), 2.60-2.35 (m, 3H), 2.36-2.07 (m, 10H), 2.04-1.92 (m, 2H), 1.93-1.75 (m, 3H), 1.62 (dd, J=28.4, 20.5, 13.7, 5.1 Hz, 6H), 1.43 (tdd, J=15.8, 9.3, 6.1 Hz, 3H), 1.36-1.09 (m, 14H), 1.04 (d, J=7.1 Hz, 4H), 0.96-0.66 (m, 6H). ¹³C NMR (101 MHz, CDCl₃) δ 178.7, 172.2, 100.6, 94.4, 82.9, 78.1, 74.2, 73.6, 73.0, 71.5, 70.0, 68.2, 65.5, 63.9, 62.4, 56.5, 49.4, 45.2, 42.0, 40.7, 40.2, 38.5, 36.2, 34.7, 30.1, 28.6, 27.5, 26.6, 24.6, 21.9, 21.6, 21.2, 18.2, 16.2, 14.6, 11.2, 8.9, 7.3. HRMS (ESI) m/z Calcd. C₄₆H₈₅ O₁₃ N₂ S₂ [M+H⁺]: 937.5488, found 937.5445.

### Synthesis of AO-02-47:

AO-02-41 (25 mg, 0.026 mmol) was dissolved into the 1N HCl (15 mL) and stirred at rt for 20 h. The reaction was washed with DCM (25 mL×3) and the aqueous layer was basified to pH=8-9 with NaOH and extracted with DCM (25 mL×2). TLC indicated that the product was in the first organic layer, which was dried over Na₂SO₄ and evaporated in vacuo. The crude was purified using prep TLC eluting with DCM:MeOH=12:1 to furnish AO-02-47 as white solid (13 mg, 65%). ¹H NMR (400 MHz, CDCl₃) δ 5.12 (dd, J=11.2, 2.4 Hz, 1H), 4.99-4.85 (m, 1H), 4.82 (d, J=7.4 Hz, 1H), 3.90 (s, 1H), 3.83-3.66 (m, 4H), 3.58-3.49 (m, 1H), 3.44-3.28 (m, 2H), 3.22 (d, J=8.3 Hz, 2H), 3.20-3.03 (m, 3H), 3.03-2.93 (m, 2H), 2.90 (s, 4H), 2.75 (s, 8H), 2.63 (dq, J=10.3, 6.6 Hz, 2H), 2.55-2.39 (m, 3H), 2.39-2.24 (m, 2H), 2.19 (dt, J=12.9, 4.9 Hz, 2H), 2.14-1.98 (m, 1H), 1.98-1.82 (m, 3H), 1.67 (dddd, J=18.5, 16.1, 13.9, 9.2 Hz, 6H), 1.59-1.36 (m, 6H), 1.36-1.24 (m, 7H), 1.24-0.97 (m, 19H), 0.88 (d, J=7.4 Hz, 3H), 0.79 (t, J=7.3 Hz, 3H). ¹³C NMR (101 MHz, CDCl₃) δ 174.9, 173.0, 98.4, 81.5, 74.1, 69.7, 68.9, 67.6, 63.2, 56.5, 49.7, 45.5, 44.1, 40.3, 38.5, 37.4, 35.6, 34.7, 28.9, 24.0, 20.8, 19.3, 17.9, 16.3, 15.5, 12.6, 10.4, 8.4. HRMS (ESI) m/z Calcd. C₃₈ H₆₈ O₁₁ N S₂ [M+H⁺]: 778.4228, found 778.4193.

### Synthesis of AO-02-48:

AO-02-45 (50 mg, 0.053 mmol) was dissolved into the 0.25 N HCl (10 mL) and stirred at rt for 20 h. The reaction was washed with DCM (25 mL×3) and the aqueous layer was basified to pH=8-9 with NaOH and extracted with DCM (25 mL×2). The organic layer was dried over Na₂SO₄ and TLC indicated it to be a single spot. Solvent was evaporated in vacuo to furnish AO-02-48 as white solid (29 mg, 70%). ¹H NMR (400 MHz, CDCl₃) δ 4.75-4.54 (m, 1H), 3.78-3.58 (m, 2H), 3.54 (dd, J=19.5, 13.2 Hz, 2H), 3.30-2.95 (m, 2H), 2.82-2.55 (m, 2H), 2.46 (dq, J=12.3, 6.3 Hz, 1H), 2.30 (d, J=48.4 Hz, 6H), 2.00 (d, J=38.8 Hz, 1H), 1.90 (dt, J=13.8, 6.9 Hz, 2H), 1.79-1.33 (m, 14H), 1.33-1.17 (m, 7H), 1.17-0.99 (m, 4H), 0.99-0.73 (m, 6H). ¹³C NMR (101 MHz, CDCl₃) δ 177.4, 172.2, 99.9, 86.2, 78.4, 77.8, 74.2, 73.1, 71.6, 68.9, 63.5, 62.4, 56.5, 43.9, 41.3, 40.7, 38.5, 36.9, 35.9, 34.7, 34.3, 30.5, 28.7, 26.1, 24.7, 21.3, 21.1, 20.8, 16.1, 11.0, 7.6. HRMS (ESI) m/z Calcd. C₃₈ H₇₁ O₁₀ N₂ S₂ [M+H⁺]: 779.4545, found 779.4516.

### Synthesis of AC0NL:

Compound 7 (180 mg, 0.25 mmol) was dissolved in Hunig's base (0.5 mL)/DCM (5 mL) mixture with stirring. In a separate flask, ALA (100 mg, 0.48 mmol) and EDCI (200 mg, 1.29 mmol) were dissolved in DCM (5 mL). After 5 minutes stirring under argon, the ALA solution was added to the solution of 7 dropwisely with stirring, and DMAP (10 mg, 0.08 mmol) was subsequently added. The reaction was stirred at rt under argon atmosphere for 24 h. The reaction was partitioned between DCM (50 mL) and water (40 mL) and the two layers were separated. Then, the organic layer was washed with water (30 mL), dried over Na₂SO₄ and evaporated in vacuo. The crude was purified using prep TLC eluting with EtOAc:MeOH=13:1 to furnish AC0NL as whitish-yellow foam (166 mg, 83%). ¹H NMR (700 MHz, CDCl₃) δ 5.11 (d, J=3.1 Hz, 1H), 4.78-4.74 (m, 1H), 4.72-4.66 (m, 1H), 4.49 (dd, J=15.9, 7.3 Hz, 1H), 4.19 (dt, J=6.6, 3.3 Hz, 1H), 4.07 (td, J=9.8, 4.9 Hz, 1H), 3.68 (s, 1H), 3.62-3.55 (m, 2H), 3.38 (s, 2H), 3.34 (s, 1H), 3.17 (tdd, J=7.9, 5.3, 2.7 Hz, 1H), 3.14-3.02 (m, 3H), 2.91 (s, 2H), 2.83 (s, 1H), 2.74-2.67 (m, 1H), 2.58-2.51 (m, 1H), 2.51-2.42 (m, 3H), 2.41-2.29 (m, 2H), 1.97-1.86 (m, 2H), 1.77-1.56 (m, 5H), 1.47 (dd, J=19.4, 12.9, 9.9, 6.3, 2.6 Hz, 3H), 1.37 (s, 2H), 1.34 (d, J=6.3 Hz, 3H), 1.27-1.14 (m, 8H), 1.11 (d, J=7.9 Hz, 3H), 1.04-0.94 (m, 5H), 0.88 (t, J=7.4 Hz, 3H). ¹³C NMR (176 MHz, CDCl₃) δ 174.3, 103.5, 94.9, 84.2, 78.0, 78.0, 77.3, 76.90, 74.5, 74.2, 73.9, 73.1, 71.6, 70.9, 69.3, 69.1, 68.4, 65.9, 62.3, 62.0, 57.5, 56.6, 53.9, 49.5, 42.3, 40.3, 38.5, 36.6, 36.4, 35.8, 34.8, 34.1, 33.8, 33.2, 31.9, 31.7, 29.7, 29.40, 29.10, 26.9, 26.4, 25.3, 25.0, 24.8, 24.7, 22.6, 22.6, 22.1, 21.6, 21.6, 21.4, 21.3, 21.0, 20.9, 20.7, 18.1, 16.5, 14.7, 14.1, 11.2, 9.4, 7.5. HRMS (ESI) m/z Calcd. C₄₅ H₈₂ O₁₃ N₂ S₂ [M+H⁺]: 923.5331, found 923.5334.

### Synthesis of CC0NL:

Compound 4 (180 mg, 0.25 mmol) was dissolved in Hunig's base (0.5 mL) DCM (5 mL) mixture with stirring. In a separate flask, ALA (100 mg, 0.48 mmol) was mixed with EDCI (200 mg, 1.29 mmol) in DCM (5 mL). After 5 minutes stirring under argon, the ALA solution was added to the solution of 4 dropwisely with stirring, and DMAP (10 mg, 0.08 mmol) was subsequently added. The reaction was stirred at rt under argon atmosphere for 24 h. The reaction was partitioned between DCM (50 mL) and water (40 mL) and the two layers were separated. Then, the organic layer was washed with water (30 mL), dried over Na₂SO₄ and evaporated in vacuo. The crude was purified using Prep TLC eluting with EtOAc:hexanes=8:2 to furnish CC0NL as whitish-yellow foam (186 mg, 80%). ¹H NMR (700 MHz, CDCl₃) δ 5.03 (dd, J=11.4, 3.9 Hz, 1H), 4.90 (dt, J=8.4, 3.9 Hz, 1H), 4.68-4.61 (m, 1H), 4.49 (dd, J=7.4, 2.5 Hz, 1H), 4.00 (q, J=7.4 Hz, 1H), 3.95 (d, J=4.9 Hz, 1H), 3.74-3.69 (m, 2H), 3.68-3.61 (m, 2H), 3.55 (q, J=7.0 Hz, 1H), 3.34-3.27 (m, 3H), 3.19 (d, J=6.0 Hz, 1H), 3.15 (dt, J=11.7, 6.4 Hz, OH), 3.09 (ddd, J=11.1, 7.1, 3.6 Hz, 1H), 3.00 (d, J=3.4 Hz, 3H), 2.96 (d, J=7.1 Hz, 1H), 2.88 (d, J=2.8 Hz, 2H), 2.84 (h, J=8.1, 7.5 Hz, 1H), 2.80 (d, J=3.2 Hz, 1H), 2.59-2.52 (m, 2H), 2.44 (dp, J=12.5, 6.4, 4.7 Hz, 1H), 2.36-2.17 (m, 4H), 1.89 (qd, J=17.3, 13.9, 5.3 Hz, 1H), 1.79-1.51 (m, OH), 1.50-1.41 (m, 2H), 1.39 (d, J=3.2 Hz, 2H), 1.28 (d, J=5.7 Hz, 2H), 1.25-1.13 (m, 3H), 1.14-1.08 (m, 8H), 1.05-0.98 (m, 3H), 0.81 (dt, J=10.6, 5.2 Hz, 3H). ¹³C NMR (176 MHz, CDCl₃) δ 220.8, 175.6, 174.3, 103.5, 96.2, 82.1, 76.6, 74.2, 72.8, 71.8, 69.0, 68.3, 65.7, 56.4, 53.7, 50.6, 49.4, 45, 40.2, 39.2, 38.8, 37.2, 35.7, 34.7, 33.8, 29.5, 29, 24.7, 21.4, 21, 19.6, 18.5, 17.9, 16, 12.3, 10.5, 9.5. HRMS (ESI) m/z Calcd. C₄₅H₇₉O₁₄NNaS₂ [M+Na⁺]: 944.4834, found 944.4843.

### Synthesis of C2NL:

2-Bromoethylamine. HBr (500 mg, 2.44 mmol) and NaN₃ (400 mg, 6.15 mmol) were mixed in DMF (20 mL). The mixture was heated to 80-90° C. overnight and concentrated in vacuo to reduce DMF to about 5 mL. The crude was used for the next reaction without purification. In a separated flask, ALA (480 mg, 2.32 mmol) was dissolved in DCM (20 mL), followed by EDCI (400 mg, 2.11 mmol) and DMAP (50 mg, 0.41 mmol). The mixture was stirred for 30 minutes and the concentrated crude intermediate in DMF was added to the reaction. The mixture was stirred in room temperature overnight. The crude was partitioned between water (50 mL) and DCM (30 mL). The two layers were separated and the aqueous layer was extracted with DCM (30 mL×2). The combined DCM layer was dried Na₂SO₄ and evaporated in vacuo. The crude was purified using column chromatography, eluting with EtOAc to furnish C2NL as oil (188 mg, 29%). ¹H NMR (400 MHz, CDCl₃) δ 4.12-4.02 (m, 1H), 3.69-3.55 (m, 2H), 3.48 (s, 3H), 3.42 (d, J=0.5 Hz, 3H), 2.94 (dtd, J=13.1, 6.6, 5.4 Hz, 1H), 2.80 (t, J=7.5 Hz, 2H), 2.39 (dq, J=12.7, 6.9 Hz, 1H), 2.24-2.03 (m, 6H), 1.97 (ddd, J=13.5, 8.7, 5.4, 2.6 Hz, 2H).

### Synthesis of C₃NL:

3-Bromopropylamine. HBr (400 mg, 1.83 mmol) and NaN₃ (200 mg, 3.07 mmol) were mixed in DMF (10 mL). The mixture was heated to 80-90° C. overnight and concentrated in vacuo to reduce DMF to about 5 mL. The crude was used for the next reaction without purification. In a separated flask, ALA (400 mg, 1.94 mmol) was dissolved in DCM (20 mL), followed by the addition of EDCI (400 mg, 2.11 mmol) and DMAP (60 mg, 0.49 mmol). The mixture was stirred for 30 minutes and the concentrated crude intermediate in DMF was added to the reaction. The mixture was stirred in room temperature overnight. The crude was partitioned between water (50 mL) and DCM (30 mL). The two layers were separated and the aqueous layer was extracted with DCM (30 mL×2). The combined DCM layer was dried Na₂SO₄ and evaporated in vacuo. The crude was purified using column chromatography, eluting with EtOAc to furnish C₃NL as oil (450 mg, 85%). ¹H NMR (400 MHz, CDCl₃) δ 3.59 (dq, J=8.5, 6.4 Hz, 1H), 3.44-3.36 (m, 2H), 3.23-3.08 (m, 3H), 3.01 (s, 3H), 2.94 (s, 3H), 2.47 (dtd, J=13.0, 6.6, 5.4 Hz, 1H), 2.33 (t, J=7.5 Hz, 2H), 1.99-1.80 (m, 2H), 1.77-1.61 (m, 3H), 1.49 (ddd, J=8.8, 7.6, 5.6, 2.8 Hz, 2H).

### Synthesis of CPC2NL:

Compound 5 (50 mg, 0.06 mmol) and C2NL (50 mg, 0.18 mmol) were dissolved in THF/DMSO solution (1:1 mL). CuI (10 mg, 0.05 mmol) was added and the mixture was purged with argon with stirring for 5 minutes. Hunig's base (0.2 mL) was added and the mixture was stirred overnight. The reaction was partitioned between NH₄OH (1M, 30 mL) and DCM (30 mL) and the two layers were separated. The aqueous layer was extracted with DCM (20 mL×2), the combined DCM layer was dried Na₂SO₄ and evaporated in vacuo. The crude was purified using prep TLC, eluting with DCM:MeOH=15:1 to furnish CPC2NL as whitish-yellow solid (60 mg, 90%). ¹H NMR (700 MHz, CDCl₃) δ 7.71 (t, J=4.1 Hz, 2H), 7.30 (d, J=8.1 Hz, 2H), 4.98 (dd, J=11.0, 2.3 Hz, 1H), 4.84 (d, J=4.6 Hz, 1H), 4.50-4.45 (m, 2H), 4.37 (d, J=7.2 Hz, 1H), 3.92-3.85 (m, 2H), 3.79-3.75 (m, 2H), 3.75-3.68 (m, 2H), 3.68 (dd, J=9.4, 1.4 Hz, 1H), 3.60-3.54 (m, 1H), 3.48-3.36 (m, 2H), 3.28-3.23 (m, 1H), 3.13-3.06 (m, 5H), 3.02 (dt, J=11.1, 6.9 Hz, 1H), 2.96 (s, 2H), 2.95-2.90 (m, 1H), 2.83-2.78 (m, 1H), 2.54-2.48 (m, 1H), 2.36 (dtd, J=13.1, 6.7, 5.3 Hz, 1H), 2.30-2.22 (m, 1H), 2.19 (s, 2H), 2.11 (td, J=7.3, 1.3 Hz, 2H), 1.88-1.74 (m, 5H), 1.66-1.50 (m, 7H), 1.47-1.34 (m, 2H), 1.34 (s, 3H), 1.21 (d, J=6.1 Hz, 4H), 1.18 (d, J=5.9 Hz, 10H), 1.12 (d, J=7.3 Hz, 3H), 1.08-1.03 (m, 11H), 1.02 (d, J=7.6 Hz, 3H), 0.84-0.79 (m, 3H), 0.81-0.75 (m, 6H). ¹³C NMR (176 MHz, CDCl₃) δ 174.8, 172.3, 146.6, 128.4, 124.8, 119.4, 101.7, 94.9, 79.9, 77.3, 76.9, 75.6, 73.2, 71.5, 69.8, 68.0, 64.7, 63.2, 56.5, 55.3, 49.6, 48.6, 48.4, 44.2, 39.2, 38.2, 37.4, 36.2, 35.9, 35.2, 33.5, 30.9, 30.6, 28.7, 27.8, 24.2, 21.7, 20.5, 20.0, 18.8, 17.6, 17.0, 15.0, 14.9, 13.1, 11.3, 9.6, 8.1. HRMS (ESI) m/z Calcd. C₅₆ H₉₂ O₁₄ N₅ S₂ [M+H⁺]: 1122.6077, found 1122.6064.

### Synthesis of APC2NL:

Compound 8 (50 mg, 0.06 mmol) and C2NL (50 mg, 0.18 mmol) were dissolved in THF/DMSO solution (2:2 mL). CuI (10 mg, 0.05 mmol) was added and the mixture was purged with argon with stirring for 5 minutes. Hunig's base (0.4 mL) was added and the mixture was stirred overnight. The reaction was partitioned between NH₄OH (1M, 30 mL) and DCM (30 mL) and the two layers were separated. The aqueous layer was extracted with DCM (20 mL×2), the combined DCM layer was dried Na₂SO₄ and evaporated in vacuo. The crude was purified using prep TLC, eluting with DCM:MeOH=9:1 to furnish APC2NL as whitish-yellow solid (46 mg, 80%). ¹H NMR (700 MHz, CDCl₃) δ 8.03 (s, 1H), 7.83-7.73 (m, 3H), 7.38 (d, J=7.8 Hz, 2H), 6.09 (t, J=6.0 Hz, 1H), 5.12 (d, J=5.0 Hz, 1H), 4.75-4.64 (m, 2H), 4.61-4.52 (m, 2H), 4.45 (d, J=7.3 Hz, 1H), 4.27 (dd, J=4.4, 2.0 Hz, 1H), 4.11-4.03 (m, 1H), 3.91-3.77 (m, 3H), 3.77-3.59 (m, 3H), 3.59-3.45 (m, 4H), 3.45-3.32 (m, 2H), 3.26 (d, J=14.9 Hz, 1H), 3.24-3.13 (m, 4H), 3.10 (dt, J=11.1, 6.9 Hz, 1H), 3.02 (t, J=9.7 Hz, 1H), 3.00-2.83 (m, 6H), 2.83-2.66 (m, 3H), 2.62 (ddd, J=13.6, 10.3, 3.8 Hz, 1H), 2.55 (d, J=11.7 Hz, 2H), 2.44 (dtd, J=13.1, 6.6, 5.3 Hz, 1H), 2.34 (d, J=6.9 Hz, 4H), 2.29 (d, J=26.8 Hz, 4H), 2.19 (td, J=7.4, 1.4 Hz, 2H), 2.16-1.96 (m, 5H), 1.96-1.85 (m, 3H), 1.85-1.73 (m, 3H), 1.73-1.60 (m, 5H), 1.56 (dd, J=15.3, 5.1 Hz, 2H), 1.52-1.37 (m, 5H), 1.37-1.30 (m, 8H), 1.30-1.22 (m, 8H), 1.22-1.13 (m, 7H), 1.11 (d, J=8.8 Hz, 6H), 1.05 (d, J=7.5 Hz, 4H), 1.01-0.78 (m, 9H). ¹³C NMR (176 MHz, CDCl₃) δ 178.8, 173.4, 162.5, 147.7, 139.3, 129.3, 129.3, 125.7, 120.4, 102.9, 94.6, 83.7, 78.1, 77.9, 77.2, 77.0, 76.9, 74.3, 73.7, 72.9, 70.8, 68.7, 65.5, 64.6, 57.7, 56.4, 49.6, 49.4, 45.2, 42.3, 42.1, 40.2, 39.2, 38.5, 37.0, 36.5, 36.3, 36.2, 34.7, 34.6, 31.4, 29.8, 29.7, 29.7, 29.4, 28.8, 27.5, 26.8, 25.3, 22.7, 22.0, 21.5, 21.4, 21.3, 18.2, 16.2, 14.8, 11.3, 9.1, 7.5. HRMS (ESI) m/z Calcd. C₅₆ H₉₅ O₁₃ N₆ S₂ [M+H⁺]: 1123.6393, found 1123.6383.

### Synthesis of CPC3NL:

Compound 5 (50 mg, 0.06 mmol) and C3NL (100 mg, 0.35 mmol) were dissolved in THF/DMSO solution (2:1 mL). CuI (20 mg, 0.05 mmol) was added and the mixture was purged with argon with stirring for 5 minutes. Hunig's base (0.3 mL) was added and the mixture was stirred for overnight. The reaction was partitioned between NH₄OH (1M, 30 mL) and DCM (30 mL) and the two layers were separated. The aqueous layer was extracted with DCM (20 mL×2), the combined DCM layer was dried Na₂SO₄ and evaporated in vacuo. The crude was purified using prep TLC, eluting with DCM:MeOH=15:1 to furnish CPC3NL as whitish-yellow solid (40 mg, 59%). ¹H NMR (700 MHz, CDCl₃) δ 8.23 (s, 1H), 8.05-7.68 (m, 3H), 7.38 (d, J=7.7 Hz, 2H), 6.05 (t, J=6.6 Hz, 1H), 5.06 (d, J=11.0 Hz, 1H), 4.92 (d, J=5.1 Hz, 1H), 4.70-4.34 (m, 3H), 4.08-3.87 (m, 2H), 3.87-3.67 (m, 3H), 3.65 (d, J=7.3 Hz, 1H), 3.48 (dd, J=18.1, 9.5 Hz, 2H), 3.41-3.25 (m, 3H), 3.25-2.81 (m, 12H), 2.59 (dq, J=19.1, 11.4, 9.0 Hz, 2H), 2.50-2.06 (m, 8H), 2.06-1.62 (m, 12H), 1.56-1.33 (m, 7H), 1.33-1.01 (m, 29H), 0.87 (dt, J=27.8, 7.1 Hz, 5H). ¹³C NMR (176 MHz, CDCl₃) δ 175.8, 161.6, 147.8, 129.4, 125.8, 120.0, 102.8, 96.0, 80.9, 78.3, 78.3, 77.9, 77.2, 77.0, 76.9, 74.3, 72.5, 70.8, 69.1, 68.7, 65.7, 64.0, 57.6, 50.6, 49.4, 47.6, 45.3, 45.1, 39.3, 39.1, 37.2, 36.9, 35.1, 34.8, 30.1, 29.7, 29.5, 21.5, 21.3, 21.0, 19.8, 18.7, 18.0, 16.0, 14.1, 12.3, 10.6, 9.1. HRMS (ESI) m/z Calcd. C₅₇ H₉₄ O₁₄ N₅ S₂ [M+H⁺]: 1136.6233, found 1136.6259.

### Synthesis of APC3NL:

Compound 8 (50 mg, 0.06 mmol) and C3NL (100 mg, 0.35 mmol) were dissolved in THF/DMSO solution (2:1 mL). CuI (20 mg, 0.05 mmol) and the mixture was purged with argon with stirring for 5 minutes. Hunig's base (0.3 mL) was added and the mixture was stirred overnight. The reaction was partitioned between NH₄OH (1M, 30 mL) and DCM (30 mL) and the two layers were separated. The aqueous layer was extracted with DCM (20 mL×2), the combined DCM layer was dried Na₂SO₄ and evaporated in vacuo. The crude was purified using prep TLC, eluting with DCM:MeOH=10:1 to furnish APC3NL as whitish-yellow solid (33 mg, 49%). ¹H NMR (700 MHz, CDCl₃) δ 8.25 (d, J=1.6 Hz, 1H), 7.97-7.68 (m, 3H), 7.38 (d, J=7.9 Hz, 2H), 6.07 (t, J=6.2 Hz, 1H), 5.32 (s, 1H), 5.15 (d, J=4.9 Hz, 2H), 4.71 (dd, J=9.9, 2.7 Hz, 1H), 4.60-4.36 (m, 3H), 4.26 (dd, J=3.9, 2.0 Hz, 1H), 4.06 (dq, J=8.9, 6.2 Hz, 2H), 3.82 (d, J=13.0 Hz, 1H), 3.77-3.58 (m, 3H), 3.58-3.43 (m, 4H), 3.41-3.25 (m, 4H), 3.25-2.86 (m, 7H), 2.84-2.47 (m, 7H), 2.47-2.16 (m, 14H), 2.16-1.87 (m, 7H), 1.87-1.59 (m, 4H), 1.59-1.41 (m, 3H), 1.41-0.99 (m, 17H), 0.99-0.79 (m, 9H). ¹³C NMR (176 MHz, CDCl₃) δ 178.9, 161.8, 147.8, 139.1, 129.4, 129.3, 125.8, 120.0, 102.9, 94.5, 83.6, 78.1, 77.7, 77.2, 77.0, 76.8, 74.2, 73.7, 73.6, 72.9, 70.7, 70.1, 68.7, 65.9, 65.6, 64.4, 62.5, 57.7, 49.3, 47.6, 45.3, 42.3, 42.3, 40.3, 36.9, 36.2, 35.1, 34.6, 31.9, 30.1, 29.7, 29.7, 27.6, 26.8, 22.7, 22.0, 21.5, 21.4, 21.3, 18.2, 16.2, 15.3, 14.8, 14.6, 14.1, 11.3, 9.0, 7.3. HRMS (ESI) m/z Calcd. C₅₇ H₉₇ O₁₃ N₆ S₂ [M+H⁺]: 1137.6550, found 1137.6565.

### Synthesis of AZM-613:

Compound 7 (200 mg, 0.27 mmol) was dissolved in Hunig's base (0.5 mL)/DCM (5 mL) mixture with stirring. In a separate flask, CPI-613 (110 mg, 0.28 mmol) and EDCI (216 mg, 1.14 mmol) were dissolved in DCM (5 mL). After 5 minutes stirring under argon, the CPI-613 mixture was added to the mixture of 7 dropwisely with stirring, and DMAP (10 mg, 0.082 mmol) was subsequently added. The reaction was stirred at rt under argon atmosphere for 24 h. The reaction was partitioned between DCM (50 mL) and water (40 mL) and the two layers were separated. Then, the organic layer was washed with water (30 mL), dried over Na₂SO₄ and evaporated in vacuo. The crude was purified using prep TLC eluting with DCM:MeOH=15:1 to furnish AZM-613 as white foam (293 mg, 97%). ¹H NMR (700 MHz, DMSO) δ 7.32-7.27 (m, 8H), 7.22 (dtd, J=12.2, 6.2, 5.4, 2.5 Hz, 2H), 4.86 (t, J=80.6 Hz, 4H), 4.60 (s, 1H), 4.46 (dd, J=25.0, 7.1 Hz, 2H), 4.30 (s, 2H), 4.25-4.13 (m, 2H), 4.10 (ddt, J=16.0, 12.2, 6.1 Hz, 2H), 4.03 (q, J=7.1 Hz, 1H), 3.82 (s, 1H), 3.67 (dd, J=16.0, 3.0 Hz, 8H), 3.55-3.50 (m, 2H), 3.45 (s, 2H), 3.33 (s, 5H), 3.26 (d, J=14.1 Hz, 4H), 3.21-3.09 (m, 1H), 2.94 (s, 1H), 2.79 (s, 2H), 2.66 (s, 2H), 2.57 (tt, J=12.8, 7.3 Hz, 2H), 2.50 (dt, J=3.6, 1.8 Hz, 5H), 2.48-2.39 (m, 3H), 2.32-2.13 (m, 8H), 1.99 (s, 1H), 1.96-1.83 (m, 2H), 1.83-1.72 (m, 2H), 1.72-1.62 (m, 4H), 1.60-1.55 (m, 5H), 1.44 (dddd, J=47.1, 37.7, 20.8, 10.4 Hz, 7H), 1.36-1.29 (m, 2H), 1.29-1.22 (m, 3H), 1.17 (dd, J=15.3, 8.2 Hz, 4H), 1.12 (s, 1H), 1.08 (d, J=6.0 Hz, 4H), 1.05 (d, J=6.0 Hz, 1H), 1.02 (s, 2H), 0.98-0.92 (m, 5H), 0.85 (s, 2H), 0.80 (t, J=6.8 Hz, 3H). ¹³C NMR (176 MHz, DMSO) δ 177.6, 172.7, 139.3, 129.3, 128.6, 127.2, 102.6, 94.8, 77.9, 74.1, 73.3, 70.2, 67.0, 60.2, 49.3, 45.2, 44.6, 44.4, 36.9, 35.5, 34.5, 34.4, 34.2, 33.7, 33.1, 28.5, 26.8, 26.6, 26.4, 25.2, 24.9, 21.6, 21.5, 21.2, 19.0, 18.2, 14.6, 11.4. HRMS (ESI) m/z Calcd. C₅₉ H₉₆ O₁₃ N₂ S₂ [M+H⁺]: 1105.6427, found 1105.6417.

### Synthesis of AO-03-032:

CPI-613 (200 mg, 0.51 mmol) and EDCI (60 mg, 0.31 mmol) were dissolved in DCM (5 mL) and stirred at rt for about 3 h. AZM (385 mg, 0.51 mmol) and DCM (5 mL) was added and the mixture was stirred at rt overnight. DCM (40 mL) and water (30 mL) were added and the two layers separated. The organic layer was extracted with water (30 mL), sat. NaHCO₃ (30 mL), brine (30 mL) and dried over Na₂SO₄. The crude was purified on silica gel column, eluting with DCM:MeOH ratio 40:1 to 30:1 to 20:1 to 15:1 to 10:1 to furnish AO-03-032 as thick pasty foam (89 mg, 31%). ¹H NMR (700 MHz, CDCl₃) δ 7.35-7.28 (m, 7H), 7.27-7.21 (m, 2H), 5.08 (s, 1H), 4.85-4.70 (m, 1H), 4.57 (d, J=7.4 Hz, 1H), 4.24 (s, 1H), 4.04 (dq, J=12.4, 6.1 Hz, 1H), 3.84-3.50 (m, 6H), 3.37 (s, 2H), 3.06 (t, J=9.6 Hz, 1H), 2.75 (s, 1H), 2.59 (dp, J=11.8, 5.9 Hz, 1H), 2.54-2.48 (m, 3H), 2.45-2.14 (m, 9H), 2.05-1.98 (m, 1H), 1.96-1.86 (m, 2H), 1.75 (tq, J=14.4, 7.1 Hz, 3H), 1.68 (d, J=14.7 Hz, 1H), 1.64-1.58 (m, 1H), 1.55 (ddd, J=20.7, 14.0, 7.5 Hz, 2H), 1.52-1.43 (m, 4H), 1.35 (dd, J=20.2, 6.4 Hz, 7H), 1.28-1.19 (m, 8H), 1.08 (s, 2H), 0.99-0.87 (m, 7H). ¹³C NMR (176 MHz, CDCl₃) δ 172.4, 138.6, 138.5, 128.9, 128.9, 128.8, 128.5, 128.5, 127.0, 127.0, 100.6, 94.9, 83.3, 78.1, 77.2, 77.1, 76.9, 74.4, 73.1, 68.2, 65.7, 63.9, 53.5, 50.5, 49.4, 45.1, 44.3, 41.6, 40.7, 36.6, 36.4, 35.1, 34.8, 34.6, 34.5, 34.4, 30.2, 29.7, 28.7, 27.2, 26.5, 26.1, 24.7, 22.2, 21.6, 21.4, 21.2, 18.3, 16.4, 15.0, 11.3, 9.1. MS (LC) m/z cal for C₆₀ H₉₈ O₁₃ N₂ S₂ [M+H⁺]: 1119.66, found 1119.81.

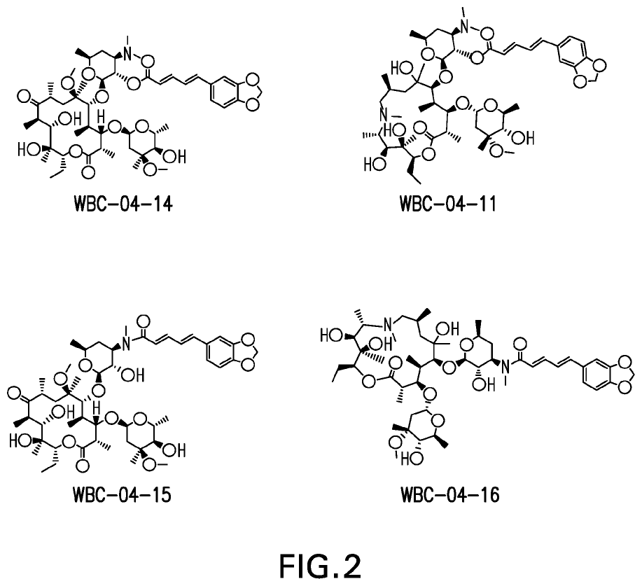

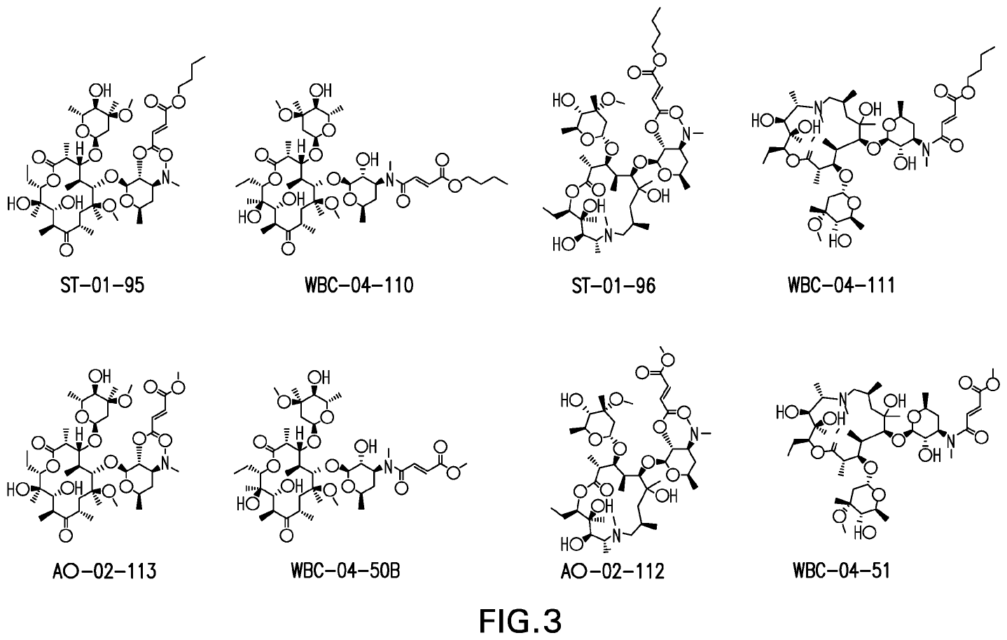

The target piperine- and fumaryl-compounds (FIG. 2 and FIG. 3) are synthesized as outlined in Schemes 11-15. WBC-04-11 and WBC-04-14 were synthesized from piperic acid anhydride as described for the analogous lipoyl-compounds (Scheme 11). Similarly, WBC-04-15 and WBC-04-16 were synthesized from EDCI-mediated coupling of compound 4 or 7 to piperic acid as described the lipoyl congeners (Scheme 12).

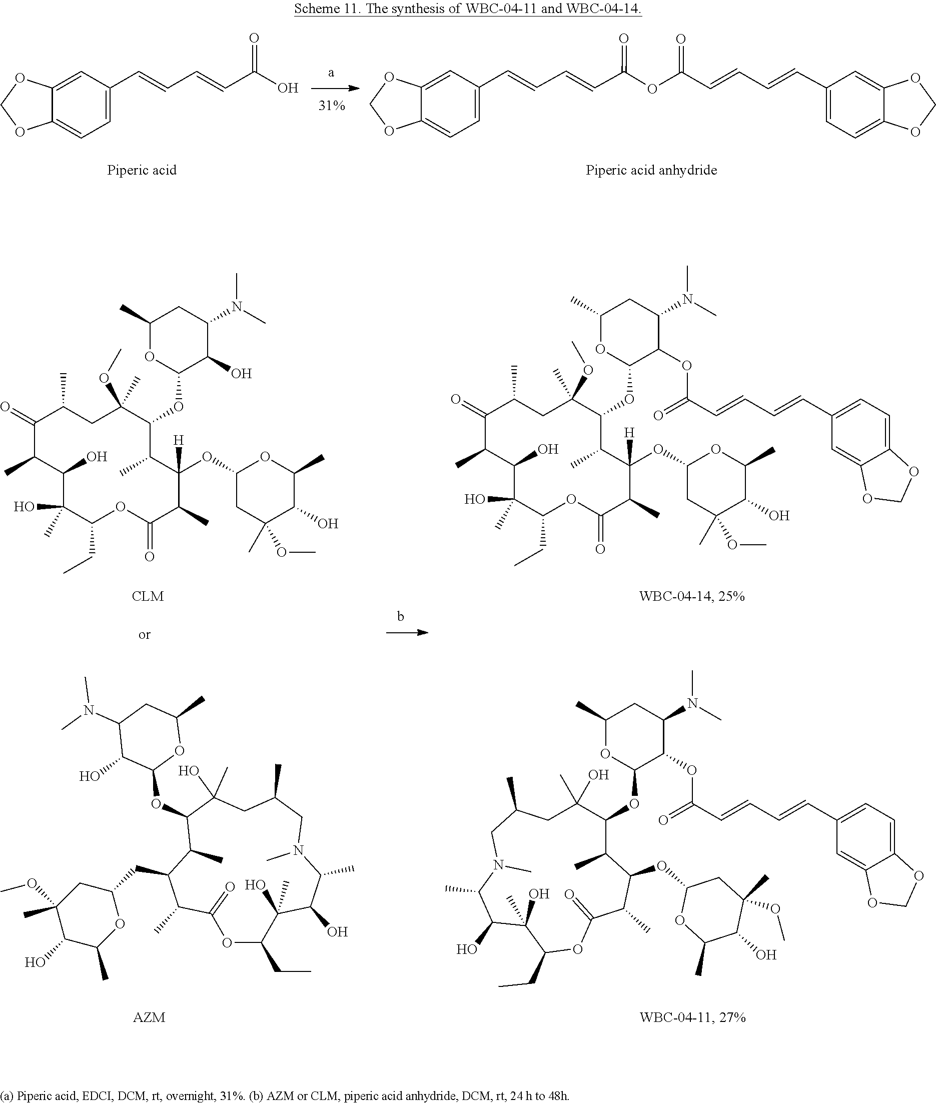

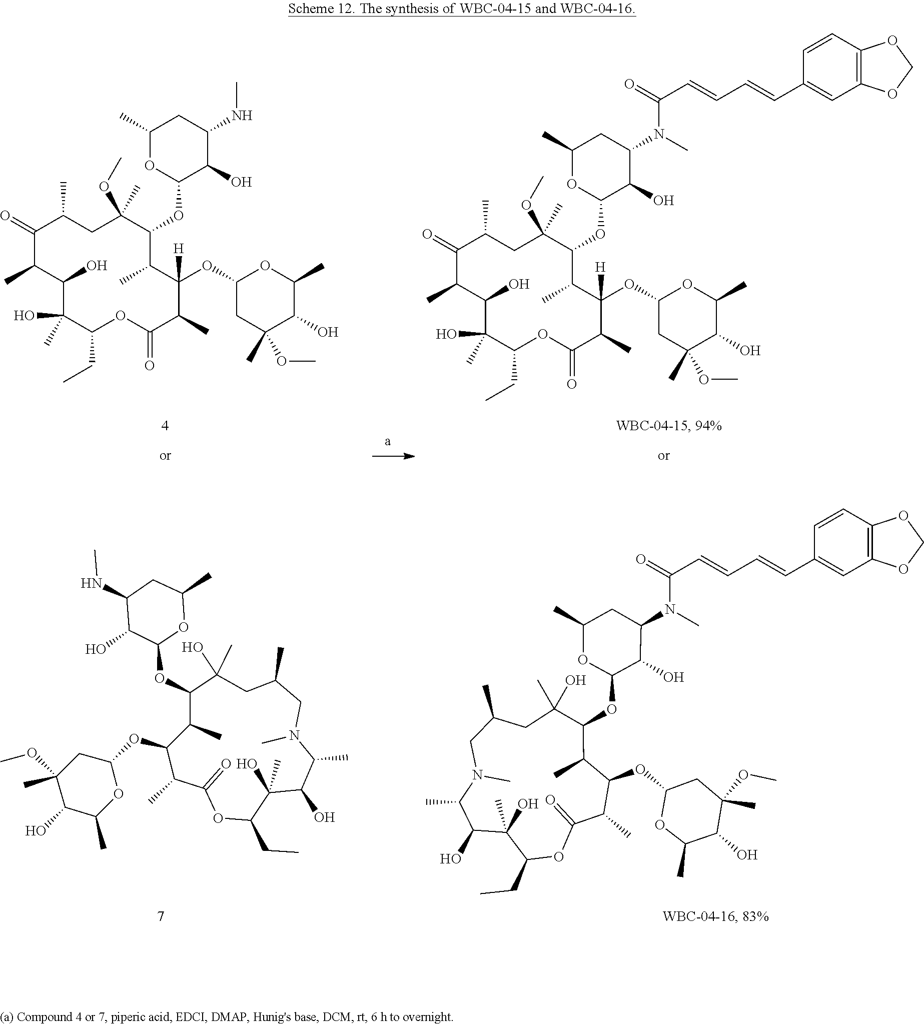

The synthesis of the target furmaryl compounds requires monomethyl fumarate and monobutyl fumarate intermediates. The monomethyl fumarate was obtained from a commercial source monobutyl fumarate was synthesized from maleic anhydride adapting a patent protocol (US 2014/0364604) (Scheme 13). The target furmaryl compounds were synthesized using similar protocols described for the synthesis of lipoyl and piperine analogs (Schemes 14-15).

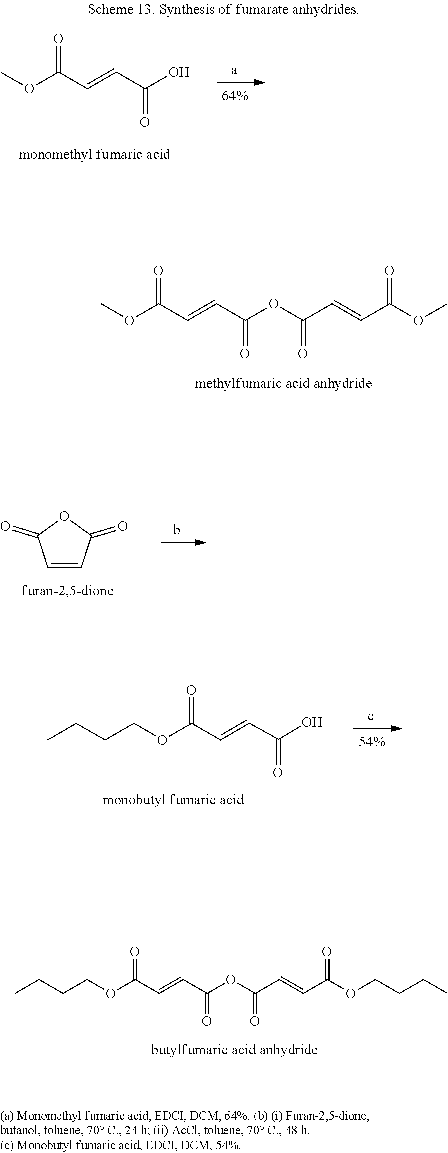

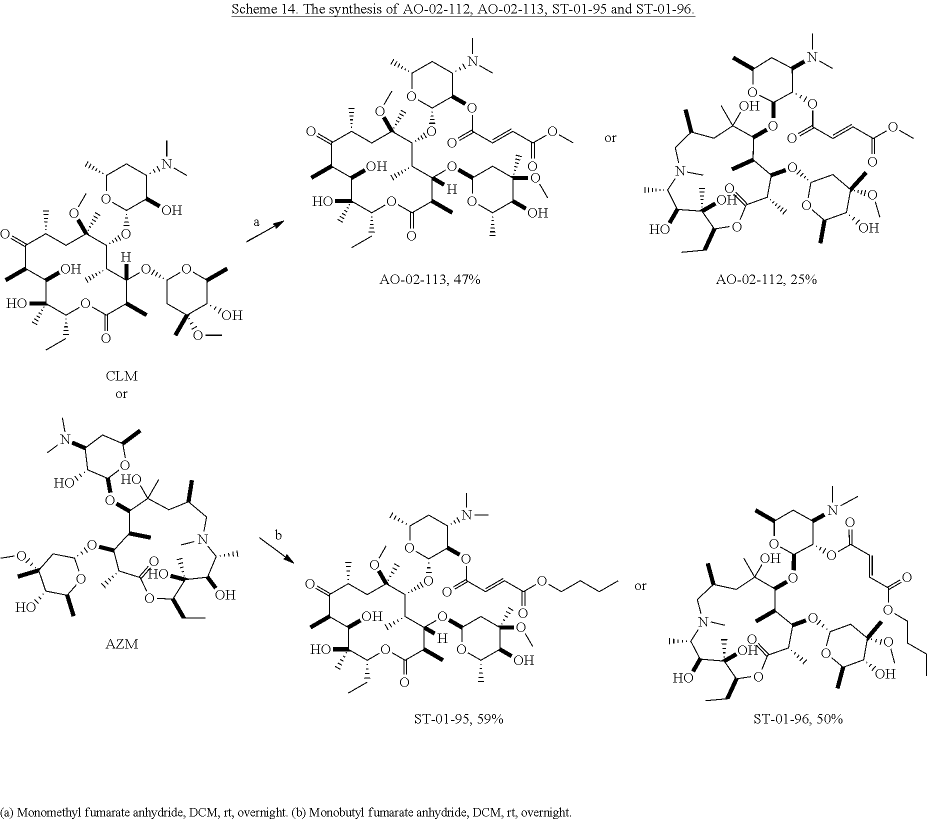

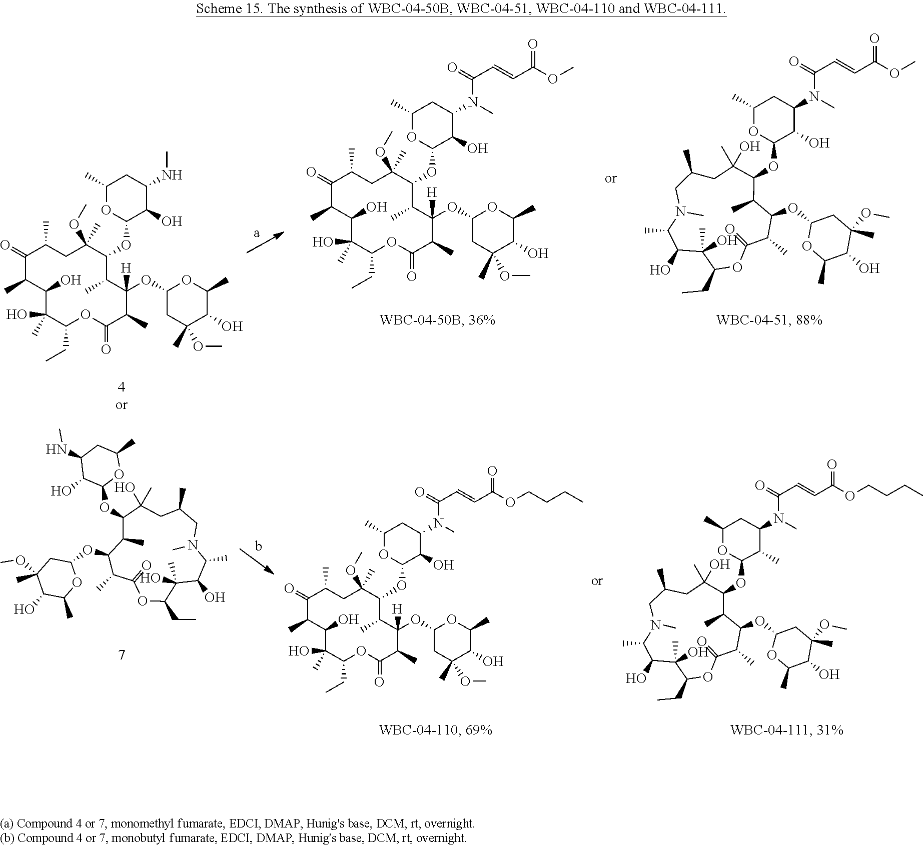

Synthesis Procedures for target compounds AO-02-112, AO-02-113, ST-01-95, ST-01-96, WBC-04-50B, WBC-04-51, WBC-04-110 and WBC-04-111.

### Synthesis of Piperic Acid Anhydride:

Piperic acid (400 mg, 1.83 mmol) was dissolved in DCM (10 mL) and EDCI (181 mg, 1.16 mmol) was added. The mixture was stirred at rt overnight under argon, water (30 mL) and DCM (20 mL) were added and the two layers separated. The DCM layer was extracted with water (20 mL×3), dried over Na₂SO₄ and evaporated in vacuo to yield crude Piperic acid anhydride as yellow solid (235 mg, 31%).

### Synthesis of WBC-04-11:

AZM (170 mg, 0.23 mmol) and the crude piperic acid anhydride (235 mg, 0.56 mmol) were dissolved in DCM (10 mL). The mixture was stirred at rt overnight under argon. The reaction was partitioned between water (30 mL) and (30 mL), the two layers were separated and the DCM layer extracted with water (30 mL×2). The organic layer was dried over Na₂SO₄ and evaporated in vacuo. The crude was purified using prep TLC eluting with DCM:MeOH=10:1 to furnish WBC-04-11 as whitish-yellow solid (58 mg, 27%). ¹H NMR (700 MHz, CDCl₃) δ 7.41 (dd, J=15.2, 10.8 Hz, 1H), 7.01 (d, J=1.6 Hz, 1H), 6.93 (d, J=8.0 Hz, 1H), 6.87-6.77 (m, 2H), 6.72 (dd, J=15.5, 10.9 Hz, 1H), 6.01 (s, 2H), 5.95 (d, J=15.2 Hz, 1H), 5.09 (d, J=4.8 Hz, 1H), 4.92 (q, J=10.0, 9.5 Hz, 1H), 4.69 (d, J=9.6 Hz, 1H), 4.63 (d, J=7.5 Hz, 1H), 4.25 (d, J=5.2 Hz, 1H), 4.07 (dt, J=12.2, 6.1 Hz, 1H), 3.68 (s, 1H), 3.62 (d, J=7.1 Hz, 1H), 3.57 (t, J=9.6 Hz, 1H), 3.50 (q, J=7.0 Hz, 2H), 3.43 (s, 3H), 3.40-3.33 (m, 1H), 3.08 (t, J=8.3 Hz, 1H), 2.77-2.69 (m, 3H), 2.52 (d, J=11.9 Hz, 1H), 2.38 (d, J=15.0 Hz, 4H), 2.34 (s, 4H), 2.18 (d, J=10.2 Hz, 1H), 1.94-1.85 (m, 2H), 1.71 (d, J=14.6 Hz, 1H), 1.62 (dd, J=15.2, 5.3 Hz, 2H), 1.45 (tdd, J=13.8, 8.2, 4.9 Hz, 2H), 1.42-1.36 (m, 2H), 1.36-1.32 (m, 6H), 1.31-1.26 (m, 9H), 1.26 (s, 2H), 1.25-1.18 (m, 8H), 1.12 (tq, J=15.2, 6.8 Hz, 4H), 1.05 (s, 4H), 0.95 (d, J=6.8 Hz, 3H), 0.94-0.82 (m, 9H). ¹³C NMR (176 MHz, CDCl₃) δ 178.4, 166.0, 148.6, 148.3, 139.9 130.6, 124.7, 123.6, 122.9, 109.2, 105.8, 101.4, 101.3, 100.8, 94.8, 83.3, 78.1, 77.2, 77.0, 76.8, 74.3, 73.8, 73.1, 71.6, 70.1, 68.2, 65.9, 65.7, 63.8, 49.4, 45.1, 42.0, 41.6, 40.8, 36.5, 34.9, 31.9, 31.2, 29.7, 29.4, 27.3, 26.6, 22.7, 22.0, 21.7, 21.6, 21.2, 18.3, 18.3, 16.2, 15.3, 15.1, 14.1, 11.3, 9.3, 7.6. HRMS (ESI) m/z Calcd. C₅₀ H₈₁ O₁₅ N₂ [M+H⁺]: 949.5631, found 949.5605.

### Synthesis of WBC-04-14:

CLM (100 mg, 0.13 mmol) and piperic acid anhydride (200 mg, 0.48 mmol) were dissolved in DCM (10 mL). The mixture was stirred at rt overnight under argon. The reaction was partitioned between water (30 mL) and (30 mL), the two layers were separated and the DCM layer extracted with water (30 mL×2). The organic layer was dried over Na₂SO₄ and evaporated in vacuo. The crude was purified using prep TLC eluting with DCM:MeOH=10:1 to furnish WBC-04-14 as whitish-yellow solid (30 mg, 25%). ¹H NMR (700 MHz, CDCl₃) δ 7.41 (dd, J=15.2, 10.8 Hz, 1H), 7.01 (d, J=1.7 Hz, 1H), 6.93 (dd, J=8.0, 1.8 Hz, 1H), 6.85-6.77 (m, 2H), 6.72 (dd, J=15.6, 10.8 Hz, 1H), 6.01 (s, 2H), 5.93 (d, J=15.2 Hz, 1H), 5.31 (s, 1H), 5.04 (dd, J=11.0, 2.4 Hz, 1H), 4.94 (d, J=4.9 Hz, 1H), 4.88 (dd, J=10.6, 7.5 Hz, 1H), 4.64 (d, J=7.4 Hz, 1H), 4.02 (dq, J=8.8, 6.2 Hz, 1H), 3.98 (s, 1H), 3.78-3.74 (m, 1H), 3.74 (d, J=1.7 Hz, 1H), 3.63 (d, J=6.9 Hz, 1H), 3.53 (ddd, J=10.8, 6.2, 1.9 Hz, 1H), 3.44 (s, 3H), 3.20 (s, 1H), 3.07 (d, J=9.0 Hz, 1H), 3.02 (s, 3H), 2.97 (tt, J=7.0, 3.9 Hz, 1H), 2.87-2.80 (m, 1H), 2.70 (d, J=12.4 Hz, 1H), 2.58 (dtd, J=13.6, 7.0, 2.1 Hz, 1H), 2.41-2.36 (m, 1H), 2.30 (s, 6H), 2.24-2.18 (m, 1H), 1.94-1.86 (m, 1H), 1.86-1.80 (m, 1H), 1.79-1.74 (m, 1H), 1.69 (dd, J=14.8, 11.7 Hz, 1H), 1.62 (ddd, J=14.8, 6.3, 3.7 Hz, 2H), 1.44 (dtd, J=14.2, 7.2, 4.3 Hz, 2H), 1.38-1.32 (m, 3H), 1.31 (d, J=8.3 Hz, 5H), 1.26 (dd, J=12.6, 4.9 Hz, 6H), 1.21 (dd, J=7.3, 3.9 Hz, 4H), 1.15 (d, J=7.0 Hz, 3H), 1.14-1.08 (m, 7H), 0.88 (d, J=7.5 Hz, 3H), 0.83 (t, J=7.4 Hz, 3H). ¹³C NMR (176 MHz, CDCl₃) δ 175.7, 166.0, 148.6, 148.33, 144.7, 134.0, 130.5, 124.6, 122.9, 120.9, 108.6, 105.8, 101.4, 100.8, 95.8, 95.8, 80.6, 78.4, 78.1, 77.9, 77.2, 77.0, 76.6, 74.2, 72.9, 72.8, 71.6, 69.1, 68.2, 65.9, 65.8, 63.6, 53.4, 50.4, 49.5, 49.3, 45.2, 45.0, 40.8, 39.1, 38.7, 37.3, 34.9, 31.0, 29.7, 21.6, 21.3, 21.0, 19.9, 18.7, 17.9, 16.0, 12.3, 10.6, 9.2. HRMS (ESI) m/z Calcd. C₅₀ H₇₈ O₁₆ N [M+H⁺]: 948.5315, found 948.5293.

### Synthesis of WBC-04-15:

Compound 4 (200 mg, 0.27 mmol) was dissolved in Hunig's base (0.5 mL)/DCM (5 mL) mixture with stirring at rt. In a separate flask, piperic acid (75 mg, 0.34 mmol) and EDCI (210 mg, 1.35 mmol) were dissolved in DCM (5 mL). After 5 minutes stirring, the piperic acid mixture was added to the compound 4 solution dropwisely with stirring, and DMAP (10 mg, 0.08 mmol) was subsequently added. The reaction was stirred under argon atmosphere at rt for 24 h. The reaction was partitioned between DCM (50 mL) and water (40 mL) and the two layers were separated. Then, the organic layer was washed with water (30 mL), dried over Na₂SO₄ and evaporated in vacuo. The crude was purified using prep TLC eluting with EtOAc:MeOH=20:1 to furnish WBC-04-15 as whitish-yellow foam (240 mg, 94%). ¹H NMR (700 MHz, CDCl₃) δ 7.46 (dd, J=14.5, 10.1 Hz, 1H), 7.01 (s, 1H), 6.92 (d, J=8.0 Hz, 1H), 6.82-6.79 (m, 2H), 6.77 (d, J=10.4 Hz, 1H), 6.41 (d, J=14.6 Hz, 1H), 6.00 (s, 2H), 5.32 (d, J=1.8 Hz, 1H), 5.08 (d, J=10.8 Hz, 1H), 4.95 (d, J=5.1 Hz, 1H), 4.76 (td, J=11.9, 4.1 Hz, 1H), 4.56 (d, J=7.1 Hz, 1H), 4.05 (q, J=7.1, 6.6 Hz, 1H), 3.99 (s, 1H), 3.77 (d, J=8.6 Hz, 2H), 3.73-3.69 (m, 1H), 3.69 (s, 1H), 3.39 (d, J=11.0 Hz, 4H), 3.21 (s, 1H), 3.06 (s, 4H), 3.04-2.98 (m, 4H), 2.95 (s, 1H), 2.89 (q, J=7.7 Hz, 1H), 2.73 (d, J=4.6 Hz, 1H), 2.61 (dq, J=13.9, 7.5, 6.6 Hz, 2H), 2.39 (d, J=15.1 Hz, 1H), 2.01-1.94 (m, 1H), 1.94-1.89 (m, 1H), 1.81 (t, J=13.4 Hz, 1H), 1.72 (d, J=18.3 Hz, 2H), 1.69-1.59 (m, 3H), 1.56-1.45 (m, 2H), 1.44 (s, 3H), 1.34 (d, J=6.1 Hz, 3H), 1.30-1.21 (m, 10H), 1.19-1.13 (m, 9H), 1.08 (dd, J=22.7, 7.6 Hz, 3H), 0.86 (t, J=7.4 Hz, 3H). ¹³C NMR (176 MHz, CDCl₃) δ 220.9, 175.7, 168.6, 148.3, 148.2, 143.8, 139.3, 130.8, 125.0, 122.8, 119.9, 108.5, 105.7, 103.7, 101.3, 96.2, 82.1, 78.5, 78.3, 78.0, 77.2, 74.2, 72.8, 72.1, 69.1, 68.4, 65.8, 54.4, 53.4, 50.7, 49.5, 45.2, 45.0, 39.2, 38.9, 37.3, 35.8, 35.01, 29.8, 21.6, 21.1, 21.0, 19.7, 18.7, 18.0, 16.0, 12.3, 10.6, 9.5. HRMS (ESI) m/z Calcd. C₄₉ H₇₅ O₁₆N Na [M+Na⁺]: 956.4978, found 956.4958.

### Synthesis of WBC-04-16:

Compound 7 (180 mg, 0.25 mmol) was mixed with Hunig's base (0.5 mL) in 5 ml DCM solution with stirring. In a separate flask, piperic acid (85 mg, 0.39 mmol) and EDCI (200 mg, 1.29 mmol) were dissolved in DCM (5 mL). After 5 minutes stirring, the piperic acid mixture was added to the compound 4 solution dropwisely with stirring, and DMAP (10 mg, 0.08 mmol) was subsequently added. The reaction was stirred under argon atmosphere at rt for 24 h. The reaction was partitioned between DCM (50 mL) and water (40 mL) and the two layers were separated. Then, the organic layer was washed with water (30 mL), dried over Na₂SO₄ and evaporated in vacuo. The crude was purified using prep TLC eluting with EtOAc:MeOH=13:1 to furnish WBC-04-16 as whitish-yellow foam (190 mg, 83%). ¹H NMR (700 MHz, CDCl₃) δ 7.46 (dd, J=14.6, 9.4 Hz, 1H), 7.00 (d, J=12.1 Hz, 1H), 6.92 (d, J=8.2 Hz, 1H), 6.82-6.77 (m, 2H), 6.77-6.73 (m, 1H), 6.42 (d, J=14.6 Hz, 1H), 6.00 (s, 2H), 5.32 (d, J=1.7 Hz, 1H), 5.05 (dd, J=18.2, 4.7 Hz, 1H), 4.79 (td, J=11.6, 4.1 Hz, 1H), 4.69 (d, J=10.2 Hz, 1H), 4.56 (dd, J=25.0, 7.3 Hz, 1H), 4.25 (d, J=5.7 Hz, 1H), 4.14-4.08 (m, 1H), 3.72-3.67 (m, 2H), 3.52-3.48 (m, 2H), 3.41 (s, 2H), 3.37 (s, 1H), 3.08 (q, J=9.9 Hz, 1H), 3.03 (s, 2H), 2.95 (s, 1H), 2.80 (d, J=14.2 Hz, 2H), 2.73 (t, J=6.8 Hz, 1H), 2.68-2.65 (m, 1H), 2.55 (t, J=10.5 Hz, 2H), 2.36 (d, J=3.7 Hz, 3H), 2.08 (t, J=11.8 Hz, 1H), 2.05-2.00 (m, 2H), 1.90 (dq, J=15.0, 7.6 Hz, 1H), 1.81-1.67 (m, 3H), 1.61 (dd, J=15.1, 5.4 Hz, 1H), 1.58-1.47 (m, 2H), 1.38-1.34 (m, 6H), 1.32-1.20 (m, 13H), 1.12 (d, J=6.7 Hz, 3H), 1.09 (s, 3H), 1.04 (d, J=7.3 Hz, 2H), 1.01 (d, J=7.8 Hz, 1H), 0.93 (dt, J=24.4, 7.3 Hz, 6H). ¹³C NMR (176 MHz, CDCl₃) δ 178.5, 168.3, 148.3, 148.2, 143.5, 139.0, 130.9, 125.2, 122.7, 120.2, 108.5, 105.7, 103.7, 101.3, 95.3, 85.2, 78.7, 78.1, 77.2, 77.0, 76.9, 74.5, 74.3, 73.5, 73.0, 71.8, 70.1, 68.4, 65.9, 65.8, 62.2, 54.5, 53.4, 49.5, 45.0, 42.3, 41.1, 35.8, 35.0, 29.8, 27.3, 26.7, 22.0, 21.7, 21.1, 18.3, 16.2, 15.3, 15.3, 11.2, 9.6, 7.6. HRMS (ESI) m/z Calcd. C₄₉ H₇₉ O₁₅ N₂ [M+H⁺]: 935.5475, found 935.5466.

### Synthesis of Methyl Fumaric Acid Anhydride:

Monomethyl fumaric acid (929 mg, 5.47 mmol) was dissolved into DCM (25 mL) and EDCI (630 mg, 3.28 mmol) was added. The mixture was stirred at rt overnight under argon, water (30 mL) and DCM (20 mL) were added and the two layers separated. The DCM layer was extracted with water (20 mL×3), dried over Na₂SO₄ and evaporated in vacuo to yield crude methyl fumaric acid anhydride (458 mg, 64%).

### Synthesis of Monobutyl Fumaric Acid (US 2014/0364604):

Furan-2,5-dione (1.5 g, 15.3 mmol) was dissolved 1-butanol (7.5 mL) and Toluene (7.5 mL) and the resulting mixture was heated to 70° C. for 24 h. Solvent was evaporated off and the crude was purified using column chromatography, eluting EtOAc:hexane:MeOH 4:1:0.5 to furnish to give intermediate product (2.26 g). The intermediate (1.57 g) was dissolved in toluene (7.5 mL), acetyl chloride (100 μL, 0.28 mmol) was added and the mixture was heated to 70° C. again for 48 h. EtOAc (25 mL) and water (40 mL) were added to the reaction and the two layers separated. The organic layer was washed with water (30 mL), dried by Na₂SO₄ and evaporated in vacuo to furnish monobutyl fumaric acid as white solid (1.40 g, 54%).

Butyl fumaric acid anhydride was synthesized as described for the methyl fumaric acid anhydride.

### Synthesis of A0-02-112:

A mixture of AZM (500 mg, 0.67 mmol) and fumaric anhydride (1.05 mmol dissolved in 10 mL DCM) in DCM (5 mL) was stirred overnight at rt under argon atmosphere. DCM (40 mL) and water (30 mL) were added and the two layers separated. The organic layer was extracted with water (30 mL), sat. NaHCO₃ (30 mL), brine (30 mL) and dried over Na₂SO₄. The crude was purified using prep TLC eluting with DCM:MeOH 14:1 to furnish AO-02-112 as whitish solid (147 mg, 25%). ¹H NMR (700 MHz, CDCl₃) δ 6.86-6.78 (m, 2H), 5.29 (s, 1H), 4.96 (s, 1H), 4.84 (dd, J=10.7, 7.5 Hz, 1H), 4.67-4.64 (m, 1H), 4.61 (d, J=7.5 Hz, 1H), 4.18 (d, J=5.6 Hz, 1H), 4.03-3.98 (m, 1H), 3.79 (s, 4H), 3.67 (s, 2H), 3.67-3.62 (m, 1H), 3.63 (s, 1H), 3.61-3.57 (m, 1H), 3.53 (s, 1H), 3.34 (s, 3H), 3.03 (t, J=9.3 Hz, 1H), 2.75 (s, 3H), 2.68 (d, J=12.6 Hz, 1H), 2.48-2.45 (m, 2H), 2.35-2.25 (m, 1H), 2.23 (s, 6H), 1.95 (s, 1H), 1.89 (s, 1H), 1.85 (ddd, J=14.1, 7.4, 2.3 Hz, 1H), 1.76-1.71 (m, 1H), 1.66 (d, J=14.7 Hz, 1H), 1.58 (dd, J=15.1, 5.1 Hz, 1H), 1.45 (ddt, J=17.2, 14.4, 7.2 Hz, 1H), 1.33 (dd, J=12.8, 11.1 Hz, 1H), 1.32-1.26 (m, 6H), 1.25 (s, 3H), 1.23 (s, 2H), 1.23-1.18 (m, 5H), 1.11 (s, 3H), 1.09 (d, J=8.0 Hz, OH), 1.02 (s, 3H), 0.92 (d, J=7.1 Hz, 3H), 0.87 (t, J=7.5 Hz, 3H), 0.79 (d, J=7.6 Hz, 3H). ¹³C NMR (176 MHz, CDCl₃) δ 178.1, 165.6, 165.3, 163.9, 134.5, 133.4, 132.8, 100.4, 95.2, 83.7, 78.7, 78.0, 77.4, 77.3, 77.1, 76.9, 74.4, 73.6, 73.0, 72.8, 70.5, 68.3, 65.6, 63.6, 53.5, 52.3, 52.3, 49.4, 44.8, 42.0, 40.7, 36.6, 35.0, 30.3, 27.1, 26.5, 22.0, 21.6, 21.6, 21.2, 18.4, 18.3, 16.3, 15.5, 11.2, 9.6. HRMS (ESI) m/z Calcd. C₄₃ H₇₆ O₁₅ N₂ [M+2H+]: 431.2695, found 431.2696.

### Synthesis of AO-02-113:

A mixture of CLM (500 mg, 0.67 mmol) and fumaric anhydride (1.05 mmol dissolved in 10 mL DCM) in DCM (5 mL) was stirred overnight at rt under argon atmosphere. DCM (40 mL) and water (30 mL) were added and the two layers separated. The organic layer was extracted with water (30 mL), sat. NaHCO₃ (30 mL), brine (30 mL) and dried over Na₂SO₄. The crude was purified using prep TLC eluting with DCM:MeOH 16:1 to furnish Synthesis of AO-02-113 as whitish solid (273 mg, 47%). ¹H NMR (700 MHz, CDCl₃) δ 6.79 (s, 2H), 5.26 (s, 1H), 4.98 (dd, J=11.1, 2.4 Hz, 1H), 4.88-4.85 (m, 1H), 4.78 (dd, J=10.7, 7.4 Hz, 1H), 4.58 (d, J=7.4 Hz, 1H), 3.96-3.90 (m, 1H), 3.91 (s, 1H), 3.77 (s, 3H), 3.70-3.65 (m, 2H), 3.57 (d, J=6.8 Hz, 1H), 3.49 (dqd, J=12.1, 5.9, 1.9 Hz, 1H), 3.33 (s, 3H), 3.17 (s, 1H), 3.00 (t, J=9.1 Hz, 1H), 2.95 (s, 3H), 2.91 (tt, J=6.9, 3.3 Hz, 1H), 2.76 (dq, J=9.4, 7.3 Hz, 1H), 2.68-2.62 (m, 1H), 2.54-2.47 (m, 1H), 2.33-2.26 (m, 2H), 1.85 (dqd, J=15.1, 7.5, 2.2 Hz, 1H), 1.80-1.70 (m, 2H), 1.64-1.53 (m, 2H), 1.51 (dd, J=14.8, 2.1 Hz, 1H), 1.40 (ddq, J=14.4, 11.1, 7.2 Hz, 1H), 1.32 (s, 3H), 1.31-1.25 (m, 1H), 1.26-1.22 (m, 6H), 1.19 (d, J=6.1 Hz, 3H), 1.14 (d, J=7.4 Hz, 3H), 1.06 (dd, J=17.2, 7.1 Hz, 6H), 1.05 (s, 3H), 0.77 (t, J=7.3 Hz, 6H). ¹³C NMR (176 MHz, CDCl₃) δ 175.7, 165.5, 163.9, 134.1, 133.0, 100.2, 95.8, 80.5, 78.2, 78.0, 77.8, 77.3, 77.1, 76.9, 76.6, 74.1, 72.8, 72.6, 69.1, 68.1, 65.8, 63.4, 53.5, 52.3, 50.4, 49.5, 45.1, 44.9, 40.6, 38.9, 38.6, 37.2, 34.9, 30, 3, 21.4, 21.2, 21.0, 19.8, 18.6, 17.8, 16.1, 15.9, 12.2, 10.5, 9.3. HRMS (ESI) m/z Calcd. C₄₃ H₇₃ O₁₆ N [M+H⁺]: 860.5002, found 860.5005.

### Synthesis of ST-01-95:

CLM (235 mg, 0.32 mmol) and butyl fumarate anhydride (182 mg, 0.98 mmol) were stirred in DCM (10 mL) at room temperature for 24 h. The reaction was stopped by adding sat. NaHCO₃ (30 mL) and extracted with DCM (40 mL). The organic layer was washed sat. NaHCO₃ (30 mL×2), brine (25 mL), dried over Na₂SO₄ and evaporated in vacuo. The crude was purified using column chromatography, eluting with a gradient of DCM:MeOH 20:1 to 16:1 to furnish ST-01-95 as white foam (170 mg, 59%). ¹H NMR (700 MHz, CDCl₃) δ 6.80 (s, 2H), 5.00 (dd, J=11.0, 2.5 Hz, 1H), 4.88 (d, J=5.2 Hz, 1H), 4.79 (dd, J=10.8, 7.4 Hz, 1H), 4.59 (d, J=7.5 Hz, 1H), 4.18 (td, J=6.7, 2.6 Hz, 2H), 3.98-3.92 (m, 2H), 3.72-3.68 (m, 2H), 3.59 (d, J=6.9 Hz, 1H), 3.49 (ddt, J=12.2, 8.0, 3.8 Hz, 1H), 3.34 (s, 3H), 3.04-2.99 (m, 1H), 2.97 (s, 3H), 2.94 (d, J=7.3 Hz, 1H), 2.81-2.74 (m, 1H), 2.63 (td, J=11.5, 4.2 Hz, 1H), 2.53 (ddt, J=14.1, 8.9, 4.5 Hz, 1H), 2.33 (d, J=15.1 Hz, 1H), 2.26 (dd, J=7.7, 4.2 Hz, 1H), 2.21 (s, 6H), 1.87 (dqd, J=15.1, 7.5, 2.4 Hz, 1H), 1.79 (q, J=7.5 Hz, 1H), 1.74-1.69 (m, 1H), 1.63 (dt, J=17.3, 8.8 Hz, 3H), 1.60-1.51 (m, 2H), 1.46-1.39 (m, 2H), 1.39-1.36 (m, 2H), 1.28-1.21 (m, 8H), 1.20 (d, J=6.1 Hz, 3H), 1.16 (d, J=7.3 Hz, 3H), 1.12-1.08 (m, 5H), 1.07 (s, 5H), 0.93 (t, J=7.5 Hz, 3H), 0.79 (dt, J=7.6, 3.7 Hz, 6H). ¹³C NMR (176 MHz, CDCl₃) δ 175.7, 165.2, 164.0, 133.9, 133.5, 100.3, 95.9, 80.5, 78.3, 78.1, 77.8, 77.3, 77.1, 76.9, 76.6, 74.1, 72.8, 72.7, 69.1, 68.2, 65.8, 65.2, 50.4, 49.5, 45.1, 45.0, 40.6, 39.0, 38.7, 37.2, 34.9, 30.1, 19.8, 19.1, 18.7, 17.9, 16.1, 15.9, 13.6, 13.4, 12.3, 10.6, 9.3. HRMS (ESI) m/z Calcd. C₄₆ H₇₉ O₁₆ N [M+H⁺]: 902.5472, found 902.5475.

### Synthesis of ST-01-96:

AZM (240 mg, 0.32 mmol) and butyl fumarate anhydride (182 mg, 0.98 mmol) were stirred in DCM (6 mL) at room temperature for 24 h. The reaction was stopped by adding sat. NaHCO₃ (30 mL) and extracted with DCM (40 mL). The organic layer was washed with sat. NaHCO₃ (30 mL×2), brine (25 mL), dried over Na₂SO₄ and evaporated in vacuo. The crude was purified using column chromatography eluting with a gradient of DCM:MeOH 10:1 to 8:1 to furnish ST-01-96 as white foam (145 mg, 50%). ¹H NMR (700 MHz, CDCl₃) δ 6.83 (d, J=2.3 Hz, 2H), 5.24 (td, J=6.0, 3.0 Hz, 1H), 5.15 (d, J=4.9 Hz, 1H), 4.89-4.82 (m, 1H), 4.68 (dd, J=9.9, 2.8 Hz, 1H), 4.58 (d, J=7.5 Hz, 1H), 4.28 (ddd, J=13.7, 6.7, 4.4 Hz, 1H), 4.21 (dd, J=5.9, 3.4 Hz, 2H), 4.20-4.17 (m, 1H), 4.15 (ddd, J=12.0, 6.0, 3.4 Hz, 1H), 4.06 (dq, J=9.5, 6.2 Hz, 1H), 3.63 (d, J=1.3 Hz, 1H), 3.57 (d, J=7.3 Hz, 1H), 3.53 (qd, J=6.1, 1.9 Hz, 1H), 3.38 (s, 3H), 3.05 (t, J=9.3 Hz, 2H), 2.67 (qd, J=7.5, 4.2 Hz, 3H), 2.53 (dd, J=11.8, 2.2 Hz, 1H), 2.39-2.33 (m, 1H), 2.33-2.30 (m, 3H), 2.24 (s, 5H), 2.17 (dd, J=10.8, 3.9 Hz, 1H), 2.09-2.05 (m, 2H), 2.05-1.95 (m, 3H), 1.89 (dtd, J=15.2, 7.6, 4.9 Hz, 2H), 1.76 (ddd, J=13.0, 4.4, 2.0 Hz, 1H), 1.70-1.63 (m, 3H), 1.60 (dd, J=10.7, 4.6 Hz, 2H), 1.49 (d, J=2.7 Hz, 1H), 1.44-1.38 (m, 3H), 1.33 (d, J=6.2 Hz, 4H), 1.32-1.20 (m, 20H), 1.17 (t, J=8.3 Hz, 5H), 1.10-1.01 (m, 6H), 0.95 (t, J=7.4 Hz, 4H), 0.92-0.85 (m, 8H), 0.80 (d, J=7.6 Hz, 3H). ¹³C NMR (176 MHz, CDCl₃) δ 178.80, 172.9, 170.5, 170.5, 170.1, 165.3, 164.0, 134.1, 133.4, 100.5, 94.4, 83.0, 78.1, 77.4, 76.9, 74.4, 73.1, 72.7, 70.1, 69.1, 68.3, 65.2, 63.6, 62.0, 52.3, 49.5, 45.2, 42.2, 41.9, 40.7, 36.2, 34.1, 34.0, 31.7, 30.3, 29.2, 27.5, 26.6, 25.3, 24.8, 22.6, 20.9, 19.1, 18.2, 16.2, 14.6, 13.7, 11.2, 9.11, 7.2. HRMS (ESI) m/z Calcd. C₄₆ H₈₂ O₁₅ N₂ [M+H⁺]: 903.5788, found 903.5798.

### Synthesis of WBC-04-50B:

Monomethyl fumaric acid (100 mg, 0.77 mmol) and EDCI (250 mg, 1.3 mmol) were dissolved in DCM (10 mL). The mixture was stirred for 5-10 minutes under argon atmosphere, compound 4 (200 mg, 0.27 mmol) was added to the solution and stirring continued at rt overnight. The reaction was partitioned between water (50 mL) and DCM (40 mL) and the two layers separated. The aqueous layer was extracted with DCM (25 mL×2), the organic layer was dried over Na₂SO₄ and evaporated in vacuo. The crude was purified using prep TLC eluting with EtOAC:MeOH 20:1.5 to furnish WBC-04-50B as whitish solid (81 mg, 36%). ¹H NMR (700 MHz, CDCl₃) δ 7.47 (dd, J=65.4, 15.4 Hz, 1H), 6.77 (dd, J=68.4, 15.4 Hz, 1H), 5.08 (dd, J=11.2, 8.6 Hz, 1H), 4.94 (t, J=6.1 Hz, 1H), 4.59-4.54 (m, 1H), 4.02 (dt, J=8.9, 5.4 Hz, 1H), 3.98 (s, 1H), 3.90-3.74 (m, 6H), 3.73-3.66 (m, 2H), 3.42 (ddd, J=17.0, 10.3, 7.2 Hz, 1H), 3.35 (d, J=16.8 Hz, 3H), 3.21 (d, J=5.8 Hz, 1H), 3.10-3.06 (m, 1H), 3.05 (s, 5H), 3.03-2.97 (m, 1H), 2.95 (s, 1H), 2.92-2.85 (m, 1H), 2.64-2.60 (m, 1H), 2.60-2.55 (m, 1H), 2.39-2.32 (m, 2H), 2.01-1.89 (m, 2H), 1.83-1.78 (m, 1H), 1.78-1.74 (m, 1H), 1.69-1.62 (m, 2H), 1.62-1.58 (m, 1H), 1.56-1.45 (m, 2H), 1.42 (d, J=10.9 Hz, 3H), 1.32 (dd, J=11.2, 6.3 Hz, 3H), 1.28 (d, J=10.1 Hz, 3H), 1.27-1.24 (m, 4H), 1.22 (dt, J=5.7, 4.2 Hz, 4H), 1.20-1.17 (m, 1H), 1.15 (dd, J=8.0, 6.4 Hz, 9H), 1.05 (dd, J=17.7, 7.6 Hz, 3H), 1.01-0.96 (m, 2H), 0.90 (d, J=6.8 Hz, 1H), 0.88-0.82 (m, 3H). ¹³C NMR (176 MHz, CDCl₃) δ 220.8, 175.6, 166.2, 135.7, 134.4, 131.9, 131.3, 129.8, 120.7, 103.4, 102.6, 96.2, 82.2, 81.8, 79.5, 78.5, 78.2, 77.9, 77.0, 74.3, 74.2, 72.9, 71.1, 69.8, 69.1, 68.4, 68.0, 66.1, 65.9, 58.2, 54.5, 52.2, 52.1, 50.7, 50.6, 49.5, 49.3, 48.6, 45.1, 45.0, 39.1, 38.8, 38.6, 37.3, 36.5, 35.5, 35.0, 34.4, 34.1, 30.1, 29.7, 28.3, 27.3, 23.1, 21.6, 21.3, 21.1, 19.7, 18.6, 18.0, 17.8, 16.1, 16.0, 16.0, 12.4, 10.6, 9.9. HRMS (ESI) m/z Calcd. C₄₂ H₇₁ O₁₆ N Na [M+Na⁺]: 868.4665, found 868.4637.

### Synthesis of WBC-04-51:

Monomethyl fumaric acid (30 mg, 0.23 mmol) and EDCI (200 mg, 1.04 mmol) were dissolved in DCM (4 mL). The mixture was stirred for 5-10 minutes under argon atmosphere. Compound 7 (200 mg, 0.272 mmol) was added and stirring continued at rt overnight. The reaction was partitioned between water (50 mL) and DCM (40 mL) and the two layers separated. The aqueous layer was extracted with DCM (25 mL×2), the organic layer was dried over Na₂SO₄ and evaporated in vacuo. The crude was purified using prep TLC eluting with EtOAC:MeOH 20:1.5 to furnish WBC-04-51 as whitish solid (170 mg, 88%). ¹H NMR (700 MHz, CDCl₃) δ 7.46 (dd, J=67.1, 15.4 Hz, 1H), 6.74 (dd, J=72.7, 15.4 Hz, 1H), 5.01 (s, 1H), 4.74-4.66 (m, 2H), 4.56 (t, J=7.7 Hz, 1H), 4.18 (dt, J=27.1, 6.3 Hz, 2H), 4.06 (td, J=8.9, 6.3 Hz, 1H), 3.89-3.81 (m, 1H), 3.80 (s, 2H), 3.78 (s, 2H), 3.75-3.68 (m, 2H), 3.68-3.60 (m, 7H), 3.45 (dq, J=13.2, 7.0 Hz, 1H), 3.36 (s, 2H), 3.32 (s, 1H), 3.07 (dd, J=15.2, 6.6 Hz, 1H), 3.03 (s, 2H), 2.94 (s, 2H), 2.78 (td, J=6.9, 2.9 Hz, 1H), 2.63-2.52 (m, 2H), 2.36 (s, 2H), 2.35-2.29 (m, 2H), 2.13-1.97 (m, 5H), 1.88 (tdq, J=10.3, 7.3, 3.4, 2.7 Hz, 1H), 1.76-1.64 (m, 3H), 1.59 (tt, J=10.1, 4.7 Hz, 2H), 1.54-1.44 (m, 3H), 1.32 (dd, J=11.4, 7.3 Hz, 7H), 1.30-1.17 (m, 22H), 1.17 (s, 1H), 1.14-1.10 (m, 3H), 1.07 (d, J=3.1 Hz, 3H), 1.02-0.92 (m, 9H), 0.89 (h, J=4.2, 3.7 Hz, 7H). ¹³C NMR (176 MHz, CDCl₃) δ 177.0, 176.6, 173.3, 171.0, 170.5, 170.1, 166.2, 166.1, 165.7, 135.8, 134.6, 131.1, 130.9, 129.7, 129.5, 103.5, 102.9, 95.4, 85.5, 79.5, 78.9, 77.8, 74.3, 73.4, 71.4, 71.0, 70.0, 68.4, 66.1, 62.3, 62.3, 62.0, 58.4, 54.64, 52.2, 52.0, 49.4, 45.1, 42.3, 40.9, 38.9, 36.5, 35.5, 34.9, 34.1, 31.9, 29.7, 28.3, 27.1, 26.1, 25.3, 24.8, 22.6, 21.3, 20.7, 19.7, 18.9, 18.2, 17.8, 16.2, 15.3, 14.1, 14.1, 13.7, 11.2, 9.9, 9.7, 7.6. HRMS (ESI) m/z Calcd. C₄₂ H₇₅ O₁₅ N₂ [M+H⁺]: 847.5162, found 847.5139.

### Synthesis of WBC-04-110:

Monobutyl fumaric acid (100 mg, 0.58 mmol) and EDCI (400 mg, 2.1 mmol) were dissolved in DCM (5 mL). The mixture was stirred for 5-10 minutes at rt under argon atmosphere. Compound 4 (180 mg, 0.24 mmol) was added to the solution and stirring continued at rt overnight. The reaction was partitioned between water (50 mL) and DCM (40 mL) and the two layers separated. The aqueous layer was extracted with DCM (25 mL×2), the organic layer was dried over Na₂SO₄ and evaporated in vacuo. The crude was purified using prep TLC eluting with EtOAC:hexane 7:3 to furnish WBC-04-110 as white solid (140 mg, 69%). ¹H NMR (700 MHz, CDCl₃) δ 7.44 (dd, J=71.3, 15.4 Hz, 1H), 6.75 (dd, J=65.1, 15.4 Hz, 1H), 5.07 (ddd, J=11.2, 5.9, 2.4 Hz, 1H), 4.93 (t, J=4.6 Hz, 1H), 4.69 (ddd, J=12.5, 10.8, 4.4 Hz, 1H), 4.55 (dd, J=9.9, 7.2 Hz, 1H), 4.19 (dt, J=19.1, 6.7 Hz, 2H), 4.01 (dt, J=8.9, 5.7 Hz, 1H), 3.96 (s, 1H), 3.77-3.73 (m, 2H), 3.73-3.64 (m, 2H), 3.41 (dddd, J=15.0, 11.2, 7.4, 4.6 Hz, 1H), 3.35 (s, 2H), 3.32 (s, 1H), 3.19 (d, J=5.5 Hz, 1H), 3.10-3.00 (m, 5H), 2.99 (s, 1H), 2.94 (s, 1H), 2.87 (pd, J=7.2, 3.5 Hz, 1H), 2.63-2.54 (m, 1H), 2.44-2.40 (m, 1H), 2.38-2.29 (m, 1H), 2.00-1.88 (m, 2H), 1.82-1.75 (m, 1H), 1.75-1.68 (m, 2H), 1.68-1.63 (m, 3H), 1.60 (ddd, J=15.1, 4.8, 1.9 Hz, 2H), 1.55-1.44 (m, 2H), 1.41 (d, J=11.4 Hz, 4H), 1.34-1.19 (m, 12H), 1.17-1.11 (m, 7H), 1.04 (dd, J=18.5, 7.5 Hz, 3H), 0.95 (td, J=7.4, 3.9 Hz, 3H), 0.84 (td, J=7.4, 1.7 Hz, 3H). ¹³C NMR (176 MHz, CDCl₃) δ 220.7, 175.6, 166.2, 165.8, 135.5, 134.1, 131.8, 130.3, 103.5, 102.6, 96.0, 82.2, 81.8, 78.5, 78.1 76.7, 74.2, 72.9, 71.7, 71.2, 69.1, 68.4, 68.0, 66.0, 65.0, 58.2, 54.5, 50.6, 49.4, 45.0, 39.2, 38.6, 37.3, 36.5, 35.5, 35.0, 30.6, 30.1, 21.5, 21.0, 19.8, 19.1, 18.6, 18.6, 18.0, 16.0, 13.7, 12.4, 10.6, 9.9, 9.6. HRMS (ESI) m/z Calcd. C₄₅ H₇₇ O₁₆ N [M+H⁺]: 888.5315, found 888.5337.

### Synthesis of WBC-04-111:

Monobutyl fumaric acid (100 mg, 0.58 mmol) and EDCI (400 mg, 2.1 mmol) were dissolved in DCM (5 mL). The mixture was stirred for 5-10 minutes under argon atmosphere. Compound 7 (180 mg, 0.24 mmol) and DMAP (45 mg, 0.36 mmol) were added and stirring continued at rt overnight. The reaction was partitioned between water (50 mL) and DCM (40 mL) and the two layers separated. The aqueous layer was extracted with DCM (25 mL×2), the organic layer was dried over Na₂SO₄ and evaporated in vacuo. The crude was purified using prep TLC eluting with EtOAC:MeOH 10:1 to furnish WBC-04-111 as white solid (65 mg, 31%). ¹H NMR (700 MHz, CDCl₃) δ 7.45 (dd, J=78.0, 15.4 Hz, 1H), 6.74 (dd, J=74.1, 15.4 Hz, 1H), 5.26 (dp, J=10.1, 5.2 Hz, 1H), 5.13 (t, J=5.4 Hz, 1H), 4.85 (p, J=6.3 Hz, 1H), 4.72 (td, J=11.9, 4.5 Hz, 1H), 4.29 (td, J=6.9, 4.6 Hz, 1H), 4.23-4.13 (m, 4H), 4.07 (ddd, J=13.3, 9.4, 6.4 Hz, 1H), 3.68 (s, 1H), 3.64 (t, J=6.1 Hz, 1H), 3.45 (tt, J=12.6, 6.5 Hz, 1H), 3.39 (s, 1H), 3.34 (s, 1H), 3.07 (t, J=8.9 Hz, 1H), 3.03 (s, 2H), 2.94 (s, 1H), 2.77-2.69 (m, 2H), 2.59 (d, J=13.3 Hz, 1H), 2.39-2.26 (m, 5H), 2.10-2.05 (m, 4H), 2.04 (s, 2H), 1.99 (p, J=8.3 Hz, 1H), 1.90 (dtt, J=11.9, 7.6, 3.9 Hz, 1H), 1.74-1.63 (m, 4H), 1.60 (td, J=9.6, 9.0, 4.5 Hz, 2H), 1.51 (q, J=8.0, 6.8 Hz, 3H), 1.40 (tt, J=7.5, 3.9 Hz, 2H), 1.36-1.33 (m, 5H), 1.26 (d, J=7.5 Hz, 13H), 1.20 (d, J=7.5 Hz, 3H), 1.13 (d, J=7.1 Hz, 2H), 1.09 (s, 2H), 1.01-0.92 (m, 8H), 0.88 (q, J=7.4 Hz, 5H). ¹³C NMR (176 MHz, CDCl₃) δ 173.35, 173.0, 170.5, 170.1, 166.3, 165.8, 135.8, 134.3, 131.6, 130.2, 103.2, 102.7, 94.8, 84.5, 77.9, 77.5, 77.2, 76.9, 74.5, 74.2, 73.6, 73.1, 71.3, 70.9, 69.1, 68.7, 68.4, 68.1, 65.9, 64.9, 62.30, 62.0, 58.4, 54.6, 49.5, 45.3, 41.9, 36.3, 35.5, 34.7, 34.0, 31.7, 30.6, 29.5, 29.0, 27.3, 26.6, 25.3, 22.6, 22.1, 21.6, 21.3, 20.7, 18.9, 18.1, 16.3, 14.7, 14.1, 13.7, 11.2, 9.7, 9.5. HRMS (ESI) m/z Calcd. C₄₅H₅₀N₂O₁₅ [M+H⁺]: 889.5631, found 889.5624.

### Anti-Proliferation IC₅₀ for the New Compounds

| | MRC-5 | A549 | Hep-G2 | VERO | MDA-MB-231 | MCF-7 |
|---|---|---|---|---|---|---|
| Compound | (IC₅₀ μM) | (IC₅₀ μM) | (IC₅₀ μM) | (IC₅₀ μM) | (IC₅₀ μM) | (IC₅₀ μM) |
| AZM-613 | NT | NT | 2.9 | 6.5 | 2.1 | 2.8 |
| AO-03-032 | NT | 7.6 | 4.2 | 10.4 | 6.4 | NT |
| CIP-613 | NT | NT | 232.4 | 223.6 | 235.6 | 206.2 |

NT = Not tested

TABLE 1
Anti-proliferation IC₅₀ for compounds of formula I

| | | | | | MDA- | |
|---|---|---|---|---|---|---|
| | | | | | MB-231 | MCF-7 |
| | MRC-5 | A549 | Hep-G2 | VERO | (IC₅₀, | (IC₅₀, |
| | (IC₅₀, μM) | (IC₅₀, μM) | (IC₅₀, μM) | (IC₅₀, μM) | μM) | μM) |
| 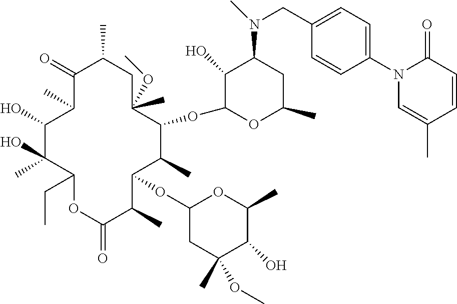 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3ccc(-n4cc(C)ccc4=O)cc3)[C@H]2O)[C@@](C)(OC)C[C@@H](C)C(=O)[C@H](C)[C@@H](O)[C@]1(C)O] | >500 | >500 | >500 | >500 | NT | NT |
| 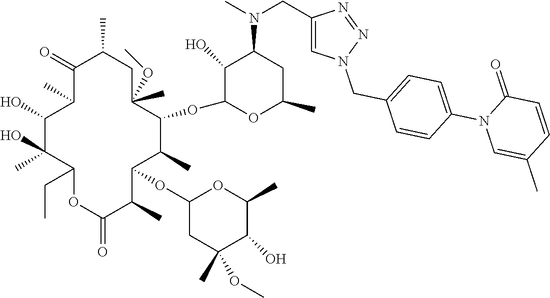 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3cn(Cc4ccc(-n5cc(C)ccc5=O)cc4)nn3)[C@H]2O)[C@@](C)(OC)C[C@@H](C)C(=O)[C@H](C)[C@@H](O)[C@]1(C)O] | 102.6 ± 0.8 | 118.3 ± 28.4 | 116.5 ± 16.0 | 153.2 ± 19.0 | NT | NT |
| 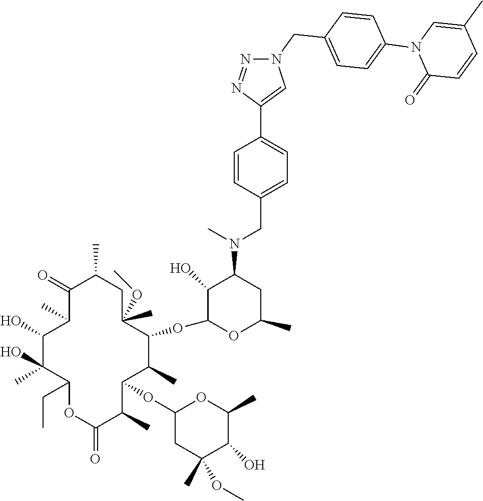 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3ccc(-c4cn(Cc5ccc(-n6cc(C)ccc6=O)cc5)nn4)cc3)[C@H]2O)[C@@](C)(OC)C[C@@H](C)C(=O)[C@H](C)[C@@H](O)[C@]1(C)O] | 12.3 ± 1.8 | 11.1 ± 2.7 | 20.3 ± 1.0 | 5.1 ± 0.2 | NT | NT |
| 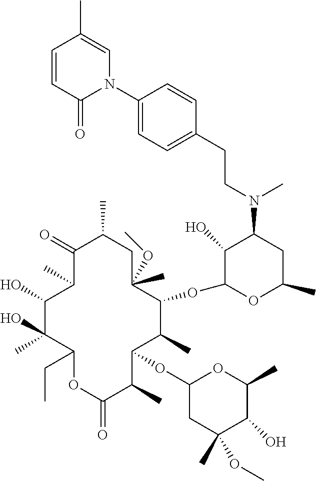 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCc3ccc(-n4cc(C)ccc4=O)cc3)[C@H]2O)[C@@](C)(OC)C[C@@H](C)C(=O)[C@H](C)[C@@H](O)[C@]1(C)O] | 119 | >500 | >500 | >500 | NT | NT |
| 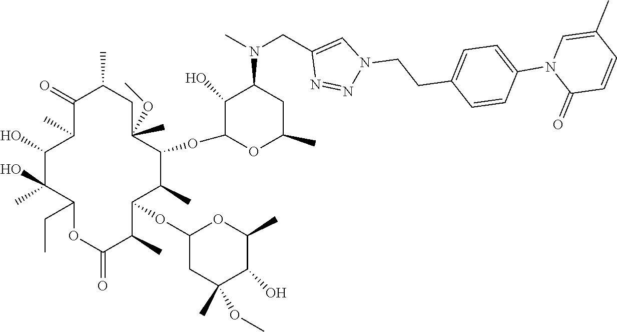 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3cn(CCc4ccc(-n5cc(C)ccc5=O)cc4)nn3)[C@H]2O)[C@@](C)(OC)C[C@@H](C)C(=O)[C@H](C)[C@@H](O)[C@]1(C)O] | 84.0 ± 4.5 | 115.9 ± 4.1 | 121.7 ± 3.7 | 104.5 ± 20.3 | NT | NT |
| 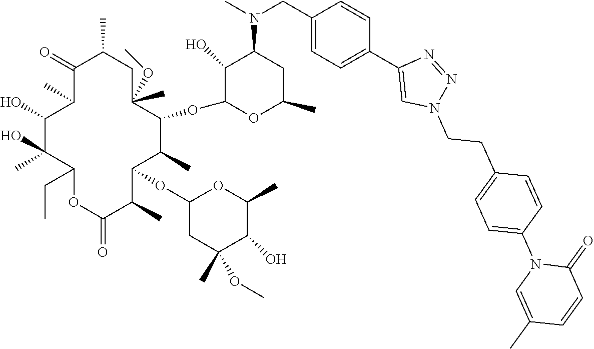 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3ccc(-c4cn(CCc5ccc(-n6cc(C)ccc6=O)cc5)nn4)cc3)[C@H]2O)[C@@](C)(OC)C[C@@H](C)C(=O)[C@H](C)[C@@H](O)[C@]1(C)O] | 13.1 ± 1.2 | 35.3 ± 1.5 | 28.1 ± 1.8 | 14.2 ± 0.3 | NT | NT |
| 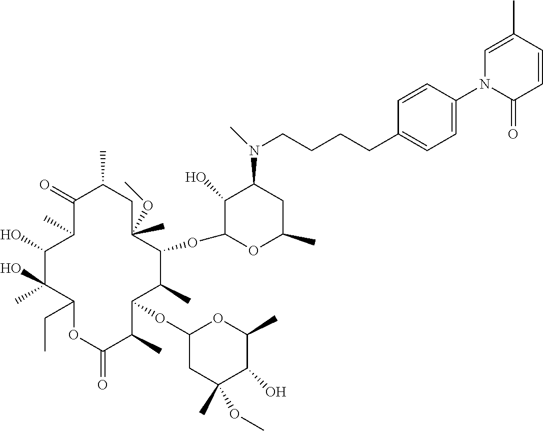 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCCCc3ccc(-n4cc(C)ccc4=O)cc3)[C@H]2O)[C@@](C)(OC)C[C@@H](C)C(=O)[C@H](C)[C@@H](O)[C@]1(C)O] | 15.7 ± 2.3 | 16.3 ± 1.6 | 30.9 ± 1.4 | 19.0 ± 3.7 | NT | NT |
| 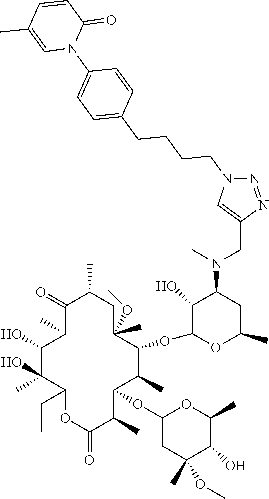 | 32.8 ± 5.1 | 28.7 ± 3.4 | 44.8 ± 3.6 | 33.3 ± 1.9 | NT | NT |
| 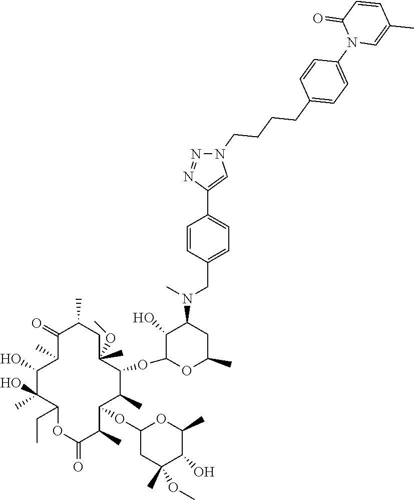 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)Cc3ccc(-c4cn(CCCCc5ccc(-n6cc(C)ccc6=O)cc5)nn4)cc3)[C@H]2O)[C@@](C)(OC)C[C@@H](C)C(=O)[C@H](C)[C@@H](O)[C@]1(C)O] | 11.6 ± 0.3 | 10.9 ± 0.4 | 36.4 ± 6.6 | 12.4 ± 0.2 | NT | NT |
| 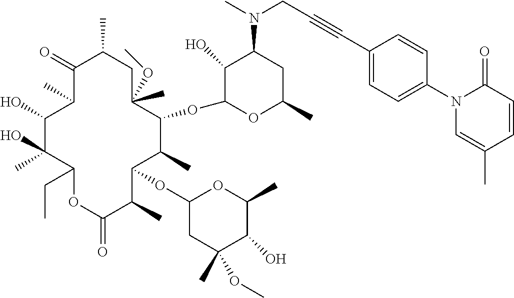 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CC#Cc3ccc(-n4cc(C)ccc4=O)cc3)[C@H]2O)[C@@](C)(OC)C[C@@H](C)C(=O)[C@H](C)[C@@H](O)[C@]1(C)O] | 82.6 ± 0.3 | 44.1 ± 10.8 | 50.7 ± 3.0 | 55.5 ± 2.6 | NT | NT |
| 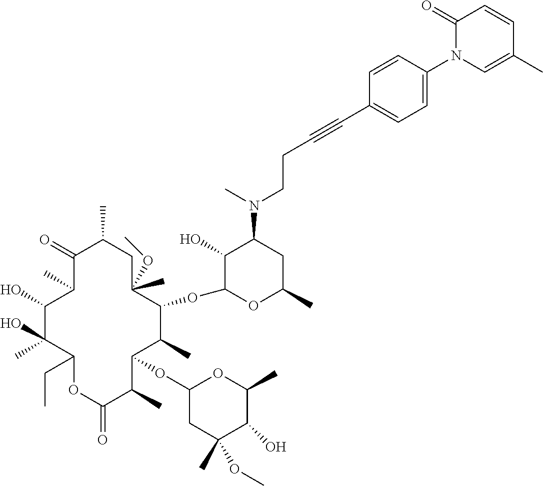 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCC#Cc3ccc(-n4cc(C)ccc4=O)cc3)[C@H]2O)[C@@](C)(OC)C[C@@H](C)C(=O)[C@H](C)[C@@H](O)[C@]1(C)O] | >500 | 59.9 ± 0.8 | 60.9 ± 9.0 | 130.4 ± 21.2 | NT | NT |
| 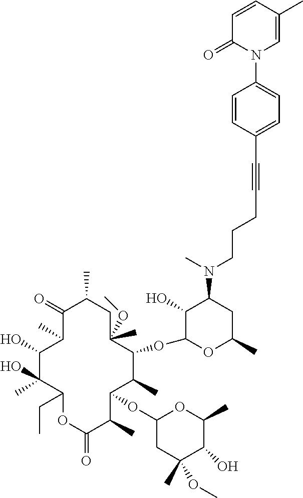 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCCC#Cc3ccc(-n4cc(C)ccc4=O)cc3)[C@H]2O)[C@@](C)(OC)C[C@@H](C)C(=O)[C@H](C)[C@@H](O)[C@]1(C)O] | 15.2 ± 0.1 | 58.6 ± 1.4 | 24.0 ± 1.2 | 55.5 ± 0.4 | NT | NT |
| 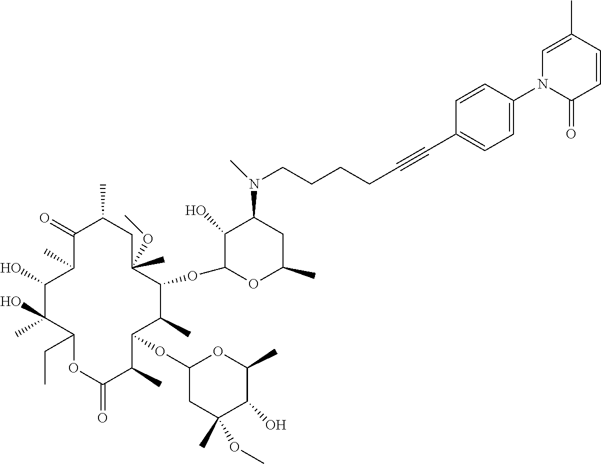 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCCCC#Cc3ccc(-n4cc(C)ccc4=O)cc3)[C@H]2O)[C@@](C)(OC)C[C@@H](C)C(=O)[C@H](C)[C@@H](O)[C@]1(C)O] | 28.9 ± 1.9 | 35.4 ± 2.1 | 32.0 ± 0.1 | 24.3 ± 0.1 | NT | NT |
| 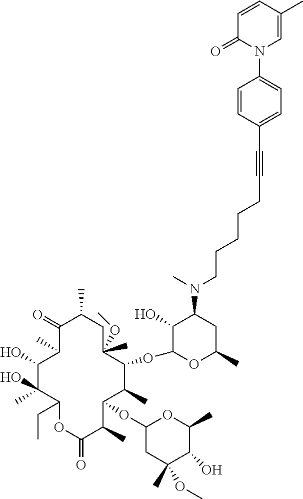 [SMILES: CCC1OC(=O)[C@H](C)[C@H](OC2C[C@@](C)(OC)[C@@H](O)[C@H](C)O2)[C@H](C)[C@@H](OC2O[C@H](C)C[C@H](N(C)CCCCCC#Cc3ccc(-n4cc(C)ccc4=O)cc3)[C@H]2O)[C@@](C)(OC)C[C@@H](C)C(=O)[C@H](C)[C@@H](O)[C@]1(C)O] | 24.2 ± 0.6 | 23.8 ± 2.1 | 23.7 ± 2.0 | 13.2 ± 0.7 | NT | NT |
| 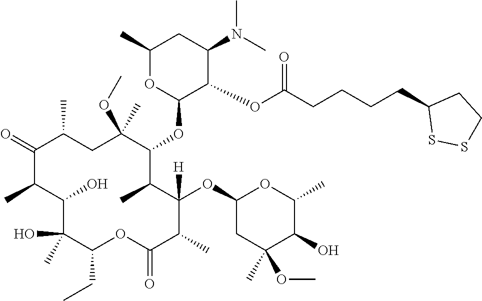 [SMILES: CC[C@H]1OC(=O)[C@@H](C)[C@@H](O[C@@H]2C[C@](C)(OC)[C@H](O)[C@@H](C)O2)[C@@H](C)[C@@H](O[C@H]2O[C@@H](C)C[C@@H](N(C)C)[C@@H]2OC(=O)CCCC[C@H]2CCSS2)[C@](C)(OC)C[C@@H](C)C(=O)[C@H](C)[C@H](O)[C@]1(C)O] | 44.8 ± 7.1 | NI | NI | NI | 65.0 ± 0.9 | NI |
| 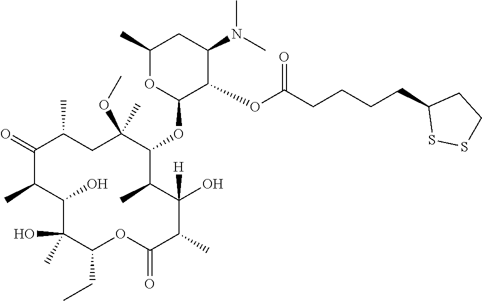 [SMILES: CC[C@H]1OC(=O)[C@@H](C)[C@@H](O)[C@@H](C)[C@@H](O[C@H]2O[C@@H](C)C[C@@H](N(C)C)[C@@H]2OC(=O)CCCC[C@H]2CCSS2)[C@](C)(OC)C[C@@H](C)C(=O)[C@H](C)[C@H](O)[C@]1(C)O] | 262.6 ± 0.4 | >500 | 221.8 ± 5.3 | >500 | 414.4 ± 1.7 | 477.3 ± 3.4 |
| 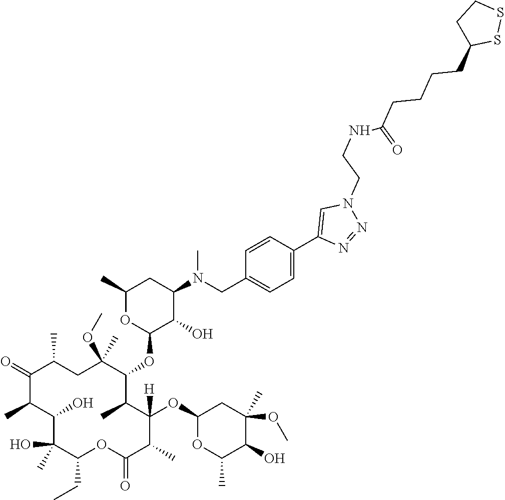 [SMILES: CC[C@H]1OC(=O)[C@@H](C)[C@@H](O[C@H]2C[C@@](C)(OC)[C@@H](O)[C@H](C)O2)[C@@H](C)[C@@H](O[C@H]2O[C@@H](C)C[C@@H](N(C)Cc3ccc(-c4cn(CCNC(=O)CCCC[C@H]5CCSS5)nn4)cc3)[C@@H]2O)[C@](C)(OC)C[C@@H](C)C(=O)[C@H](C)[C@H](O)[C@]1(C)O] | 56.1 ± 1.2 | 58.6 ± 0.6 | 57.3 ± 1.2 | 65.0 ± 12.2 | 56.9 ± 1.3 | 42.3 ± 4.4 |
| 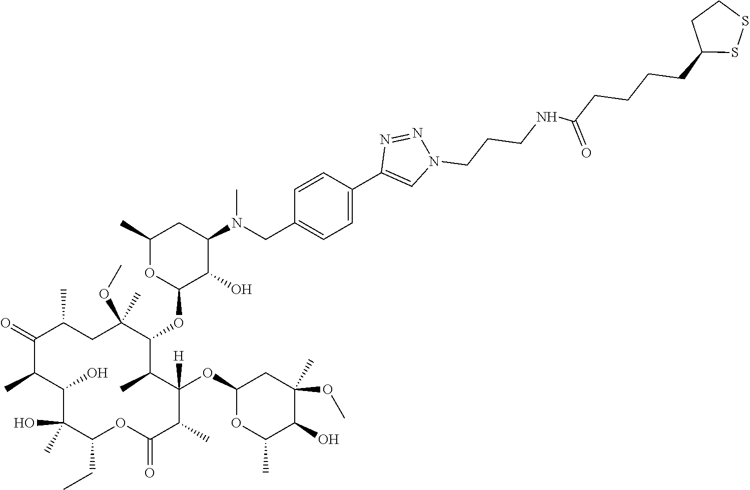 [SMILES: CC[C@H]1OC(=O)[C@@H](C)[C@@H](O[C@H]2C[C@@](C)(OC)[C@@H](O)[C@H](C)O2)[C@@H](C)[C@@H](O[C@H]2O[C@@H](C)C[C@@H](N(C)Cc3ccc(-c4cn(CCCNC(=O)CCCC[C@H]5CCSS5)nn4)cc3)[C@@H]2O)[C@](C)(OC)C[C@@H](C)C(=O)[C@H](C)[C@H](O)[C@]1(C)O] | 51.6 ± 5.0 | 56.9 ± 0.8 | 49.2 ± 1.8 | 58.1 ± 2.7 | 58.5 ± 3.2 | 53.6 ± 0.2 |
| 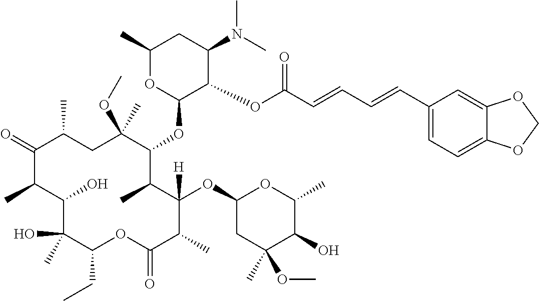 [SMILES: CC[C@H]1OC(=O)[C@@H](C)[C@@H](O[C@@H]2C[C@](C)(OC)[C@H](O)[C@@H](C)O2)[C@@H](C)[C@@H](O[C@H]2O[C@@H](C)C[C@@H](N(C)C)[C@@H]2OC(=O)/C=C/C=C/c2ccc3c(c2)OCO3)[C@](C)(OC)C[C@@H](C)C(=O)[C@H](C)[C@H](O)[C@]1(C)O] | 7.8 ± 0.7 | 16.9 ± 0.7 | 5.6 ± 0.1 | 21.0 ± 2.3 | NT | NT |
| 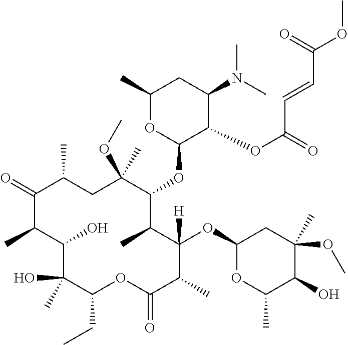 | 88.4 | 278.5 | 133.2 | 219.4 ± 45.8 | 139.9 | 127.5 ± 16.5 |
| 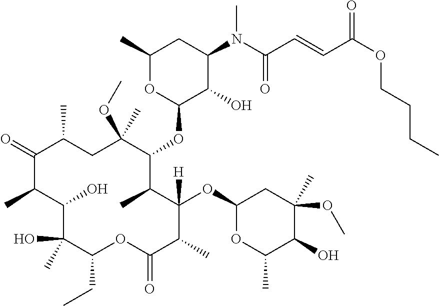 [SMILES: CCCCOC(=O)/C=C/C(=O)N(C)[C@@H]1C[C@H](C)O[C@H](O[C@@H]2[C@H](C)[C@H](O[C@H]3C[C@@](C)(OC)[C@@H](O)[C@H](C)O3)[C@H](C)C(=O)O[C@H](CC)[C@@](C)(O)[C@@H](O)[C@@H](C)C(=O)[C@H](C)C[C@@]2(C)OC)[C@H]1O] | 44.9 ± 0.6 | >500 | 208.9 ± 11.2 | 68.3 ± 16.8 | 28.4 ± 3.9 | 72.3 ± 5.2 |
| Pirfenidone (Pir) | 5302 ± 177.9 | 5409 ± 241 | 10920 ± | 10788 ± 0.6 | NT | NT |
| | | | 1090 | | | |
| Azithromycin (AZM) | 127 ± 1 | 203.5 ± 4.5 | 85.3 ± 0.9 | 222.0 ± 18.1 | NT | NT |
| Clarithromycin (CLM) | 138.1 ± 1.1 | >500 | 130 | >500 | NT | NT |
| Lipoic acid (ALA) | NI | NI | NI | NI | NI | NI |
| Piperic acid (PPA) | NI | NI | NI | NI | NT | NT |
| Dimethyl Fumarate | 258.9 | NI | NI | 844 | NI | NI |

TABLE 2
Anti-proliferation IC₅₀ for compounds of formula II

| | | | | | MDA-MB- | |
|---|---|---|---|---|---|---|
| | MRC-5 | A549 | Hep-G2 | VERO | 231 | MCF-7 |
| 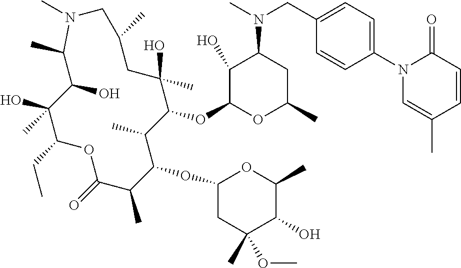 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3ccc(-n4cc(C)ccc4=O)cc3)[C@H]2O)[C@](C)(O)C[C@@H](C)CN(C)[C@H](C)[C@@H](O)[C@]1(C)O] | 55.8 ± 1.9 | 47.2 ± 5.1 | 33.0 ± 5.4 | 47.2 ± 0.4 | NT | NT |
| 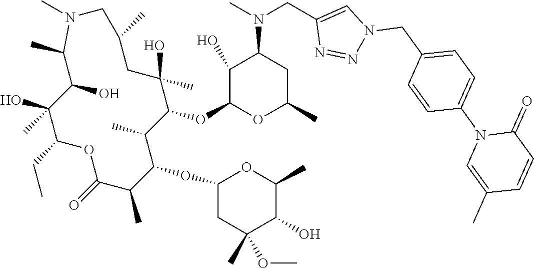 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3cn(Cc4ccc(-n5cc(C)ccc5=O)cc4)nn3)[C@H]2O)[C@](C)(O)C[C@@H](C)CN(C)[C@H](C)[C@@H](O)[C@]1(C)O] | 121.0 ± 14.1 | 265.1 ± 4.0 | 233.6 ± 31.1 | 121.6 ± 19.4 | NT | NT |
| 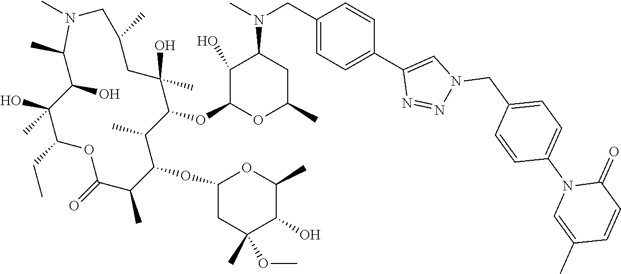 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3ccc(-c4cn(Cc5ccc(-n6cc(C)ccc6=O)cc5)nn4)cc3)[C@H]2O)[C@](C)(O)C[C@@H](C)CN(C)[C@H](C)[C@@H](O)[C@]1(C)O] | 13.4 ± 0.1 | 18.4 ± 4.4 | 11.1 ± 0.3 | 16.2 ± 2.8 | NT | NT |
| 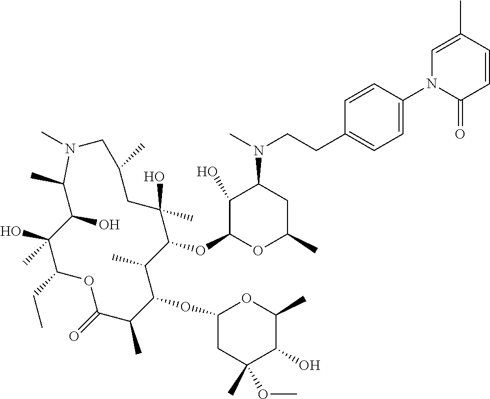 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCc3ccc(-n4cc(C)ccc4=O)cc3)[C@H]2O)[C@](C)(O)C[C@@H](C)CN(C)[C@H](C)[C@@H](O)[C@]1(C)O] | 36.1 ± 3.3 | 40.8 ± 4.0 | 27.2 ± 2.3 | 30.3 ± 5.2 | NT | NT |
| 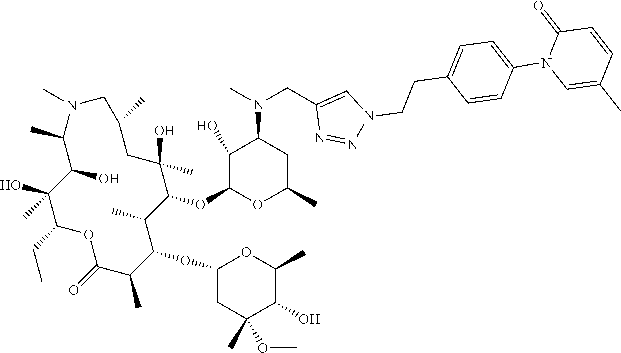 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3cn(CCc4ccc(-n5cc(C)ccc5=O)cc4)nn3)[C@H]2O)[C@](C)(O)C[C@@H](C)CN(C)[C@H](C)[C@@H](O)[C@]1(C)O] | 172.1 ± 26.9 | 143.5 ± 24.5 | 163.4 ± 1.6 | 150.6 ± 30.6 | NT | NT |
| 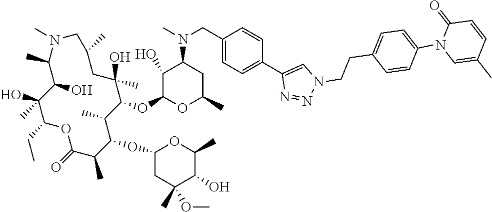 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3ccc(-c4cn(CCc5ccc(-n6cc(C)ccc6=O)cc5)nn4)cc3)[C@H]2O)[C@](C)(O)C[C@@H](C)CN(C)[C@H](C)[C@@H](O)[C@]1(C)O] | 13.3 ± 0.9 | 15.8 ± 3.0 | 21.8 ± 0.4 | 22.6 ± 4.9 | NT | NT |
| 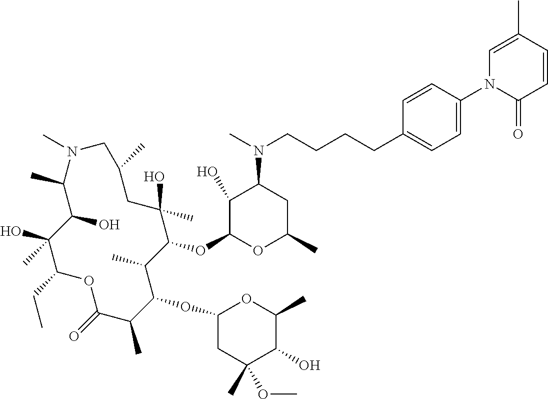 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCCCc3ccc(-n4cc(C)ccc4=O)cc3)[C@H]2O)[C@](C)(O)C[C@@H](C)CN(C)[C@H](C)[C@@H](O)[C@]1(C)O] | 5.9 ± 0.1 | 43.2 ± 5.7 | 52.2 ± 1.0 | 23.9 ± 1.7 | NT | NT |
| 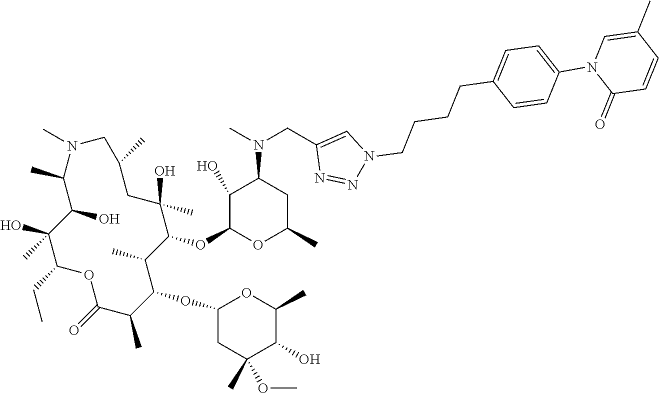 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3cn(CCCCc4ccc(-n5cc(C)ccc5=O)cc4)nn3)[C@H]2O)[C@](C)(O)C[C@@H](C)CN(C)[C@H](C)[C@@H](O)[C@]1(C)O] | 36.1 ± 6.1 | 35.2 ± 5.4 | 27.7 ± 3.3 | 53.2 ± 0.10 | NT | NT |
| 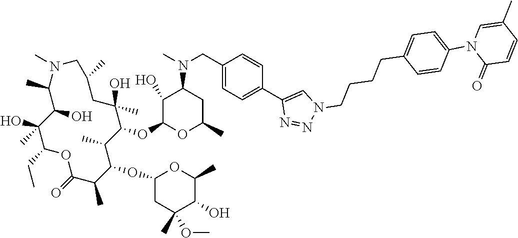 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3ccc(-c4cn(CCCCc5ccc(-n6cc(C)ccc6=O)cc5)nn4)cc3)[C@H]2O)[C@](C)(O)C[C@@H](C)CN(C)[C@H](C)[C@@H](O)[C@]1(C)O] | 5.7 ± 0.1 | 5 ± 0.2 | 2.6 ± 0.1 | 5.9 ± 0.1 | NT | NT |
| 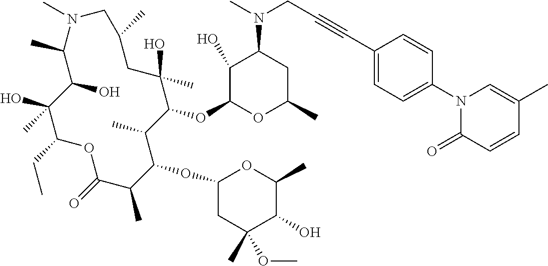 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CC#Cc3ccc(-n4cc(C)ccc4=O)cc3)[C@H]2O)[C@](C)(O)C[C@@H](C)CN(C)[C@H](C)[C@@H](O)[C@]1(C)O] | 23.3 ± 3.3 | 55.9 ± 6.4 | 53.4 ± 3.3 | 71.4 ± 6.3 | NT | NT |
| 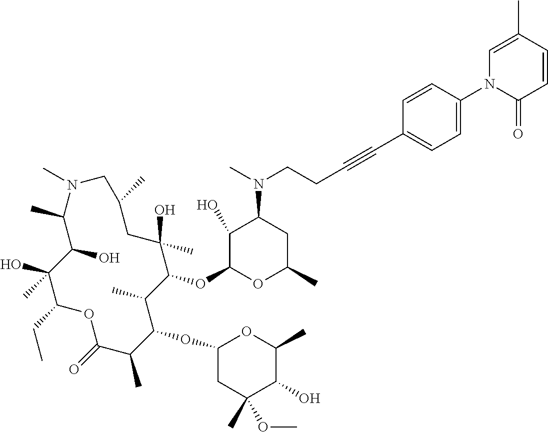 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCC#Cc3ccc(-n4cc(C)ccc4=O)cc3)[C@H]2O)[C@](C)(O)C[C@@H](C)CN(C)[C@H](C)[C@@H](O)[C@]1(C)O] | 19.1 ± 2.6 | 31.2 ± 4.8 | 24.8 ± 1.7 | 28.5 ± 1.8 | NT | NT |
| 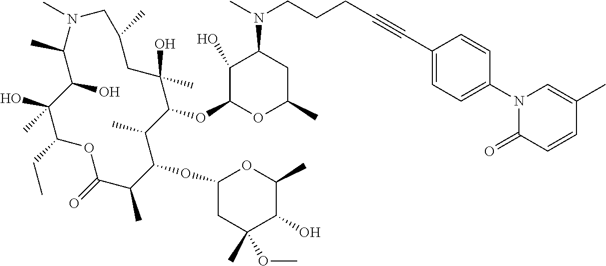 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCCC#Cc3ccc(-n4cc(C)ccc4=O)cc3)[C@H]2O)[C@](C)(O)C[C@@H](C)CN(C)[C@H](C)[C@@H](O)[C@]1(C)O] | 41.7 ± 0.1 | 42.7 ± 8.4 | 45.2 ± 7.4 | 36.4 ± 3.8 | NT | NT |
| 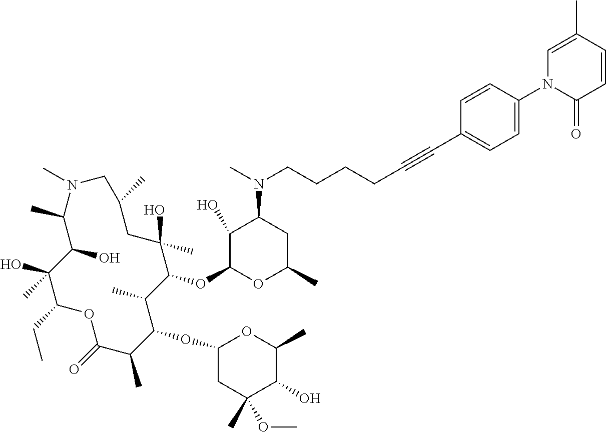 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCCCC#Cc3ccc(-n4cc(C)ccc4=O)cc3)[C@H]2O)[C@](C)(O)C[C@@H](C)CN(C)[C@H](C)[C@@H](O)[C@]1(C)O] | 5.5 ± 0.2 | 15.5 ± 1.1 | 12.7 ± 1.3 | 16.2 ± 3.1 | NT | NT |
| 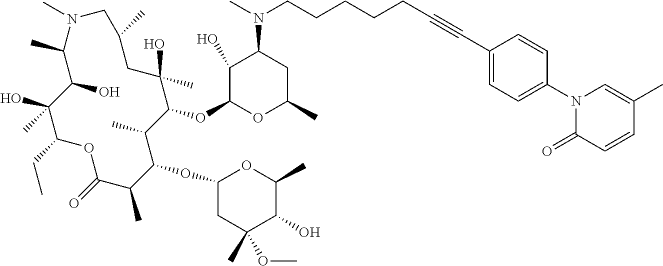 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)CCCCCC#Cc3ccc(-n4cc(C)ccc4=O)cc3)[C@H]2O)[C@](C)(O)C[C@@H](C)CN(C)[C@H](C)[C@@H](O)[C@]1(C)O] | 2.2 ± 0.1 | 6.1 ± 0.3 | 2.8 | 4.7 ± 0.3 | NT | NT |
| 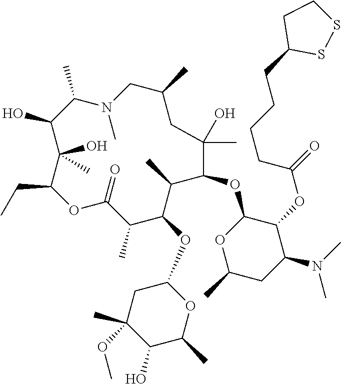 [SMILES: CC[C@@H]1OC(=O)[C@@H](C)[C@H](O[C@H]2C[C@@](C)(OC)[C@@H](O)[C@H](C)O2)[C@@H](C)[C@H](O[C@@H]2O[C@H](C)C[C@H](N(C)C)[C@H]2OC(=O)CCCC[C@H]2CCSS2)C(C)(O)C[C@H](C)CN(C)[C@@H](C)[C@H](O)[C@@]1(C)O] | 13.4 ± 0.4 | 23.0 ± 0.5 | 29.5 ± 4.4 | 54.5 ± 0.8 | 45.0 ± 1.3 | 38.2 ± 0.8 |
| 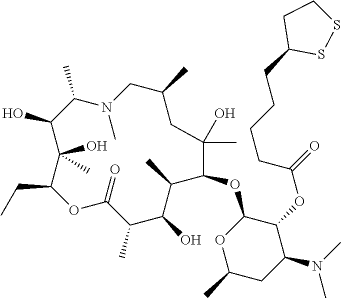 [SMILES: CC[C@@H]1OC(=O)[C@@H](C)[C@H](O)[C@@H](C)[C@H](O[C@@H]2O[C@H](C)C[C@H](N(C)C)[C@H]2OC(=O)CCCC[C@H]2CCSS2)C(C)(O)C[C@H](C)CN(C)[C@@H](C)[C@H](O)[C@@]1(C)O] | 143.1 ± 17.1 | 281.9 ± 14.3 | 224.7 ± 12.8 | 273.1 ± 26.9 | 400.3 ± 32.4 | NT |
| 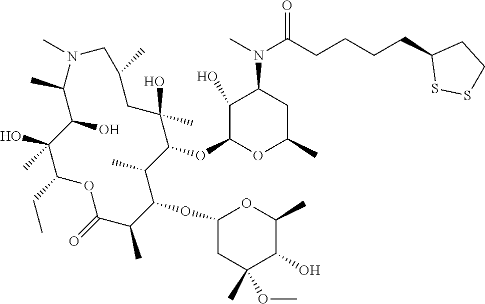 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C(=O)CCCC[C@H]3CCSS3)[C@H]2O)[C@](C)(O)C[C@@H](C)CN(C)[C@H](C)[C@@H](O)[C@]1(C)O] | 20.2 ± 3.7 | 18.8 ± 4.1 | 13.6 ± 0.1 | 24.4 ± 2.4 | 11.9 ± 1.7 | 11.2 |
| 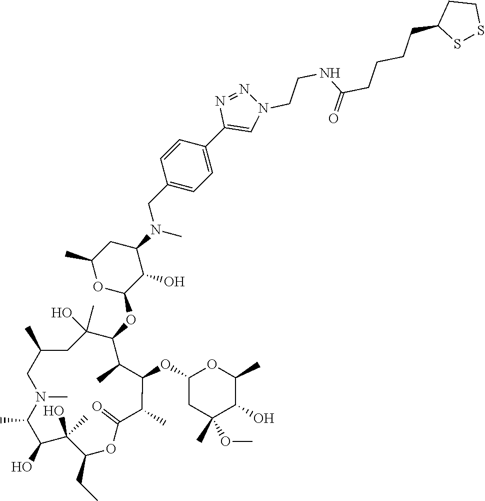 [SMILES: CC[C@@H]1OC(=O)[C@@H](C)[C@H](O[C@H]2C[C@@](C)(OC)[C@@H](O)[C@H](C)O2)[C@@H](C)[C@H](O[C@H]2O[C@@H](C)C[C@@H](N(C)Cc3ccc(-c4cn(CCNC(=O)CCCC[C@H]5CCSS5)nn4)cc3)[C@@H]2O)C(C)(O)C[C@H](C)CN(C)[C@@H](C)[C@H](O)[C@@]1(C)O] | 6.1 | 10.3 ± 0.9 | 8.5 ± 1.1 | 16.9 ± 0.4 | 11.8 ± 0.1 | 11.7 ± 0.4 |
| 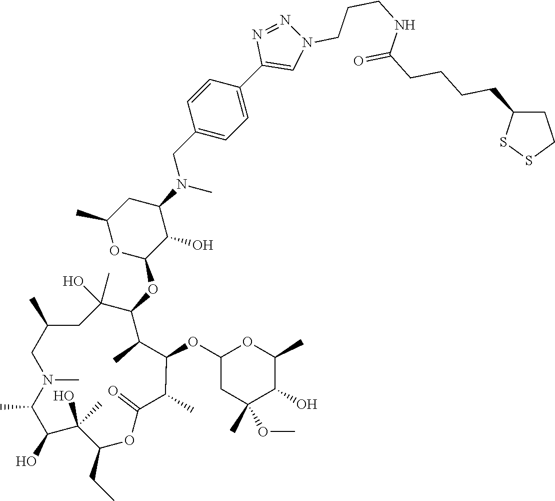 [SMILES: CC[C@@H]1OC(=O)[C@@H](C)[C@H](OC2C[C@@](C)(OC)[C@@H](O)[C@H](C)O2)[C@@H](C)[C@H](O[C@H]2O[C@@H](C)C[C@@H](N(C)Cc3ccc(-c4cn(CCCNC(=O)CCCC[C@H]5CCSS5)nn4)cc3)[C@@H]2O)C(C)(O)C[C@H](C)CN(C)[C@@H](C)[C@H](O)[C@@]1(C)O] | 24.5 ± 2.6 | 26.9 | 14.4 ± 0.6 | 40.3 ± 4.9 | 21.3 ± 1.5 | 26.8 ± 7.1 |
| 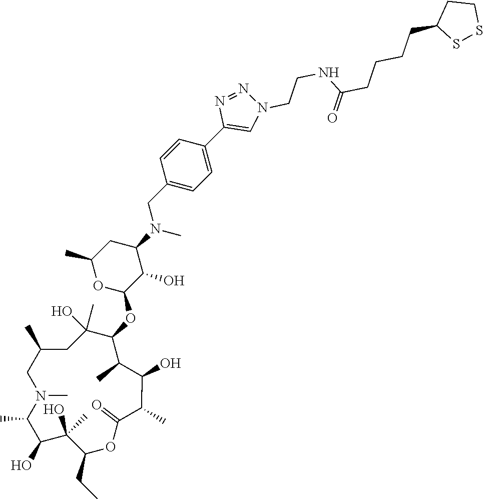 [SMILES: CC[C@@H]1OC(=O)[C@@H](C)[C@H](O)[C@@H](C)[C@H](O[C@H]2O[C@@H](C)C[C@@H](N(C)Cc3ccc(-c4cn(CCNC(=O)CCCC[C@H]5CCSS5)nn4)cc3)[C@@H]2O)C(C)(O)C[C@H](C)CN(C)[C@@H](C)[C@H](O)[C@@]1(C)O] | 17.9 ± 0.5 | 27.4 ± 0.5 | 14.8 ± 2.1 | 47.7 ± 6.2 | NT | NT |
| 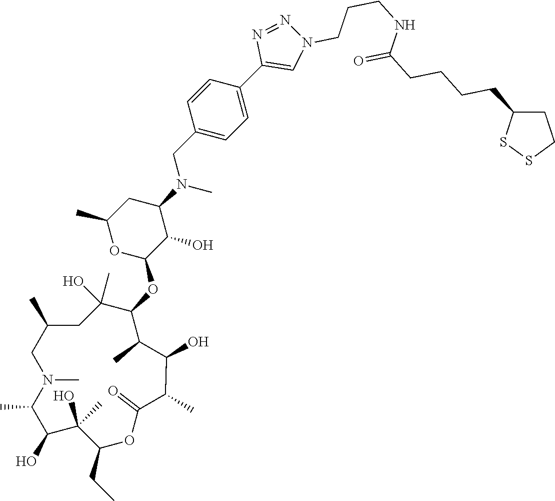 [SMILES: CC[C@@H]1OC(=O)[C@@H](C)[C@H](O)[C@@H](C)[C@H](O[C@H]2O[C@@H](C)C[C@@H](N(C)Cc3ccc(-c4cn(CCCNC(=O)CCCC[C@H]5CCSS5)nn4)cc3)[C@@H]2O)C(C)(O)C[C@H](C)CN(C)[C@@H](C)[C@H](O)[C@@]1(C)O] | 144.8 ± 3.5 | 92.9 ± 13.0 | 157.0 ± 11.4 | 168.3 ± 19.7 | NT | NT |
| 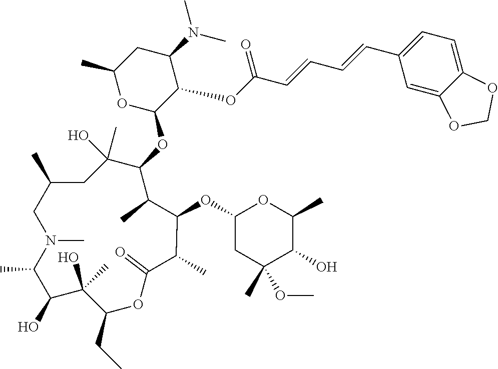 [SMILES: CC[C@@H]1OC(=O)[C@@H](C)[C@H](O[C@H]2C[C@@](C)(OC)[C@@H](O)[C@H](C)O2)[C@@H](C)[C@H](O[C@H]2O[C@@H](C)C[C@@H](N(C)C)[C@@H]2OC(=O)/C=C/C=C/c2ccc3c(c2)OCO3)C(C)(O)C[C@H](C)CN(C)[C@@H](C)[C@H](O)[C@@]1(C)O] | 3.8 ± 0.8 | 15.6 ± 2.6 | 5.0 ± 0.5 | 13.2 ± 1.5 | NT | NT |
| 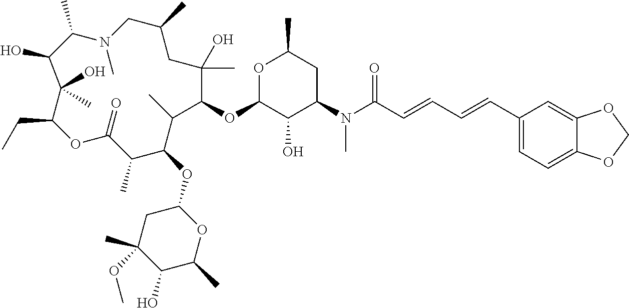 [SMILES: CC[C@@H]1OC(=O)[C@@H](C)[C@H](O[C@H]2C[C@@](C)(OC)[C@@H](O)[C@H](C)O2)C(C)[C@H](O[C@H]2O[C@@H](C)C[C@@H](N(C)C(=O)/C=C/C=C/c3ccc4c(c3)OCO4)[C@@H]2O)C(C)(O)C[C@H](C)CN(C)[C@@H](C)[C@H](O)[C@@]1(C)O] | 11.4 ± 0.4 | 19.6 ± 1.8 | 10.2 ± 1.9 | 12.9 | NT | NT |
| 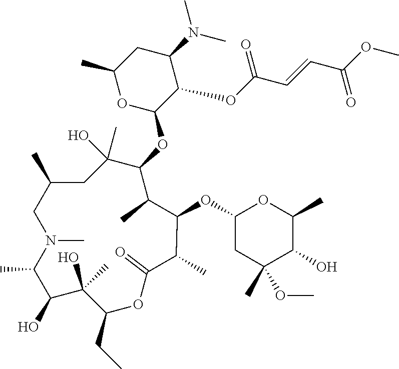 | 68.3 | 284.5 | 117.7 | 238.3 ± 29.1 | 117.9 | 278 |
| 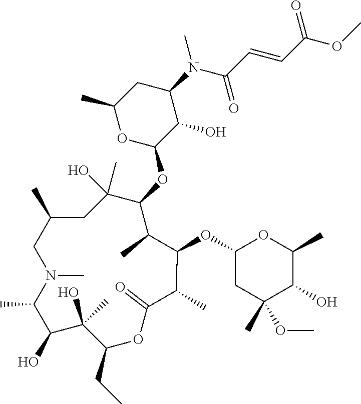 | NT | 214 | 126.5 | 211.5 | NT | NT |
| 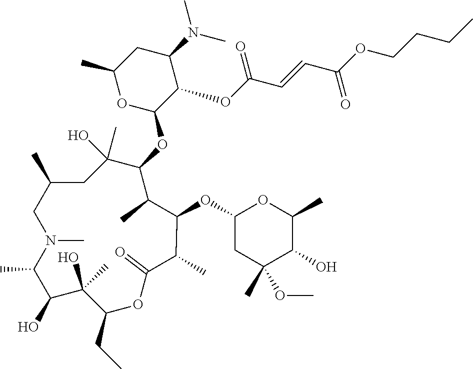 [SMILES: CCCCOC(=O)/C=C/C(=O)O[C@@H]1[C@@H](O[C@H]2[C@H](C)[C@@H](O[C@H]3C[C@@](C)(OC)[C@@H](O)[C@H](C)O3)[C@H](C)C(=O)O[C@@H](CC)[C@](C)(O)[C@@H](O)[C@H](C)N(C)C[C@@H](C)CC2(C)O)O[C@@H](C)C[C@H]1N(C)C] | 43.6 ± 8.5 | 140.9 ± 4.3 | 63.6 ± 7.7 | 126.2 ± 2.4 | 29.6 ± 3.0 | 50.0 ± 1.5 |
| 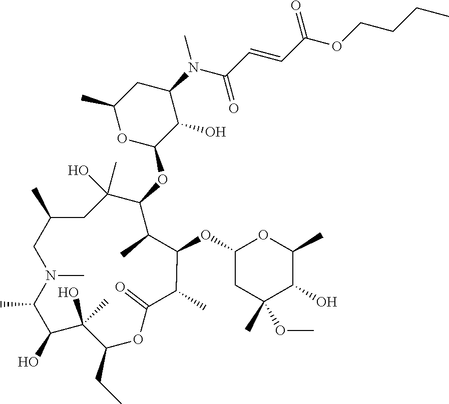 [SMILES: CCCCOC(=O)/C=C/C(=O)N(C)[C@@H]1C[C@H](C)O[C@H](O[C@H]2[C@H](C)[C@@H](O[C@H]3C[C@@](C)(OC)[C@@H](O)[C@H](C)O3)[C@H](C)C(=O)O[C@@H](CC)[C@](C)(O)[C@@H](O)[C@H](C)N(C)C[C@@H](C)CC2(C)O)[C@H]1O] | 78.9 ± 2.6 | 111.1 ± 4.6 | 64.9 ± 2.4 | 97.2 ± 15.5 | 34.8 ± 2.8 | 39.3 ± 7.1 |

Note
all IC₅₀ concentrations are in μM. NI = no inhibition up to 500 μM; NT = not tested.

Intracellular Target Validation: Anti-Fibrosis and Anti-Cancer Activities Data. The data presented revealed cellular targets whose expressions are perturbed by the claimed macrocycle-based compounds relative to standards and controls

Note: COL1A1=Collagen I or collagen; p-STAT3=phosphorylated STAT3; T-STAT3=total STAT3; α-SMA (or a-SMA)=alpha smooth muscle actin; GAPDH=glyceraldehyde 3-phosphate dehydrogenase (used to control for sample loading and commonly expressed or housekeeper protein whose expression isn't anticipated to be affected by the macrocycle-based compounds); p-P38=phosphorylated P38; T-P38=total P38; HO-1=Heme Oxygenase-1; Ela=pyruvate dehydrogenase E1 alpha; p-E1α=phosphorylated pyruvate dehydrogenase E1 alpha; pro-Caspase3=zymogen form of caspase 3; clv-Caspase3=activated form of caspase 3.

### Western Blot Procedure for MDA-MB-231 Cells:

The MDA-MB-231 cells (1*10⁶/well) were cultured in the 6-well plate and incubated for 48 h prior to the treatment. The cells were treated with DMSO (control), or 0.1% DMSO solution of selective compounds for 24 or 48 h. The cells were lysed by RIPA buffer (100 μLVWR, VWRVN653-100ML) and collected after washing twice with 2 mL/well of iced 1×PBS. The lysates were diluted to make equal protein concentration and 20-40 μg of each lysate was loaded to each well of TGX MIDI 4-20% gel (Biorad, cat. 5671093) and electrophoresis was ran at 150V for 65-70 min. The gel was electro-blotted on to the Turbo PDVF membrane (Bio-rad, 1704273). After blocking with 5% BSA for 1-2 h, the PDVF membrane was incubated with p-E1α (MA535866, Invitrogen), Ela (459400, Invitrogen) and GAPDH (MA116757, Invitrogen), HO-1 (70081S, Cell Signaling), p-STAT3 (#9145, Cell signaling), STAT3 (9139, Cell Signaling), Clv-caspase3, and pro-caspase3 antibodies. After incubation overnight, the membrane was washed with TB ST (3×5 min). Secondary antibody was added, and the membrane was incubated with agitation for 1 h. Bands were quantified using the Odyssey CLx Image system.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A compound of Formula I: 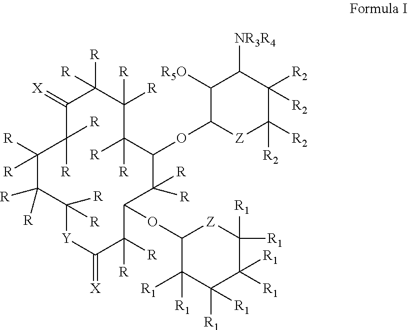
wherein
R is H, OH, CH₃, OCH₃, OCOCH₃, —CH₂OH, C₂H₅, C₁₋₁₀ alkyl, or C₁₋₁₀ alkoxy;
X is O;
R₁ and R₂ are each independently H, OH, OCOCH₃, CH₃, or OCH₃;
Y is O;
R₃ and R₄ are each independently H, CH₃, C₁₋₁₀ alkyl, C₁₋₁₀ alkoxy, aryl, heteroaryl, 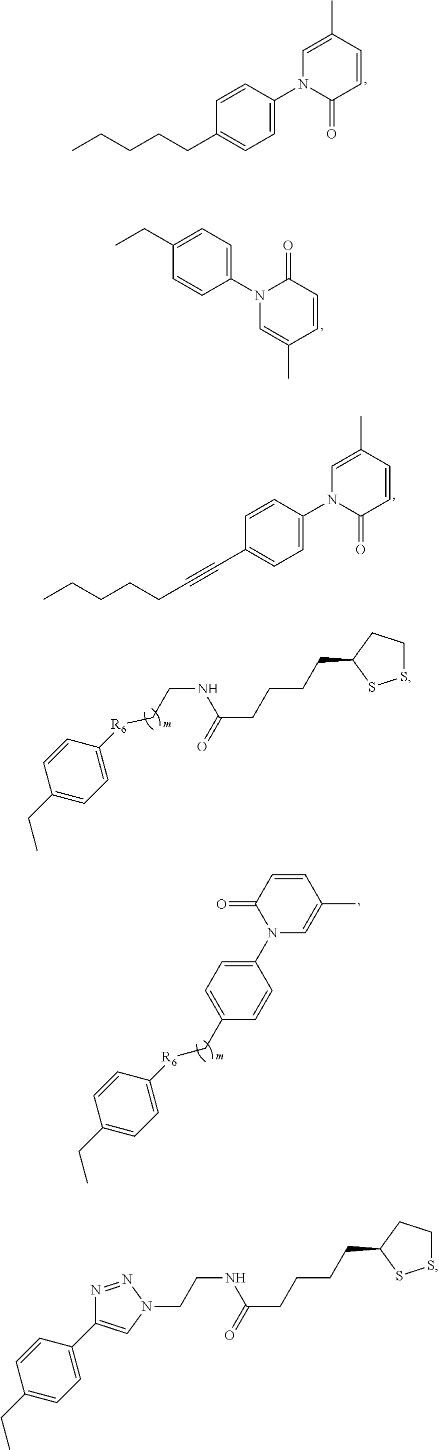 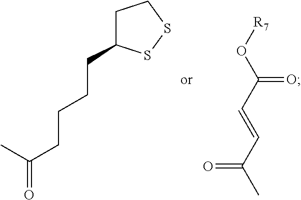
R₅ is H, C₁₋₁₀ alkanoates, C₁₋₁₀ arylalkanoates, C₁₋₁₀ heteroarylalkanoates, 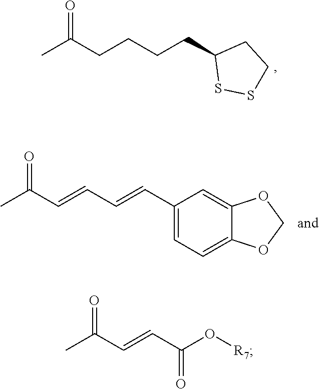
R₆ is amide, 5 or 6-membered heteroaryl ring;
R₇ is C₁₋₁₀ alkyl, C₁₋₁₀ arylalkyl, or C₁₋₁₀ heteroarylalkyl;
m is 1, 2, 3, 4, 5, or 6;
n is C₁₋₆ alkyl group, optionally substituted with double or triple bond; and
Z is O;
or diastereomers, solvate, or a pharmaceutically acceptable salt thereof.

2. A compound of Formula II: 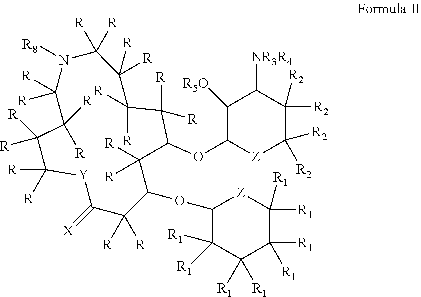
wherein
R is H, OH, CH₃, OCH₃, OCOCH₃, —CH₂OH, C₂H₅, C₁₋₁₀ alkyl, or C₁₋₁₀ alkoxy;
X is O;
R₁ and R₂ are each independently H, OH, OCOCH₃, CH₃, or OCH₃;
Y is O;
R₃ and R₄ are each independently H, C₁₋₁₀ alkyl, C₁₋₁₀ alkoxy, aryl, heteroaryl, 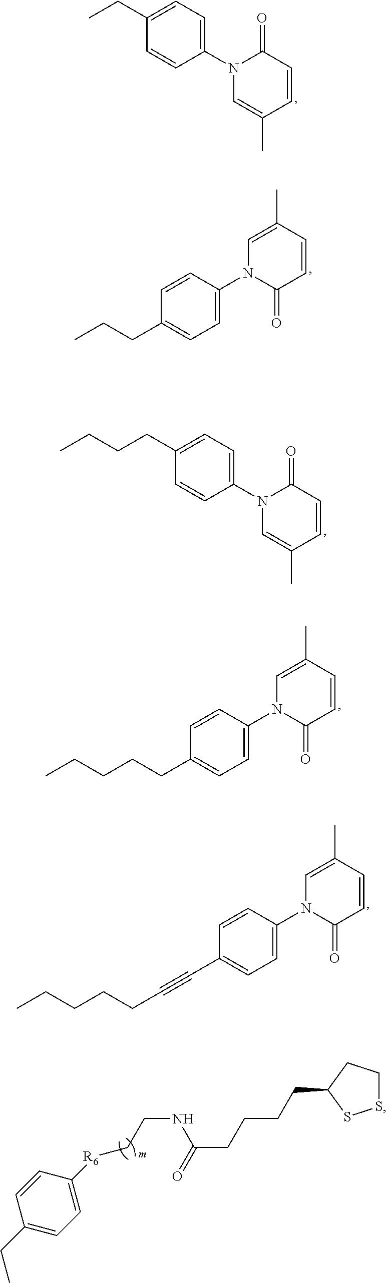 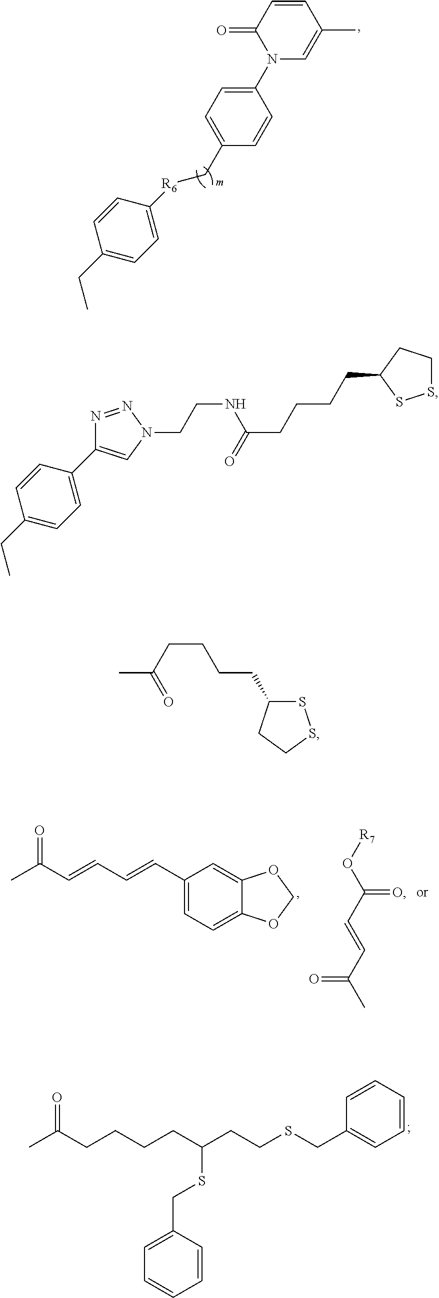
R₅ is H, C₁₋₁₀ alkanoates, C₁₋₁₀ arylalkanoates, C₁₋₁₀ heteroarylalkanoates, 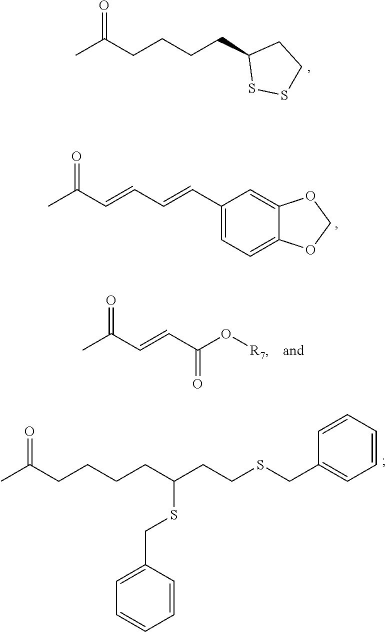
R₆ is amide, 5 or 6-membered heteroaryl ring;
R₇ is C₁₋₁₀ alkyl, C₁₋₁₀ arylalkyl, or C₁₋₁₀ heteroarylalkyl;
m is 1, 2, 3, 4, 5, or 6;
n is C₁₋₆ alkyl group, optionally substituted with double or triple bond; and
Z is O; and
R₈ is H or C₁₋₁₀ alkyl;
or diastereomers, solvate, or a pharmaceutically acceptable salt thereof.

3. The compound of claim 1, wherein one or more of:
(i) R is H, OH, CH₃, OCH₃, or C₂H₅;
(ii) R₁ is H, OH, CH₃, or OCH₃;
(iii) R₂ is H, OH, CH₃, or OCH₃;
(iv) R₃ is CH₃;
(V) R₄ is CH₃;
(vi) R₅ is H, C₁₋₁₀ alkanoates, C₁₋₁₀ arylalkanoates, or C₁₋₁₀ heteroarylalkanoates; and/or
(vii) R₇ is C₁₋₁₀ alkyl.

4.-14. (canceled)

15. The compound of claim 2, wherein one or more of:
(i) R is H, OH, CH₃, OCH₃, or C₂H₅;
(ii) R₁ is H, OH, CH₃, or OCH₃;
(iii) R₂ is H, OH, CH₃, or OCH₃;
(iv) R₃ is CH₃;
(V) R₄ is CH₃;
(vi) R₅ is H, C₁₋₁₀ alkanoates, C₁₋₁₀ arylalkanoates, or C₁₋₁₀ heteroarylalkanoates;
(vii) R₇ is C₁₋₁₀ alkyl; and/or
(viii) R₈ is CH₃.

16.-27. (canceled)

28. The compound of claim 1, wherein the compound is selected from the group consisting of: 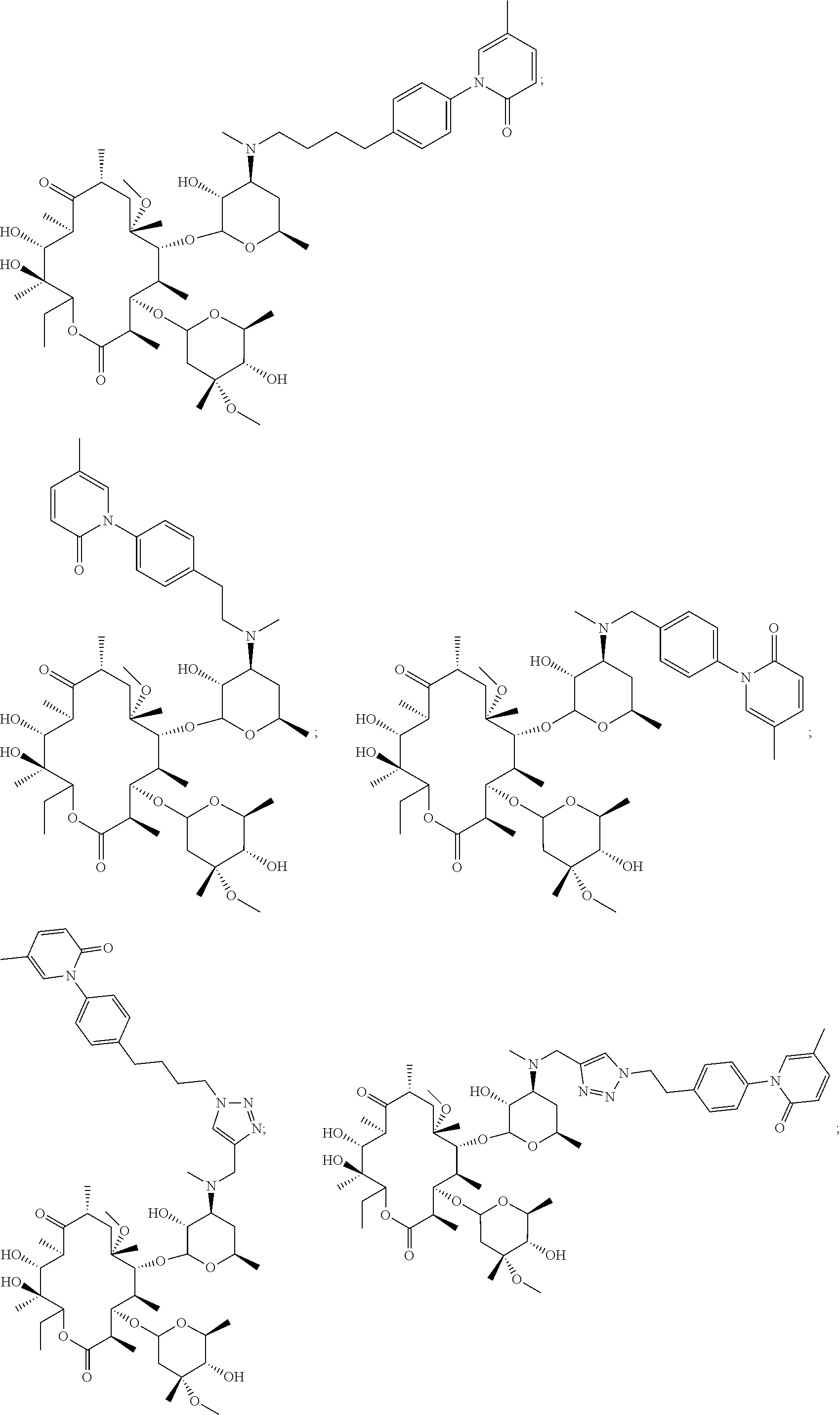 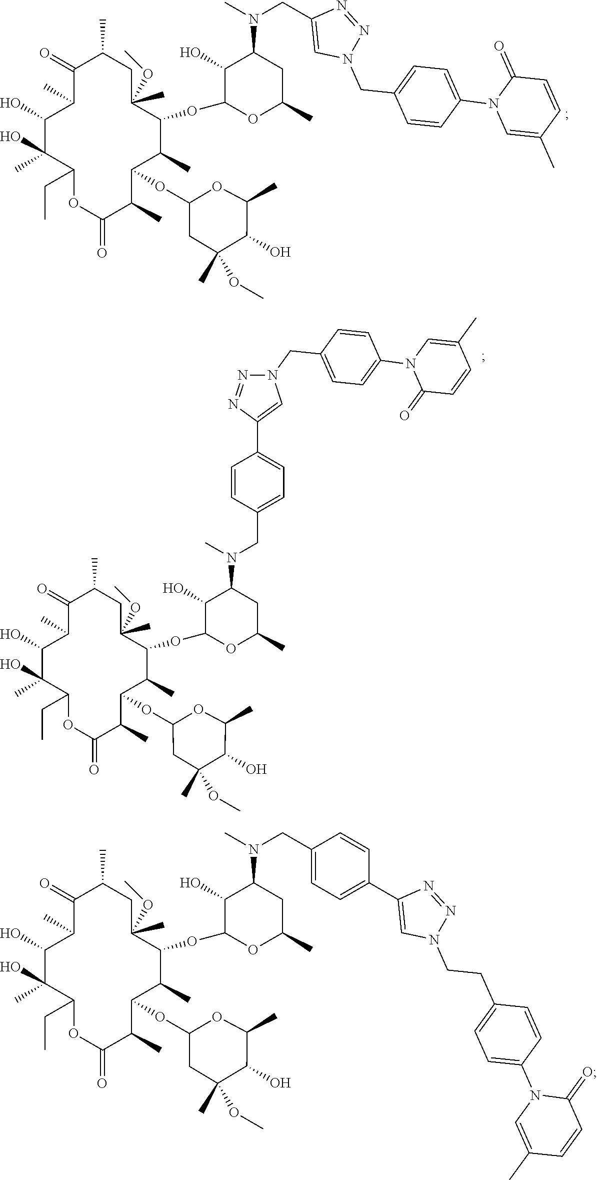 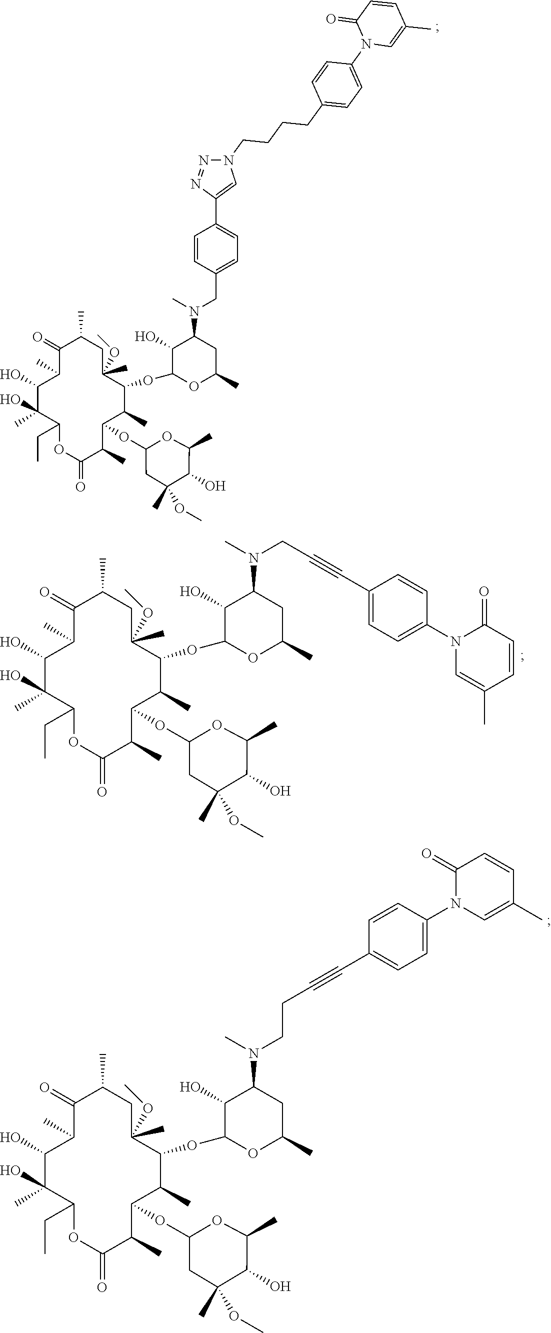 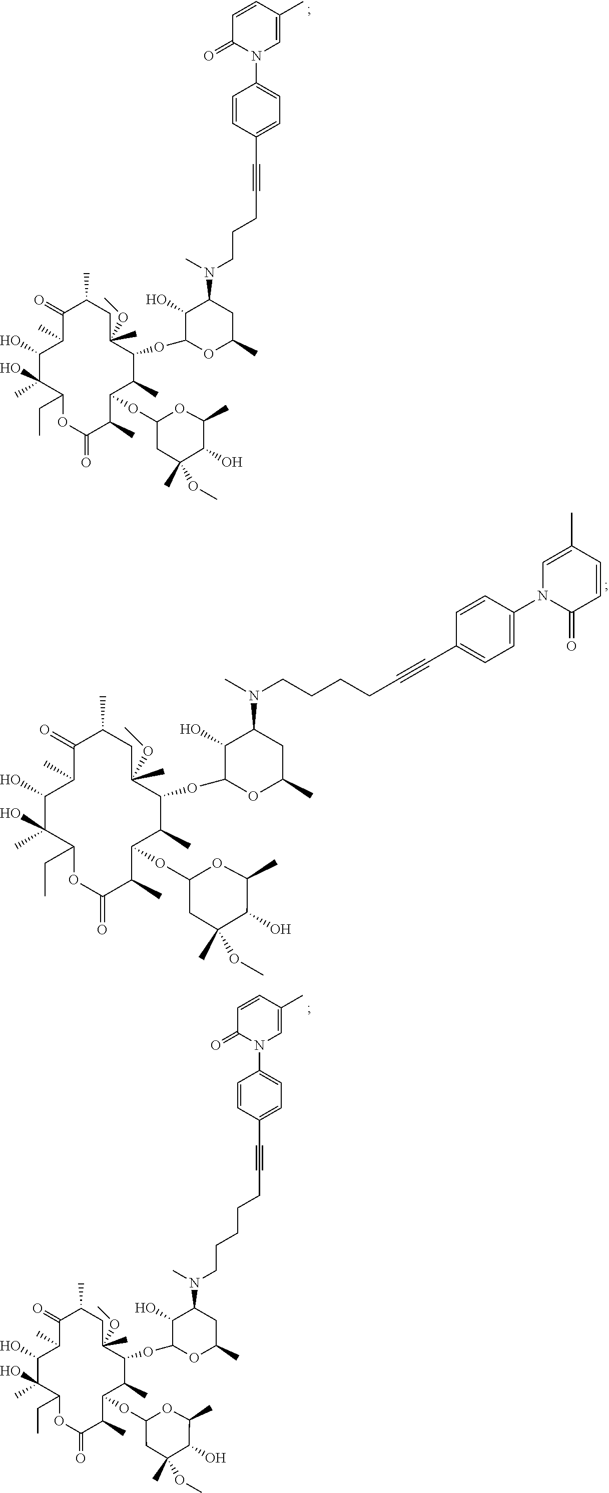 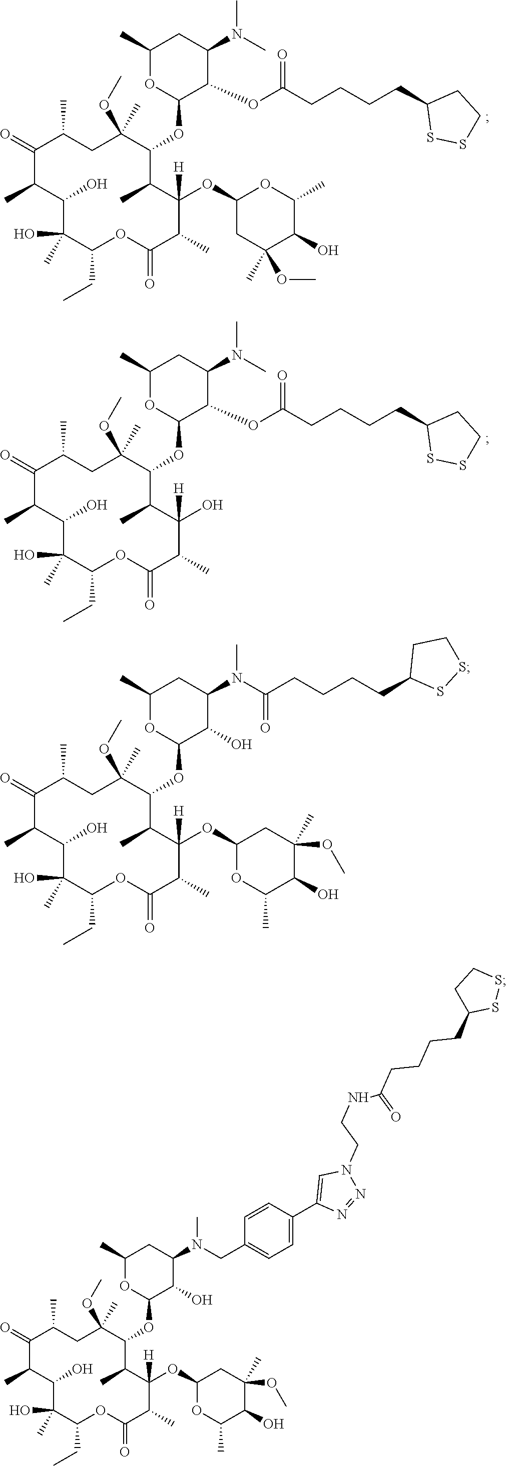 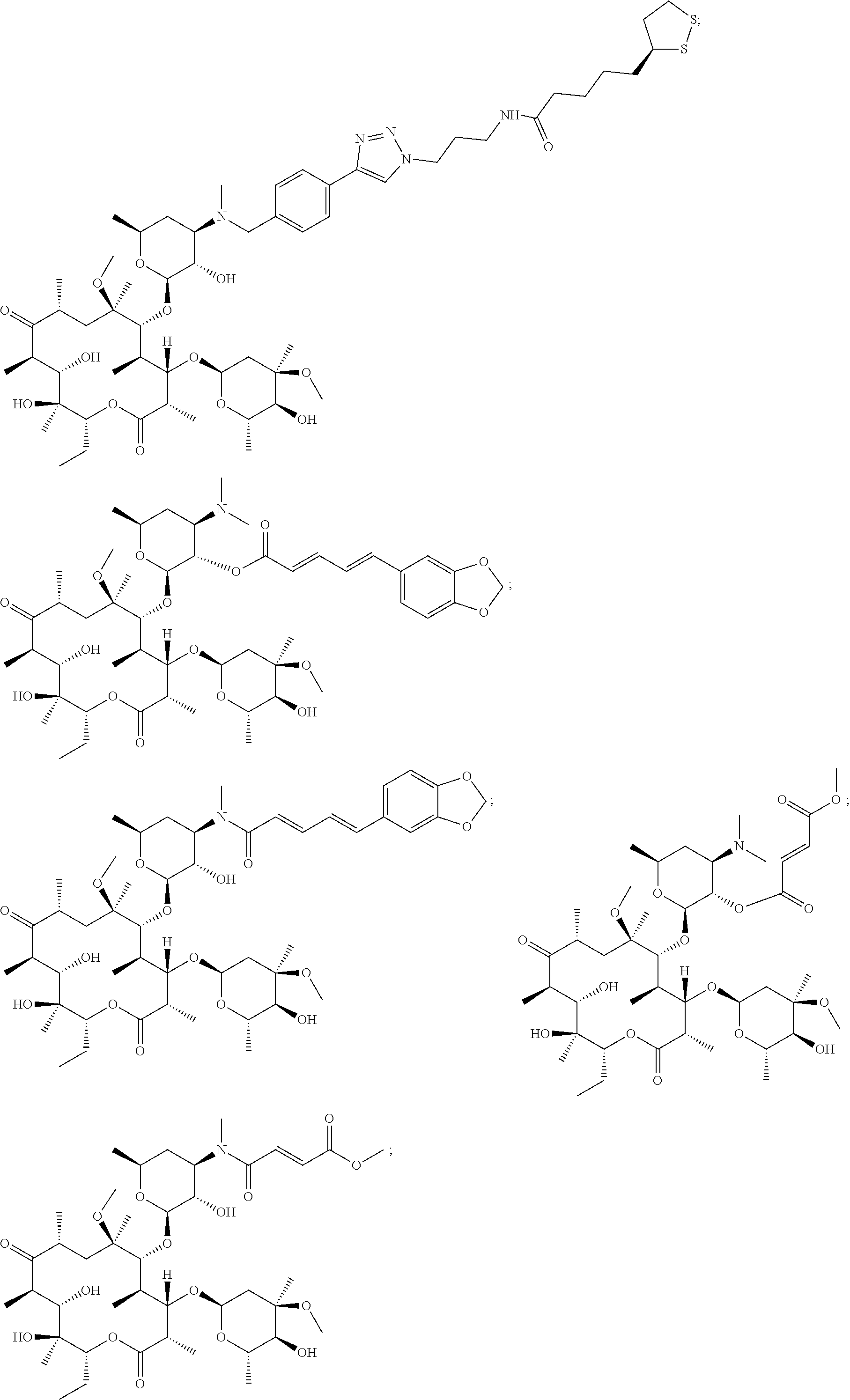 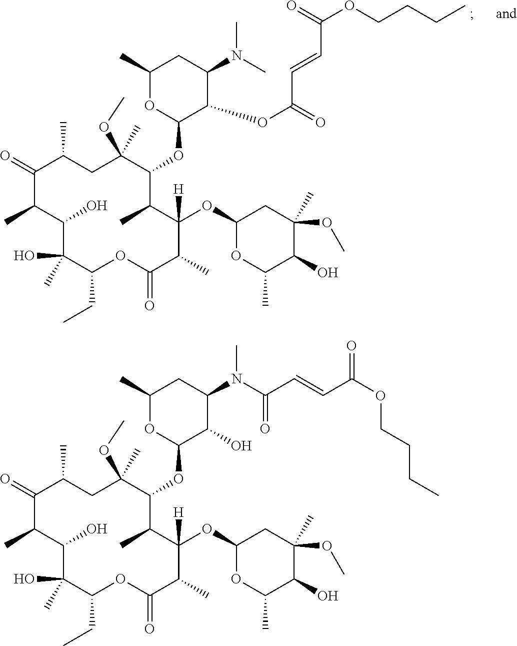
or diastereomers, solvate, or a pharmaceutically acceptable salt thereof.

29. The compound of claim 2, wherein the compound is selected from the group consisting of: 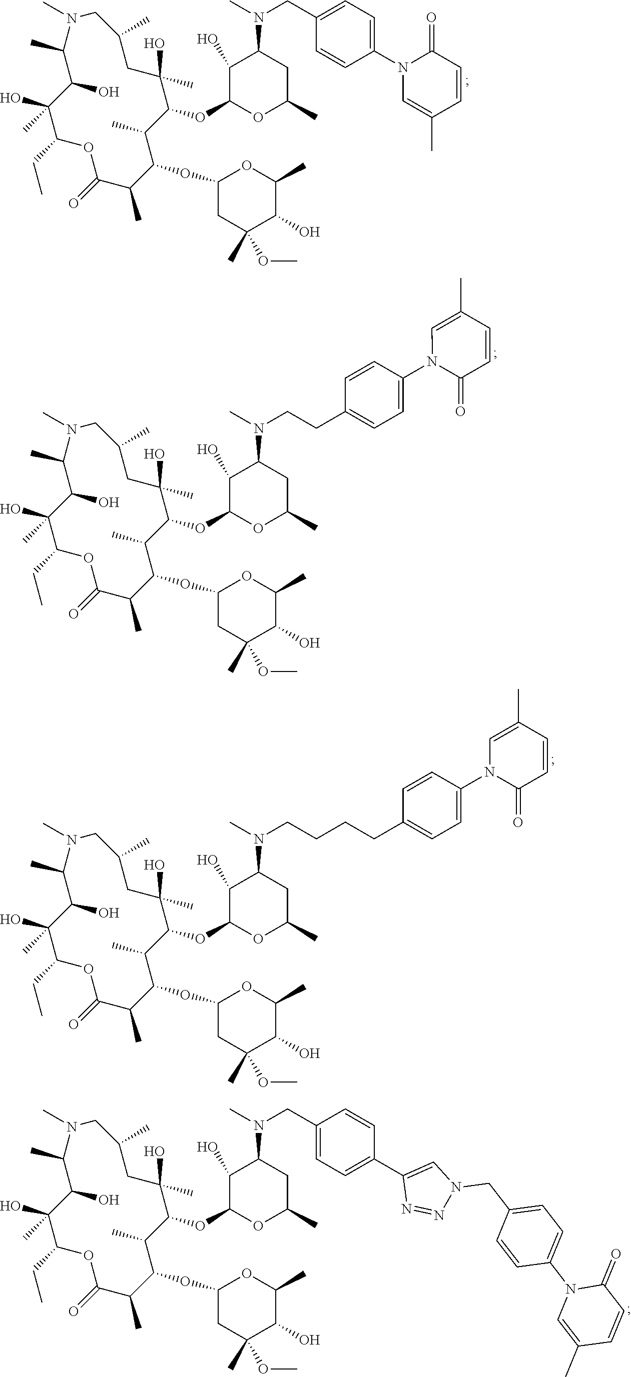 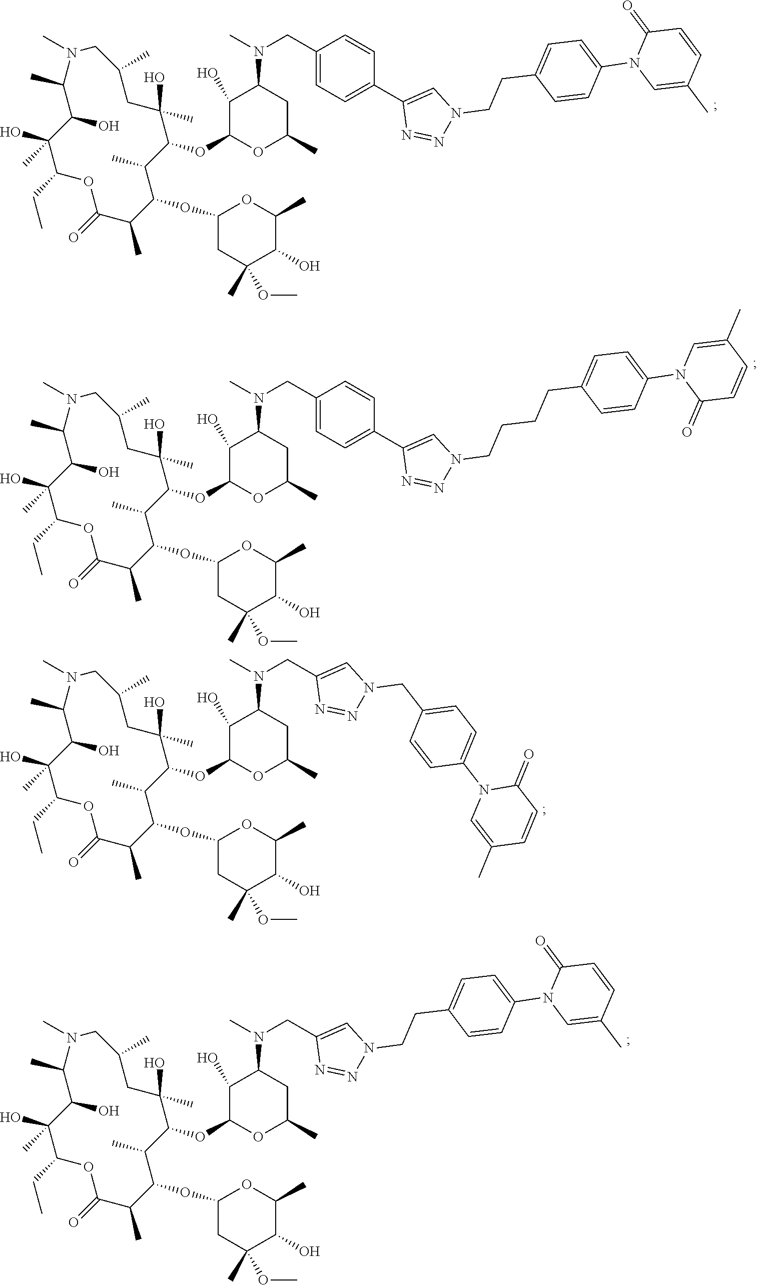 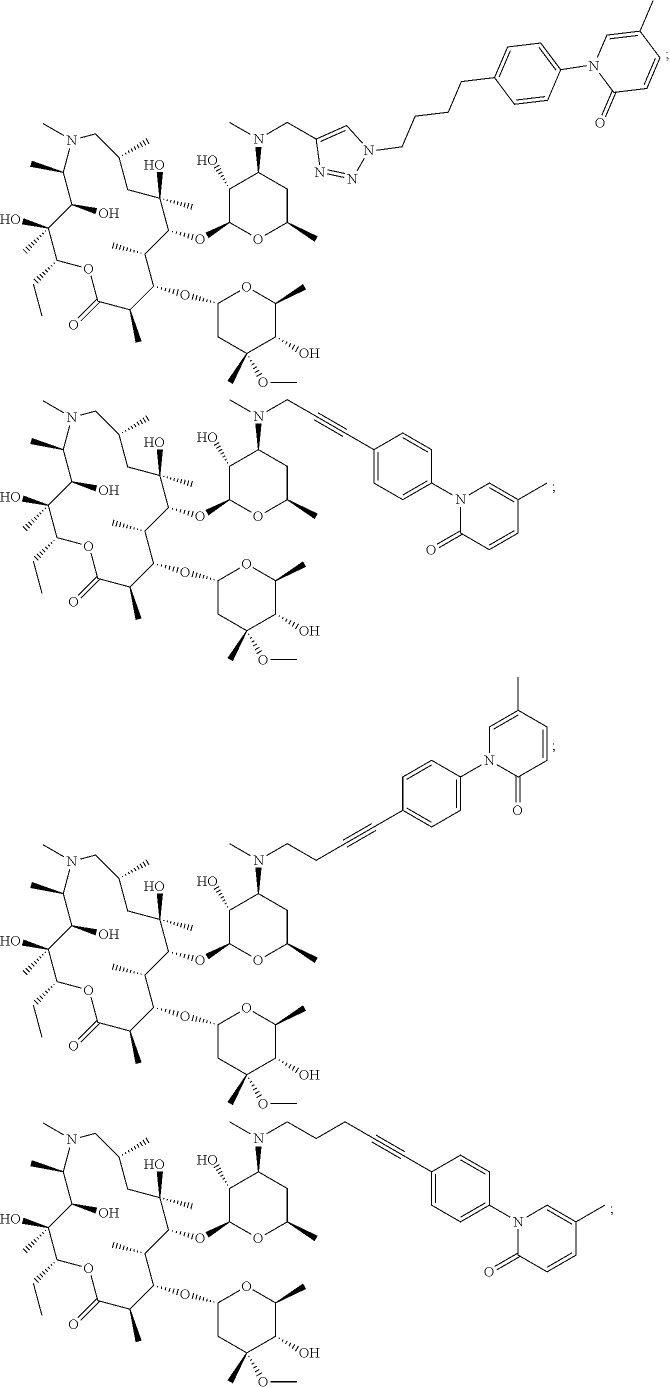 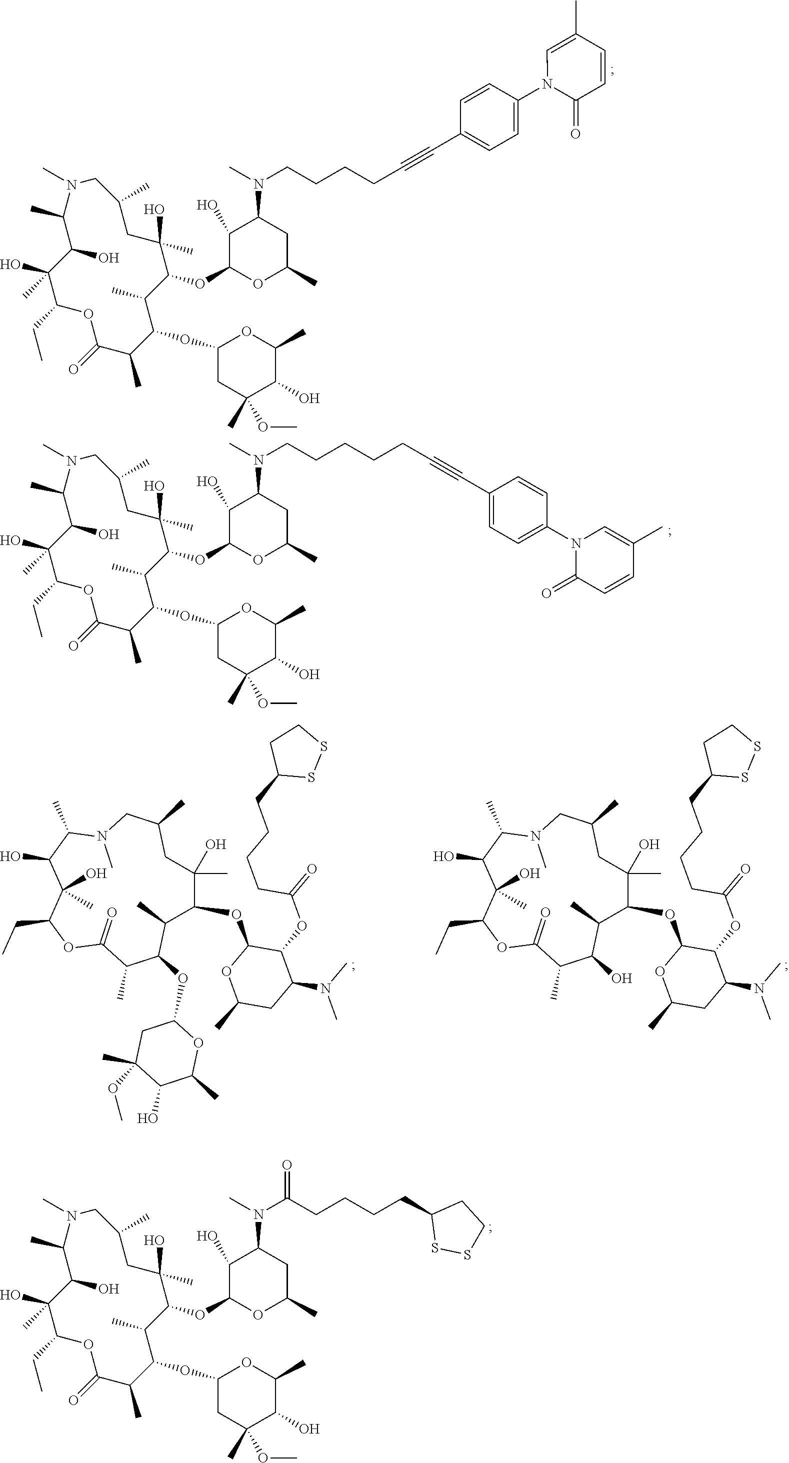 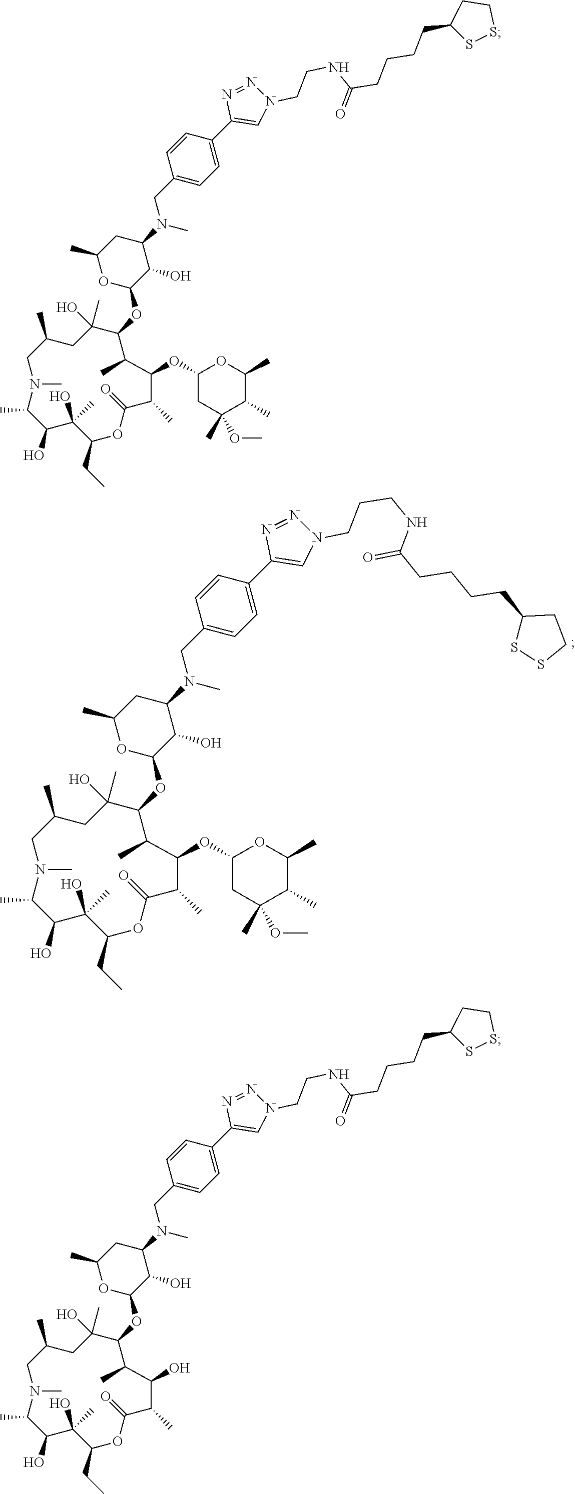 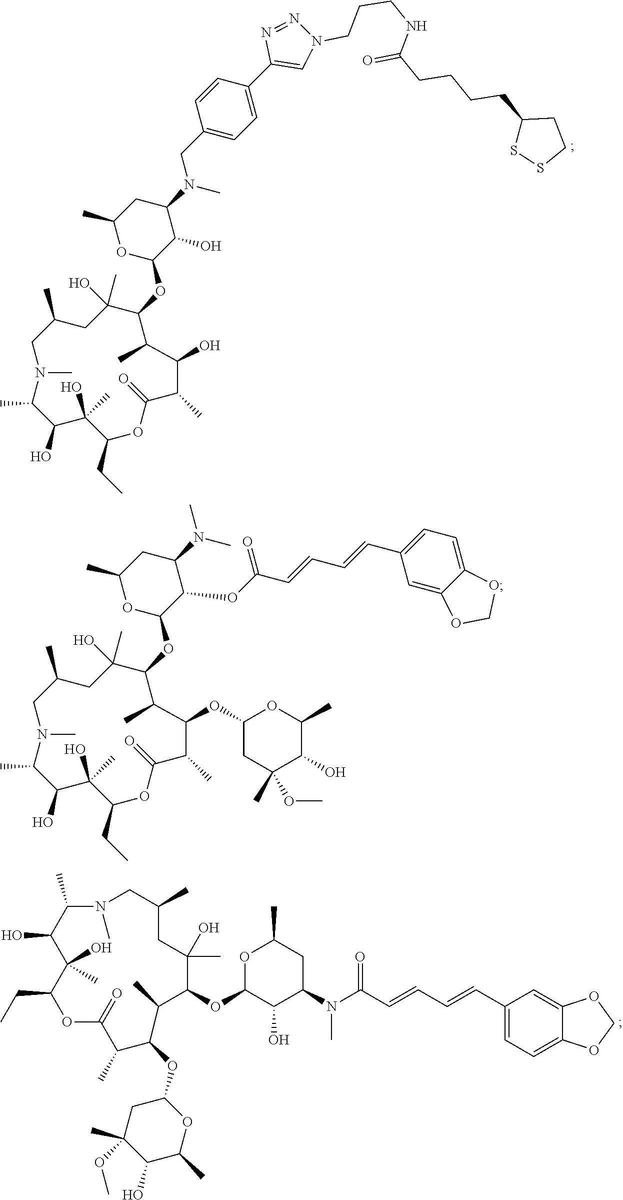 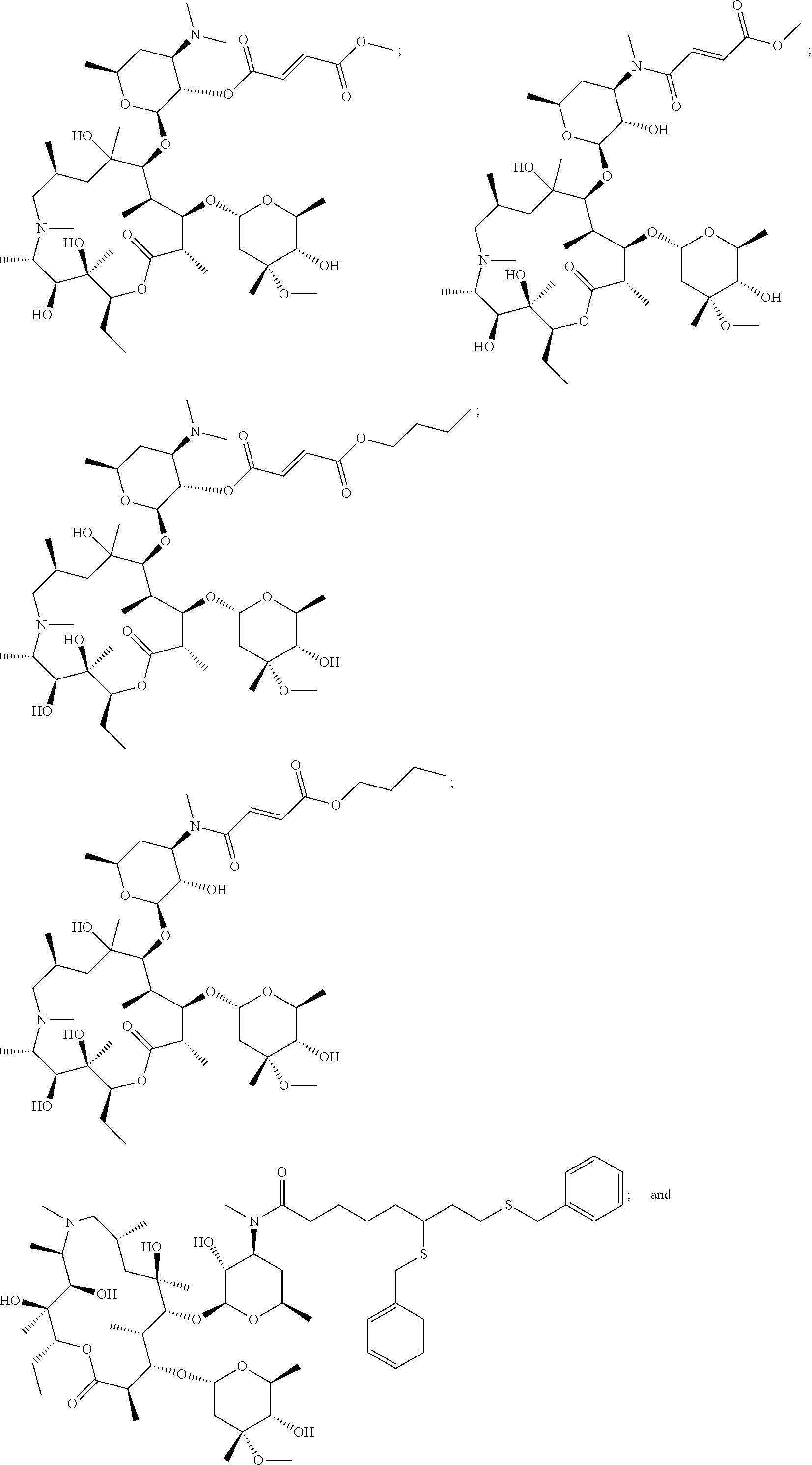 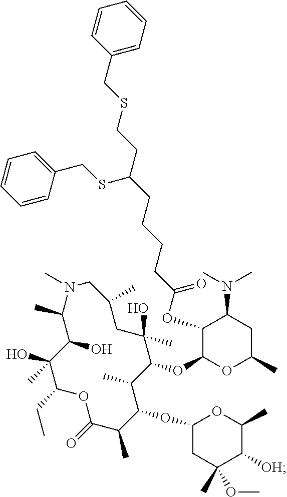 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=O)CCCCC(CCSCc2ccccc2)SCc2ccccc2)[C@](C)(O)C[C@@H](C)CN(C)[C@H](C)[C@@H](O)[C@]1(C)O]
or diastereomers, solvate, or a pharmaceutically acceptable salt thereof.

30. A pharmaceutical composition comprising:
the compound of claim 1 or a pharmaceutically acceptable salt thereof; and
a pharmaceutically acceptable carrier, adjuvant or vehicle.

31. A method of treating a fibrotic disease, disorder or condition in a subject in need thereof comprising:
administering to the subject a therapeutically effective amount of the compound of claim 1 or a pharmaceutical composition thereof.

32. The method of claim 31, wherein the fibrotic disease is selected from the group consisting of pulmonary fibrosis, liver fibrosis, skin fibrosis, renal fibrosis, pancreas fibrosis, systemic sclerosis, cardiac fibrosis and macular degeneration.

33. The method of claim 32, wherein the pulmonary fibrosis is idiopathic pulmonary fibrosis (IPF).

34. A method of treating an inflammatory disease, disorder or condition in a subject in need thereof comprising:
administering to the subject a therapeutically effective amount of the compound of claim 1 or a pharmaceutical composition thereof.

35. The method of claim 34, wherein the inflammatory disease is selected from the group consisting of acute and chronic inflammatory disorders, sepsis, acute endotoxemia, encephalitis, Chronic Obstructive Pulmonary Disease (COPD), asthma, liver fibrosis, pulmonary fibrosis, pulmonary hypertension, ulcerative colitis, Crohn's disease, and inflammatory bowel disease (IBD).

36. (canceled)

37. A method of treating a cancer in a subject in need thereof comprising:
administering to the subject a therapeutically effective amount of the compound of claim 1 or a pharmaceutical composition thereof.

38. The method of claim 37, wherein the cancer is selected from the group consisting of squamous cell carcinoma, small-cell lung cancer, non-small cell lung cancer (NSCLC), lung adenocarcinoma, squamous cell lung cancer, hepatocellular cancer, liver cancer, breast cancer, and hepatocellular carcinoma (HCC).

39. (canceled)

40. A pharmaceutical composition comprising:
the compound of claim 2 or a pharmaceutically acceptable salt thereof; and
a pharmaceutically acceptable carrier, adjuvant or vehicle.

41. A method of treating a fibrotic disease, disorder or condition in a subject in need thereof comprising:
administering to the subject a therapeutically effective amount of the compound of claim 2 or a pharmaceutical composition thereof.

42. The method of claim 41, wherein the fibrotic disease is selected from the group consisting of pulmonary fibrosis, liver fibrosis, skin fibrosis, renal fibrosis, pancreas fibrosis, systemic sclerosis, cardiac fibrosis and macular degeneration.

43. The method of claim 42, wherein the pulmonary fibrosis is idiopathic pulmonary fibrosis (IPF).

44. A method of treating an inflammatory disease, disorder or condition in a subject in need thereof comprising:
administering to the subject a therapeutically effective amount of the compound of claim 2or a pharmaceutical composition thereof.

45. The method of claim 44, wherein the inflammatory disease is selected from the group consisting of acute and chronic inflammatory disorders, sepsis, acute endotoxemia, encephalitis, Chronic Obstructive Pulmonary Disease (COPD), asthma, liver fibrosis, pulmonary fibrosis, pulmonary hypertension, ulcerative colitis, Crohn's disease, and inflammatory bowel disease (IBD).

46. A method of treating a cancer in a subject in need thereof comprising:
administering to the subject a therapeutically effective amount of the compound of claim 2 or a pharmaceutical composition thereof.

47. The method of claim 46, wherein the cancer is selected from the group consisting of squamous cell carcinoma, small-cell lung cancer, non-small cell lung cancer (NSCLC), lung adenocarcinoma, squamous cell lung cancer, hepatocellular cancer, liver cancer, breast cancer, and hepatocellular carcinoma (HCC).
